SEQUENCE LISTING (39 sequences)

SEQ ID NO: 1 - protein, 274 aa, Homo sapiens
MWGWKCLLFWAVLVTATLCTARPAPTLPEQAQQSTRADLGPGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - DNA, 822 bp, Homo sapiens
atgtggggctggaagtgcctcctcttctgggctgtgctggtcacagccactctctgcactgccaggccagccccaaccttgcccgaacaagctcagcagtcgacgcgcgcagatctgggcccgggcgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 3 - protein, 35 aa, Homo sapiens
GSPRFQDSSSSKAPPPSLPSPSRLPGPSDTPILPQ

SEQ ID NO: 4 - DNA, 108 bp, Homo sapiens
ggatcaccacgcttccaggactcctcttcctcaaaggcccctcctcctagccttccaagcccatcccgactcccggggccctcggacactccgatcctcccacaataa

SEQ ID NO: 5 - DNA, 20 bp, Artificial Sequence
  Description of Artificial Sequence Synthetic primer
cagaactctaggtcaggggc

SEQ ID NO: 6 - DNA, 20 bp, Artificial Sequence
  Description of Artificial Sequence Synthetic primer
cttatgcctcatccctccgc

SEQ ID NO: 7 - DNA, 20 bp, Artificial Sequence
  Description of Artificial Sequence Synthetic primer
cggtcaccctggagctatgc

SEQ ID NO: 8 - DNA, 20 bp, Artificial Sequence
  Description of Artificial Sequence Synthetic primer
cttatgcctcatccctccgc

SEQ ID NO: 9 - DNA, 20 bp, Artificial Sequence
  Description of Artificial Sequence Synthetic primer
gaggaaacgagctgcaggtg

SEQ ID NO: 10 - DNA, 20 bp, Artificial Sequence
  Description of Artificial Sequence Synthetic primer
ctcttgcaaaaaagccttcg

SEQ ID NO: 11 - DNA, 20 bp, Artificial Sequence
  Description of Artificial Sequence Synthetic primer
ttctttgcctcgttctctgc

SEQ ID NO: 12 - DNA, 15 bp, Artificial Sequence
  Description of Artificial Sequence Synthetic primer
gtctggcagtgtagc

SEQ ID NO: 13 - DNA, 20 bp, Artificial Sequence
  Description of Artificial Sequence Synthetic primer
ttctttgcctcgttctctgc

SEQ ID NO: 14 - DNA, 20 bp, Artificial Sequence
  Description of Artificial Sequence Synthetic primer
cgaggcccagagcaagagag

SEQ ID NO: 15 - DNA, 21 bp, Artificial Sequence
  Description of Artificial Sequence Synthetic primer
ctcgtagatgggcacagtgtg

SEQ ID NO: 16 - protein, 309 aa, Artificial Sequence
  Description of Artificial Sequence Synthetic polypeptide
MWGWKCLLFWAVLVTATLCTARPAPTLPEQAQQSTRADLGPGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSPRFQDSSSSKAPPPSLPSPSRLPGPSDTPILPQ

SEQ ID NO: 17 - DNA, 930 bp, Artificial Sequence
  Description of Artificial Sequence Synthetic polynucleotide
atgtggggctggaagtgcctcctcttctgggctgtgctggtcacagccactctctgcactgccaggccagccccaaccttgcccgaacaagctcagcagtcgacgcgcgcagatctgggcccgggcgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggatcaccacgcttccaggactcctcttcctcaaaggcccctcctcctagccttccaagcccatcccgactcccggggccctcggacactccgatcctcccacaataa

SEQ ID NO: 18 - protein, 564 aa, Homo sapiens
MATAERRALGIGFQWLSLATLVLICAGQGGRREDGGPACYGGFDLYFILDKSGSVLHHWNEIYYFVEQLAHKFISPQLRMSFIVFSTRGTTLMKLTEDREQIRQGLEELQKVLPGGDTYMHEGFERASEQIYYENRQGYRTASVIIALTDGELHEDLFFYSEREANRSRDLGAIVYCVGVKDFNETQLARIADSKDHVFPVNDGFQALQGIIHSILKKSCIEILAAEPSTICAGESFQVVVRGNGFRHARNVDRVLCSFKINDSVTLNEKPFSVEDTYLLCPAPILKEVGMKAALQVSMNDGLSFISSSVIITTTHCSDGSILAIALLILFLLLALALLWWFWPLCCTVIIKEVPPPPAEESEEEDDDGLPKKKWPTVDASYYGGRGVGGIKRMEVRWGEKGSTEEGAKLEKAKNARVKMPEQEYEFPEPRNLNNNMRRPSSPRKWYSPIKGKLDALWVLLRKGYDRVSVMRPQPGDTGRCINFTRVKNNQPAKYPLNNAYHTSSPPPAPIYTPPPPAPHCPPPPPSAPTPPIPSPPSTLPPPPQAPPPNRAPPPSRPPPRPSV

SEQ ID NO: 19 - DNA, 5909 bp, Homo sapiens
atcatatttaaaatctgggacaaagaaccgtcgggacggaactccttccattgcaaaagctcggcgcggcctcgggagctgcccggcggccccggaccgaggcagccctcccctttaaaagaagcggaggacaggattgggatccttgaaacccgaaacccagaaacagcatcggagcggaaaccagaggggaaaccttgaactcctccagacaattgcttccggggagttgcgagggagcgagggggaataaaggacccgcgaggaagggcccgcggatggcgcgtccctgagggtcgtggcgagttcgcggagcgtgggaaggagcggaccctgctctccccgggctgcgggccatggccacggcggagcggagagccctcggcatcggcttccagtggctctctttggccactctggtgctcatctgcgccgggcaagggggacgcagggaggatgggggtccagcctgctacggcggatttgacctgtacttcattttggacaaatcaggaagtgtgctgcaccactggaatgaaatctattactttgtggaacagttggctcacaaattcatcagcccacagttgagaatgtcctttattgttttctccacccgaggaacaaccttaatgaaactgacagaagacagagaacaaatccgtcaaggcctagaagaactccagaaagttctgccaggaggagacacttacatgcatgaaggatttgaaagggccagtgagcagatttattatgaaaacagacaagggtacaggacagccagcgtcatcattgctttgactgatggagaactccatgaagatctctttttctattcagagagggaggctaataggtctcgagatcttggtgcaattgtttactgtgttggtgtgaaagatttcaatgagacacagctggcccggattgcggacagtaaggatcatgtgtttcccgtgaatgacggctttcaggctctgcaaggcatcatccactcaattttgaagaagtcctgcatcgaaattctagcagctgaaccatccaccatatgtgcaggagagtcatttcaagttgtcgtgagaggaaacggcttccgacatgcccgcaacgtggacagggtcctctgcagcttcaagatcaatgactcggtcacactcaatgagaagcccttttctgtggaagatacttatttactgtgtccagcgcctatcttaaaagaagttggcatgaaagctgcactccaggtcagcatgaacgatggcctctcttttatctccagttctgtcatcatcaccaccacacactgttctgacggttccatcctggccatcgccctgctgatcctgttcctgctcctagccctggctctcctctggtggttctggcccctctgctgcactgtgattatcaaggaggtccctccaccccctgccgaggagagtgaggaagaagatgatgatggtctgcctaagaaaaagtggccaacggtagacgcctcttattatggtgggagaggcgttggaggcattaaaagaatggaggttcgttggggagaaaagggctccacagaagaaggtgctaagttggaaaaggcaaagaatgcaagagtcaagatgccggagcaggaatatgaattccctgagccgcgaaatctcaacaacaatatgcgtcggccttcttccccccggaagtggtactctccaatcaagggaaaactcgatgccttgtgggtcctactgaggaaaggatatgatcgtgtgtctgtgatgcgtccacagccaggagacacggggcgctgcatcaacttcaccagggtcaagaacaaccagccagccaagtacccactcaacaacgcctaccacacctcctcgccgcctcctgcccccatctacactcccccacctcctgcgccccactgccctcccccgccccccagcgcccctacccctcccatcccgtccccaccttccacccttccccctcctccccaggctccacctcccaacagggcacctcctccctcccgccctcctccaaggccttctgtctagagcccaaagttcctgctctgggctctctcagaaacttcaggagatgttagaacaagtctttccagttagagaagaggagtggtgataaagcccactgaccttcacacattctaaaaattggttggcaatgccagtataccaacaatcatgatcagctgaaagaaacagatattttaaattgccagaaaacaaatgatgaggcaactacagtcagatttatagccagccatctatcacctctagaaggttccagagacagtgaaactgcaagatgctctcaacaggattatgtctcatggagaccagtaagaaaatcatttatctgaaggtgaaatgcagagttggataagaaatacattgctgggtttctaaaatgctgccttcctgcctctactccacctccatccctggactttggacccttggcctaggagcctaaggaccttcacccctgtgcaccacccaagaaagaggaaaactttgcctacaactttggaaatgctggggtccctggtgtggtaagaaactcaacatcagacgggtatgcagaaggatgttcttctgggatttgcaggtacataaaaaatgtatggcatcttttccttgcaaattcttccagtttccaagtgagaaggggagcaggtgtttactgatggaaaaggtatgttgctatgttgatgtgtaagtgaaatcagttgtgtgcaatagacaggggcgtattcatgggagcatcagccagtttctaaaacccacaggccatcagcagctagaggtggctggctttggccagacatggaccctaaatcaacagacaatggcattgtcgaagagcaacctgttaatgaatcatgttaaaaatcaaggtttggcttcagtttaaatcacttgaggtatgaagtttatcctgttttccagagataaacataagttgatcttcccaaaataccatcattaggacctatcacacaatatcactagttttttttgtttgtttgttttttgttttttttcttggtaaagccatgcaccacagacttctgggcagagctgagagacaatggtcctgacataataaggatctttgattaacccccataaggcatgtgtgtgtatacaaatatacttctctttggcttttcgacatagaacctcagctgttaaccaaggggaaatacatcagatctgcaacacagaaatgctctgcctgaaatttccaccatgcctaggactcaccccatttatccaggtctttctggatctgtttaatcaataagccctataatcacttgctaaacactgggcttcatcacccagggataaaaacagagatcattgtcttggacctcctgcatcagcctattcaaaattatctctctctctagctttccacaaatcctaaaattcctgtcccaagccacccaaattctcagatcttttctggaacaaggcagaatataaaataaatatacatttagtggcttgggctatggtctccaaagatccttcaaaaatacatcaagccagcttcattcactcactttacttagaacagagatataagggcctgggatgcatttattttatcaataccaatttttgtggccatggcagacattgctaatcaatcacagcactatttcctattaagcccactgatttcttcacaatccttctcaaattacaattccaaagagccgccactcaacagtcagatgaacccaacagtcagatgagagaaatgaaccctacttgctatctctatcttagaaagcaaaaacaaacaggagtttccagggagaatgggaaagccagggggcataaaaggtacagtcaggggaaaatagatctaggcagagtgccttagtcagggaccacgggcgctgaatctgcagtgccaacaccaaactgacacatctccaggtgtacctccaaccctagccttctcccacagctgcctacaacagagtctcccagccttctcagagagctaaaaccagaaatttccagactcatgaaagcaaccccccagcctctccccaaccctgccgcattgtctaatttttagaacactaggcttcttctttcatgtagttcctcataagcaggggccagaatatctcagccacctgcagtgacattgctggacccctgaaaaccattccataggagaatgggttccccaggctcacagtgtagagacattgagcccatcacaactgttttgactgctggcagtctaaaacagtccacccaccccatggcactgccgcgtgattcccgcggccattcagaagttcaagccgagatgctgacgttgctgagcaacgagatggtgagcatcagtgcaaatgcaccattcagcacatcagtcatatgcccagtgcagttacaagatgttgtttcggcaaagcattttgatggaatagggaactgcaaatgtatgatgattttgaaaaggctcagcaggatttgttcttaaaccgactcagtgtgtcatccccggttatttagaattacagttaagaaggagaaacttctataagactgtatgaacaaggtgatatcttcatagtgggctattacaggcaggaaaatgttttaactggtttacaaaatccatcaatacttgtgtcattccctgtaaaaggcaggagacatgtgattatgatcaggaaactgcacaaaattattgttttcagcccccgtgttattgtccttttgaactgtttttttttttattaaagccaaatttgtgttgtatatattcgtattccatgtgttagatggaagcatttcctatccagtgtgaataaaaagaacagttgtagtaaattattataaagccgatgatatttcatggcaggttattctaccaagctgtgcttgttggtttttcccatgactgtattgcttttataaatgtacaaatagttactgaaatgacgagacccttgtttgcacagcattaataagaaccttgataagaaccatattctgttgacagccagctcacagtttcttgcctgaagcttggtgcaccctccagtgagacacaagatctctcttttaccaaagttgagaacagagctggtggattaattaatagtcttcgatatctggccatgggtaacctcattgtaactatcatcagaatgggcagagatgatcttgaagtgtcacatacactaaagtccaaacactatgtcagatgggggtaaaatccattaaagaacaggaaaaaataattataagatgataagcaaatgtttcagcccaatgtcaacccagttaaaaaaaaaattaatgctgtgtaaaatggttgaattagtttgcaaactatataaagacatatgcagtaaaaagtctgttaatgcacatcctgtgggaatggagtgttctaaccaattgccttttcttgttatctgagctctcctatattatcatactcagataaccaaattaaaagaattagaatatgatttttaatacacttaacattaaactcttctaactttcttctttctgtgataattcagaagatagttatggatcttcaatgcctctgagtcattgttataaaaaatcagttatcactataccatgctataggagactgggcaaaacctgtacaatgacaaccctggaagttgctttttttaaaaaaataataaatttcttaaatcaactcttttttctggttgtctgtttgttataaagtgcaacgtattcaagtcctcaatatcctgatcataataccatgctataggagactgggcaaaacctgtacaatgacaaccctggaagttgcttttttaaaaaaaataataaatttcttaaatcaaaaaaaaaaaaaaaaaa

SEQ ID NO: 20 - protein, 488 aa, Homo sapiens
MVAERSPARSPGSWLFPGLWLLVLSGPGGLLRAQEQPSCRRAFDLYFVLDKSGSVANNWIEIYNFVQQLAERFVSPEMRLSFIVFSSQATIILPLTGDRGKISKGLEDLKRVSPVGETYIHEGLKLANEQIQKAGGLKTSSIIIALTDGKLDGLVPSYAEKEAKISRSLGASVYCVGVLDFEQAQLERIADSKEQVFPVKGGFQALKGIINSILAQSCTEILELQPSSVCVGEEFQIVLSGRGFMLGSRNGSVLCTYTVNETYTTSVKPVSVQLNSMLCPAPILNKAGETLDVSVSFNGGKSVISGSLIVTATECSNGIAAIIVILVLLLLLGIGLMWWFWPLCCKVVIKDPPPPPAPAPKEEEEEPLPTKKWPTVDASYYGGRGVGGIKRMEVRWGDKGSTEEGARLEKAKNAVVKIPEETEEPIRPRPPRPKPTHQPPQTKWYTPIKGRLDALWALLRRQYDRVSLMRPQEGDEGRCINFSRVPSQ

SEQ ID NO: 21 - DNA, 8058 bp, Homo sapiens
agtcatcttcaactcggcaggaacccacaagtgtgcatgtgtggctcggaggcttcagctggggccccgccctcgtccccaggcgcacactgacacacgcagcccagacccggcccgagcgggctcctgccctcggcgtggcttctctccagccgggagtcccagggccagctagcctcctcccctaaaggggacggcctgtcagcgcagtgccagagtccagcaccgggaggaaagtttcggagtgcggagggagttggggccgccggaggagaagagtctccactcctagtttgttctgccgtcgccgcgtcccagggaccccttgtcccgaagcgcacggcagcgggggggacttcagccctccaggcggggtgggttccaggtccgggtccgaggcgggcgctggaggctcggccccaggccggagaggaactcctttcgcgagctgtcgccgtgggcccgcattgtctgcaggaactctccggaatcgggagggggaggactggatcgcgcttccactgggattcgtcaagagttccggcggcagctgcggcggtggcggagactccctttgtcctctcaggacctccctctctccctccctgtcagctggtgggtcccgctgccgcaggcgccggcgtctcagctgctcgccgccccccaccccagagtgcgtgccgggtgactcccgccacctttgcgaccctcctgagcttaggggactgcgagcgggagggagtctcaggcccccggccgcaggatggtggcggagcggtccccggcccgcagccccgggagctggctgttccccgggctgtggctgttggtgctcagcggtcccggggggctgctgcgcgcccaggagcagccctcctgcagaagagcctttgatctctacttcgtcctggacaagtctgggagtgtggcaaataactggattgaaatttataatttcgtacagcaacttgcggagagatttgtgagccctgaaatgagattatctttcattgtgttttcttctcaagcaactattattttgccattaactggagacagaggcaaaatcagtaaaggcttggaggatttaaaacgtgttagtccagtaggagagacatatatccatgaaggactaaagctagcgaatgaacaaattcagaaagcaggaggcttgaaaacctccagtatcataattgctctgacagatggcaagttggacggtctggtgccatcatatgcagagaaagaggcaaagatatccaggtcacttggggctagtgtttattgtgttggtgtccttgattttgaacaagcacagcttgaaagaattgctgattccaaggagcaagttttccctgtcaaaggtggatttcaggctcttaaaggaataattaattctatactagctcagtcatgtactgaaatcctagaattgcagccctcaagtgtctgtgtgggggaggaatttcagattgtcttaagtggaagaggattcatgctgggcagtcggaatggcagtgttctctgcacttacactgtaaatgaaacatatacaacgagtgtaaaaccagtaagtgtacagcttaattctatgctttgtcctgcacctatcctgaataaagctggagaaactcttgatgtttcagtgagctttaatggaggaaaatctgtcatttcaggatcattaattgtcacagccacagaatgttctaacgggatcgcagccatcattgttattttggtgttactgctactcctggggatcggtttgatgtggtggttttggcccctttgctgcaaagtggttattaaggatcctccaccaccacccgcccctgcaccaaaagaggaggaagaagaacctttgcctactaaaaagtggccaactgtggatgcttcctattatggtggtcgaggggttggaggaattaaaagaatggaggttcgttggggtgataaaggatctactgaggaaggtgcaaggctagagaaagccaaaaatgctgtggtgaagattcctgaagaaacagaggaacccatcaggcctagaccacctcgacccaaacccacacaccagcctcctcagacaaaatggtacaccccaattaagggtcgtcttgatgctctctgggctttgttgaggcggcagtatgaccgggtttctttgatgcgacctcaggaaggagatgagggccggtgcataaacttctcccgagttccatctcagtaaaagggaagcaggaagaccaagaaggtacgaagatggcacattttcacatagctgattttcaaccaaatgaaaaaaatcaagtgcatttcagaagcttttggaagagcagcttaattcctctcagtcgggaaatgttttctctgccttctgctttgcttgcaccaaacatttctaaacacttgttctgccatctacatgggaggtgatgaaactcagtggtaactcatgatttatgacattgaaaataaagaggaacattgacctgcagactatggtttgtacaagaaagtttgtttgaatgtgtagaagaggaaaaagcaacaacagcaacaacacgaagatgataccaaaacaaggaccacaaaacaactagccatgatgggagacaggagttttttacatggaaacatggcacttgtgtttttatgtggcaagatctttatccataggcagagtatgaaatttcccaccaggctaagcaaataaagaagtccattgccttatagctatgtcagatcacagaatccttccaagtgctctatcacagtgtgccttatgggaagtttctgactggaaaatcttgtcattctaacactgaaaagtgcacacgcatgacaaaatgtagacaagatgcctcaaggtattggtagcaagcaagattttgccctttagttttcgaagacacctttctttcattatgcactcgggacaagaaaattaatagagcgttattccacagaaggcctctagccagagatcttgagtgtagtgcaagggactcattttttgcgaacttgtccctgtgactagtagattcccccttttcctgtgtttaggatttagtagtgcataaagcattaatatccataaacatacctagaagtttgttttgcttttaatttaaaggaagcagtaaccacaaagcttccgctcagggttttttctttcttcaagtctccaagggctcttcagcgtcacaagccagcaactctctttgcattaaaatttcaaagtttaattaatataattaaaagcaacagcaagcagcagcctgtgaagattttgctcatcttttttatgccttttgacattgaatgacctattactgtatgcgcattacttggattttgaggggcactctaccttggttatgattcagtagaggaaaaagaccacctttcttcaatttacaaattaaatcttctggagggtcgctatcacaaaacattgacgatgtatgtattataattttttagaaaaaccaccatcgtgtcacgtcgacgatgccaaattatgttagcgtgagcagaaacaccgtgggggaggaaggcagcagctgaagaaaaaagctcaaatgatctagtcactttcgatactgtacttcagatgcgaaatggatattcgagtggaaacctgacaaagtgcgcctgctttgatgtgaactggtatagacaatgaccagtggctgggtcagtgggatgtctctctgtgagcacaaaggcttatcaaatgacactaaaaataagttcaacaaccatcacattggaagggagaaggcgaacatttcatgtttggcgggcatgtgagtgcacaagatggaaagagcgattggagcatcctggtataattacccccattgtgctcttaatggaaatttcaaaggacgggagtattctgttggttggtgtccaggtttgtggcactgttccaagaggccttacacacacacacaaatatataattttctatacatatatatcctctagcttgaaacttttgctcaagtttatttatgtcactggctggctggatccaaagtcatgtgtccacacattcataaataaaaattttacctatgcctgtgctaccatttctttaagcccacataaagctaatgccttcttgggttgaatatgcagttgttatgcttagtttcagggatggaattctattcagttccaaaatgtccctcatgttcacagatgatactattggcagtagctgcatatgcaagtgtaacttggaagcctgatatagtatacccaaccctttctgcaatgagttattgtatccctttgttctgcatttgcatctggaacaagacctaatgtggcttccttaaatgggcacttcatttctgatgagtgcacaccaattattgctattttaatatcattgagaatactacagaagctaaggctatgtttctttggtaactacttcaaatttctctagaattatgtagcatacttacattttgctgcatgaacaatgaaaattaacctctgcctttaaatggaaaaataactttctgtagattgttatacagctactatgaagagctcttaatgaagaggaagtggcagtggtctatatgtagactgaaaaaatatcattaataaagttcaaagactgatggaatttcattgcccttaaagatatgaaccagatctgttttatgcttttctccaattatactaactagcagttgaaaagaatttgttttgaagagtgttattttcttaccaaaagaccagatcaaaaatccctatacttttccaccaaaaaaagattaaaatgaagccatttctataaatttaatggcactttatattttatatagagaaatagcttctgtttgattttctggacaccatgtcagtcaagatctactttctgtgtaaaaatgaataagtcttgcacataatccattcgtgttgcataaggttgcaaaaacaattaagtgggaaatgactcctaacttatgtgtctactctgcctagaaagaaagttttgaaggcatctatatatatttcataacaatgtctgttctttatggatttgatccataaatatttatctatctgaaaactttgatacttggaatgttttccttagagcatttggtgctccaagaaaaaacatttggtgctaagagttgcagcactgatgcctttccctaagttaattctattttaaaaatttatatttccttcccaatgagatgcagagtaaaatttgagaccctccattgactagggagcagcatctgtcatctatggttaattactattttgacaaaaatatagaaggtttaaacccccatgtatttcagattgttagagaaagaagtagtttgcttatatttcccttgaactttattctaaccatttaaataataaattattcaatatttgttataataaatccttactagatacaagatttgggtgtttctgtgaagttaactttattatctaaatatcactaaccattgtttactgctcagaggcagatacatttgtttagtttaaagttgaaacaaattctatattgtggataattaggtgtttaaaacattttgatagaccagcttatatatgttatttttgaaatttaagaaggcagaatgtaattaggtcacatttacaagtagaaggaattataacttcctactccatgcatatattgtatattggttagcttttactagcattttctagcatcacattactttagaaagtttttgagagtttcattactactggaaaagtttggtgggtatatgtacacaggaaagaaatatatgcatcatttcaatatgaaaggaaatacataatctagaaacatagaataactcttggtacatctgttacatagttaaggaaagaaacaggcaggtgaggctggagaatgttgtctatttgtaggaaataaagtcagtgcagacgcatattctagtttagagttagattaatattgtgcttagtgaatttctatgacatatttctgagaacactttaatatatttccacacctactggaatctaattcagtcgatcggataaaagaaagacttttttgttgttgtttttgaaaatccataatgtagcaggttctgcgctggttttttaatgtattttatttttgtgctctcctgccaaaagaaaaggctgataagtaagtacacagtacaacatgtaaatttgtataaaaatatgcttgtttagattgctctacaataactatattaaggtaaattgtgtgtgtgtgtgtgagagagagagagagagagagaaacagagggagaattgactcaatgtgttttctatgaagagtcttagcttaggaatcaggtaacagagaggtcctagaccatagacccagccatcaactagcttgctgatgttgaacagatcaactctcaaccatagactgaagatggaatgaagatatcaaaagttccttttagcactgactagattttttttcttttttttgaaacagagtctcactctgtcacccaggctggagttcaatggcatggtctgggctgactgtaacctcctcttcctgggttcaagtgattctcatccctcagcctcctgagtagctgggattacaggcacataacaccacacccagctaactcttgtatttttgtagaaatggggtttcgctatgttgctcagactggtctcaaacacctggcctcaagtgatcctcctgcctcagtctcccaaatgctgtgattacaggcacaagctactgcaccaggcctctgactacatttctattaatatggttaggttggaggttttagtatttttgtatctcatatttgtatcaatatgactggcttctttgtctgtagtgtgtggtaatattagttctgtaaactgtcagttgcaaaaaaaaaaaataccttgaactatagtatatgttgataattagccataataatttcttagttaatttcttataattaaatttgtcaaagaggaaacttacagtttatatctgatgaaatctctaaaaagatgggtaaaacattgggaaatgtatgcatgtacttcactctggtttcatagggttagcaagtgtcttaaaaacatatataaagaagcacagagattgttaggagatatttatgctcccagttttaataattgggatactttgtataccacagaaagaaaaattactaaactcctctttttttagtcaaaattggaaaaaaagtcttaattgacagttactatgcctgtgctacccatagcaagtattcagtggaaaatactttactaagtaagtaatttgaacacagcttaaaatccatagtatgttacaattgctagcctttcacaaagtttgcattgtcttaatgtagaaggatactgtgatctaagaattcacaattttaaaaagtggaacctaaatagggtttcctaattgccatgaagttatttgtatcttagatgaattatatttacaacattgtaaatgtcagtgggtgatccaaaataaattgttaaagttattaaaatgtacatttaagtaggtttcagtttgactagaaataattggcaagaaggcaagaactagtcttctagagcagggatcccatcccccaggtcatggactggtactggtccatggcctgttagaaaccaggccacacagcaggagatgagtggaaagcaagtgaaacttcatgggtatttacagcaattccccgtcgctcgcattaccacctgagctgtgtctcctgtgagatcagcagcagcattagattctcaaggagcacaaacccttttggaactgtgtgtgagggatctaagttgcgcatttcttatgagaatctaatacctgatgatctgttgttgtctcccaccacccccagatgggaccatctagttgcaggaaaacaagctcaggctcccactgattctacattatagtgagttgtgtaattatttcattatatataacaatgtaataataatagaaataaagtacataataaatgtaaaaaaaaaaaaaaaaaa

SEQ ID NO: 22 - protein, 368 aa, Homo sapiens
MATAERRALGIGFQWLSLATLVLICAGQGGRREDGGPACYGGFDLYFILDKSGSVLHHWNEIYYFVEQLAHKFISPQLRMSFIVFSTRGTTLMKLTEDREQIRQGLEELQKVLPGGDTYMHEGFERASEQIYYENRQGYRTASVIIALTDGELHEDLFFYSEREANRSRDLGAIVYCVGVKDFNETQLARIADSKDHVFPVNDGFQALQGIIHSILKKSCIEILAAEPSTICAGESFQVVVRGNGFRHARNVDRVLCSFKINDSVTLNEKPFSVEDTYLLCPAPILKEVGMKAALQVSMNDGLSFISSSVIITTTHCSDGSILAIALLILFLLLALALLWWFWPLCCTVIIKEVPPPPAEESEENKIK

SEQ ID NO: 23 - DNA, 1667 bp, Homo sapiens
atcatatttaaaatctgggacaaagaaccgtcgggacggaactccttccattgcaaaagctcggcgcggcctcgggagctgcccggcggccccggaccgaggcagccctcccctttaaaagaagcggaggacaggattgggatccttgaaacccgaaacccagaaacagcatcggagcggaaaccagaggggaaaccttgaactcctccagacaattgcttccggggagttgcgagggagcgagggggaataaaggacccgcgaggaagggcccgcggatggcgcgtccctgagggtcgtggcgagttcgcggagcgtgggaaggagcggaccctgctctccccgggctgcgggccatggccacggcggagcggagagccctcggcatcggcttccagtggctctctttggccactctggtgctcatctgcgccgggcaagggggacgcagggaggatgggggtccagcctgctacggcggatttgacctgtacttcattttggacaaatcaggaagtgtgctgcaccactggaatgaaatctattactttgtggaacagttggctcacaaattcatcagcccacagttgagaatgtcctttattgttttctccacccgaggaacaaccttaatgaaactgacagaagacagagaacaaatccgtcaaggcctagaagaactccagaaagttctgccaggaggagacacttacatgcatgaaggatttgaaagggccagtgagcagatttattatgaaaacagacaagggtacaggacagccagcgtcatcattgctttgactgatggagaactccatgaagatctctttttctattcagagagggaggctaataggtctcgagatcttggtgcaattgtttactgtgttggtgtgaaagatttcaatgagacacagctggcccggattgcggacagtaaggatcatgtgtttcccgtgaatgacggctttcaggctctgcaaggcatcatccactcaattttgaagaagtcctgcatcgaaattctagcagctgaaccatccaccatatgtgcaggagagtcatttcaagttgtcgtgagaggaaacggcttccgacatgcccgcaacgtggacagggtcctctgcagcttcaagatcaatgactcggtcacactcaatgagaagcccttttctgtggaagatacttatttactgtgtccagcgcctatcttaaaagaagttggcatgaaagctgcactccaggtcagcatgaacgatggcctctcttttatctccagttctgtcatcatcaccaccacacactgttctgacggttccatcctggccatcgccctgctgatcctgttcctgctcctagccctggctctcctctggtggttctggcccctctgctgcactgtgattatcaaggaggtccctccaccccctgccgaggagagtgaggaaaataaaataaaataacaagaagaagaaagaaagaaatcccacagaaacagataacctaacacagcccgtgcaacgtattttatacaatgctctgaaaatcatagtctcaatctagacagtcttttcctctagttccctgtattcaaatcccagtgtctaacattcaataaatagctatatgaaatcaaaaaaaaaaaaaaaaaaaaaaaaaaaaaaaaa

SEQ ID NO: 24 - protein, 234 aa, Homo sapiens
MATAERRALGIGFQWLSLATLVLICAGQGGRREDGGPACYGGFDLYFILDKSGSVLHHWNEIYYFVEQLAHKFISPQLRMSFIVFSTRGTTLMKLTEDREQIRQGLEELQKVLPGGDTYMHEGFERASEQIYYENRQGYRTASVIIALTDGELHEDLFFYSEREANRSRDLGAIVYCVGVKDFNETQLARIADSKDHVFPVNDGFQALQGIIHSILKKSCIEILAAEPSTICAG

SEQ ID NO: 25 - DNA, 1667 bp, Homo sapiens; identical to SEQ ID NO: 23

SEQ ID NO: 26 - protein, 489 aa, Homo sapiens
MVAERSPARSPGSWLFPGLWLLVLSGPGGLLRAQEQPSCRRAFDLYFVLDKSGSVANNWIEIYNFVQQLAERFVSPEMRLSFIVFSSQATIILPLTGDRGKISKGLEDLKRVSPVGETYIHEGLKLANEQIQKAGGLKTSSIIIALTDGKLDGLVPSYAEKEAKISRSLGASVYCVGVLDFEQAQLERIADSKEQVFPVKGGFQALKGIINSILAQSCTEILELQPSSVCVGEEFQIVLSGRGFMLGSRNGSVLCTYTVNETYTTSVKPVSVQLNSMLCPAPILNKAGETLDVSVSFNGGKSVISGSLIVTATECSNGIAAIIVILVLLLLLGIGLMWWFWPLCCKVVIKDPPPPPAPAPKEEEEEPLPTKKWPTVDASYYGGRGVGGIKRMEVRWGDKGSTEEGARLEKAKNAVVKIPEETEEPIRPRPPRPKPTHQPPQTKWYTPIKGRLDALWALLRRQYDRVSLMRPQEGDEVCIWECIEKELTA

SEQ ID NO: 27 - DNA, 2314 bp, Homo sapiens
agtcatcttcaactcggcaggaacccacaagtgtgcatgtgtggctcggaggcttcagctggggccccgccctcgtccccaggcgcacactgacacacgcagcccagacccggcccgagcgggctcctgccctcggcgtggcttctctccagccgggagtcccagggccagctagcctcctcccctaaaggggacggcctgtcagcgcagtgccagagtccagcaccgggaggaaagtttcggagtgcggagggagttggggccgccggaggagaagagtctccactcctagtttgttctgccgtcgccgcgtcccagggaccccttgtcccgaagcgcacggcagcgggggggacttcagccctccaggcggggtgggttccaggtccgggtccgaggcgggcgctggaggctcggccccaggccggagaggaactcctttcgcgagctgtcgccgtgggcccgcattgtctgcaggaactctccggaatcgggagggggaggactggatcgcgcttccactgggattcgtcaagagttccggcggcagctgcggcggtggcggagactccctttgtcctctcaggacctccctctctccctccctgtcagctggtgggtcccgctgccgcaggcgccggcgtctcagctgctcgccgccccccaccccagagtgcgtgccgggtgactcccgccacctttgcgaccctcctgagcttaggggactgcgagcgggagggagtctcaggcccccggccgcaggatggtggcggagcggtccccggcccgcagccccgggagctggctgttccccgggctgtggctgttggtgctcagcggtcccggggggctgctgcgcgcccaggagcagccctcctgcagaagagcctttgatctctacttcgtcctggacaagtctgggagtgtggcaaataactggattgaaatttataatttcgtacagcaacttgcggagagatttgtgagccctgaaatgagattatctttcattgtgttttcttctcaagcaactattattttgccattaactggagacagaggcaaaatcagtaaaggcttggaggatttaaaacgtgttagtccagtaggagagacatatatccatgaaggactaaagctagcgaatgaacaaattcagaaagcaggaggcttgaaaacctccagtatcataattgctctgacagatggcaagttggacggtctggtgccatcatatgcagagaaagaggcaaagatatccaggtcacttggggctagtgtttattgtgttggtgtccttgattttgaacaagcacagcttgaaagaattgctgattccaaggagcaagttttccctgtcaaaggtggatttcaggctcttaaaggaataattaattctatactagctcagtcatgtactgaaatcctagaattgcagccctcaagtgtctgtgtgggggaggaatttcagattgtcttaagtggaagaggattcatgctgggcagtcggaatggcagtgttctctgcacttacactgtaaatgaaacatatacaacgagtgtaaaaccagtaagtgtacagcttaattctatgctttgtcctgcacctatcctgaataaagctggagaaactcttgatgtttcagtgagctttaatggaggaaaatctgtcatttcaggatcattaattgtcacagccacagaatgttctaacgggatcgcagccatcattgttattttggtgttactgctactcctggggatcggtttgatgtggtggttttggcccctttgctgcaaagtggttattaaggatcctccaccaccacccgcccctgcaccaaaagaggaggaagaagaacctttgcctactaaaaagtggccaactgtggatgcttcctattatggtggtcgaggggttggaggaattaaaagaatggaggttcgttggggtgataaaggatctactgaggaaggtgcaaggctagagaaagccaaaaatgctgtggtgaagattcctgaagaaacagaggaacccatcaggcctagaccacctcgacccaaacccacacaccagcctcctcagacaaaatggtacaccccaattaagggtcgtcttgatgctctctgggctttgttgaggcggcagtatgaccgggtttctttgatgcgacctcaggaaggagatgaggtttgtatatgggaatgtattgagaaagagctaactgcttgagtcagtataatggaggcagggaaatagtaataaaaaatgattttaaagccctattgcacttgaggaaggaaaaaaaaaaaa

SEQ ID NO: 28 - protein, 232 aa, Homo sapiens
MVAERSPARSPGSWLFPGLWLLVLSGPGGLLRAQEQPSCRRAFDLYFVLDKSGSVANNWIEIYNFVQQLAERFVSPEMRLSFIVFSSQATIILPLTGDRGKISKGLEDLKRVSPVGETYIHEGLKLANEQIQKAGGLKTSSIIIALTDGKLDGLVPSYAEKEAKISRSLGASVYCVGVLDFEQAQLERIADSKEQVFPVKGGFQALKGIINSILAQSCTEILELQPSSVCVG

SEQ ID NO: 29 - DNA, 2314 bp, Homo sapiens; identical to SEQ ID NO: 27

SEQ ID NO: 30 - DNA, 930 bp, Homo sapiens; identical to SEQ ID NO: 17

SEQ ID NO: 31 - protein, 309 aa, Homo sapiens; identical to SEQ ID NO: 16

SEQ ID NO: 32 - DNA, 1831 bp, Homo sapiens
atggtcagctactgggacaccggggtcctgctgtgcgcgctgctcagctgtctgcttctcacaggatctagttcaggttcaaaattaaaagatcctgaactgagtttaaaaggcacccagcacatcatgcaagcaggccagacactgcatctccaatgcaggggggaagcagcccataaatggtctttgcctgaaatggtgagtaaggaaagcgaaaggctgagcataactaaatctgcctgtggaagaaatggcaaacaattctgcagtactttaaccttgaacacagctcaagcaaaccacactggcttctacagctgcaaatatctagctgtacctacttcaaagaagaaggaaacagaatctgcaatctatatatttattagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagcattcatcactgtgaaacaagatctgggcccgggcgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggatcaccacgcttccaggactcctcttcttcaaaggcccctcctcctagcttccaagcccatcccgactcccggggccctcggacactcgatcctcccacaataa

SEQ ID NO: 33 - protein, 610 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKQDLGPGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSPRFQDSSSSKAPPPSLPSPSRLPGPSDTPILPQ

SEQ ID NO: 34 - DNA, 1840 bp, Homo sapiens
atgcagagcaaggtgctgctggccgtcgccctgtggctctgcgtggagacccgggccgcctctgtgggtttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggcaagatctgggcccgggcgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggatcaccacgcttccaggactcctcttcctcaaaggcccctcctcctagccttccaagcccatcccgactcccggggccctcggacactccgatcctcccacaataa

SEQ ID NO: 35 - protein, 610 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLLGPGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSPRFQDSSSSKAPPPSLPSPSRLPGPSDTPILPQ

SEQ ID NO: 36 - DNA, 108 bp, Homo sapiens; identical to SEQ ID NO: 4

SEQ ID NO: 37 - protein, 35 aa, Homo sapiens; identical to SEQ ID NO: 3

SEQ ID NO: 38 - DNA, 20 bp, Artificial Sequence
  Description of Artificial Sequence Synthetic primer
tagagatccctcgagggcgc

SEQ ID NO: 39 - DNA, 134 bp, Artificial Sequence
  Description of Artificial Sequence Synthetic primer
tctcagcgccggcgaccttattgtgggaggatcggagtgtccgagggccccgggagtcgggatgggcttggaaggctaggaggagggggcctttgaggaagaggagtcctggaagcgtggtgatcctttacccg

(19) United States
(12) Patent Application Publication
(10) Pub. No.: US 2015/0175675 A1
(43) Pub. Date: Jun. 25, 2015

(54) FUSION POLYPEPTIDES AND METHODS OF USE THEREOF

(71) Applicant: The Trustees Of Columbia University In The City Of New York, New York, NY (US)

(72) Inventors: Jan K. KITAJEWSKI, Ridgewood, NJ (US); Claire V. Reeves, New York, NY (US); Timothy Wang, New York, NY (US)

(21) Appl. No.: 14/507,785

(22) Filed: Oct. 6, 2014

Related U.S. Application Data
(63) Continuation-in-part of application No. PCT/US13/35633, filed on Apr. 8, 2013.
(60) Provisional application No. 61/621,171, filed on Apr. 6, 2012.

(51) Int. Cl. C07K 14/705; C07K 14/59

(52) U.S. Cl. CPC C07K 14/705; C07K 14/59; C07K 2319/30; C07K 2319/31

(57) ABSTRACT

The present invention also provides fusion polypeptides with a carboxy-terminal or N-terminal peptide domain (e.g., Fc, CTP, or Fc-CTP), and nucleic acid molecules encoding these polypeptides. The present invention further provides for methods of using the compositions of the invention for treatment of cancer and fibrotic diseases. The present invention also provides isolated polypeptides with a carboxy-terminal peptide (CTP) domain fused to an antibody fragment, and nucleic acid molecules encoding these polypeptides. The present invention also provides isolated polypeptides with a carboxy-terminal peptide (CTP) domain fused to the ectodomain of a receptor, and nucleic acid molecules encoding these polypeptides. Also provided are isolated fusion polypeptide molecules, with an isolated polypeptide attached to a carboxy terminus of a second polypeptide, and to nucleic acid molecules encoding these isolated fusion polypeptide molecules. Finally, methods of increasing a biological half-life of a polypeptide, methods of stabilizing a polypeptide, pharmaceutical compositions including the polypeptides and fusion polypeptides, and methods of treating or preventing a disorder using these polypeptides are provided

This application is a continuation-in-part of International Application No. PCT/US2013/35633, filed on Apr. 8, 2013, which itself claims the benefit of and priority to U.S. Provisional Patent Application No. 61/621,171, filed on Apr. 6, 2012, the contents of which are hereby incorporated by reference in their entirety. This application also claims priority to International Application No. PCT/US2013/34981, filed on Apr. 2, 2013, which itself claims the benefit of Application No. 61/649,767, filed on May 21, 2012, the contents of which are hereby incorporated by reference in their entirety.

### GOVERNMENT INTERESTS

This invention was made with government support under ROI AI064654 awarded by the National Institutes of Health/National Institute of Allergy and Infectious Diseases. The Government has certain rights in the invention.

All patents, patent applications and publications cited herein are hereby incorporated by reference in their entirety. The disclosures of these publications in their entireties are hereby incorporated by reference into this application.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND OF THE INVENTION

In recent years scientific research has begun to focus on the role of a tumor's surrounding environment, or microenvironment in promoting cancer progression. Within the microenvironment there are extracellular matrix proteins and many different types of cells that send signals to tumors that either promote or inhibit tumor growth. For example, dysregulation in extracellular matrix or tissue organization increases the chance of tumor initiation.

A polypeptide is a single linear chain of amino acids bonded together by peptide bonds, with an amino (NH₂) group at one end (its N-terminus) and a carboxyl (COOH) group at its other end (its C-terminus). Polypeptides, including antibodies, are susceptible to denaturation or enzymatic degradation in the blood, liver or kidney. Accordingly, polypeptides are unstable and have short biological half-lives. Because of their low stability, therapeutic polypeptides are often delivered in a sustained frequency to maintain an effective plasma concentration of the active peptide. Moreover, therapeutic polypeptides, including antibody-based therapeutics, are often administrated by infusion, and such an administration causes considerable discomfort to a subject.

### SUMMARY OF THE INVENTION

An aspect of the invention encompasses breakdown of the extracellular matrix by regulation of matrix metalloproteinase (MMP14, also known as MT1-MMP) activity, as well as MMP1 and MMP2 activity via the extracellular domains of Anthrax Toxin Receptors (e.g., ANTXR1 and ANTXR2). Various Anthrax Toxin Receptor Extracellular Domain constructs can be used to both positively and negatively regulate the MMP cascade. In one embodiment, the positive effects on the MMP cascade can be used to break down the extracellular matrix, and thus the fusion polypeptides of the invention may be used for the treatment of fibrotic diseases, such as arthritis, inflammatory fibrosis, and in damaged tissue with a high level of fibrosis.

An aspect of the invention provides for fusion polypeptides that positively regulate MMPs, wherein the fusion polypeptide comprises ANTXR1. In one embodiment, the fusion polypeptide comprises a secreted protein comprising the extracellular domain of ANTXR1 fused to an Fc domain. In a further embodiment, the fusion polypeptide comprises a secreted protein comprising the extracellular domain of ANTXR1 fused to a CTP domain. In yet another embodiment, the fusion polypeptide comprises a secreted protein comprising the extracellular domain of ANTXR1 fused to a Fc-CTP domain. In one embodiment, the fusion polypeptide comprises a secreted protein comprising the vWF domain of ANTXR1 fused to an Fc domain. In a further embodiment, the fusion polypeptide comprises a secreted protein comprising the vWF domain of ANTXR1 fused to a CTP domain. In yet another embodiment, the fusion polypeptide comprises a secreted protein comprising the vWF domain of ANTXR1 fused to a Fc-CTP domain. In further embodiments, the CTP, Fc, or Fc-CTP domain is fused to the N-terminus of the ANTXR1 extracellular domain, while in other embodiments the CTP, Fc, or Fc-CTP domain is fused to the C-terminus of the ANTXR1 extracellular domain. In further embodiments, the CTP, Fc, or Fc-CTP domain is fused to the N-terminus of the ANTXR1 vWF domain, while in other embodiments the CTP, Fc, or Fc-CTP domain is fused to the C-terminus of the ANTXR1 vWF domain. In some embodiments, the polypeptide of the invention comprises the extracellular domain of ANTXR1 alone. In other embodiments, the polypeptide of the invention comprises the vWF domain of ANTXR1 alone.

An aspect of the invention provides for fusion polypeptides that positively regulate MMPs, wherein the fusion polypeptide comprises ANTXR2. In one embodiment, the fusion polypeptide comprises a secreted protein comprising the extracellular domain of ANTXR2 fused to an Fc domain. In a further embodiment, the fusion polypeptide comprises a secreted protein comprising the extracellular domain of ANTXR2 fused to a CTP domain. In yet another embodiment, the fusion polypeptide comprises a secreted protein comprising the extracellular domain of ANTXR2 fused to a Fc-CTP domain. In one embodiment, the fusion polypeptide comprises a secreted protein comprising the vWF domain of ANTXR2 fused to an Fc domain. In a further embodiment, the fusion polypeptide comprises a secreted protein comprising the vWF domain of ANTXR2 fused to a CTP domain. In yet another embodiment, the fusion polypeptide comprises a secreted protein comprising the vWF domain of ANTXR2 fused to a Fc-CTP domain. In further embodiments, the CTP, Fc, or Fc-CTP domain is fused to the N-terminus of the ANTXR2 extracellular domain, while in other embodiments the CTP, Fc, or Fc-CTP domain is fused to the C-terminus of the ANTXR2 extracellular domain. In further embodiments, the CTP, Fc, or Fc-CTP domain is fused to the N-terminus of the ANTXR2 vWF domain, while in other embodiments the CTP, Fc, or Fc-CTP domain is fused to the C-terminus of the ANTXR2 vWF domain. In some embodiments, the polypeptide of the invention comprises the extracellular domain of ANTXR2 alone. In other embodiments, the polypeptide of the invention comprises the vWF domain of ANTXR2 alone.

In further embodiments, fusion polypeptides comprising an Fc domain, a CTP domain, or a Fc-CTP domain can be used as tags for affinity purification of the moiety. In other embodiments, fusion polypeptides comprising an Fc domain, a CTP domain, or a Fc-CTP domain can be used for detection of the construct. In yet further embodiments, fusion polypeptides comprising an Fc domain, a CTP domain, or a Fc-CTP domain can be used for stabilization of the ANTXR2 and/or ANTXR2 fusion constructs.

An aspect of the invention provides for an ANTXR fusion polypeptide that negatively regulates MMPs. In one embodiment, the ANTXR fusion polypeptide comprises cysteine mutants in its vWF domain that would bind to MMP14 but no longer activate the MMP cascade (for example, MMP14, MMP1, MMP2). In one embodiment, the fusion polypeptide comprises a secreted protein comprising the vWF domain of ANTXR1 with cysteine mutant(s) fused to an Fc domain. In a further embodiment, the fusion polypeptide comprises a secreted protein comprising the vWF domain of ANTXR1 with cysteine mutant(s) fused to a CTP domain. In yet another embodiment, the fusion polypeptide comprises a secreted protein comprising the vWF domain of ANTXR1 with cysteine mutant(s) fused to a Fc-CTP domain. In one embodiment, the fusion polypeptide comprises a secreted protein comprising the vWF domain of ANTXR2 with cysteine mutant(s) fused to an Fc domain. In a further embodiment, the fusion polypeptide comprises a secreted protein comprising the vWF domain of ANTXR2 with cysteine mutant(s) fused to a CTP domain. In yet another embodiment, the fusion polypeptide comprises a secreted protein comprising the vWF domain of ANTXR2 with cysteine mutant(s) fused to a Fc-CTP domain. These ANTXR fusion polypeptides would act as dominant inhibitors of the native ANTXR/MMP14 interaction. These inhibitors would be used to reduce the activity of MMP in situations where MMPs have pathological promoting activities, such as during tumor cell invasion.

An aspect of the invention is directed to an isolated polypeptide comprising an Anthrax Toxin Receptor (ANTXR) fused to an Fc domain, a CTP domain, an Fc-CTP domain, or a combination thereof. In one embodiment, the ANTXR is ANTXR1 or ANTXR2. In another embodiment, the ANTXR comprises the extracellular domain of ANTXR1 or ANTXR2. In a further embodiment, the Fc domain is about 95% identical to SEQ ID NO: 1. In some embodiments, the Fc domain is about 96% identical to SEQ ID NO: 1. In further embodiments, the Fc domain is about 97% identical to SEQ ID NO: 1. In other embodiments, the Fc domain is about 98% identical to SEQ ID NO: 1. In another embodiment, the Fc domain is about 99% identical to SEQ ID NO: 1. In yet other embodiments, the Fc domain is SEQ ID NO: 1. In a further embodiment, the Fc domain is about 95% identical to SEQ ID NO: 3. In some embodiments, the Fc domain is about 96% identical to SEQ ID NO: 3. In further embodiments, the Fc domain is about 97% identical to SEQ ID NO: 3. In other embodiments, the Fc domain is about 98% identical to SEQ ID NO: 3. In another embodiment, the Fc domain is about 99% identical to SEQ ID NO: 3. In yet other embodiments, the Fc domain is SEQ ID NO: 3. In a further embodiment, the Fc domain is about 95% identical to SEQ ID NO: 16. In some embodiments, the Fc domain is about 96% identical to SEQ ID NO: 16. In further embodiments, the Fc domain is about 97% identical to SEQ ID NO: 16. In other embodiments, the Fc domain is about 98% identical to SEQ ID NO: 16. In another embodiment, the Fc domain is about 99% identical to SEQ ID NO: 16. In yet other embodiments, the Fc domain is SEQ ID NO: 16. In one embodiment, the CTP domain, Fc domain, Fc-CTP domain, or combination thereof, is fused to the C-terminus of the ANTXR. In another embodiment, the CTP domain, Fc domain, Fc-CTP domain, or combination thereof, is fused to the N-terminus of the ANTXR. In a further embodiment, the CTP domain, Fc domain, Fc-CTP domain, or combination thereof, is fused to the C-terminus and the N-terminus of the ANTXR.

An aspect of the invention is directed to an isolated polypeptide comprising a vWF domain of an Anthrax Toxin Receptor (ANTXR) fused to an Fc domain, a CTP domain, an Fc-CTP domain, or a combination thereof. In one embodiment, the ANTXR is ANTXR1 or ANTXR2. In another embodiment, the ANTXR comprises the extracellular domain of ANTXR1 or ANTXR2. In a further embodiment, the Fc domain is about 95% identical to SEQ ID NO: 1. In some embodiments, the Fc domain is about 96% identical to SEQ ID NO: 1. In further embodiments, the Fc domain is about 97% identical to SEQ ID NO: 1. In other embodiments, the Fc domain is about 98% identical to SEQ ID NO: 1. In another embodiment, the Fc domain is about 99% identical to SEQ ID NO: 1. In yet other embodiments, the Fc domain is SEQ ID NO: 1. In a further embodiment, the Fc domain is about 95% identical to SEQ ID NO: 3. In some embodiments, the Fc domain is about 96% identical to SEQ ID NO: 3. In further embodiments, the Fc domain is about 97% identical to SEQ ID NO: 3. In other embodiments, the Fc domain is about 98% identical to SEQ ID NO: 3. In another embodiment, the Fc domain is about 99% identical to SEQ ID NO: 3. In yet other embodiments, the Fc domain is SEQ ID NO: 3. In a further embodiment, the Fc domain is about 95% identical to SEQ ID NO: 16. In some embodiments, the Fc domain is about 96% identical to SEQ ID NO: 16. In further embodiments, the Fc domain is about 97% identical to SEQ ID NO: 16. In other embodiments, the Fc domain is about 98% identical to SEQ ID NO: 16. In another embodiment, the Fc domain is about 99% identical to SEQ ID NO: 16. In yet other embodiments, the Fc domain is SEQ ID NO: 16. In one embodiment, the CTP domain, Fc domain, Fc-CTP domain, or combination thereof, is fused to the C-terminus of the ANTXR. In another embodiment, the CTP domain, Fc domain, Fc-CTP domain, or combination thereof, is fused to the N-terminus of the ANTXR. In a further embodiment, the CTP domain, Fc domain, Fc-CTP domain, or combination thereof, is fused to the C-terminus and the N-terminus of the ANTXR.

An aspect of the invention is directed to an isolated polypeptide comprising an extracellular domain of an Anthrax Toxin Receptor (ANTXR) fused to an Fc domain, a CTP domain, an Fc-CTP domain, or a combination thereof. In one embodiment, the ANTXR is ANTXR1 or ANTXR2. In another embodiment, the ANTXR comprises the extracellular domain of ANTXR1 or ANTXR2. In a further embodiment, the Fc domain is about 95% identical to SEQ ID NO: 1. In some embodiments, the Fc domain is about 96% identical to SEQ ID NO: 1. In further embodiments, the Fc domain is about 97% identical to SEQ ID NO: 1. In other embodiments, the Fc domain is about 98% identical to SEQ ID NO: 1. In another embodiment, the Fc domain is about 99% identical to SEQ ID NO: 1. In yet other embodiments, the Fc domain is SEQ ID NO: 1. In a further embodiment, the Fc domain is about 95% identical to SEQ ID NO: 3. In some embodiments, the Fc domain is about 96% identical to SEQ ID NO: 3. In further embodiments, the Fc domain is about 97% identical to SEQ ID NO: 3. In other embodiments, the Fc domain is about 98% identical to SEQ ID NO: 3. In another embodiment, the Fc domain is about 99% identical to SEQ ID NO: 3. In yet other embodiments, the Fc domain is SEQ ID NO: 3. In a further embodiment, the Fc domain is about 95% identical to SEQ ID NO: 16. In some embodiments, the Fc domain is about 96% identical to SEQ ID NO: 16. In further embodiments, the Fc domain is about 97% identical to SEQ ID NO: 16. In other embodiments, the Fc domain is about 98% identical to SEQ ID NO: 16. In another embodiment, the Fc domain is about 99% identical to SEQ ID NO: 16. In yet other embodiments, the Fc domain is SEQ ID NO: 16. In one embodiment, the CTP domain, Fc domain, Fc-CTP domain, or combination thereof, is fused to the C-terminus of the ANTXR. In another embodiment, the CTP domain, Fc domain, Fc-CTP domain, or combination thereof, is fused to the N-terminus of the ANTXR. In a further embodiment, the CTP domain, Fc domain, Fc-CTP domain, or combination thereof, is fused to the C-terminus and the N-terminus of the ANTXR.

An aspect of the invention is directed to an isolated nucleic acid encoding the isolated polypeptide comprising an Anthrax Toxin Receptor (ANTXR) fused to an Fc domain, a CTP domain, an Fc-CTP domain, or a combination thereof.

An aspect of the invention is directed to an isolated nucleic acid encoding the isolated polypeptide comprising a vWF domain of an Anthrax Toxin Receptor (ANTXR) fused to an Fc domain, a CTP domain, an Fc-CTP domain, or a combination thereof.

An aspect of the invention is directed to an isolated nucleic acid encoding the isolated polypeptide comprising an extracellular domain of an Anthrax Toxin Receptor (ANTXR) fused to an Fc domain, a CTP domain, an Fc-CTP domain, or a combination thereof.

An aspect of the invention is directed to a pharmaceutical composition comprising the isolated polypeptide comprising an Anthrax Toxin Receptor (ANTXR) fused to an Fc domain, a CTP domain, an Fc-CTP domain, or a combination thereof, and a pharmaceutically acceptable carrier.

An aspect of the invention is directed to a pharmaceutical composition comprising the isolated polypeptide comprising a vWF domain of an Anthrax Toxin Receptor (ANTXR) fused to an Fc domain, a CTP domain, an Fc-CTP domain, or a combination thereof, and a pharmaceutically acceptable carrier.

An aspect of the invention is directed to a pharmaceutical composition comprising the isolated polypeptide comprising an extracellular domain of an Anthrax Toxin Receptor (ANTXR) fused to an Fc domain, a CTP domain, an Fc-CTP domain, or a combination thereof, and a pharmaceutically acceptable carrier.

An aspect of the invention is directed to a method of decreasing fibrosis in a tissue of a subject, the method comprising administering to a subject an ANTXR molecule, thereby decreasing fibrosis in a tissue. In one embodiment, the ANTXR molecule is the isolated polypeptide comprising an Anthrax Toxin Receptor (ANTXR) fused to an Fc domain, a CTP domain, an Fc-CTP domain, or a combination thereof. In one embodiment, the ANTXR molecule is the isolated polypeptide comprising a vWF domain of an Anthrax Toxin Receptor (ANTXR) fused to an Fc domain, a CTP domain, an Fc-CTP domain, or a combination thereof. In a further embodiment, the ANTXR molecule is the isolated polypeptide comprising an extracellular domain of an Anthrax Toxin Receptor (ANTXR) fused to an Fc domain, a CTP domain, an Fc-CTP domain, or a combination thereof. In another embodiment, the ANTXR molecule comprises an ANTXR protein having SEQ ID NO: 18, 22, or 24. In yet another embodiment, the ANTXR molecule comprises an ANTXR protein having SEQ ID NO: 20, 26, or 28. In a further embodiment, the ANTXR molecule is the isolated nucleic acid encoding the isolated polypeptide comprising an Anthrax Toxin Receptor (ANTXR) fused to an Fc domain, a CTP domain, an Fc-CTP domain, or a combination thereof. In yet a further embodiment, the ANTXR molecule is the isolated nucleic acid encoding the isolated polypeptide comprising a vWF domain of an Anthrax Toxin Receptor (ANTXR) fused to an Fc domain, a CTP domain, an Fc-CTP domain, or a combination thereof. In one embodiment, fibrosis results from damaged tissue. In some embodiments, the damaged tissue is lung, bladder, esophageal, small intestine, large intestine, or colon.

An aspect of the invention is directed to a method of treating or preventing a fibrotic disease in a subject, the method comprising administering to a subject an ANTXR molecule, thereby treating or preventing the fibrotic disease. In one embodiment, the ANTXR molecule is the isolated polypeptide comprising an Anthrax Toxin Receptor (ANTXR) fused to an Fc domain, a CTP domain, an Fc-CTP domain, or a combination thereof. In one embodiment, the ANTXR molecule is the isolated polypeptide comprising a vWF domain of an Anthrax Toxin Receptor (ANTXR) fused to an Fc domain, a CTP domain, an Fc-CTP domain, or a combination thereof. In a further embodiment, the ANTXR molecule is the isolated polypeptide comprising an extracellular domain of an Anthrax Toxin Receptor (ANTXR) fused to an Fc domain, a CTP domain, an Fc-CTP domain, or a combination thereof. In another embodiment, the ANTXR molecule comprises an ANTXR protein having SEQ ID NO: 18, 22, or 24. In yet another embodiment, the ANTXR molecule comprises an ANTXR protein having SEQ ID NO: 20, 26, or 28. In a further embodiment, the ANTXR molecule is the isolated nucleic acid encoding the isolated polypeptide comprising an Anthrax Toxin Receptor (ANTXR) fused to an Fc domain, a CTP domain, an Fc-CTP domain, or a combination thereof. In yet a further embodiment, the ANTXR molecule is the isolated nucleic acid encoding the isolated polypeptide comprising a vWF domain of an Anthrax Toxin Receptor (ANTXR) fused to an Fc domain, a CTP domain, an Fc-CTP domain, or a combination thereof. In some embodiments, the fibrotic disease comprises arthritis, inflammatory fibrosis, systemic hyalinosis, juvenile hyaline fibromatosis, or infantile systemic hyalinosis.

An aspect of the invention is directed to a method of treating or preventing an epithelial cancer in a subject, the method comprising administering to a subject an ANTXR molecule, thereby treating the epithelial cancer. In one embodiment, the ANTXR molecule is the isolated polypeptide comprising an Anthrax Toxin Receptor (ANTXR) fused to an Fc domain, a CTP domain, an Fc-CTP domain, or a combination thereof. In one embodiment, the ANTXR molecule is the isolated polypeptide comprising a vWF domain of an Anthrax Toxin Receptor (ANTXR) fused to an Fc domain, a CTP domain, an Fc-CTP domain, or a combination thereof. In a further embodiment, the ANTXR molecule is the isolated polypeptide comprising an extracellular domain of an Anthrax Toxin Receptor (ANTXR) fused to an Fc domain, a CTP domain, an Fc-CTP domain, or a combination thereof. In another embodiment, the ANTXR molecule comprises an ANTXR protein having SEQ ID NO: 18, 22, or 24. In yet another embodiment, the ANTXR molecule comprises an ANTXR protein having SEQ ID NO: 20, 26, or 28. In a further embodiment, the ANTXR molecule is the isolated nucleic acid encoding the isolated polypeptide comprising an Anthrax Toxin Receptor (ANTXR) fused to an Fc domain, a CTP domain, an Fc-CTP domain, or a combination thereof. In yet a further embodiment, the ANTXR molecule is the isolated nucleic acid encoding the isolated polypeptide comprising a vWF domain of an Anthrax Toxin Receptor (ANTXR) fused to an Fc domain, a CTP domain, an Fc-CTP domain, or a combination thereof. In one embodiment, the epithelial cancer is breast cancer or ovarian cancer. In another embodiment, the subject is a human, horse, dog or cat.

An aspect of the invention is directed to a method of decreasing or preventing tumor cell invasion into a tissue free from tumor cells in a subject, the method comprising administering to a subject an ANTXR molecule, thereby decreasing or preventing tumor cell invasion. In one embodiment, the ANTXR molecule is the isolated polypeptide comprising an Anthrax Toxin Receptor (ANTXR) fused to an Fc domain, a CTP domain, an Fc-CTP domain, or a combination thereof. In one embodiment, the ANTXR molecule is the isolated polypeptide comprising a vWF domain of an Anthrax Toxin Receptor (ANTXR) fused to an Fc domain, a CTP domain, an Fc-CTP domain, or a combination thereof. In a further embodiment, the ANTXR molecule is the isolated polypeptide comprising an extracellular domain of an Anthrax Toxin Receptor (ANTXR) fused to an Fc domain, a CTP domain, an Fc-CTP domain, or a combination thereof. In another embodiment, the ANTXR molecule comprises an ANTXR protein having SEQ ID NO: 18, 22, or 24. In yet another embodiment, the ANTXR molecule comprises an ANTXR protein having SEQ ID NO: 20, 26, or 28. In a further embodiment, the ANTXR molecule is the isolated nucleic acid encoding the isolated polypeptide comprising an Anthrax Toxin Receptor (ANTXR) fused to an Fc domain, a CTP domain, an Fc-CTP domain, or a combination thereof. In yet a further embodiment, the ANTXR molecule is the isolated nucleic acid encoding the isolated polypeptide comprising a vWF domain of an Anthrax Toxin Receptor (ANTXR) fused to an Fc domain, a CTP domain, an Fc-CTP domain, or a combination thereof. In one embodiment, the tumor is a breast tumor or an ovarian tumor. In another embodiment, the subject is a human, horse, dog or cat.

An aspect of the invention is directed to a method of decreasing or preventing cancer metastasis in a subject, the method comprising administering to a subject an ANTXR molecule, thereby decreasing or preventing cancer metastasis. In one embodiment, the ANTXR molecule is the isolated polypeptide comprising an Anthrax Toxin Receptor (ANTXR) fused to an Fc domain, a CTP domain, an Fc-CTP domain, or a combination thereof. In one embodiment, the ANTXR molecule is the isolated polypeptide comprising a vWF domain of an Anthrax Toxin Receptor (ANTXR) fused to an Fc domain, a CTP domain, an Fc-CTP domain, or a combination thereof. In a further embodiment, the ANTXR molecule is the isolated polypeptide comprising an extracellular domain of an Anthrax Toxin Receptor (ANTXR) fused to an Fc domain, a CTP domain, an Fc-CTP domain, or a combination thereof. In another embodiment, the ANTXR molecule comprises an ANTXR protein having SEQ ID NO: 18, 22, or 24. In yet another embodiment, the ANTXR molecule comprises an ANTXR protein having SEQ ID NO: 20, 26, or 28. In a further embodiment, the ANTXR molecule is the isolated nucleic acid encoding the isolated polypeptide comprising an Anthrax Toxin Receptor (ANTXR) fused to an Fc domain, a CTP domain, an Fc-CTP domain, or a combination thereof. In yet a further embodiment, the ANTXR molecule is the isolated nucleic acid encoding the isolated polypeptide comprising a vWF domain of an Anthrax Toxin Receptor (ANTXR) fused to an Fc domain, a CTP domain, an Fc-CTP domain, or a combination thereof. In one embodiment, the cancer is epithelial cancer. In another embodiment, the epithelial cancer is breast cancer or ovarian cancer. In another embodiment, the subject is a human, horse, dog or cat.

An aspect of the invention is directed to a method for decreasing or preventing angiogenesis in a tumor, the method comprising (a) delivering an ANTXR molecule to a cell in a tumor; and (b) expressing in the cell of the tumor the ANTXR molecule, thereby decreasing or preventing angiogenesis. In one embodiment, the ANTXR molecule is the isolated polypeptide comprising an Anthrax Toxin Receptor (ANTXR) fused to an Fc domain, a CTP domain, an Fc-CTP domain, or a combination thereof. In one embodiment, the ANTXR molecule is the isolated polypeptide comprising a vWF domain of an Anthrax Toxin Receptor (ANTXR) fused to an Fc domain, a CTP domain, an Fc-CTP domain, or a combination thereof. In a further embodiment, the ANTXR molecule is the isolated polypeptide comprising an extracellular domain of an Anthrax Toxin Receptor (ANTXR) fused to an Fc domain, a CTP domain, an Fc-CTP domain, or a combination thereof. In another embodiment, the ANTXR molecule comprises an ANTXR protein having SEQ ID NO: 18, 22, or 24. In yet another embodiment, the ANTXR molecule comprises an ANTXR protein having SEQ ID NO: 20, 26, or 28. In a further embodiment, the ANTXR molecule is the isolated nucleic acid encoding the isolated polypeptide comprising an Anthrax Toxin Receptor (ANTXR) fused to an Fc domain, a CTP domain, an Fc-CTP domain, or a combination thereof. In yet a further embodiment, the ANTXR molecule is the isolated nucleic acid encoding the isolated polypeptide comprising a vWF domain of an Anthrax Toxin Receptor (ANTXR) fused to an Fc domain, a CTP domain, an Fc-CTP domain, or a combination thereof. In one embodiment, the tumor is a breast tumor or an ovarian tumor.

An aspect of the invention is directed to an isolated fusion polypeptide comprising a secreted protein comprising the vWF domain of ANTXR1 with cysteine mutant(s) fused to an Fc domain, a CTP domain, or an Fc-CTP domain. In one embodiment, variants of the ANTXR molecule comprise a polypeptide comprising the vWF domain of ANTXR1 with cysteine mutant(s) fused to an Fc domain, a CTP domain, an Fc-CTP domain, or a combination thereof. In another embodiment, variants of the ANTXR molecule comprise a polypeptide comprising the extracellular domain of ANTXR1 with cysteine mutant(s) fused to an Fc domain, a CTP domain, an Fc-CTP domain, or a combination thereof. In a further embodiment, variants of the ANTXR molecule comprise a polypeptide comprising SEQ ID NO: 18 or 22 (or a fusion thereof that further comprises SEQ ID NO: 1, 3 and/or 16), wherein the cysteine residues at positions 25, 39, 177, 220, 232, 257, 281, and 317 of ANTXR1 are mutated. In one embodiment, the cysteine mutations in ANTXR1 can occur at positions 25, 39, 177, 220, 232, 257, 281, 317, or a combination thereof. In one embodiment, at least one cysteine residue is mutated. In another embodiment, at least two cysteine residues are mutated. In yet another embodiment, at least three cysteine residues are mutated. In yet another embodiment, at least four cysteine residues are mutated. In a further embodiment, at least five cysteine residues are mutated. In yet a further embodiment, at least six cysteine residues are mutated. In some embodiments, at least seven cysteine residues are mutated. In other embodiments, at least eight cysteine residues are mutated. In one embodiment, Cys177 in the vWF domain of SEQ ID NO 18 or 22 is mutated. In another embodiment, Cys220, Cys232, Cys257, Cys281, Cys317, or a combination thereof, in the extracellular domain of SEQ ID NO: 18 or 22 is mutated. In some embodiments, the cysteine residue is mutated to any one of the following: Cys to Ser, Cys to Tyr, Cys to Thr, Cys to Pro, Cys to Ala, Cys to Gly, Cys to Asn, Cys to Asp, Cys to Glu, Cys to Arg, or Cys to Lys. In a further embodiment, the cysteine residue is mutated to a serine residue or an alanine residue.

An aspect of the invention is directed to an isolated fusion polypeptide comprising a secreted protein comprising the vWF domain of ANTXR2 with cysteine mutant(s) fused to an Fc domain, a CTP domain, or an Fc-CTP domain. In one embodiment, variants of the ANTXR molecule comprise a polypeptide comprising the vWF domain of ANTXR2 with cysteine mutant(s) fused to an Fc domain, a CTP domain, an Fc-CTP domain, or a combination thereof. In another embodiment, variants of the ANTXR molecule comprise a polypeptide comprising the extracellular domain of ANTXR2 with cysteine mutant(s) fused to an Fc domain, a CTP domain, an Fc-CTP domain, or a combination thereof. In a further embodiment, variants of the ANTXR molecule comprise a polypeptide comprising SEQ ID NO: 20 or 26 (or a fusion thereof that further comprises SEQ ID NO: 1, 3 and/or 16), wherein the cysteine residues at positions 39, 175, 218, 230, 255, 279, and 315 of ANTXR2 are mutated. In one embodiment, the cysteine mutations in ANTXR2 can occur at positions 39, 175, 218, 230, 255, 279, 315, or a combination thereof. In one embodiment, at least one cysteine residue is mutated. In another embodiment, at least two cysteine residues are mutated. In yet another embodiment, at least three cysteine residues are mutated. In yet another embodiment, at least four cysteine residues are mutated. In a further embodiment, at least five cysteine residues are mutated. In yet a further embodiment, at least six cysteine residues are mutated. In some embodiments, at least seven cysteine residues are mutated. In other embodiments, at least eight cysteine residues are mutated. In one embodiment, Cys175 in the vWF domain of SEQ ID NO: 20 or 26 is mutated. In another embodiment, Cys218, Cys230, Cys255, Cys279, Cys315, or a combination thereof, in the extracellular domain of SEQ ID NO: 20 or 26 is mutated. In some embodiments, the cysteine residue is mutated to any one of the following: Cys to Ser, Cys to Tyr, Cys to Thr, Cys to Pro, Cys to Ala, Cys to Gly, Cys to Asn, Cys to Asp, Cys to Glu, Cys to Arg, or Cys to Lys. In a further embodiment, the cysteine residue is mutated to a serine residue or an alanine residue.

The invention is also based, at least in part, on the discovery of an isolated polypeptide comprising a carboxy-terminal peptide (CTP) domain fused to an antibody fragment. Accordingly, in one embodiment, the present invention relates to an isolated polypeptide comprising a CTP domain having at least about 90% identity to SEQ ID NO: 37, and wherein the CTP domain is fused to an antibody fragment.

This invention is also based, at least in part, on the discovery of an isolated polypeptide comprising a carboxy-terminal peptide (CTP) domain fused to an ectodomain of a receptor. In one embodiment, fusing a CTP domain to the ectodomain of a receptor (for example, a cell surface receptor) results in increased glycosylation and/or protein stability. In one embodiment, the receptor comprises extracellular domains 1-3 of human VEGFR1, as depicted in the shaded regions of FIG. 17B. In another embodiment, the receptor comprises extracellular domains 1-3 of human VEGFR2, as depicted in the shaded regions of FIG. 17B. In further embodiments, the ectodomain of a receptor can comprise a signal peptide sequence, as depicted in FIGS. 17B-C. In some embodiments, one CTP domain is added to the N-terminus of the ectodomain of a receptor. In other embodiments, two CTP domains are added to the N-terminus of the ectodomain of a receptor. In further embodiments, three CTP domains are added to the N-terminus of the ectodomain of a receptor. In some embodiments, one CTP domain is added to the C-terminus of the ectodomain of a receptor. In other embodiments, two CTP domains are added to the C-terminus of the ectodomain of a receptor. In further embodiments, three CTP domains are added to the C-terminus of the ectodomain of a receptor. In some embodiments, at least one CTP domain is added to the N-terminus and/or C-terminus of the ectodomain of a receptor. In other embodiments, at least two CTP domains are added to the N-terminus and/or C-terminus of the ectodomain of a receptor. In further embodiments, at least three CTP domains are added to the N-terminus and/or C-terminus of the ectodomain of a receptor. In some embodiments, the CTP domains are added in tandem.

In one embodiment, the antibody fragment is selected from the group consisting of an Fab′ fragment, an F(ab′)₂ fragment, an Fv fragment, an Fc fragment, a diabody, a single-chain variable fragment (scFv) and any combination thereof.

In one embodiment, the antibody fragment is an Fc fragment or a Fab′ fragment. In another embodiment, the antibody fragment is selected from an immunoglobulin isotype consisting of IgG, IgA, IgE, IgM, and IgD antibody. In another embodiment, the IgG immunoglobulin isotype is IgG1, IgG2, IgG3, or IgG4. In another embodiment, the IgG immunoglobulin isotype is IgG1.

In one embodiment, the CTP domain of the isolated polypeptides is glycosylated. In another embodiment, the antibody fragment is glycosylated. In another embodiment, the CTP domain is N-linked glycosylated, O-linked glycosylated, or a combination thereof. In another embodiment, the antibody fragment is N-linked glycosylated, O-linked glycosylated, or a combination thereof.

In one embodiment, the CTP domain of the isolated polypeptides is the CTP domain of human chorionic gonadotropin. In another embodiment, the CTP domain of the isolated polypeptides comprises at least about 95% identity to SEQ ID NO: 37. In another embodiment, the CTP domain comprises at least about 98% identity to SEQ ID NO: 37. In another embodiment, the CTP domain comprises at least about 99% identity to SEQ ID NO: 37. In another embodiment, the CTP domain of the isolated polypeptide is SEQ ID NO: 37. In another embodiment, the isolated polypeptide comprises SEQ ID NO: 31.

In another aspect, the invention provides a nucleic acid encoding the isolated polypeptide of the invention. In one embodiment, an isolated nucleic acid has the sequence in SEQ ID NO: 30.

In another aspect, an isolated fusion polypeptide molecule comprises the isolated polypeptide of the invention attached to a carboxy terminus of a second polypeptide. In one embodiment, the second polypeptide is a hormone, a receptor, a binding protein, or a soluble factor. In one embodiment, the receptor in the isolated fusion polypeptide molecule comprises extracellular domains 1-3 of human VEGFR1. In one embodiment, the receptor in the isolated fusion polypeptide molecule comprises extracellular domains 1-3 of human VEGFR2. In one embodiment, the isolated fusion polypeptide molecule comprises the amino acid sequence of SEQ ID NO: 33. In one embodiment, the isolated fusion polypeptide molecule comprises the amino acid sequence of SEQ ID NO: 35.

In one embodiment, the receptor is a Notch receptor. In one embodiment, the isolated fusion polypeptide molecule further comprises a Notch receptor ligand bound to the Notch receptor.

In another aspect, an isolated nucleic acid encodes the isolated fusion polypeptide molecule. In one embodiment, an isolated nucleic acid has the sequence in SEQ ID NO: 32 or 34.

In another aspect, a method of increasing a biological half-life of a polypeptide comprises:

(a) attaching a carboxy-terminal peptide (CTP) domain comprising at least about 90% identity to SEQ ID NO: 37, fused to an antibody fragment to the carboxy terminus of the polypeptide, thereby increasing the biological half-life of the polypeptide.

In another aspect, a method of stabilizing a polypeptide comprises:

(a) attaching a carboxy-terminal peptide (CTP) domain comprising at least about 90% identity to SEQ ID NO: 37 fused to an antibody fragment to the carboxy terminus of the polypeptide, thereby stabilizing the polypeptide.

In one aspect, the invention provides for methods of increasing a biological half-life of a polypeptide where the methods comprise attaching a carboxy-terminal peptide (CTP) domain comprising at least about 90% identity to SEQ ID NO: 37, fused to an ectodomain of a receptor, thereby increasing the biological half-life of the polypeptide.

In yet a further aspect, the invention provides for methods of stabilizing a polypeptide where the methods comprise attaching a carboxy-terminal peptide (CTP) domain comprising at least about 90% identity to SEQ ID NO: 37, fused to an ectodomain of a receptor, thereby increasing the biological half-life of the polypeptide.

In another aspect, the methods further comprise:

(b) further modifying the CTP domain to change the quantity or type of glycosylation.

In one embodiment of the methods, the polypeptide is an antibody, a fusion protein, a hormone, a receptor, a binding protein, or a soluble factor. In another embodiment, the antibody fragment is selected from the group consisting of an Fab′ fragment, an F(ab′)₂ fragment, an Fv fragment, an Fc fragment, a diabody, and a single-chain variable fragment (scFv). In one embodiment, the antibody fragment is an Fc fragment or a Fab′ fragment. In one embodiment, the antibody fragment is selected from an immunoglobulin isotype consisting of IgG, IgA, IgE, IgM, and IgD antibody.

In one embodiment of the methods, the IgG immunoglobulin isotype is IgG1, IgG2, IgG3, or IgG4. In one embodiment, the IgG immunoglobulin isotype is IgG1. In one embodiment, the CTP domain is glycosylated. In one embodiment, the antibody fragment is glycosylated. In one embodiment, the CTP domain is N-linked glycosylated, O-linked glycosylated, or a combination thereof. In one embodiment, the antibody fragment is N-linked glycosylated, O-linked glycosylated, or a combination thereof. In one embodiment, the CTP domain is the CTP domain of human chorionic gonadotropin.

In one embodiment of the methods, the CTP domain comprises at least about 95% identity to SEQ ID NO: 37. In one embodiment, the CTP domain comprises at least about 98% identity to SEQ ID NO: 37. In one embodiment, the CTP domain comprises at least about 99% identity to SEQ ID NO: 37. In one embodiment, the polypeptide comprises SEQ ID NO: 31.

In another aspect, a pharmaceutical composition comprises the isolated polypeptide and a pharmaceutically acceptable carrier. In one embodiment, the isolated polypeptide comprises a CTP domain having at least about 90% identity to SEQ ID NO: 37, and the CTP domain is fused to an antibody fragment. In one embodiment, the isolated polypeptide is encoded by the isolated nucleic acid having the sequence in SEQ ID NO: 30. In one embodiment, a pharmaceutical composition comprises the isolated fusion polypeptide molecule encoded by the nucleic acid having the sequence in SEQ ID NO: 32 or 34, and a pharmaceutically acceptable carrier.

In another aspect, a method of treating or preventing a disorder comprises administering an effective dose of the pharmaceutical composition to a subject in need of treatment or prevention of a disorder selected from the group consisting of a cancer, immune-related disorder, cardiovascular disease, obesity, diabetes, metabolic disorders and blindness. In one embodiment, the subject is a human.

These and other aspects and embodiments of the disclosure are illustrated and described herein.

### BRIEF DESCRIPTION OF THE FIGURES

The patent or application file contains at least one drawing executed in color. Copies of this patent or patent application publication with color drawing(s) will be provided by the United States Patent and Trademark Office upon request and payment of the necessary fee. See also Reeves et al., (2012) PLoS One. 7(4):e34862. doi: 10.1371 (including the accompanying Supplementary Information available in the on-line version of the manuscript available on the PLoS One web site), which is incorporated by reference in its entirety.

FIGS. 1A-D shows that Antxr2 is required for murine parturition. (1A) Genotyping of offspring from Antxr2+/− intercrosses revealed that Antxr2−/− mice are viable. (1B) RT-PCR analysis of Antxr2 transcript expression in Antxr2+/+ and Antxr2−/− MEFs. (1C) Fertility analysis of 6-week-old female mice revealed that 100% of Antxr2−/− mice are unable to give birth. (1D) Left panel— Analysis of uterine tissue isolated on GD18.5 showed that Antxr2−/− uterine horns lack muscle striations (black arrows in right panel) and exhibit poor uterine tone (asterisk). Right panel—blow ups of boxed areas in left panel to highlight lack of striations in Antxr2−/− uterine horns.

FIGS. 1E-F shows that Antxr2 is required for murine parturition. (1E) H&E staining and immunofluorescence for the smooth muscle cell marker, α-SMA, demonstrated that the circular and longitudinal myometrial cell layers are disrupted in GD18.5 Antxr2−/− uterine tissue (Ep, endometrial epithelium; LM, longitudinal myometrium; CM, circular myometrium). Arrows point to remaining bundles of myometrial cells in Antxr2−/− uterus. I mmunostaining for Antxr2 revealed that the protein is expressed on myometrial cells in Antxr2+/+ tissue (brown stain in image). Asterisks indicate bundles of myometrial cells that are negative for Antxr2 expression in Antxr2−/− tissue. Masson's Trichrome staining demonstrated high collagen content (blue color in image) in area of Antxr2−/− uterus where myometrial cell layers are usually present. (1F) Masson's Trichrome staining of GD18.5 cervical tissue revealed a dense collagen network (blue color in image) in Antxr2−/− cervices. Scale bars, 150 μm.

FIG. 2A shows reproductive tracts isolated from aged nulliparous Antxr2−/− female mice exhibit an altered morphology with severe fibrosis. Comparison of reproductive tracts isolated from Antxr2+/+ and Antxr2−/− mice at one month and three months of age. Sexually mature, (three-month-old) Antxr2−/− uteri are shortened and thickened compared to Antxr2+/+. Tissue lying between the uterine horns in the three-month images is the colon.

FIG. 2B shows reproductive tracts isolated from aged nulliparous Antxr2−/− female mice exhibit an altered morphology with severe fibrosis. Masson's Trichrome staining of reproductive tracts demonstrated that there is progressive collagen fibrosis (blue color in image) in the uterus as Antxr2−/− mice age. Ep, endometrial epithelium; S, stroma; M, myometrium; GE, glandular epithelium. Scale bars, 150 μm.

FIG. 2C shows reproductive tracts isolated from aged nulliparous Antxr2−/− female mice exhibit an altered morphology with severe fibrosis. Masson's Trichrome staining of fifteen-month-old cervical tissue demonstrated that Antxr2−/− mice have cervices with increased collagen content (blue color in image). Bottom panel is boxed image at higher magnification. Top panel scale bars, 400 μm. Bottom panel scale bars, 150 μm. Three mice of each genotype (Antxr2+/+ and Antxr2−/−) were evaluated for each time point. Representative images for each time point are shown.

FIGS. 3A-B shows the myometrium is disrupted in aged nulliparous Antxr2−/− reproductive tracts. (3A) Immunofluorescence for α-SMA (red in image) demonstrated well-defined circular and longitudinal myometrial cell layers that were beginning to disassociate in 6.5 week Antxr2−/− tissue. Space between myometrial cell bundles is indicated by asterisks in the Antxr2−/− tissue. (3B) α-SMA (red in image) staining of three-month-old tissue demonstrated that the disassociation between the myometrial layers had progressed in Antxr2−/− uterine and cervical tissue. In the uterus, the dispersal was such that the remaining muscle bundles could not be captured together in the same photographic frame. Arrows indicate where two images were placed together in order to demonstrate the distance between the longitudinal and circular myometiral cell layers in the Antxr2−/− uterus. DAPI (blue color in image) is used for nuclear staining. Scale bars, 150 μm.

FIGS. 4A-B show uterine fibrosis in aged nulliparous Antxr2−/− mice is accompanied by atypical vasculature and inflammation. (4A) CD31 immunostaining (brown color in image) of three-month-old reproductive tracts reveal atypical/open blood vessels (arrows) throughout the Antxr2−/− uterus and cervix. Boxed areas are blown up to highlight vasculature. Ep, endometrial epithelium; CM, circular myometrium; LM, longitudinal myometrium. Scale bars on uterus photos, 200 μm. Scale bars on cervix photos, 150 μm. (4B) Coimmunofluoresence for blood endothelial cell marker, endomucin (green color in image), and lymphatic endothelial cell marker, lyve-1 (red color in image) on three-month-old uterine tissue. DAPI (blue color in image) is used for nuclear staining. Lymphatic vessels (arrowheads) in Antxr2−/− tissue are enlarged. Scale bar, 100 μm.

FIG. 4C show uterine fibrosis in aged nulliparous Antxr2−/− mice is accompanied by atypical vasculature and inflammation Immunofluorescent staining for macrophage marker, F4/80 (red color in image), revealed an increased inflammatory response in three-month-old and ten-month-old Antxr2−/− uterine tissue. DAPI (blue color in image) is used for nuclear staining. Scale bars, 200 μm.

FIGS. 5A-C shows Increased collagen and fibronectin content in aged nulliparous Antxr2−/− uterine tissue. (5A) Immunofluorescent staining of uterine tissue isolated from six-month-old mice demonstrated increased type I collagen, type IV collagen and fibronectin deposition in the Antxr2−/− tissue. DAPI (blue color in image) was used for nuclear staining. Negative controls demonstrate specificity of the antibodies. Ep, endometrial epithelium; S, stroma; M, myometrium; GE, glandular epithelium. Scale bars, 150 μm. (5B) Uterine lysates from six-month-old mice, Antxr2+/+ (n=2) and Antxr2−/− (n=2), were immunoblotted for type I collagen (precursor type I collagen indicated by arrow and mature type I collagen indicated by arrowhead), type VI collagen and fibronectin. Alpha tubulin is shown as a loading control. (5C) Densitometric analysis of blots in panel B presented as relative levels of designated ECM protein normalized to respective alpha tubulin. The mean±the standard deviation are represented, *=P<0.05.

FIGS. 6A-C shows reduced MMP2 activity in Antxr2 deficient tissue and cells. (6A) Uterine lysates from six-month-old mice, Antxr2+/+ (n=2) and Antxr2−/− (n=2), were immunoblotted for MMP2 and demonstrated that there are reduced levels of active MMP2 in the Antxr2−/− tissue (P=the pro form of MMP2, I=the intermediate form of MMP2 and A=the active form of MMP2). Alpha tubulin was used as a loading control. (6B) Gelatin zymography revealed reduced levels of active MMP2 in conditioned medium from Antxr2−/− MEFs. A representative of two independent experiments is shown. For each experiment, samples were run in duplicate. The graph below the zymogram gel represents the relative levels of active to total MMP-2 (pro+intermediate+active) as quantified by densitometry and shows the mean±standard deviation (P=0.06). (6C) Conditioned medium from HUVEC cell lines with knock down of ANTXR2 expression (shANTXR2 HUVEC) had reduced MMP2 activity as determined by gelatin zymography. The vertical dotted line reflects the fact that different parts of the same gel were placed next to each other in the figure for ease of comparison. A representative of two independent experiments is shown. For each experiment, the samples were run in quadruplicate. The graph below the zymogram gel represents the relative levels of active to total MMP-2 (pro+intermediate+active) as quantified by densitometry and shows the mean±standard deviation (P<0.05). The bottom panel is a histogram from flow cytometry analysis of retrovirally-infected HUVEC scrambled shRNA (control) or ANTXR2 shRNA (shANTXR2) cell lines. The histogram shows decreased ANTXR2 expression at the cell surface of the shANTXR2 HUVEC line.

FIGS. 7A-C show ANTXR2 positively regulate MT1-MMP activity. (7A) Zymographic analysis of conditioned medium from 293T cells transfected with empty vector (lane 1), MT1-MMP (lane 2), MT1-ΔC (lane 3), ANTXR2-GFP (lane 4), ANTXR2-vWF (lane 5), MT1-MMP and ANTXR2-GFP (lane 6), MT1-MMP and ANTXR2-vWF (lane 7), MT1-ΔC and ANTXR2-GFP (lane 8), or MT1-ΔC and ANTXR2-vWF (lane 9) revealed that co-expression of either MT1-MMP or MT1-ΔC and ANTXR2-GFP or ANTXR2-vWF led to enhanced pro MMP2 activation over expression of either MT1-MMP or MT1-ΔC alone. (7B) Table under the zymogram in panel A represents densitometric quantification of the pro and active MMP2 bands. Numbers are in percentile of relative intensity in relation to the empty vector control, lane 1. (7C) Immunoblots for MT1-MMP, ANTXR2-GFP, ANTXR2-vWF and Tubulin from the 293T cell lysates corresponding to the zymography experiment in panel A.

FIGS. 7D-F show ANTXR2 positively regulate MT1-MMP activity. (7D) Zymographic analysis of conditioned medium from 293T cells co-expressing MT1-MMP and varying concentrations of ANTXR2-GFP or ANTXR2-vWF revealed that MT1-MMP activity is dependent on ANTXR2 expression levels. (7E) Table under the zymogram represents densitometric quantification of the pro and active MMP2 bands. Numbers are in percentile of relative intensity in relation to the empty vector control, lane 1. (7F) Immunoblots for MT1-MMP, ANTXR2-GFP, ANTXR2-vWF and Tubulin from the 293T cell lysates corresponding to the zymography experiment in panel D. For each zymogram panel, a representative of two independent experiments is shown.

FIGS. 8A-B shows ANTXR2 and MT1-MMP colocalize and are found in complex. (8A) Coimmunofluorescence for Mt1-mmp (green color in image) and Antxr2 (red color in image) on Antxr2+/+ and Antxr2−/− MEFs demonstrate that MT1-MMP and ANTXR2 colocalize at the cell surface (orange color in image). DAPI (blue color in image) is used for nuclear staining Scale bars, 5 μm. (8B) 293T cells were transfected with empty vector, MT1-MMP, ANTXR2-GFP or MT1-MMP and ANTXR2-GFP. Cell lysates were immunoprecipitated with antibody against ANTXR2 followed by western blotting to detect MT1-MMP. The coimmunoprecipitation revealed that ANTXR2 and MT1-MMP are found together in complex. A representative of two independent experiments is shown.

FIG. 9A is a diagram of the first three exons of the Antxr2 wild-type allele, the targeting vector, the triloxP allele in which a loxP site (arrowhead) was inserted upstream of exon 1 and a floxed Neo cassette was inserted within intron 1, and the knockout allele. The dark grey box under exon 3 indicates the external probe used for Southern Blot analysis. The grey arrows represent PCR primers used to detect the single loxP site upstream of exon 1.

FIG. 9B (Upper panel) shows a Southern blot analysis of properly targeted ES cells. The wild-type allele is 8.174 Kb and the TriloxP allele is 4.4 kb. Lower panel—PCR analysis on gDNA to detect the loxP site upstream of exon 1. The 672 bp band represents the loxP allele and the 600 bp band represents the wild-type allele.

FIG. 9C are photomicrographs showing Masson's trichrome staining of Antxr2+/+ and Antxr2−/− ovaries isolated on GD18.5 that did not reveal differences in collagen content. CL, corpeus luteum. Scale bars, 400 μm.

FIG. 9D is a bar graph of an ELISA analysis of sera from Antxr2+/+ and Antxr2−/− mice on GD15.5 and 18.5, which revealed that serum progesterone levels declined as the animals approached term (GD19). Sera from three Antxr2+/+ mice and five Antxr2−/− mice were analyzed. The graph presents the mean±the standard deviation. P>0.2 when comparing Antxr2+/+ and Antxr2−/− progesterone levels at either time point.

FIG. 10 are photomicrographs showing Masson's Trichrome staining, which did not reveal differences in collagen content between Antxr2+/+ and Antxr2−/− ovaries isolated from three-month-old animals or six-month-old animals. 3 month scale bars, 150 μm. 6 month scale bars, 200 μm.

FIG. 11 are photomicrographs Immunofluorescent staining of uterine tissue isolated from ten-month-old mice demonstrated increased type I collagen (green color in image), type VI collagen (red color in image) and fibronectin (red color in image) deposition in the Antxr2−/− tissue. L, uterine lumen. DAPI (blue color in image) is used for nuclear staining Scale bars, 150 μm.

FIGS. 12A-B shows DNA gels (12A) Zymographic analysis of conditioned medium from 293T cells transfected with empty vector (lane 1), MT1-MMP (lane 2), ANTXR1-GFP (lane 3), ANTXR1-vWF (lane 4), ANTXR2-GFP (lane 5), MT1-MMP and ANTXR1-GFP (lane 6), MT1-MMP and ANTXR1-vWF (lane 7), MT1-MMP and ANTXR2-GFP (lane 8), MT1-ΔC (lane 9), MT1-ΔC and ANTXR1-GFP (lane 10), or MT1-ΔC and ANTXR1-vWF (lane 11), or MT1-ΔC and ANTXR2-GFP (lane 12) revealed that co-expression of either MT1-MMP or MT1-ΔC and ANTXR1-GFP or ANTXR1-vWF led to enhanced pro MMP2 activation over expression of either MT1-MMP or MT1-ΔC alone. Table under the zymogram represents densitometric quantification of the pro and active MMP2 bands. Numbers are in percentile of relative intensity in relation to the empty vector control, lane 1. (12B) Zymographic analysis of conditioned medium from 293T cells co-expressing MT1-ΔC and varying concentrations of ANTXR2-GFP or ANTXR2-vWF revealed that MT1-ΔC activity is dependent on ANTXR2 expression levels. Table under the zymogram represents densitometric quantification of the pro and active MMP2 bands. Numbers are in percentile of relative intensity in relation to the empty vector control, lane 1. For each zymogram panel, a representative of two independent experiments is shown.

FIG. 13 shows Mammary glands from 15-month-old Antxr2-1− mice exhibit severe fibrosis characterized by increased collagen deposition around ducts.

FIG. 14 is a blot showing MMP activation is reduced in Antxr2−/− MEFs.

FIGS. 15A-B shows photomicrographs (15A) and western blots (15B) depicting that MTI-MMP and ANTXR2 physically interact.

FIG. 16 is a blot showing that coexpression of MT1-MMP and ANTXR2 enhances the activation of MMP2.

FIG. 17A is a schematic representation of “durable Fc,” containing the Fc portion of human IgG1 fused to the CTP domain of the beta subunit of human chorionic gonadotrophin protein (hCG). This durable Fc polypeptide comprises SEQ ID NO: 31.

FIG. 17B is a schematic representation of VEGFR-1 containing the first three Ig extracellular domains fused to “durable Fc,” which is Fc fused to the CTP domain. This VEGFR1-FcCTP polypeptide comprises SEQ ID NO: 33.

FIG. 17C is a schematic representation of VEGFR-2 containing the first three Ig extracellular domains fused to “durable Fc,” which is Fc fused to the CTP domain. This VEGFR2-FcCTP polypeptide comprises SEQ ID NO: 35.

FIG. 18A is a Western blot showing the detection of Fc and FcCTP expression in 293 T-cells transfected with pcDNA3-FcCTP, pAdlox-FcCTP, and pAdlox-Fc. Protein standards are depicted on the left as kD. Anti-human Fc antibody was used for detection.

FIG. 18B is a Western blot to detect FcCTP expression in 293 cells transduced with three Ad-FcCTP positive plaques. Protein standards are depicted on the left in kD. Anti-human Fc HRP was used for detection.

### DETAILED DESCRIPTION OF THE INVENTION

### Abbreviations and Definitions

All scientific and technical terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent or later-developed techniques which would be apparent to one of skill in the art. In addition, to more clearly and concisely describe the subject matter which is the invention, the following definitions are provided for certain terms which are used in the specification and claims.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

The terms “treating”, “treatment” and the like, are used herein to include the management and care of a subject or patient (e.g., a mammal, such as a human, dog, or cat) for the purpose of combating a disease, condition, or disorder. The terms include the administration of a composition of the present invention to prevent the onset of the symptoms or complications, alleviate the symptoms or complications, or eliminate the disease, condition, or disorder. Any alleviation of any undesired signs or symptoms of a disease, disorder, or condition, to any extent, can be considered treatment. The terms also mean affecting a subject, tissue or cell to obtain a desired pharmacologic and/or physiologic effect. The effect may be prophylactic in terms of completely or partially preventing a disorder, disease, or condition. “Treating” as used herein covers any treatment of, or prevention of a disorder, disease, or condition in a subject, and includes: (a) preventing the disorder from occurring in a subject that may be predisposed to the disorder, but has not yet been diagnosed as having it; (b) inhibiting the disorder, i.e., arresting its development; or (c) relieving or ameliorating the disorder, i.e., cause regression of the disorder. In one embodiment, the subject is an animal. In another embodiment, the subject is an animal that has or is diagnosed with a disease, condition, or disorder. In one embodiment, the subject is a human. In other embodiments, the subject is a mammal. In one embodiment, the subject is a dog. In another embodiment, the subject is a cat. In some embodiments, the subject is a rodent, such as a mouse or a rat. In some embodiments, the subject is a cow, pig, sheep, goat, cat, horse, dog, and/or any other species of animal used as livestock or kept as pets.

The term “or” is used herein to mean, and is used interchangeably with, the term “and/or,” unless context clearly indicates otherwise.

The term “subject,” as used herein, means any subject for whom diagnosis, prognosis, or therapy is desired. For example, a subject can be a mammal, e.g., a human or non-human primate (such as an ape, monkey, orangutan, or chimpanzee), a dog, cat, guinea pig, rabbit, rat, mouse, horse, cattle, or cow.

The term “biological half-life” is the time required for the activity of a substance taken into the body to lose one half its initial pharmacologic, physiologic, or biologic activity.

The ectodomain refers to a protein region of a membrane protein that extends into the extracellular space. Ectodomain regions initiate contact with the cell surface, which can lead to signal transduction events.

As would be apparent to one of ordinary skill in the art, any method or composition described herein can be implemented with respect to any other method or composition described herein.

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

Fibrotic Diseases

Fibrosis is the formation of excess fibrous connective tissue in an organ or tissue in a reparative or reactive process. Fibrosis affects nearly every tissue and organ in the body. Fibrosis is a pathological feature of most chronic inflammatory diseases. Fibrosis, if highly progressive, can lead to organ malfunction and death of a subject. For example, this is seen in end-stage liver disease, kidney disease, idiopathic pulmonary fibrosis (IPF) and heart failure (see Wynn et al., (2012) Nat Med. 18(7): 1028-1040).

MT1-MMP expression is associated with multiple pathophysiological conditions and has been demonstrated to have roles in tumor progression and metastasis. Current MMP inhibitors target a conserved active site in the catalytic domain of the protein and, as a result, repress the proteolytic activity of multiple MMPs instead of MT1-MMP alone. Additionally, loss of proper MT1-MMP activation may contribute to connective tissue disorders, which are characterized by abnormal accumulation of extracellular matrix proteins. In these cases, it would be useful to have a way to selectively enhance MT1-MMP activity, for example by administering to a subject a ANTXR molecule of the invention. The fusion polypeptides of the invention, for example ANTXR, ANTXR Extracellular Domain constructs, and ANTXR vWF constructs, comprise various domains (e.g., either Fc, CTP Fc-CTP, or a combination thereof) as described herein. In some embodiments, the domains can serve as tags. In other embodiments, testing them on cells and in animal models for their ability to activate or inhibit MT1-MMP based proteolysis can be assessed.

The present invention provides methods for treating fibrotic diseases in a subject. In one embodiment, the method comprises administering an ANTXR molecule to the subject. In another embodiment, the fibrotic disease comprises chronic autoimmune diseases, including but not limited to scleroderma, rheumatoid arthritis, Crohn's disease, Type 1 diabetes mellitus, ulcerative colitis, myelofibrosis, plaque psoriasis, and systemic lupus erythematosus. In a further embodiment, the fibrotic disease comprises an inflammatory disease of the digestive system. In a further embodiment, the inflammatory disease of the digestive system includes, but is not limited to, esophagitis, inflammatory bowel disease, Crohn's disease, ulcerative colitis, colitis, irritable bowel syndrome, celiac disease, and gastritis. In another embodiment, the fibrotic disease comprises arthritis, inflammatory fibrosis, systemic hyalinosis, juvenile hyaline fibromatosis, infantile systemic hyalinosis, Barrett syndrome, wound healing disorder, or celiac disease.

The present invention is further directed to methods of decreasing fibrosis in a tissue of a subject, for example a subject having a severe tissue injury, or is subjected to a repetitive tissue injury, or if the wound-healing response becomes dysregulated. In one embodiment, the fibrosis results from severely or repetitively damaged tissue. In another embodiment, the method comprises administering an ANTXR molecule to the subject. In one embodiment, the fibrosis results in target tissues from chronic autoimmune diseases, including but not limited to scleroderma, rheumatoid arthritis, Crohn's disease, Type 1 diabetes mellitus, ulcerative colitis, myelofibrosis, plaque psoriasis, and systemic lupus erythematosus. In a further embodiment, fibrosis results from an inflammatory disease of the digestive system. In a further embodiment, the inflammatory disease of the digestive system includes, but is not limited to, esophagitis, inflammatory bowel disease, Crohn's disease, ulcerative colitis, colitis, irritable bowel syndrome, celiac disease, and gastritis. In another embodiment, the fibrosis results from a subject afflicted with arthritis, inflammatory fibrosis, systemic hyalinosis, juvenile hyaline fibromatosis, infantile systemic hyalinosis, Barrett syndrome, wound healing disorder, or celiac disease.

In some embodiments, the subject is already suspected to have a fibrotic disease. In other embodiments, the subject is being treated for a fibrotic disease, before being treated according to the methods of the invention. In other embodiments, the subject is not being treated for a fibrotic disease, before being treated according to the methods of the invention.

Fibrosis can be measured in a variety of ways, known to one of skill in the art including, but not limited to, tissue biopsies, and qRT-PCR assays described by Kauschke et al., in Anal Biochem. (1999) 275(2):131-140, which is incorporated by reference in its entirety.

Epithelial Cancer

The present invention provides methods for treating an epithelial cancer in a subject comprising administering an ANTXR molecule. An epithelial cancer is a malignant neoplasm originating from the epithelium, for example a carcinoma. Non-limiting examples of epithelial cancers include: colon cancer, liver cancer, breast cancer, pancreatic cancer, ovarian cancer, kidney cancer, lung cancer, colorectal cancer, renal cancer, bladder cancer, testicular cancer, uterine cancer, cervical cancer, gastrointestinal cancer (such as esophageal cancer, stomach cancer, small intestine cancer, large intestine cancer, colon cancer, rectal cancer), prostate cancer, and uterine cancer. In one embodiment, the epithelial cancer is renal cell carcinoma, progressive lung adenocarcinoma, hepatoma, adenocarcinoma, pancreatic cancer, ductal carcinoma, lobular, carcinoma, head and neck carcinoma, thyroid carcinoma, squamous cell carcinoma, basal cell carcinoma, colon carcinoma, basal cell carcinoma, sweat gland carcinoma, sebaceous gland carcinoma, papillary carcinoma, papillary adenocarcinomas, cystadenocarcinoma, bronchogenic carcinoma, bile duct carcinoma, choriocarcinoma, embryonal carcinoma, lung carcinoma, epithelial carcinoma, small cell lung carcinoma, bladder carcinoma, or medullary carcinoma. In another embodiment, the epithelial cancer is breast cancer, ovarian cancer, prostate cancer, lung cancer, esophageal cancer, stomach cancer, small intestine cancer, large intestine cancer, or colon cancer. In some embodiments, the epithelial cancer is breast cancer or ovarian cancer.

In some embodiments, the subject is already suspected to have an epithelial cancer. In other embodiments, the subject is being treated for an epithelial cancer, before being treated according to the methods of the invention. In other embodiments, the subject is not being treated for an epithelial cancer, before being treated according to the methods of the invention.

For example, mammographically dense breast tissue, which is characterized by increases in the extracellular matrix protein, collagen, is a risk factor for developing breast cancer. On the other hand, myoepithelial cells that surround mammary ducts and aveoli are thought to have a role in tumor and metastasis suppression due to the fact that they form a natural barrier between the luminal epithelial cells (the cells from which tumor form) and the surrounding environment. Myoepithelial cells also secrete proteins that limit cancer growth, invasiveness and blood vessel formation. Nevertheless, the role of both the extracellular matrix and myoepithelial cells during tumor progression remains poorly defined.

The present invention provides methods for decreasing or preventing in a subject tumor cell invasion into a tissue free from tumor cells comprising administering to a subject an ANTXR molecule. In one embodiment, the tumor is a colon tumor, liver tumor, breast tumor, pancreatic tumor, ovarian tumor, kidney tumor, lung tumor, colorectal tumor, renal tumor, bladder tumor, testicular tumor, uterine tumor, cervical tumor, gastrointestinal tumor (such as an esophageal tumor, stomach tumor, small intestine tumor, large intestine tumor, colon tumor, rectal tumor), prostate tumor, or uterine tumor. In another embodiment, the tumor is a breast tumor, an ovarian tumor, a prostate tumor, a lung tumor, an esophageal tumor, a stomach tumor, a small intestine tumor, a large intestine tumor, or a colon tumor. In some embodiments, the tumor is a breast tumor or an ovarian tumor. Tumor cell invasion can be measured in a variety of ways, known to one of skill in the art. For example, tumor cell invasion can be measured by monitoring and measuring the amount of circulating tumor cells in the blood of a subject. Tumor cell invasion can be measured in a variety of ways, known to one of skill in the art including, but not limited to 2D- and 3D culturing assays and cell adhesion matrix (CAM) assays (see U.S. Patent Application Publication Nos. 20130034558 and 20130022624, Fan et al., (2009) Gynecol Oncol. 112(1): 185-191), as well as those described by Shaw in Methods Mol Biol. (2005) 294:97-105, each of which are hereby incorporated by reference in their entireties. Tumor cell invasion can be measured in a subject according to methods practiced in the art, positron emission tomography and computed tomography (PET-CT), single-photon emission computed tomography (SPECT-CT), magnetic resonance spectroscopy (MR), X-ray computed tomography (CT), and molecular imaging, as well as lymph node biopsies in order to assess if the tumor has spread from a primary site.

The present invention provides methods for decreasing or preventing cancer metastasis in a subject comprising administering to a subject an ANTXR molecule. In one embodiment, the cancer is an epithelial cancer. Non-limiting examples of epithelial cancers include: colon cancer, liver cancer, breast cancer, pancreatic cancer, ovarian cancer, kidney cancer, lung cancer, colorectal cancer, renal cancer, bladder cancer, testicular cancer, uterine cancer, cervical cancer, gastrointestinal cancer (such as esophageal cancer, stomach cancer, small intestine cancer, large intestine cancer, colon cancer, rectal cancer), prostate cancer, and uterine cancer. In one embodiment, the epithelial cancer is renal cell carcinoma, progressive lung adenocarcinoma, hepatoma, adenocarcinoma, pancreatic cancer, ductal carcinoma, lobular, carcinoma, head and neck carcinoma, thyroid carcinoma, squamous cell carcinoma, basal cell carcinoma, colon carcinoma, basal cell carcinoma, sweat gland carcinoma, sebaceous gland carcinoma, papillary carcinoma, papillary adenocarcinomas, cystadenocarcinoma, bronchogenic carcinoma, bile duct carcinoma, choriocarcinoma, embryonal carcinoma, lung carcinoma, epithelial carcinoma, small cell lung carcinoma, bladder carcinoma, or medullary carcinoma. In another embodiment, the cancer is breast cancer, ovarian cancer, prostate cancer, lung cancer, esophageal cancer, stomach cancer, small intestine cancer, large intestine cancer, or colon cancer. In some embodiments, the cancer is breast cancer or ovarian cancer. Metastasis can be measured in a variety of ways, known to one of skill in the art. For example, metastasis can be measured by conducting a biopsy, monitoring and measuring the amount of circulating tumor cells in the blood of a subject, as well as detecting the presence of tumor markers for metastatic cancer in the blood of a subject. Metastasis can be measured in a variety of ways, known to one of skill in the art including, but not limited to, positron emission tomography and computed tomography (PET-CT), single-photon emission computed tomography (SPECT-CT), magnetic resonance spectroscopy (MR), X-ray computed tomography (CT), and molecular imaging. Metastasis can also be measure in a subject by way of lymph node biopsies in order to assess if the tumor has spread from a primary site See also, U.S. Patent Application Publication Nos. 20130034558 and 20130022624, each of which are hereby incorporated by reference in their entireties.

The present invention provides methods for decreasing or preventing angiogenesis in a tumor. The method comprises delivering an ANTXR molecule to a cell in a tumor; and expressing in the cell of the tumor the ANTXR molecule. In one embodiment, the tumor is a colon tumor, liver tumor, breast tumor, pancreatic tumor, ovarian tumor, kidney tumor, lung tumor, colorectal tumor, renal tumor, bladder tumor, testicular tumor, uterine tumor, cervical tumor, gastrointestinal tumor (such as an esophageal tumor, stomach tumor, small intestine tumor, large intestine tumor, colon tumor, rectal tumor), prostate tumor, or uterine tumor. In another embodiment, the tumor is a breast tumor, an ovarian tumor, a prostate tumor, a lung tumor, an esophageal tumor, a stomach tumor, a small intestine tumor, a large intestine tumor, or a colon tumor. In some embodiments, the tumor is a breast tumor or an ovarian tumor. Angiogenesis can be measured in a variety of ways, known to one of skill in the art. For example, angiogenesis can be measured by published or commercially available assays that are practiced in the art. These include, but are not limited to, the in vivo Matrigel plug and corneal neovascularization assays, the in vivo/in vitro chick chorioallantoic membrane (CAM) assay, the in vitro cellular (proliferation, migration, tube formation) assays, the in vitro organotypic (aortic ring) assays, the chick aortic arch assay, and the Matrigel sponge assays (see Jensen et al., (2009) Curr Mol Med. 9(8):982-91;Staton et al., (2009) Int J Exp Pathol. 90(3):195-221; Auerbach et al., (2003) Clin Chem. 49(1):32-40, and U.S. Pat. No. 6,444,434, each of which are hereby incorporated by reference in their entireties).

The present invention also provides methods for decreasing tumor growth in a subject comprising administering an ANTXR molecule. In one embodiment, the tumor is an epithelial tumor. Tumor growth can be measured in a variety of ways, known to one of skill in the art. For example, tumor growth can be measured by measuring the tumor volume over time. Tumor volume can be measured in a variety of ways, known to one of skill in the art including, but not limited to, positron emission tomography and computed tomography (PET-CT), single-photon emission computed tomography (SPECT-CT), magnetic resonance spectroscopy (MR), X-ray computed tomography (CT), and molecular imaging.

Anthrax Toxin Receptor (ANTXR) Molecules

The Anthrax Toxin Receptor genes, Anthrax Toxin Receptor 1 (ANTXR1) and Anthrax Toxin Receptor 2 (ANTXR2) encode highly homologous proteins believed to function as cell surface receptors and contain an extracellular von Willebrand Factor Type A (vWFA) domain, a transmembrane domain and a cytosolic tail with putative signaling motifs (S1, S2). vWFA domains are known to facilitate protein-protein interactions when found on extracellular matrix (ECM) constituents or cell adhesion proteins like α-integrin subunits and constitute ligand binding sites on ANTXRs (S3, S4). In vitro assays have demonstrated that both ANTXR1 and ANTXR2 interact with ECM proteins. Type I and VI collagens may be endogenous ligands for ANTXR1 and type IV collagen and laminin may be endogenous ligands for ANTXR2 (S5-S7). Despite these insights, a concerted model for ANTX Receptor action is undefined and their precise functions remains to be elucidated.

The proteins also bind anthrax toxin, however, the ANTXR genes were originally identified based on expression in endothelium. In one embodiment, the ANTXR genes have a physiological role in angiogenesis. The inventors demonstrated that ANTXR2 is required for angiogenic processes such as endothelial proliferation and capillary-like network formation in vitro [6]. Similarly, ANTXR1 has been demonstrated to be important for endothelial cell migration and network formation [4,7]. Despite these studies, the physiological function of the ANTXR proteins remains to be fully elucidated.

A role has been proposed for ANTXR2 in ECM homeostasis [5,8] based on ANTXR2 protein structure and ECM binding capability. Antxr1−/− mice exhibit defects in ECM deposition in organs such as the ovaries, uterus, skin, teeth and skull [9]. Furthermore, a rare human disease is caused by mutations in the ANTXR2 gene. Systemic Hyalinosis is an autosomal recessive disease that encompasses two syndromes, infantile systemic hyalinosis (ISH) and juvenile hyaline fibromatosis (JHF) [8,10,11]. ISH and JHF are characterized by gingival hypertrophy, progressive joint contractures, osteolysis, osteoporosis, recurrent subcutaneous fibromas, and hyaline depositions which are thought to form as a result of abnormal collagen and glycosaminoglycan accumulation [12].

As used herein, an “anthrax toxin receptor molecule” (ANTXR) refers to an anthrax toxin receptor protein, or a fragment thereof. An “anthrax toxin receptor molecule” can also refer to a nucleic acid (including, for example, genomic DNA, complementary DNA (cDNA), synthetic DNA, as well as any form of corresponding RNA) which encodes a polypeptide corresponding to an anthrax toxin receptor protein, or fragment thereof. For example, an anthrax toxin receptor molecule can include ANTXR1 (e.g., comprising the amino acid sequence shown in SEQ ID NO: 18 or 24, or comprising the nucleic acid sequence shown in SEQ ID NO: 19 or 23), or ANTXR2 (e.g., comprising the amino acid sequence shown in SEQ ID NO: 20 or 26, or comprising the nucleic acid sequence shown in SEQ ID NO: 21 or 27).

For example, an anthrax toxin receptor molecule can be encoded by a recombinant nucleic acid encoding an anthrax toxin receptor protein, or fragment thereof. The anthrax toxin receptor molecules of the invention can be obtained from various sources and can be produced according to various techniques known in the art. For example, a nucleic acid that encodes an anthrax toxin receptor molecule can be obtained by screening DNA libraries, or by amplification from a natural source. An anthrax toxin receptor molecule can include a fragment or portion of an anthrax toxin receptor molecule. For example, the fragment of the anthrax toxin receptor molecule can be the extracellular domain of an anthrax toxin receptor molecule, or the vWF domain of an anthrax toxin receptor molecule (see SEQ ID NO: 18, 20, 22, 24, 26, or 28). An anthrax toxin receptor molecule can include a variant of the above described examples, such as a fragment thereof. Such a variant can comprise a naturally-occurring variant due to allelic variations between individuals (e.g., polymorphisms), mutated alleles, or alternative splicing forms. In one embodiment, an anthrax toxin receptor molecule is encoded by a nucleic acid variant of the nucleic acid having the sequence shown in SEQ ID NOS: 19, 21, 23, or 27 wherein the variant has a nucleotide sequence identity to SEQ ID NOS: 19, 21, 23, or 27 of about 70%, about 75%, about 80%, about 85%, about 90%, about 91%, about 92%, about 93%, about 94%, about 95%, about 96%, about 97%, about 98%, or about 99%. In another embodiment, a variant of the anthrax toxin receptor protein comprises a protein or polypeptide encoded by an anthrax toxin receptor nucleic acid sequence, such as the sequence shown in SEQ ID NOS: 19, 21, 23, or 27. An anthrax toxin receptor molecule can also include an anthrax toxin receptor protein, or fragment thereof, that is modified by the addition of a carboxy-terminal peptide (CTP) domain, a Fc domain, an Fc-CTP domain, or a combination thereof, for increased stability.

A further description of the Fc, CTP, and Fc-CTP tagging is provided herein. The constant region or Fc domain of antibodies have been used extensively in therapeutics entities. They are present on most antibody based therapeutics or therapeutics that use the Fc domain as a tag. In one embodiment, the invention entails the addition of a peptide domain to promote glycosylation and stabilization of Fc or Fab Fragments when fused to the vWF or extracellular domain (ECD) of Anthrax Toxin Receptors (such as ANTXR1 and ANTXR2). For example, the CTP (carboxy-terminal peptide) domain of the beta-subunit human chorionic gonadotropin (hCG) is fused in frame to the terminus of human Fc and attached to the vWF or ECD of ANTXRs. This CTP domain has been shown to confer stability and long half-life of proteins in the circulation. The CTP domain can also be in modified forms to change either the quantity of glycans added during glycosylations or the type of glycosylation events that occur.

SEQ ID NO: 1 depicts the amino acid sequence of a Fc domain:

| | |
|---|---|
| 1 | MWGWKCLLFW AVLVTATLCT ARPAPTLPEQ AQQSTRADLG PGEPKSCDKT HTCPPCPAPE |
| 61 | LLGGPSVFLF PPKPKDTLMI SRTPEVTCVV VDVSHEDPEV KFNWYVDGVE VHNAKTKPRE |
| 121 | EQYNSTYRVV SVLTVLHQDW LNGKEYKCKV SNKALPAPIE KTISKAKGQP REPQVYTLPP |
| 181 | SRDELTKNQV SLTCLVKGFY PSDIAVEWES NGQPENNYKT TPPVLDSDGS FFLYSKLTVD |
| 241 | KSRWQQGNVF SCSVMHEALH NHYTQKSLSL SPGK |

SEQ ID NO: 2 depicts the nucleic acid sequence encoding a Fc domain:

| | |
|---|---|
| 1 | atgtggggct ggaagtgcct cctcttctgg gctgtgctgg tcacagccac tctctgcact |
| 61 | gccaggccag ccccaacctt gcccgaacaa gctcagcagt cgacgcgcgc agatctgggc |
| 121 | ccgggcgagc ccaaatcttg tgacaaaact cacacatgcc caccgtgccc agcacctgaa |
| 181 | ctcctggggg gaccgtcagt cttcctcttc cccccaaaac ccaaggacac cctcatgatc |
| 241 | tcccggaccc ctgaggtcac atgcgtggtg gtggacgtga gccacgaaga ccctgaggtc |
| 301 | aagttcaact ggtacgtgga cggcgtggag gtgcataatg ccaagacaaa gccgcgggag |
| 361 | gagcagtaca acagcacgta ccgtgtggtc agcgtcctca ccgtcctgca ccaggactgg |
| 421 | ctgaatggca aggagtacaa gtgcaaggtc tccaacaaag ccctcccagc ccccatcgag |
| 481 | aaaaccatct ccaaagccaa agggcagccc cgagaaccac aggtgtacac cctgccccca |
| 541 | tcccgggatg agctgaccaa gaaccaggtc agcctgacct gcctggtcaa aggcttctat |
| 601 | cccagcgaca tcgccgtgga gtgggagagc aatgggcagc cggagaacaa ctacaagacc |
| 661 | acgcctcccg tgctggactc cgacggctcc ttcttcctct acagcaagct caccgtggac |
| 721 | aagagcaggt ggcagcaggg gaacgtcttc tcatgctccg tgatgcatga ggctctgcac |
| 781 | aaccactaca cgcagaagag cctctccctg tctccgggta aa |

SEQ ID NO: 3 depicts the amino acid sequence of a CTP domain:

GSPRFQDSSSSKAPPPSLPSPSRLPGPSDTPILPQ

SEQ ID NO: 4 depicts the nucleic acid sequence encoding a CTP domain:

ggatcaccacgcttccaggactcctcttcctcaaaggcccctcctcctag
ccttccaagcccatcccgactcccggggccctcggacactccgatcctcc
cacaataa

SEQ ID NO: 16 depicts the amino acid sequence of a Fc-CTP where the CTP domain is underlined and bold:

| | |
|---|---|
| 1 | MWGWKCLLFW AVLVTATLCT ARPAPTLPEQ AQQSTRADLG PGEPKSCDKT HTCPPCPAPE |
| 61 | LLGGPSVFLF PPKPKDTLMI SRTPEVTCVV VDVSHEDPEV KFNWYVDGVE VHNAKTKPRE |
| 121 | EQYNSTYRVV SVLTVLHQDW LNGKEYKCKV SNKALPAPIE KTISKAKGQP REPQVYTLPP |
| 181 | SRDELTKNQV SLTCLVKGFY PSDIAVEWES NGQPENNYKT TPPVLDSDGS FFLYSKLTVD |
| 241 | KSRWQQGNVF SCSVMHEALH NHYTQKSLSL SPGKGSPRFQ DSSSSKAPPP SLPSPSRLPG |
| 301 | PSDTPILPQ |

SEQ ID NO: 17 depicts the nucleic acid sequence encoding a Fc-CTP where the CTP domain is underlined and bold:

| | |
|---|---|
| 1 | atgtggggct ggaagtgcct cctcttctgg gctgtgctgg tcacagccac tctctgcact |
| 61 | gccaggccag ccccaacctt gcccgaacaa gctcagcagt cgacgcgcgc agatctgggc |
| 121 | ccgggcgagc ccaaatcttg tgacaaaact cacacatgcc caccgtgccc agcacctgaa |
| 181 | ctcctggggg gaccgtcagt cttcctcttc cccccaaaac ccaaggacac cctcatgatc |
| 241 | tcccggaccc ctgaggtcac atgcgtggtg gtggacgtga gccacgaaga ccctgaggtc |
| 301 | aagttcaact ggtacgtgga cggcgtggag gtgcataatg ccaagacaaa gccgcgggag |
| 361 | gagcagtaca acagcacgta ccgtgtggtc agcgtcctca ccgtcctgca ccaggactgg |
| 421 | ctgaatggca aggagtacaa gtgcaaggtc tccaacaaag ccctcccagc ccccatcgag |
| 481 | aaaaccatct ccaaagccaa agggcagccc cgagaaccac aggtgtacac cctgccccca |
| 541 | tcccgggatg agctgaccaa gaaccaggtc agcctgacct gcctggtcaa aggcttctat |
| 601 | cccagcgaca tcgccgtgga gtgggagagc aatgggcagc cggagaacaa ctacaagacc |
| 661 | acgcctcccg tgctggactc cgacggctcc ttcttcctct acagcaagct caccgtggac |
| 721 | aagagcaggt ggcagcaggg gaacgtcttc tcatgctccg tgatgcatga ggctctgcac |
| 781 | aaccactaca cgcagaagag cctctccctg tctccgggta aaggatcacc acgcttccag |
| 841 | gactcctctt cctcaaaggc ccctcctcct agccttccaa gcccatcccg actcccgggg |
| 901 | ccctcggaca ctccgatcct cccacaataa |

Protein glycosylation is an enzymatic process that adds a carbohydrate moiety to a polypeptide. Glycosylation is a post-translational modification for polypeptides involved in cell membrane formation. During this process, the linking of monosaccharide units to the amino acid chains sets up the stage for a series of enzymatic reactions that lead to the formation of glycoproteins. A typical glycoprotein has at least 41 bonds which involve 8 amino acids and 13 different monosaccharide units and includes the glycophosphatidylinositol (GPI) and phosphoglycosyl linkages. Protein glycosylation helps in proper folding of proteins, stability and in cell-to-cell adhesion commonly needed by cells of the immune system. The major sites of protein glycosylation in the body are endoplasmic reticulum (ER), Golgi body, nucleus, and the cell fluid. In certain embodiments, glycosylation can be N-linked or O-linked.

N-linked glycosylation begins with the addition of a 14-sugar precursor to an asparagine in the polypeptide chain of the target protein. The structure of this precursor contains glucose, mannose, and 2 N-acetylglucosamine molecules. A complex set of reactions attaches this branched chain to a carrier molecule called dolichol, and this entity is transferred to the appropriate point on the polypeptide chain as it is translocated into the ER lumen. The motif for an N-linked glycosylation site is Asn-X-Thr/Ser, where X can be any amino acid except proline. Marshall, Glycoproteins. Annu. Rev. Biochem. 41:673-702 (1972). N-linked glycosylation can be important to protein folding.

O-linked glycosylation begins with an enzyme-mediated addition of N-acetyl-galactosamine followed by other carbohydrates (such as galactose and sialic acid) to serine or threonine residues. O-linked glycosylation occurs at later stages of protein processing.

O-linked glycosylation begins with an enzyme-mediated addition of N-acetyl-galactosamine followed by other carbohydrates (such as galactose and sialic acid) to serine or threonine residues. O-linked glycosylation occurs at later stages of protein processing.

In one embodiment, the CTP domain can differ by at least 1, 2, 3, 4, 5, 6, 7, 8, 9, or 10 conservative amino acid substitutions as compared to SEQ ID NO: 3. In another embodiment, the CTP domain can differ from the native human chorionic gonadotropin CTP by 1, 2, 3, 4, or 5 conservative amino acid substitutions as described in U.S. Pat. No. 5,712,122. In one embodiment, the Fc domain can differ by at least 1, 2, 3, 4, 5, 6, 7, 8, 9, or 10 conservative amino acid substitutions as compared to SEQ ID NO: 1. In another embodiment, the Fc domain can differ from SEQ ID NO: 1 by 1, 2, 3, 4, or 5 conservative amino acid substitutions as described in U.S. Pat. No. 5,712,122. . In one embodiment, the Fc-CTP domain can differ by at least 1, 2, 3, 4, 5, 6, 7, 8, 9, or 10 conservative amino acid substitutions as compared to SEQ ID NO: 16. In another embodiment, the Fc domain can differ from SEQ ID NO: 16 by 1, 2, 3, 4, or 5 conservative amino acid substitutions as described in U.S. Pat. No. 5,712,122.

In one embodiment, conservative amino acid substitutions can be substitution combinations and their reciprocals as described in Dayhoff et al., ((1978) Atlas of Protein Sequence and Structure, ed. Dayhoff, M. (Natl. Biomed. Res. Found., Silver Spring, Md.), Vol. 5, Suppl. 3, pp. 345-352), which include, but are not limited to: Cys/Ser, Cys/Tyr, Ser/Thr, Ser/Pro, Ser/Ala, Ser/Gly, Ser/Asn, Ser/Asp, Ser/Glu, Ser/Arg, Ser/Lys, Thr/Pro, Thr/Ala, Thr/Gly, Thr/Asn, Thr/Asp, Thr/Glu, Thr/Lys, Thr/Ile, Thr/Val, Pro/Ala, Pro/Gln, Pro/His, Pro/Arg, Ala/Gly, Ala/Asn, Ala/Asp, Ala/Glu, Ala/Gln, Ala/Val, Gly/Asn, Gly/Asp, Gly/Glu, Asn/Asp, Asn/Glu, Asn/Gln, Asn/His, Asn/Arg, Asn/Lys, Asp/Glu, Asp/Gln, Asp/His, Asp/Lys, Glu/Gln, Glu/His, Glu/Lys, Gln/His, Gln/Arg, Gln/Lys, His/Arg, His/Lys, His/Tyr, Arg/Lys, Arg/Met, Arg/Trp, Lys/Met, Met/Ile, Met/Leu, Met/Val, Met/Phe, Ile/Leu, Ile/Val, Ile/Phe, Leu/Val, Leu/Phe, Phe/Tyr, Phe/Trp, and Tyr/Trp.

In one embodiment, the CTP domain comprises at least 1 glycosylation site. In one embodiment, the CTP domain comprises at least 2 glycosylation sites. In one embodiment, the CTP domain comprises at least 3 glycosylation sites. In one embodiment, the CTP domain comprises at least 4 glycosylation sites. In one embodiment, the CTP domain comprises at least 5 glycosylation sites. In some embodiments, SEQ ID NO: 3 comprises at least 1 glycosylation site, at least 2 glycosylation sites, at least 3 glycosylation sites, at least 4 glycosylation sites, at least 5 glycosylation sites, at least 6 glycosylation sites, at least 7 glycosylation sites, at least 8 glycosylation sites, at least 9 glycosylation site, or at least 10 glycosylation sites. In further embodiments, the glycosylation site can be an N-linked glycosylation site, an O-linked glycosylation site, or a combination thereof.

In one embodiment, the Fc domain comprises at least 1 glycosylation site. In one embodiment, the Fc domain comprises at least 2 glycosylation sites. In one embodiment, the Fc domain comprises at least 3 glycosylation sites. In one embodiment, the Fc domain comprises at least 4 glycosylation sites. In one embodiment, the Fc domain comprises at least 5 glycosylation sites. In some embodiments, SEQ ID NO: 1 comprises at least 1 glycosylation site, at least 2 glycosylation sites, at least 3 glycosylation sites, at least 4 glycosylation sites, at least 5 glycosylation sites, at least 6 glycosylation sites, at least 7 glycosylation sites, at least 8 glycosylation sites, at least 9 glycosylation site, or at least 10 glycosylation sites. In further embodiments, the glycosylation site can be an N-linked glycosylation site, an O-linked glycosylation site, or a combination thereof.

In one embodiment, the Fc-CTP domain comprises at least 1 glycosylation site. In one embodiment, the Fc-CTP domain comprises at least 2 glycosylation sites. In one embodiment, the Fc-CTP domain comprises at least 3 glycosylation sites. In one embodiment, the Fc-CTP domain comprises at least 4 glycosylation sites. In one embodiment, the Fc-CTP domain comprises at least 5 glycosylation sites. In some embodiments, SEQ ID NO: 16 comprises at least 1 glycosylation site, at least 2 glycosylation sites, at least 3 glycosylation sites, at least 4 glycosylation sites, at least 5 glycosylation sites, at least 6 glycosylation sites, at least 7 glycosylation sites, at least 8 glycosylation sites, at least 9 glycosylation site, or at least 10 glycosylation sites. In further embodiments, the glycosylation site can be an N-linked glycosylation site, an O-linked glycosylation site, or a combination thereof.

The nucleic acid can be any type of nucleic acid, including genomic DNA, complementary DNA (cDNA), synthetic or semi-synthetic DNA, as well as any form of corresponding RNA. For example, a nucleic acid encoding an anthrax toxin receptor protein can comprise a recombinant nucleic acid encoding such a protein. The nucleic acid can be a non-naturally occurring nucleic acid created artificially (such as by assembling, cutting, ligating or amplifying sequences). It can be double-stranded or single-stranded.

The invention further provides for nucleic acids that are complementary to an anthrax toxin receptor molecule. Complementary nucleic acids can hybridize to the nucleic acid sequence described above under stringent hybridization conditions. Non-limiting examples of stringent hybridization conditions include temperatures above 30° C., above 35° C., in excess of 42° C., and/or salinity of less than about 500 mM, or less than 200 mM. Hybridization conditions can be adjusted by the skilled artisan via modifying the temperature, salinity and/or the concentration of other reagents such as SDS or SSC.

According to the invention, protein variants can include amino acid sequence modifications. For example, amino acid sequence modifications fall into one or more of three classes: substitutional, insertional or deletional variants. Insertions can include amino and/or carboxyl terminal fusions as well as intrasequence insertions of single or multiple amino acid residues. Insertions ordinarily can be smaller insertions than those of amino or carboxyl terminal fusions, for example, on the order of one to four residues. Deletions are characterized by the removal of one or more amino acid residues from the protein sequence. These variants ordinarily are prepared by site-specific mutagenesis of nucleotides in the DNA encoding the protein, thereby producing DNA encoding the variant, and thereafter expressing the DNA in recombinant cell culture. In one embodiment, an anthrax toxin receptor molecule can be modified by mutating cysteine residues to non-cysteine residues including, but not limited to serine and alanine. In one embodiment, an anthrax toxin receptor molecule can be modified with an amino acid sequence inserted as a carboxyl terminal fusion. In another embodiment, an anthrax toxin receptor molecule can be modified with an amino acid sequence inserted as an amino terminal fusion. For example, carboxyl and/or amino terminal fusions may be used to increase the stability of an anthrax toxin receptor molecule.

In one embodiment, variants of the ANTXR molecule comprise a polypeptide comprising the vWF domain of ANTXR1 with cysteine mutant(s) fused to an Fc domain, a CTP domain, an Fc-CTP domain, or a combination thereof. In another embodiment, variants of the ANTXR molecule comprise a polypeptide comprising the extracellular domain of ANTXR1 with cysteine mutant(s) fused to an Fc domain, a CTP domain, an Fc-CTP domain, or a combination thereof. In a further embodiment, variants of the ANTXR molecule comprise a polypeptide comprising SEQ ID NO: 18 or 22 (or a fusion thereof that further comprises SEQ ID NO: 1, 3 and/or 16), wherein the cysteine residues at positions 25, 39, 177, 220, 232, 257, 281, and 317 of ANTXR1 are mutated. In one embodiment, the cysteine mutations in ANTXR1 can occur at positions 25, 39, 177, 220, 232, 257, 281, 317, or a combination thereof. In one embodiment, at least one cysteine residue is mutated. In another embodiment, at least two cysteine residues are mutated. In yet another embodiment, at least three cysteine residues are mutated. In yet another embodiment, at least four cysteine residues are mutated. In a further embodiment, at least five cysteine residues are mutated. In yet a further embodiment, at least six cysteine residues are mutated. In some embodiments, at least seven cysteine residues are mutated. In other embodiments, at least eight cysteine residues are mutated. In one embodiment, Cys177 in the vWF domain of SEQ ID NO 18 or 22 is mutated. In another embodiment, Cys220, Cys232, Cys257, Cys281, Cys317, or a combination thereof, in the extracellular domain of SEQ ID NO: 18 or 22 is mutated. In some embodiments, the cysteine residue is mutated to any one of the following: Cys to Ser, Cys to Tyr, Cys to Thr, Cys to Pro, Cys to Ala, Cys to Gly, Cys to Asn, Cys to Asp, Cys to Glu, Cys to Arg, or Cys to Lys. In a further embodiment, the cysteine residue is mutated to a serine residue or an alanine residue.

In one embodiment, variants of the ANTXR molecule comprise a polypeptide comprising the vWF domain of ANTXR2 with cysteine mutant(s) fused to an Fc domain, a CTP domain, an Fc-CTP domain, or a combination thereof. In another embodiment, variants of the ANTXR molecule comprise a polypeptide comprising the extracellular domain of ANTXR2 with cysteine mutant(s) fused to an Fc domain, a CTP domain, an Fc-CTP domain, or a combination thereof. In a further embodiment, variants of the ANTXR molecule comprise a polypeptide comprising SEQ ID NO: 20 or 26 (or a fusion thereof that further comprises SEQ ID NO: 1, 3 and/or 16), wherein the cysteine residues at positions 39, 175, 218, 230, 255, 279, and 315 of ANTXR2 are mutated. In one embodiment, the cysteine mutations in ANTXR2 can occur at positions 39, 175, 218, 230, 255, 279, 315, or a combination thereof. In one embodiment, at least one cysteine residue is mutated. In another embodiment, at least two cysteine residues are mutated. In yet another embodiment, at least three cysteine residues are mutated. In yet another embodiment, at least four cysteine residues are mutated. In a further embodiment, at least five cysteine residues are mutated. In yet a further embodiment, at least six cysteine residues are mutated. In some embodiments, at least seven cysteine residues are mutated. In other embodiments, at least eight cysteine residues are mutated. In one embodiment, Cys175 in the vWF domain of SEQ ID NO: 20 or 26 is mutated. In another embodiment, Cys218, Cys230, Cys255, Cys279, Cys315, or a combination thereof, in the extracellular domain of SEQ ID NO: 20 or 26 is mutated. In some embodiments, the cysteine residue is mutated to any one of the following: Cys to Ser, Cys to Tyr, Cys to Thr, Cys to Pro, Cys to Ala, Cys to Gly, Cys to Asn, Cys to Asp, Cys to Glu, Cys to Arg, or Cys to Lys. In a further embodiment, the cysteine residue is mutated to a serine residue or an alanine residue.

In one embodiment, variants of the ANTXR molecule comprise a polypeptide comprising the vWF domain of ANTXR1 with aspartate, serine, and/or threonine mutant(s) fused to an Fc domain, a CTP domain, an Fc-CTP domain, or a combination thereof. In another embodiment, variants of the ANTXR molecule comprise a polypeptide comprising the extracellular domain of ANTXR1 with aspartate, serine, and/or threonine mutant(s) fused to an Fc domain, a CTP domain, an Fc-CTP domain, or a combination thereof. In a further embodiment, variants of the ANTXR molecule comprise a polypeptide comprising SEQ ID NO: 18 or 22 (or a fusion thereof that further comprises SEQ ID NO: 1, 3 and/or 16), wherein Aspartate residue 50 (D50), Serine residue 52 (S52), Serine residue 54 (S54), Threonine residue 118 (T118), Aspartate residue 150 (D150), and Aspartate residue 156 (D156) of ANTXR1 are mutated. In one embodiment, the ANTXR molecule comprising SEQ ID NO: 18 or 22 (or a fusion thereof that further comprises SEQ ID NO: 1, 3 and/or 16) exhibits at least one mutation selected from the group consisting of D50, S52, S54, T118, D150, and D156. In one embodiment, the ANTXR molecule comprising SEQ ID NO: 18 or 22 (or a fusion thereof that further comprises SEQ ID NO: 1, 3 and/or 16) exhibits at least two mutations selected from the group consisting of D50, S52, S54, T118, D150, and D156. In one embodiment, the ANTXR molecule comprising SEQ ID NO: 18 or 22 (or a fusion thereof that further comprises SEQ ID NO: 1, 3 and/or 16) exhibits at least three mutations selected from the group consisting of D50, S52, S54, T118, D150, and D156. In one embodiment, the ANTXR molecule comprising SEQ ID NO: 18 or 22 (or a fusion thereof that further comprises SEQ ID NO: 1, 3 and/or 16) exhibits at least four mutations selected from the group consisting of D50, S52, S54, T118, D150, and D156. In one embodiment, the ANTXR molecule comprising SEQ ID NO: 18 or 22 (or a fusion thereof that further comprises SEQ ID NO: 1, 3 and/or 16) exhibits at least five mutations selected from the group consisting of D50, S52, S54, T118, D150, and D156. In one embodiment, the ANTXR molecule comprising SEQ ID NO: 18 or 22 (or a fusion thereof that further comprises SEQ ID NO: 1, 3 and/or 16) exhibits the D50, S52, S54, T118, D150, and D156 mutations. In some embodiments, the aspartate residue is mutated to any one of the following: Asp to Tyr, Asp to Pro, Asp to Ser, Asp to Thr, Asp to Ala, Asp to Gly, Asp to Asn, Asp to Glu, Asp to Arg, or Asp to Lys. In some embodiments, the serine residue is mutated to any one of the following: Ser to Pro, Ser to Ala, Ser to Gly, Ser to Asn, Ser to Glu, Ser to Arg Ser to Lys, Ser to Thr, or Ser to Asp. In some embodiments, the threonine residue is mutated to any one of the following: Thr to Pro, Thr to Ala, Thr to Gly, Thr to Asn, Thr to Asp, Thr to Glu, Thr to Lys, Thr to Ile, Thr to Val, or Thr to Ser. In a further embodiment, the aspartate residue is mutated to an alanine residue. In yet another embodiment, the serine residue is mutated to an alanine residue. In yet a further embodiment, the threonine residue is mutated to an alanine residue.

In one embodiment, variants of the ANTXR molecule comprise a polypeptide comprising the vWF domain of ANTXR2 with aspartate, serine, and/or threonine mutant(s) fused to an Fc domain, a CTP domain, an Fc-CTP domain, or a combination thereof. In another embodiment, variants of the ANTXR molecule comprise a polypeptide comprising the extracellular domain of ANTXR2 with aspartate, serine, and/or threonine mutant(s) fused to an Fc domain, a CTP domain, an Fc-CTP domain, or a combination thereof. In a further embodiment, variants of the ANTXR molecule comprise a polypeptide comprising SEQ ID NO: 20 or 26 (or a fusion thereof that further comprises SEQ ID NO: 1, 3 and/or 16), wherein Aspartate residue 50 (D50), Serine residue 52 (S52), Serine residue 54 (S54), Threonine residue 118 (T118), Aspartate residue 148 (D148), and Aspartate residue 152 (D152) of ANTXR2 are mutated. In one embodiment, the ANTXR molecule comprising SEQ ID NO: 20 or 26 (or a fusion thereof that further comprises SEQ ID NO: 1, 3 and/or 16) exhibits at least one mutation selected from the group consisting of D50, S52, S54, T118, D148, and D152. In one embodiment, the ANTXR molecule comprising SEQ ID NO: 20 or 26 (or a fusion thereof that further comprises SEQ ID NO: 1, 3 and/or 16) exhibits at least two mutations selected from the group consisting of D50, S52, S54, T118, D148, and D152. In one embodiment, the ANTXR molecule comprising SEQ ID NO: 20 or 26 (or a fusion thereof that further comprises SEQ ID NO: 1, 3 and/or 16) exhibits at least three mutations selected from the group consisting of D50, S52, S54, T118, D148, and D152. In one embodiment, the ANTXR molecule comprising SEQ ID NO: 20 or 26 (or a fusion thereof that further comprises SEQ ID NO: 1, 3 and/or 16) exhibits at least four mutations selected from the group consisting of D50, S52, S54, T118, D148, and D152. In one embodiment, the ANTXR molecule comprising SEQ ID NO: 20 or 26 (or a fusion thereof that further comprises SEQ ID NO: 1, 3 and/or 16) exhibits at least five mutations selected from the group consisting of D50, S52, S54, T118, D148, and D152. In one embodiment, the ANTXR molecule comprising SEQ ID NO: 20 or 26 (or a fusion thereof that further comprises SEQ ID NO: 1, 3 and/or 16) exhibits the D50, S52, S54, T118, D148, and D152 mutations. In some embodiments, the aspartate residue is mutated to any one of the following: Asp to Tyr, Asp to Pro, Asp to Ser, Asp to Thr, Asp to Ala, Asp to Gly, Asp to Asn, Asp to Glu, Asp to Arg, or Asp to Lys. In some embodiments, the serine residue is mutated to any one of the following: Ser to Pro, Ser to Ala, Ser to Gly, Ser to Asn, Ser to Glu, Ser to Arg Ser to Lys, Ser to Thr, or Ser to Asp. In some embodiments, the threonine residue is mutated to any one of the following: Thr to Pro, Thr to Ala, Thr to Gly, Thr to Asn, Thr to Asp, Thr to Glu, Thr to Lys, Thr to Ile, Thr to Val, or Thr to Ser. In a further embodiment, the aspartate residue is mutated to an alanine residue. In yet another embodiment, the serine residue is mutated to an alanine residue. In yet a further embodiment, the threonine residue is mutated to an alanine residue.

In one embodiment, an anthrax toxin receptor molecule comprises a protein or polypeptide encoded by a nucleic acid sequence encoding an anthrax toxin receptor protein, such as the sequences shown in SEQ ID NOS: 18, 20, 22, or 26. In another embodiment, the polypeptide can be modified, such as by glycosylations and/or acetylations and/or chemical reaction or coupling, and can contain one or several non-natural or synthetic amino acids. An example of an anthrax toxin receptor molecule is the polypeptide having the amino acid sequence shown in SEQ ID NOS: 18, 20, 22, 24, 26, 28 (or a fusion thereof that further comprises SEQ ID NO: 1, 3 and/or 16). Such variants can include those having at least from about 46% to about 50% identity to SEQ ID NOS: 18, 20, 22, 24, 26, 28 (or a fusion thereof that further comprises SEQ ID NO: 1, 3 and/or 16), or having at least from about 50.1% to about 55% identity to SEQ ID NOS: 18, 20, 22, 24, 26, 28 (or a fusion thereof that further comprises SEQ ID NO: 1, 3 and/or 16), or having at least from about 55.1% to about 60% identity to SEQ ID NOS: 18, 20, 22, 24, 26, 28 (or a fusion thereof that further comprises SEQ ID NO: 1, 3 and/or 16), or having from about 60.1% to about 65% identity to SEQ ID NOS: 18, 20, 22, 24, 26, 28 (or a fusion thereof that further comprises SEQ ID NO: 1, 3 and/or 16), or having from about 65.1% to about 70% identity to SEQ ID NOS: 18, 20, 22, 24, 26, 28 (or a fusion thereof that further comprises SEQ ID NO: 1, 3 and/or 16), or having at least from about 70.1% to about 75% identity to SEQ ID NOS: 18, 20, 22, 24, 26, 28 (or a fusion thereof that further comprises SEQ ID NO: 1, 3 and/or 16), or having at least from about 75.1% to about 80% identity to SEQ ID NOS: 18, 20, 22, 24, 26, 28 (or a fusion thereof that further comprises SEQ ID NO: 1, 3 and/or 16), or having at least from about 80.1% to about 85% identity to SEQ ID NOS: 18, 20, 22, 24, 26, 28 (or a fusion thereof that further comprises SEQ ID NO: 1, 3 and/or 16), or having at least from about 85.1% to about 90% identity to SEQ ID NOS: 18, 20, 22, 24, 26, 28 (or a fusion thereof that further comprises SEQ ID NO: 1, 3 and/or 16), or having at least from about 90.1% to about 95% identity to SEQ ID NOS: 18, 20, 22, 24, 26, 28 (or a fusion thereof that further comprises 1, 3 and/or 16), or having at least from about 95.1% to about 97% identity to SEQ ID NOS: 18, 20, 22, 24, 26, 28 (or a fusion thereof that further comprises SEQ ID NO: 1, 3 and/or 16), or having at least from about 97.1% to about 99% identity to SEQ ID NOS: 18, 20, 22, 24, 26, 28 (or a fusion thereof that further comprises SEQ ID NO: 1, 3 and/or 16). In another embodiment, an anthrax toxin receptor molecule can be a fragment of an anthrax toxin receptor. For example, the fragment of the anthrax toxin receptor molecule can be the extracellular domain of an anthrax toxin receptor molecule, or the vWF domain of an anthrax toxin receptor molecule (see SEQ ID NO: 18, 20, 22, 24, 26, 28).

In one embodiment, an anthrax toxin receptor molecule, according to the methods described herein can be administered to a subject as a recombinant protein. In another embodiment, an anthrax toxin receptor molecule, can be administered to a subject as a modified recombinant protein. For example, an anthrax toxin receptor protein, or fragment thereof, can be modified by the addition of a carboxy-terminal peptide (CTP) domain (SEQ ID NO: 2), a Fc domain (SEQ ID NO: 1), a Fc-CTP domain (SEQ ID NO: 16), or a combination thereof, for increased stability. In a further embodiment, an anthrax toxin receptor molecule, according to the methods described herein can be administered to a subject by delivery of a nucleic acid encoding an anthrax toxin receptor protein, or fragment thereof. For example, nucleic acids can be delivered to a subject using a viral vector.

Polypeptides can be susceptible to denaturation or enzymatic degradation in the blood, liver or kidney. Accordingly, polypeptides can be unstable and have short biological half-lives. Polypeptides can be modified to increase their stability, for example, a fusion protein can be generated for increased stability. In one embodiment, an isolated polypeptide can comprise an ANTXR carboxy-terminal peptide (CTP) domain (SEQ ID NO: 2), a Fc domain (SEQ ID NO: 1), a Fc-CTP domain (SEQ ID NO: 16), or a combination thereof, fused to an anthrax toxin receptor molecule. The addition of the CTP domain to an anthrax toxin receptor molecule can be used to stabilize the anthrax toxin receptor molecule and cause a longer biological half-life to the polypeptides in circulation. In one embodiment, the CTP comprises the C-terminal domain of the beta subunit of the human chorionic gonadotrophin (hCG). In one embodiment, the Fc domain comprises the constant region of human IgG1.

The term “biological half-life” is the time required for the activity of a substance taken into the body to lose one half its initial pharmacologic, physiologic, or biologic activity.

In one embodiment, an anthrax toxin receptor molecule of the present invention comprises an isolated polypeptide comprising a carboxy-terminal peptide (CTP) domain (e.g., SEQ ID NO: 2) fused to an anthrax toxin receptor molecule (e.g., SEQ ID NO: 18, 20, 22, 24, 26, 28). In one embodiment, fusing a CTP domain to an anthrax toxin receptor molecule (for example, ANTXR1, or ANTXR2) can result in increased glycosylation and/or protein stability. In some embodiments, at least one CTP domain is added to the N-terminus of an anthrax toxin receptor molecule. In other embodiments, at least two CTP domains are added to the N-terminus of an anthrax toxin receptor molecule. In further embodiments, at least three CTP domains are added to the N-terminus of an anthrax toxin receptor molecule. In some embodiments, at least one CTP domain is added to the C-terminus of an anthrax toxin receptor molecule. In other embodiments, at least two CTP domains are added to the C-terminus of an anthrax toxin receptor molecule. In further embodiments, at least three CTP domains are added to the C-terminus of an anthrax toxin receptor molecule. In some embodiments, at least one CTP domain is added to the N-terminus and/or C-terminus of an anthrax toxin receptor molecule. In other embodiments, at least two CTP domains are added to the N-terminus and/or C-terminus of an anthrax toxin receptor molecule. In further embodiments, at least three CTP domains are added to the N-terminus and/or C-terminus of an anthrax toxin receptor molecule. In some embodiments, the CTP domains are added in tandem.

In one embodiment, an anthrax toxin receptor molecule of the present invention comprises an isolated polypeptide comprising a Fc domain (e.g., SEQ ID NO: 1) fused to an anthrax toxin receptor molecule (e.g., SEQ ID NO: 18, 20, 22, 24, 26, 28). A Fc domain is the fragment crystallizable region of an antibody. In one embodiment, fusing a Fc domain to an anthrax toxin receptor molecule (for example, ANTXR1, or ANTXR2) can result in dimerization, and/or protein stability, and/or increased protein activity, and/or improved protein purification. In some embodiments, at least one Fc domain is added to the N-terminus of an anthrax toxin receptor molecule. In other embodiments, at least two Fc domains are added to the N-terminus of an anthrax toxin receptor molecule. In further embodiments, at least three Fc domains are added to the N-terminus of an anthrax toxin receptor molecule. In some embodiments, at least one Fc domain is added to the C-terminus of an anthrax toxin receptor molecule. In other embodiments, at least two Fc domains are added to the C-terminus of an anthrax toxin receptor molecule. In further embodiments, at least three Fc domains are added to the C-terminus of an anthrax toxin receptor molecule. In some embodiments, at least one Fc domain is added to the N-terminus and/or C-terminus of an anthrax toxin receptor molecule. In other embodiments, at least two Fc domains are added to the N-terminus and/or C-terminus of an anthrax toxin receptor molecule. In further embodiments, at least three Fc domains are added to the N-terminus and/or C-terminus of an anthrax toxin receptor molecule. In some embodiments, the Fc domains are added in tandem.

In one embodiment, an anthrax toxin receptor molecule of the present invention comprises an isolated polypeptide comprising a Fc-CTP domain (e.g., SEQ ID NO: 16) fused to an anthrax toxin receptor molecule (e.g., SEQ ID NO: 18, 20, 22, 24, 26, 28). In one embodiment, fusing a Fc-CTP domain to an anthrax toxin receptor molecule (for example, ANTXR1, or ANTXR2) can result in dimerization, and/or protein stability, and/or increased protein activity, and/or improved protein purification. In some embodiments, at least one Fc-CTP domain is added to the N-terminus of an anthrax toxin receptor molecule. In other embodiments, at least two Fc-CTP domains are added to the N-terminus of an anthrax toxin receptor molecule. In further embodiments, at least three Fc-CTP domains are added to the N-terminus of an anthrax toxin receptor molecule. In some embodiments, at least one Fc-CTP domain is added to the C-terminus of an anthrax toxin receptor molecule. In other embodiments, at least two Fc-CTP domains are added to the C-terminus of an anthrax toxin receptor molecule. In further embodiments, at least three Fc-CTP domains are added to the C-terminus of an anthrax toxin receptor molecule. In some embodiments, at least one Fc-CTP domain is added to the N-terminus and/or C-terminus of an anthrax toxin receptor molecule. In other embodiments, at least two Fc-CTP domains are added to the N-terminus and/or C-terminus of an anthrax toxin receptor molecule. In further embodiments, at least three Fc domains are added to the N-terminus and/or C-terminus of an anthrax toxin receptor molecule. In some embodiments, the Fc-CTP domains are added in tandem.

In one embodiment, an anthrax toxin receptor molecule of the present invention comprises an isolated polypeptide comprising a CTP domain, an Fc-CTP domain, and/or a Fc domain fused to an anthrax toxin receptor molecule. In one embodiment, fusing a CTP domain and a Fc domain to an anthrax toxin receptor molecule (for example, ANTXR1, or ANTXR2) can result in dimerization, and/or protein stability, and/or increased protein activity, and/or improved protein purification. In some embodiments, a CTP domain, an Fc-CTP domain, and/or a Fc domain are added to the N-terminus of an anthrax toxin receptor molecule. In some embodiments, a CTP domain, an Fc-CTP domain, and/or a Fc domain are added to the C-terminus of an anthrax toxin receptor molecule. In some embodiments, at least one Fc domain is added to the N-terminus and/or C-terminus of an anthrax toxin receptor molecule, at least one Fc-CTP domain is added to the N-terminus and/or C-terminus of an anthrax toxin receptor molecule, and at least one CTP domain is added to the N-terminus and/or C-terminus of an anthrax toxin receptor molecule. In other embodiments, at least one Fc domain is added to the N-terminus and/or C-terminus of an anthrax toxin receptor molecule, at least one Fc-CTP domain is added to the N-terminus and/or C-terminus of an anthrax toxin receptor molecule, and at least two CTP domains are added to the N-terminus and/or C-terminus of an anthrax toxin receptor molecule. In further embodiments, at least one Fc domain are added to the N-terminus and/or C-terminus of an anthrax toxin receptor molecule, at least one Fc-CTP domain is added to the N-terminus and/or C-terminus of an anthrax toxin receptor molecule, and at least three CTP domains are added to the N-terminus and/or C-terminus of an anthrax toxin receptor molecule. In some embodiments, the Fc domains, Fc-CTP domains, and CTP domains are added in tandem and can be in any order.

In some embodiments, at least two Fc domains are added to the N-terminus and/or C-terminus of an anthrax toxin receptor molecule, at least one Fc-CTP domain is added to the N-terminus and/or C-terminus of an anthrax toxin receptor molecule, and at least one CTP domain is added to the N-terminus and/or C-terminus of an anthrax toxin receptor molecule. In other embodiments, at least three Fc domains are added to the N-terminus and/or C-terminus of an anthrax toxin receptor molecule, at least one Fc-CTP domain is added to the N-terminus and/or C-terminus of an anthrax toxin receptor molecule, and at least one CTP domain is added to the N-terminus and/or C-terminus of an anthrax toxin receptor molecule. In some embodiments, at least two Fc domains are added to the N-terminus and/or C-terminus of an anthrax toxin receptor molecule, at least one Fc-CTP domain is added to the N-terminus and/or C-terminus of an anthrax toxin receptor molecule, and at least two CTP domains are added to the N-terminus and/or C-terminus of an anthrax toxin receptor molecule. In other embodiments, at least three Fc domains are added to the N-terminus and/or C-terminus of an anthrax toxin receptor molecule, at least one Fc-CTP domain is added to the N-terminus and/or C-terminus of an anthrax toxin receptor molecule, and at least three CTP domains are added to the N-terminus and/or C-terminus of an anthrax toxin receptor molecule. In some embodiments, the Fc domains, Fc-CTP domains, and CTP domains are added in tandem and can be in any order.

In some embodiments, at least one Fc domain is added to the N-terminus and/or C-terminus of an anthrax toxin receptor molecule, at least two Fc-CTP domaina are added to the N-terminus and/or C-terminus of an anthrax toxin receptor molecule, and at least one CTP domain is added to the N-terminus and/or C-terminus of an anthrax toxin receptor molecule. In some embodiments, at least one Fc domain is added to the N-terminus and/or C-terminus of an anthrax toxin receptor molecule, at least three Fc-CTP domains are added to the N-terminus and/or C-terminus of an anthrax toxin receptor molecule, and at least one CTP domain is added to the N-terminus and/or C-terminus of an anthrax toxin receptor molecule. In some embodiments, at least two Fc domains are added to the N-terminus and/or C-terminus of an anthrax toxin receptor molecule, at least two Fc-CTP domains are added to the N-terminus and/or C-terminus of an anthrax toxin receptor molecule, and at least one CTP domain is added to the N-terminus and/or C-terminus of an anthrax toxin receptor molecule. In other embodiments, at least three Fc domains are added to the N-terminus and/or C-terminus of an anthrax toxin receptor molecule, at least two Fc-CTP domains are added to the N-terminus and/or C-terminus of an anthrax toxin receptor molecule, and at least one CTP domain is added to the N-terminus and/or C-terminus of an anthrax toxin receptor molecule. In some embodiments, at least two Fc domains are added to the N-terminus and/or C-terminus of an anthrax toxin receptor molecule, at least two Fc-CTP domains are added to the N-terminus and/or C-terminus of an anthrax toxin receptor molecule, and at least two CTP domains are added to the N-terminus and/or C-terminus of an anthrax toxin receptor molecule. In other embodiments, at least three Fc domains are added to the N-terminus and/or C-terminus of an anthrax toxin receptor molecule, at least two Fc-CTP domains are added to the N-terminus and/or C-terminus of an anthrax toxin receptor molecule, and at least three CTP domains are added to the N-terminus and/or C-terminus of an anthrax toxin receptor molecule. In some embodiments, the Fc domains, Fc-CTP domains, and CTP domains are added in tandem and can be in any order.

The invention provides for a nucleic acid encoding an anthrax toxin receptor protein, or fragment thereof, such as a ANTXR1 molecule, or a ANTXR2 molecule.

The Genbank Accession ID for the ANTXR1 gene is 84168. Three isoforms are listed for ANTXR1, e.g., having Genebank Accession Nos. NP_{—}060623 (corresponding nucleotide sequence NM_{—}018153); NP_{—}115584 (corresponding nucleotide sequence NM_{—}032208); NP_{—}444262 (corresponding nucleotide sequence NM_{—}053034).

For example, the polypeptide sequence of human ANTXR1 is depicted in SEQ ID NO: 18. The nucleotide sequence of human ANTXR1 is shown in SEQ ID NO: 19. Sequence information related to ANTXR1 is accessible in public databases by GenBank Accession numbers NP_{—}115584 (protein) and NM_{—}032208 (nucleic acid).

SEQ ID NO: 18 is the human wild type amino acid sequence corresponding to ANTXR1 (residues 1-564; vWF domain; transmembrane domain highlighted in grey; predicted extracellular domain comprises residues 217-320):

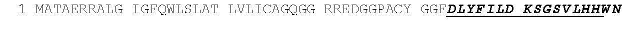

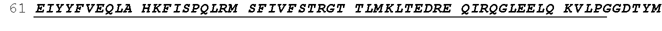

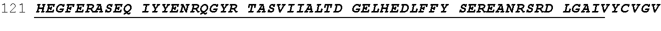

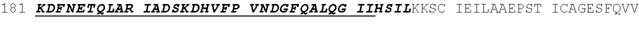

241 VRGNGFRHAR NVDRVLCSFK INDSVTLNEK PFSVEDTYLL CPAPILKEVG MKAALQVSMN

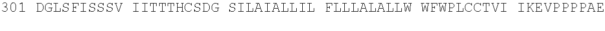

361 ESEEEDDDGL PKKKWPTVDA SYYGGRGVGG IKRMEVRWGE KGSTEEGAKL EKAKNARVKM
421 PEQEYEFPEP RNLNNNMRRP SSPRKWYSPI KGKLDALWVL LRKGYDRVSV MRPQPGDTGR
481 CINFTRVKNN QPAKYPLNNA YHTSSPPPAP IYTPPPPAPH CPPPPPSAPT PPIPSPPSTL
541 PPPPQAPPPN RAPPPSRPPP RPSV

SEQ ID NO: 19 is the human wild type nucleotide sequence corresponding to ANTXR1 (nucleotides 1-5909), wherein the underscored bolded “ATG” denotes the beginning of the open reading frame:

```
      1 atcatattta aaatctggga caaagaaccg tcgggacgga actccttcca ttgcaaaagc
    61 tcggcgcggc ctcgggagct gcccggcggc cccggaccga ggcagccctc ccctttaaaa
  121 gaagcggagg acaggattgg gatccttgaa acccgaaacc cagaaacagc atcggagcgg
  181 aaaccagagg ggaaaccttg aactcctcca gacaattgct tccggggagt tgcgagggag
  241 cgagggggaa taaaggaccc gcgaggaagg gcccgcggat ggcgcgtccc tgagggtcgt
  301 ggcgagttcg cggagcgtgg gaaggagcgg accctgctct ccccgggctg cgggccatgg
  361 ccacggcgga gcggagagcc ctcggcatcg gcttccagtg gctctctttg gccactctgg
  421 tgctcatctg cgccgggcaa gggggacgca gggaggatgg gggtccagcc tgctacggcg
  481 gatttgacct gtacttcatt ttggacaaat caggaagtgt gctgcaccac tggaatgaaa
  541 tctattactt tgtggaacag ttggctcaca aattcatcag cccacagttg agaatgtcct
  601 ttattgtttt ctccacccga ggaacaacct taatgaaact gacagaagac agagaacaaa
  661 tccgtcaagg cctagaagaa ctccagaaag ttctgccagg aggagacact tacatgcatg
  721 aaggatttga aagggccagt gagcagattt attatgaaaa cagacaaggg tacaggacag
  781 ccagcgtcat cattgctttg actgatggag aactccatga agatctcttt ttctattcag
  841 agagggaggc taataggtct cgagatcttg gtgcaattgt ttactgtgtt ggtgtgaaag
  901 atttcaatga gacacagctg gcccggattg cggacagtaa ggatcatgtg tttcccgtga
  961 atgacggctt tcaggctctg caaggcatca tccactcaat tttgaagaag tcctgcatcg
1021 aaattctagc agctgaacca tccaccatat gtgcaggaga gtcatttcaa gttgtcgtga
1081 gaggaaacgg cttccgacat gcccgcaacg tggacagggt cctctgcagc ttcaagatca
1141 atgactcggt cacactcaat gagaagccct tttctgtgga agatacttat ttactgtgtc
1201 cagcgcctat cttaaaagaa gttggcatga aagctgcact ccaggtcagc atgaacgatg
1261 gcctctcttt tatctccagt tctgtcatca tcaccaccac acactgttct gacggttcca
1321 tcctggccat cgccctgctg atcctgttcc tgctcctagc cctggctctc ctctggtggt
1381 tctggcccct ctgctgcact gtgattatca aggaggtccc tccaccccct gccgaggaga
1441 gtgaggaaga agatgatgat ggtctgccta agaaaaagtg gccaacggta gacgcctctt
1501 attatggtgg gagaggcgtt ggaggcatta aaagaatgga ggttcgttgg ggagaaaagg
1561 gctccacaga agaaggtgct aagttggaaa aggcaaagaa tgcaagagtc aagatgccgg
1621 agcaggaata tgaattccct gagccgcgaa atctcaacaa caatatgcgt cggccttctt
1681 ccccccggaa gtggtactct ccaatcaagg gaaaactcga tgccttgtgg gtcctactga
1741 ggaaaggata tgatcgtgtg tctgtgatgc gtccacagcc aggagacacg gggcgctgca
1801 tcaacttcac cagggtcaag aacaaccagc cagccaagta cccactcaac aacgcctacc
1861 acacctcctc gccgcctcct gcccccatct acactccccc acctcctgcg ccccactgcc
1921 ctcccccgcc ccccagcgcc cctacccctc ccatcccgtc cccaccttcc acccttcccc
1981 ctcctcccca ggctccacct cccaacaggg cacctcctcc ctcccgccct cctccaaggc
2041 cttctgtcta gagcccaaag ttcctgctct gggctctctc agaaacttca ggagatgtta
2101 gaacaagtct ttccagttag agaagaggag tggtgataaa gcccactgac cttcacacat
2161 tctaaaaatt ggttggcaat gccagtatac caacaatcat gatcagctga aagaaacaga
2221 tattttaaat tgccagaaaa caaatgatga ggcaactaca gtcagattta tagccagcca
2281 tctatcacct ctagaaggtt ccagagacag tgaaactgca agatgctctc aacaggatta
2341 tgtctcatgg agaccagtaa gaaaatcatt tatctgaagg tgaaatgcag agttggataa
2401 gaaatacatt gctgggtttc taaaatgctg ccttcctgcc tctactccac ctccatccct
2461 ggactttgga cccttggcct aggagcctaa ggaccttcac ccctgtgcac cacccaagaa
2521 agaggaaaac tttgcctaca actttggaaa tgctggggtc cctggtgtgg taagaaactc
2581 aacatcagac gggtatgcag aaggatgttc ttctgggatt tgcaggtaca taaaaaatgt
2641 atggcatctt ttccttgcaa attcttccag tttccaagtg agaaggggag caggtgttta
2701 ctgatggaaa aggtatgttg ctatgttgat gtgtaagtga aatcagttgt gtgcaataga
2761 caggggcgta ttcatgggag catcagccag tttctaaaac ccacaggcca tcagcagcta
2821 gaggtggctg gctttggcca gacatggacc ctaaatcaac agacaatggc attgtcgaag
2881 agcaacctgt taatgaatca tgttaaaaat caaggtttgg cttcagttta aatcacttga
2941 ggtatgaagt ttatcctgtt ttccagagat aaacataagt tgatcttccc aaaataccat
3001 cattaggacc tatcacacaa tatcactagt tttttttgtt tgtttgtttt ttgttttttt
3061 tcttggtaaa gccatgcacc acagacttct gggcagagct gagagacaat ggtcctgaca
3121 taataaggat ctttgattaa cccccataag gcatgtgtgt gtatacaaat atacttctct
3181 ttggcttttc gacatagaac ctcagctgtt aaccaagggg aaatacatca gatctgcaac
3241 acagaaatgc tctgcctgaa atttccacca tgcctaggac tcaccccatt tatccaggtc
3301 tttctggatc tgtttaatca ataagcccta taatcacttg ctaaacactg ggcttcatca
3361 cccagggata aaaacagaga tcattgtctt ggacctcctg catcagccta ttcaaaatta
3421 tctctctctc tagctttcca caaatcctaa aattcctgtc ccaagccacc caaattctca
3481 gatcttttct ggaacaaggc agaatataaa ataaatatac atttagtggc ttgggctatg
3541 gtctccaaag atccttcaaa aatacatcaa gccagcttca ttcactcact ttacttagaa
3601 cagagatata agggcctggg atgcatttat tttatcaata ccaatttttg tggccatggc
3661 agacattgct aatcaatcac agcactattt cctattaagc ccactgattt cttcacaatc
3721 cttctcaaat tacaattcca aagagccgcc actcaacagt cagatgaacc caacagtcag
3781 atgagagaaa tgaaccctac ttgctatctc tatcttagaa agcaaaaaca aacaggagtt
3841 tccagggaga atgggaaagc cagggggcat aaaaggtaca gtcaggggaa aatagatcta
3901 ggcagagtgc cttagtcagg gaccacgggc gctgaatctg cagtgccaac accaaactga
3961 cacatctcca ggtgtacctc caaccctagc cttctcccac agctgcctac aacagagtct
4021 cccagccttc tcagagagct aaaaccagaa atttccagac tcatgaaagc aaccccccag
4081 cctctcccca accctgccgc attgtctaat ttttagaaca ctaggcttct tctttcatgt
4141 agttcctcat aagcaggggc cagaatatct cagccacctg cagtgacatt gctggacccc
4201 tgaaaaccat tccataggag aatgggttcc ccaggctcac agtgtagaga cattgagccc
4261 atcacaactg ttttgactgc tggcagtcta aaacagtcca cccaccccat ggcactgccg
4321 cgtgattccc gcggccattc agaagttcaa gccgagatgc tgacgttgct gagcaacgag
4381 atggtgagca tcagtgcaaa tgcaccattc agcacatcag tcatatgccc agtgcagtta
4441 caagatgttg tttcggcaaa gcattttgat ggaataggga actgcaaatg tatgatgatt
4501 ttgaaaaggc tcagcaggat ttgttcttaa accgactcag tgtgtcatcc ccggttattt
4561 agaattacag ttaagaagga gaaacttcta taagactgta tgaacaaggt gatatcttca
4621 tagtgggcta ttacaggcag gaaaatgttt taactggttt acaaaatcca tcaatacttg
4681 tgtcattccc tgtaaaaggc aggagacatg tgattatgat caggaaactg cacaaaatta
4741 ttgttttcag cccccgtgtt attgtccttt tgaactgttt ttttttttat taaagccaaa
4801 tttgtgttgt atatattcgt attccatgtg ttagatggaa gcatttccta tccagtgtga
4861 ataaaaagaa cagttgtagt aaattattat aaagccgatg atatttcatg gcaggttatt
4921 ctaccaagct gtgcttgttg gtttttccca tgactgtatt gcttttataa atgtacaaat
4981 agttactgaa atgacgagac ccttgtttgc acagcattaa taagaacctt gataagaacc
5041 atattctgtt gacagccagc tcacagtttc ttgcctgaag cttggtgcac cctccagtga
5101 gacacaagat ctctctttta ccaaagttga gaacagagct ggtggattaa ttaatagtct
5161 tcgatatctg gccatgggta acctcattgt aactatcatc agaatgggca gagatgatct
5221 tgaagtgtca catacactaa agtccaaaca ctatgtcaga tgggggtaaa atccattaaa
5281 gaacaggaaa aaataattat aagatgataa gcaaatgttt cagcccaatg tcaacccagt
5341 taaaaaaaaa attaatgctg tgtaaaatgg ttgaattagt ttgcaaacta tataaagaca
5401 tatgcagtaa aaagtctgtt aatgcacatc ctgtgggaat ggagtgttct aaccaattgc
5461 cttttcttgt tatctgagct ctcctatatt atcatactca gataaccaaa ttaaaagaat
5521 tagaatatga tttttaatac acttaacatt aaactcttct aactttcttc tttctgtgat
5581 aattcagaag atagttatgg atcttcaatg cctctgagtc attgttataa aaaatcagtt
5641 atcactatac catgctatag gagactgggc aaaacctgta caatgacaac cctggaagtt
5701 gcttttttta aaaaaataat aaatttctta aatcaactct tttttctggt tgtctgtttg
5761 ttataaagtg caacgtattc aagtcctcaa tatcctgatc ataataccat gctataggag
5821 actgggcaaa acctgtacaa tgacaaccct ggaagttgct tttttaaaaa aaataataaa
5881 tttcttaaat caaaaaaaaa aaaaaaaaa
```

For example, the polypeptide sequence of human ANTXR1, isoform 2 is depicted in SEQ ID NO: 22. The nucleotide sequence of human ANTXR1, isoform 2 is shown in SEQ ID NO: 23. Sequence information related to ANTXR1 is accessible in public databases by GenBank Accession numbers NP_{—}444262 (protein) and NM_{—}053034 (nucleic acid).

SEQ ID NO: 22 is the human wild type amino acid sequence corresponding to ANTXR1, isoform 2 (residues 1-368; vWF domain; transmembrane domain highlighted in grey; predicted extracellular domain comprises residues 217-320):

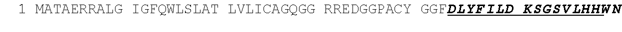

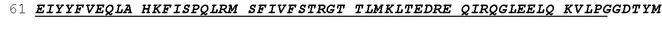

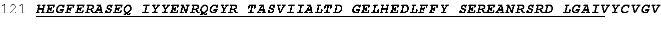

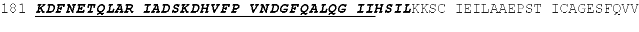

241 VRGNGFRHAR NVDRVLCSFK INDSVTLNEK PFSVEDTYLL CPAPILKEVG MKAALQVSMN

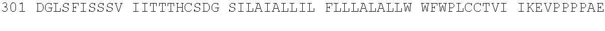

361 ESEENKIK

SEQ ID NO: 23 is the human wild type nucleotide sequence corresponding to ANTXR1, isoform 2 (nucleotides 1-1667), wherein the underscored bolded “ATG” denotes the beginning of the open reading frame:

```
      1 atcatattta aaatctggga caaagaaccg tcgggacgga actccttcca ttgcaaaagc
    61 tcggcgcggc ctcgggagct gcccggcggc cccggaccga ggcagccctc ccctttaaaa
  121 gaagcggagg acaggattgg gatccttgaa acccgaaacc cagaaacagc atcggagcgg
  181 aaaccagagg ggaaaccttg aactcctcca gacaattgct tccggggagt tgcgagggag
  241 cgagggggaa taaaggaccc gcgaggaagg gcccgcggat ggcgcgtccc tgagggtcgt
  301 ggcgagttcg cggagcgtgg gaaggagcgg accctgctct ccccgggctg cgggccatgg
  361 ccacggcgga gcggagagcc ctcggcatcg gcttccagtg gctctctttg gccactctgg
  421 tgctcatctg cgccgggcaa gggggacgca gggaggatgg gggtccagcc tgctacggcg
  481 gatttgacct gtacttcatt ttggacaaat caggaagtgt gctgcaccac tggaatgaaa
  541 tctattactt tgtggaacag ttggctcaca aattcatcag cccacagttg agaatgtcct
  601 ttattgtttt ctccacccga ggaacaacct taatgaaact gacagaagac agagaacaaa
  661 tccgtcaagg cctagaagaa ctccagaaag ttctgccagg aggagacact tacatgcatg
  721 aaggatttga aagggccagt gagcagattt attatgaaaa cagacaaggg tacaggacag
  781 ccagcgtcat cattgctttg actgatggag aactccatga agatctcttt ttctattcag
  841 agagggaggc taataggtct cgagatcttg gtgcaattgt ttactgtgtt ggtgtgaaag
  901 atttcaatga gacacagctg gcccggattg cggacagtaa ggatcatgtg tttcccgtga
  961 atgacggctt tcaggctctg caaggcatca tccactcaat tttgaagaag tcctgcatcg
1021 aaattctagc agctgaacca tccaccatat gtgcaggaga gtcatttcaa gttgtcgtga
1081 gaggaaacgg cttccgacat gcccgcaacg tggacagggt cctctgcagc ttcaagatca
1141 atgactcggt cacactcaat gagaagccct tttctgtgga agatacttat ttactgtgtc
1201 cagcgcctat cttaaaagaa gttggcatga aagctgcact ccaggtcagc atgaacgatg
1261 gcctctcttt tatctccagt tctgtcatca tcaccaccac acactgttct gacggttcca
1321 tcctggccat cgccctgctg atcctgttcc tgctcctagc cctggctctc ctctggtggt
1381 tctggcccct ctgctgcact gtgattatca aggaggtccc tccaccccct gccgaggaga
1441 gtgaggaaaa taaaataaaa taacaagaag aagaaagaaa gaaatcccac agaaacagat
1501 aacctaacac agcccgtgca acgtatttta tacaatgctc tgaaaatcat agtctcaatc
1561 tagacagtct tttcctctag ttccctgtat tcaaatccca gtgtctaaca ttcaataaat
1621 agctatatga aatcaaaaaa aaaaaaaaaa aaaaaaaaaa aaaaaaa
```

SEQ ID NO: 24 depicts the amino acid sequence of a ANTXR1 (isoform 2)-vWF variant, which comprises amino acids 1-234 of ANTXR1 (isoform 2):

MATAERRALGIGFQWLSLATLVLICAGQGGRREDGGPACYGGFDLYFILD
KSGSVLHHWNEIYYFVEQLAHKFISPQLRMSFIVFSTRGTTLMKLTEDRE
QIRQGLEELQKVLPGGDTYMHEGFERASEQIYYENRQGYRTASVIIALTD
GELHEDLFFYSEREANRSRDLGAIVYCVGVKDFNETQLARIADSKDHVFP
VNDGFQALQGIIHSILKKSCIEILAAEPSTICAG

SEQ ID NO: 25 depicts the nucleotide sequence of ANTXR1 (isoform 2), which encodes ANTXR1 (isoform 2). The sequence highlighted in grey encodes the ANTXR1-vWF protein variant:

ATCATATTTAAAATCTGGGACAAAGAACCGTCGGGACGGAACTCCTTCCATTGCAAAAGCTCGGCGCG
GCCTCGGGAGCTGCCCGGCGGCCCCGGACCGAGGCAGCCCTCCCCTTTAAAAGAAGCGGAGGACAGGA
TTGGGATCCTTGAAACCCGAAACCCAGAAACAGCATCGGAGCGGAAACCAGAGGGGAAACCTTGAACT
CCTCCAGACAATTGCTTCCGGGGAGTTGCGAGGGAGCGAGGGGGAATAAAGGACCCGCGAGGAAGGGC
CCGCGGATGGCGCGTCCCTGAGGGTCGTGGCGAGTTCGCGGAGCGTGGGAAGGAGCGGACCCTGCTCT

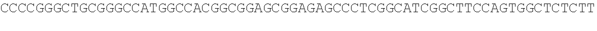

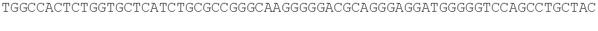

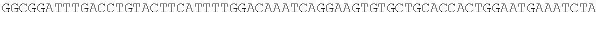

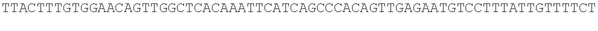

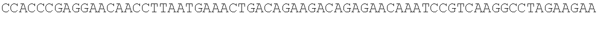

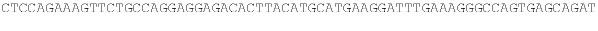

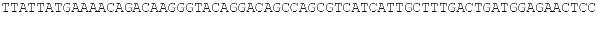

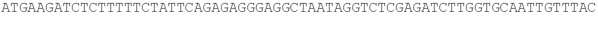

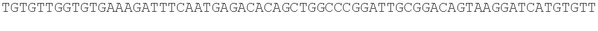

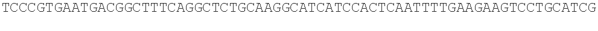

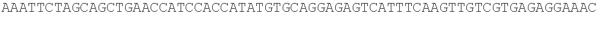

GGCTTCCGACATGCCCGCAACGTGGACAGGGTCCTCTGCAGCTTCAAGATCAATGACTCGGTCACACT
CAATGAGAAGCCCTTTTCTGTGGAAGATACTTATTTACTGTGTCCAGCGCCTATCTTAAAAGAAGTTG
GCATGAAAGCTGCACTCCAGGTCAGCATGAACGATGGCCTCTCTTTTATCTCCAGTTCTGTCATCATC
ACCACCACACACTGTTCTGACGGTTCCATCCTGGCCATCGCCCTGCTGATCCTGTTCCTGCTCCTAGC
CCTGGCTCTCCTCTGGTGGTTCTGGCCCCTCTGCTGCACTGTGATTATCAAGGAGGTCCCTCCACCCC
CTGCCGAGGAGAGTGAGGAAAATAAAATAAAATAACAAGAAGAAGAAAGAAAGAAATCCCACAGAAAC
AGATAACCTAACACAGCCCGTGCAACGTATTTTATACAATGCTCTGAAAATCATAGTCTCAATCTAGA
CAGTCTTTTCCTCTAGTTCCCTGTATTCAAATCCCAGTGTCTAACATTCAATAAATAGCTATATGAAA
TCAAAAAAAAAAAAAAAAAAAAAAAAAAAAAAAAA

The Genbank Accession ID for the ANTXR2 gene is 118429. Two isoforms are listed for ANTXR2, e.g., having Genebank Accession Nos. NP_{—}477520 (corresponding nucleotide sequence NM_{—}058172); NP_{—}001139266 (corresponding nucleotide sequence NM_{—}001145794).

For example, the polypeptide sequence of human ANTXR2 is depicted in SEQ ID NO: 20. The nucleotide sequence of human ANTXR2 is shown in SEQ ID NO: 21. Sequence information related to ANTXR2 is accessible in public databases by GenBank Accession numbers NP_{—}477520 (protein) and NM_{—}058172 (nucleic acid).

SEQ ID NO: 20 is the human wild type amino acid sequence corresponding to ANTXR2 (residues 1-488; vWF domain; predicted transmembrane domain highlighted in grey (see Sun and Collier (2010) PLoS One 5(5):e10553); predicted extracellular domain comprises residues 215-320):

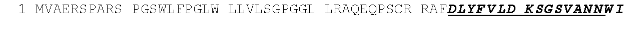

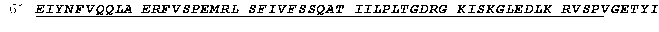

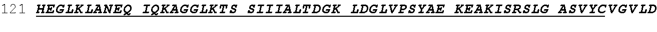

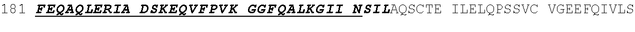

241 GRGFMLGSRN GSVLCTYTVN ETYTTSVKPV SVQLNSMLCP APILNKAGET LDVSVSFNGG

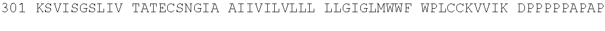

361 KEEEEEPLPT KKWPTVDASY YGGRGVGGIK RMEVRWGDKG STEEGARLEK AKNAVVKIPE
421 ETEEPIRPRP PRPKPTHQPP QTKWYTPIKG RLDALWALLR RQYDRVSLMR PQEGDEGRCI
481 NFSRVPSQ

SEQ ID NO: 21 is the human wild type nucleotide sequence corresponding to ANTXR2 (nucleotides 1-8058), wherein the underscored bolded “ATG” denotes the beginning of the open reading frame:

```
      1 agtcatcttc aactcggcag gaacccacaa gtgtgcatgt gtggctcgga ggcttcagct
    61 ggggccccgc cctcgtcccc aggcgcacac tgacacacgc agcccagacc cggcccgagc
  121 gggctcctgc cctcggcgtg gcttctctcc agccgggagt cccagggcca gctagcctcc
  181 tcccctaaag gggacggcct gtcagcgcag tgccagagtc cagcaccggg aggaaagttt
  241 cggagtgcgg agggagttgg ggccgccgga ggagaagagt ctccactcct agtttgttct
  301 gccgtcgccg cgtcccaggg accccttgtc ccgaagcgca cggcagcggg ggggacttca
  361 gccctccagg cggggtgggt tccaggtccg ggtccgaggc gggcgctgga ggctcggccc
  421 caggccggag aggaactcct ttcgcgagct gtcgccgtgg gcccgcattg tctgcaggaa
  481 ctctccggaa tcgggagggg gaggactgga tcgcgcttcc actgggattc gtcaagagtt
  541 ccggcggcag ctgcggcggt ggcggagact ccctttgtcc tctcaggacc tccctctctc
  601 cctccctgtc agctggtggg tcccgctgcc gcaggcgccg gcgtctcagc tgctcgccgc
  661 cccccacccc agagtgcgtg ccgggtgact cccgccacct ttgcgaccct cctgagctta
  721 ggggactgcg agcgggaggg agtctcaggc ccccggccgc aggatggtgg cggagcggtc
  781 cccggcccgc agccccggga gctggctgtt ccccgggctg tggctgttgg tgctcagcgg
  841 tcccgggggg ctgctgcgcg cccaggagca gccctcctgc agaagagcct ttgatctcta
  901 cttcgtcctg gacaagtctg ggagtgtggc aaataactgg attgaaattt ataatttcgt
  961 acagcaactt gcggagagat ttgtgagccc tgaaatgaga ttatctttca ttgtgttttc
1021 ttctcaagca actattattt tgccattaac tggagacaga ggcaaaatca gtaaaggctt
1081 ggaggattta aaacgtgtta gtccagtagg agagacatat atccatgaag gactaaagct
1141 agcgaatgaa caaattcaga aagcaggagg cttgaaaacc tccagtatca taattgctct
1201 gacagatggc aagttggacg gtctggtgcc atcatatgca gagaaagagg caaagatatc
1261 caggtcactt ggggctagtg tttattgtgt tggtgtcctt gattttgaac aagcacagct
1321 tgaaagaatt gctgattcca aggagcaagt tttccctgtc aaaggtggat ttcaggctct
1381 taaaggaata attaattcta tactagctca gtcatgtact gaaatcctag aattgcagcc
1441 ctcaagtgtc tgtgtggggg aggaatttca gattgtctta agtggaagag gattcatgct
1501 gggcagtcgg aatggcagtg ttctctgcac ttacactgta aatgaaacat atacaacgag
1561 tgtaaaacca gtaagtgtac agcttaattc tatgctttgt cctgcaccta tcctgaataa
1621 agctggagaa actcttgatg tttcagtgag ctttaatgga ggaaaatctg tcatttcagg
1681 atcattaatt gtcacagcca cagaatgttc taacgggatc gcagccatca ttgttatttt
1741 ggtgttactg ctactcctgg ggatcggttt gatgtggtgg ttttggcccc tttgctgcaa
1801 agtggttatt aaggatcctc caccaccacc cgcccctgca ccaaaagagg aggaagaaga
1861 acctttgcct actaaaaagt ggccaactgt ggatgcttcc tattatggtg gtcgaggggt
1921 tggaggaatt aaaagaatgg aggttcgttg gggtgataaa ggatctactg aggaaggtgc
1981 aaggctagag aaagccaaaa atgctgtggt gaagattcct gaagaaacag aggaacccat
2041 caggcctaga ccacctcgac ccaaacccac acaccagcct cctcagacaa aatggtacac
2101 cccaattaag ggtcgtcttg atgctctctg ggctttgttg aggcggcagt atgaccgggt
2161 ttctttgatg cgacctcagg aaggagatga gggccggtgc ataaacttct cccgagttcc
2221 atctcagtaa aagggaagca ggaagaccaa gaaggtacga agatggcaca ttttcacata
2281 gctgattttc aaccaaatga aaaaaatcaa gtgcatttca gaagcttttg gaagagcagc
2341 ttaattcctc tcagtcggga aatgttttct ctgccttctg ctttgcttgc accaaacatt
2401 tctaaacact tgttctgcca tctacatggg aggtgatgaa actcagtggt aactcatgat
2461 ttatgacatt gaaaataaag aggaacattg acctgcagac tatggtttgt acaagaaagt
2521 ttgtttgaat gtgtagaaga ggaaaaagca acaacagcaa caacacgaag atgataccaa
2581 aacaaggacc acaaaacaac tagccatgat gggagacagg agttttttac atggaaacat
2641 ggcacttgtg tttttatgtg gcaagatctt tatccatagg cagagtatga aatttcccac
2701 caggctaagc aaataaagaa gtccattgcc ttatagctat gtcagatcac agaatccttc
2761 caagtgctct atcacagtgt gccttatggg aagtttctga ctggaaaatc ttgtcattct
2821 aacactgaaa agtgcacacg catgacaaaa tgtagacaag atgcctcaag gtattggtag
2881 caagcaagat tttgcccttt agttttcgaa gacacctttc tttcattatg cactcgggac
2941 aagaaaatta atagagcgtt attccacaga aggcctctag ccagagatct tgagtgtagt
3001 gcaagggact cattttttgc gaacttgtcc ctgtgactag tagattcccc cttttcctgt
3061 gtttaggatt tagtagtgca taaagcatta atatccataa acatacctag aagtttgttt
3121 tgcttttaat ttaaaggaag cagtaaccac aaagcttccg ctcagggttt tttctttctt
3181 caagtctcca agggctcttc agcgtcacaa gccagcaact ctctttgcat taaaatttca
3241 aagtttaatt aatataatta aaagcaacag caagcagcag cctgtgaaga ttttgctcat
3301 cttttttatg ccttttgaca ttgaatgacc tattactgta tgcgcattac ttggattttg
3361 aggggcactc taccttggtt atgattcagt agaggaaaaa gaccaccttt cttcaattta
3421 caaattaaat cttctggagg gtcgctatca caaaacattg acgatgtatg tattataatt
3481 ttttagaaaa accaccatcg tgtcacgtcg acgatgccaa attatgttag cgtgagcaga
3541 aacaccgtgg gggaggaagg cagcagctga agaaaaaagc tcaaatgatc tagtcacttt
3601 cgatactgta cttcagatgc gaaatggata ttcgagtgga aacctgacaa agtgcgcctg
3661 ctttgatgtg aactggtata gacaatgacc agtggctggg tcagtgggat gtctctctgt
3721 gagcacaaag gcttatcaaa tgacactaaa aataagttca acaaccatca cattggaagg
3781 gagaaggcga acatttcatg tttggcgggc atgtgagtgc acaagatgga aagagcgatt
3841 ggagcatcct ggtataatta cccccattgt gctcttaatg gaaatttcaa aggacgggag
3901 tattctgttg gttggtgtcc aggtttgtgg cactgttcca agaggcctta cacacacaca
3961 caaatatata attttctata catatatatc ctctagcttg aaacttttgc tcaagtttat
4021 ttatgtcact ggctggctgg atccaaagtc atgtgtccac acattcataa ataaaaattt
4081 tacctatgcc tgtgctacca tttctttaag cccacataaa gctaatgcct tcttgggttg
4141 aatatgcagt tgttatgctt agtttcaggg atggaattct attcagttcc aaaatgtccc
4201 tcatgttcac agatgatact attggcagta gctgcatatg caagtgtaac ttggaagcct
4261 gatatagtat acccaaccct ttctgcaatg agttattgta tccctttgtt ctgcatttgc
4321 atctggaaca agacctaatg tggcttcctt aaatgggcac ttcatttctg atgagtgcac
4381 accaattatt gctattttaa tatcattgag aatactacag aagctaaggc tatgtttctt
4441 tggtaactac ttcaaatttc tctagaatta tgtagcatac ttacattttg ctgcatgaac
4501 aatgaaaatt aacctctgcc tttaaatgga aaaataactt tctgtagatt gttatacagc
4561 tactatgaag agctcttaat gaagaggaag tggcagtggt ctatatgtag actgaaaaaa
4621 tatcattaat aaagttcaaa gactgatgga atttcattgc ccttaaagat atgaaccaga
4681 tctgttttat gcttttctcc aattatacta actagcagtt gaaaagaatt tgttttgaag
4741 agtgttattt tcttaccaaa agaccagatc aaaaatccct atacttttcc accaaaaaaa
4801 gattaaaatg aagccatttc tataaattta atggcacttt atattttata tagagaaata
4861 gcttctgttt gattttctgg acaccatgtc agtcaagatc tactttctgt gtaaaaatga
4921 ataagtcttg cacataatcc attcgtgttg cataaggttg caaaaacaat taagtgggaa
4981 atgactccta acttatgtgt ctactctgcc tagaaagaaa gttttgaagg catctatata
5041 tatttcataa caatgtctgt tctttatgga tttgatccat aaatatttat ctatctgaaa
5101 actttgatac ttggaatgtt ttccttagag catttggtgc tccaagaaaa aacatttggt
5161 gctaagagtt gcagcactga tgcctttccc taagttaatt ctattttaaa aatttatatt
5221 tccttcccaa tgagatgcag agtaaaattt gagaccctcc attgactagg gagcagcatc
5281 tgtcatctat ggttaattac tattttgaca aaaatataga aggtttaaac ccccatgtat
5341 ttcagattgt tagagaaaga agtagtttgc ttatatttcc cttgaacttt attctaacca
5401 tttaaataat aaattattca atatttgtta taataaatcc ttactagata caagatttgg
5461 gtgtttctgt gaagttaact ttattatcta aatatcacta accattgttt actgctcaga
5521 ggcagataca tttgtttagt ttaaagttga aacaaattct atattgtgga taattaggtg
5581 tttaaaacat tttgatagac cagcttatat atgttatttt tgaaatttaa gaaggcagaa
5641 tgtaattagg tcacatttac aagtagaagg aattataact tcctactcca tgcatatatt
5701 gtatattggt tagcttttac tagcattttc tagcatcaca ttactttaga aagtttttga
5761 gagtttcatt actactggaa aagtttggtg ggtatatgta cacaggaaag aaatatatgc
5821 atcatttcaa tatgaaagga aatacataat ctagaaacat agaataactc ttggtacatc
5881 tgttacatag ttaaggaaag aaacaggcag gtgaggctgg agaatgttgt ctatttgtag
5941 gaaataaagt cagtgcagac gcatattcta gtttagagtt agattaatat tgtgcttagt
6001 gaatttctat gacatatttc tgagaacact ttaatatatt tccacaccta ctggaatcta
6061 attcagtcga tcggataaaa gaaagacttt tttgttgttg tttttgaaaa tccataatgt
6121 agcaggttct gcgctggttt tttaatgtat tttatttttg tgctctcctg ccaaaagaaa
6181 aggctgataa gtaagtacac agtacaacat gtaaatttgt ataaaaatat gcttgtttag
6241 attgctctac aataactata ttaaggtaaa ttgtgtgtgt gtgtgtgaga gagagagaga
6301 gagagagaaa cagagggaga attgactcaa tgtgttttct atgaagagtc ttagcttagg
6361 aatcaggtaa cagagaggtc ctagaccata gacccagcca tcaactagct tgctgatgtt
6421 gaacagatca actctcaacc atagactgaa gatggaatga agatatcaaa agttcctttt
6481 agcactgact agattttttt tctttttttt gaaacagagt ctcactctgt cacccaggct
6541 ggagttcaat ggcatggtct gggctgactg taacctcctc ttcctgggtt caagtgattc
6601 tcatccctca gcctcctgag tagctgggat tacaggcaca taacaccaca cccagctaac
6661 tcttgtattt ttgtagaaat ggggtttcgc tatgttgctc agactggtct caaacacctg
6721 gcctcaagtg atcctcctgc ctcagtctcc caaatgctgt gattacaggc acaagctact
6781 gcaccaggcc tctgactaca tttctattaa tatggttagg ttggaggttt tagtattttt
6841 gtatctcata tttgtatcaa tatgactggc ttctttgtct gtagtgtgtg gtaatattag
6901 ttctgtaaac tgtcagttgc aaaaaaaaaa aataccttga actatagtat atgttgataa
6961 ttagccataa taatttctta gttaatttct tataattaaa tttgtcaaag aggaaactta
7021 cagtttatat ctgatgaaat ctctaaaaag atgggtaaaa cattgggaaa tgtatgcatg
7081 tacttcactc tggtttcata gggttagcaa gtgtcttaaa aacatatata aagaagcaca
7141 gagattgtta ggagatattt atgctcccag ttttaataat tgggatactt tgtataccac
7201 agaaagaaaa attactaaac tcctcttttt ttagtcaaaa ttggaaaaaa agtcttaatt
7261 gacagttact atgcctgtgc tacccatagc aagtattcag tggaaaatac tttactaagt
7321 aagtaatttg aacacagctt aaaatccata gtatgttaca attgctagcc tttcacaaag
7381 tttgcattgt cttaatgtag aaggatactg tgatctaaga attcacaatt ttaaaaagtg
7441 gaacctaaat agggtttcct aattgccatg aagttatttg tatcttagat gaattatatt
7501 tacaacattg taaatgtcag tgggtgatcc aaaataaatt gttaaagtta ttaaaatgta
7561 catttaagta ggtttcagtt tgactagaaa taattggcaa gaaggcaaga actagtcttc
7621 tagagcaggg atcccatccc ccaggtcatg gactggtact ggtccatggc ctgttagaaa
7681 ccaggccaca cagcaggaga tgagtggaaa gcaagtgaaa cttcatgggt atttacagca
7741 attccccgtc gctcgcatta ccacctgagc tgtgtctcct gtgagatcag cagcagcatt
7801 agattctcaa ggagcacaaa cccttttgga actgtgtgtg agggatctaa gttgcgcatt
7861 tcttatgaga atctaatacc tgatgatctg ttgttgtctc ccaccacccc cagatgggac
7921 catctagttg caggaaaaca agctcaggct cccactgatt ctacattata gtgagttgtg
7981 taattatttc attatatata acaatgtaat aataatagaa ataaagtaca taataaatgt
8041 aaaaaaaaaa aaaaaaaa
```

For example, the polypeptide sequence of human ANTXR2, isoform 2 is depicted in SEQ ID NO: 26. The nucleotide sequence of human ANTXR2, isoform 2 is shown in SEQ ID NO: 27. Sequence information related to ANTXR2 is accessible in public databases by GenBank Accession numbers NP_{—}001139266 (protein) and NM_{—}001145794 (nucleic acid).

SEQ ID NO: 26 is the human wild type amino acid sequence corresponding to ANTXR2, isoform 2 (residues 1-489; vWF domain; predicted transmembrane domain highlighted in grey (see Sun and Collier (2010) PLoS One 5(5):e10553); predicted extracellular domain comprises residues 215-320):

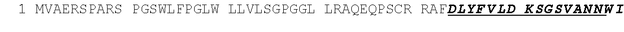

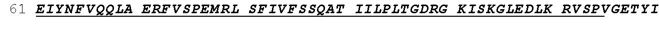

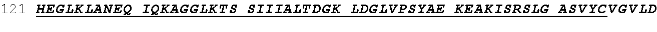

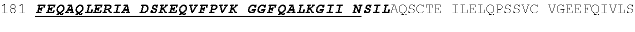

241 GRGFMLGSRN GSVLCTYTVN ETYTTSVKPV SVQLNSMLCP APILNKAGET LDVSVSFNGG

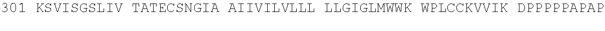

361 KEEEEEPLPT KKWPTVDASY YGGRGVGGIK RMEVRWGDKG STEEGARLEK AKNAVVKIPE
421 ETEEPIRPRP PRPKPTHQPP QTKWYTPIKG RLDALWALLR RQYDRVSLMR PQEGDEVCIW
481 ECIEKELTA

SEQ ID NO: 27 is the human wild type nucleotide sequence corresponding to ANTXR2, isoform 2 (nucleotides 1-2314), wherein the underscored bolded “ATG” denotes the beginning of the open reading frame:

```
      1 agtcatcttc aactcggcag gaacccacaa gtgtgcatgt gtggctcgga ggcttcagct
    61 ggggccccgc cctcgtcccc aggcgcacac tgacacacgc agcccagacc cggcccgagc
  121 gggctcctgc cctcggcgtg gcttctctcc agccgggagt cccagggcca gctagcctcc
  181 tcccctaaag gggacggcct gtcagcgcag tgccagagtc cagcaccggg aggaaagttt
  241 cggagtgcgg agggagttgg ggccgccgga ggagaagagt ctccactcct agtttgttct
  301 gccgtcgccg cgtcccaggg accccttgtc ccgaagcgca cggcagcggg ggggacttca
  361 gccctccagg cggggtgggt tccaggtccg ggtccgaggc gggcgctgga ggctcggccc
  421 caggccggag aggaactcct ttcgcgagct gtcgccgtgg gcccgcattg tctgcaggaa
  481 ctctccggaa tcgggagggg gaggactgga tcgcgcttcc actgggattc gtcaagagtt
  541 ccggcggcag ctgcggcggt ggcggagact ccctttgtcc tctcaggacc tccctctctc
  601 cctccctgtc agctggtggg tcccgctgcc gcaggcgccg gcgtctcagc tgctcgccgc
  661 cccccacccc agagtgcgtg ccgggtgact cccgccacct ttgcgaccct cctgagctta
  721 ggggactgcg agcgggaggg agtctcaggc ccccggccgc aggatggtgg cggagcggtc
  781 cccggcccgc agccccggga gctggctgtt ccccgggctg tggctgttgg tgctcagcgg
  841 tcccgggggg ctgctgcgcg cccaggagca gccctcctgc agaagagcct ttgatctcta
  901 cttcgtcctg gacaagtctg ggagtgtggc aaataactgg attgaaattt ataatttcgt
  961 acagcaactt gcggagagat ttgtgagccc tgaaatgaga ttatctttca ttgtgttttc
1021 ttctcaagca actattattt tgccattaac tggagacaga ggcaaaatca gtaaaggctt
1081 ggaggattta aaacgtgtta gtccagtagg agagacatat atccatgaag gactaaagct
1141 agcgaatgaa caaattcaga aagcaggagg cttgaaaacc tccagtatca taattgctct
1201 gacagatggc aagttggacg gtctggtgcc atcatatgca gagaaagagg caaagatatc
1261 caggtcactt ggggctagtg tttattgtgt tggtgtcctt gattttgaac aagcacagct
1321 tgaaagaatt gctgattcca aggagcaagt tttccctgtc aaaggtggat ttcaggctct
1381 taaaggaata attaattcta tactagctca gtcatgtact gaaatcctag aattgcagcc
1441 ctcaagtgtc tgtgtggggg aggaatttca gattgtctta agtggaagag gattcatgct
1501 gggcagtcgg aatggcagtg ttctctgcac ttacactgta aatgaaacat atacaacgag
1561 tgtaaaacca gtaagtgtac agcttaattc tatgctttgt cctgcaccta tcctgaataa
1621 agctggagaa actcttgatg tttcagtgag ctttaatgga ggaaaatctg tcatttcagg
1681 atcattaatt gtcacagcca cagaatgttc taacgggatc gcagccatca ttgttatttt
1741 ggtgttactg ctactcctgg ggatcggttt gatgtggtgg ttttggcccc tttgctgcaa
1801 agtggttatt aaggatcctc caccaccacc cgcccctgca ccaaaagagg aggaagaaga
1861 acctttgcct actaaaaagt ggccaactgt ggatgcttcc tattatggtg gtcgaggggt
1921 tggaggaatt aaaagaatgg aggttcgttg gggtgataaa ggatctactg aggaaggtgc
1981 aaggctagag aaagccaaaa atgctgtggt gaagattcct gaagaaacag aggaacccat
2041 caggcctaga ccacctcgac ccaaacccac acaccagcct cctcagacaa aatggtacac
2101 cccaattaag ggtcgtcttg atgctctctg ggctttgttg aggcggcagt atgaccgggt
2161 ttctttgatg cgacctcagg aaggagatga ggtttgtata tgggaatgta ttgagaaaga
2221 gctaactgct tgagtcagta taatggaggc agggaaatag taataaaaaa tgattttaaa
2281 gccctattgc acttgaggaa ggaaaaaaaa aaaa
```

SEQ ID NO: 28 depicts the amino acid sequence of a ANTXR2 (isoform 2)-vWF variant, which comprises amino acids 1-232 of ANTXR2 (isoform 2):

MVAERSPARSPGSWLFPGLWLLVLSGPGGLLRAQEQPSCRRAFDLYFVL
DKSGSVANNWIEIYNFVQQLAERFVSPEMRLSFIVFSSQATIILPLTGD
RGKISKGLEDLKRVSPVGETYIHEGLKLANEQIQKAGGLKTSSIIIALT
DGKLDGLVPSYAEKEAKISRSLGASVYCVGVLDFEQAQLERIADSKEQV
FPVKGGFQALKGIINSILAQSCTEILELQPSSVCVG

SEQ ID NO: 29 depicts the nucleotide sequence of ANTXR2 (isoform 2), which encodes amino acids 1-489 of ANTXR2 (isoform 2). The sequence encoding amino acids 1-232 of the ANTXR2-vWF variant is highlighted in grey:

AGTCATCTTCAACTCGGCAGGAACCCACAAGTGTGCATGTGTGGCTCGGAGGCTTCAGCTGGGGCCCCGCCCTCG
TCCCCAGGCGCACACTGACACACGCAGCCCAGACCCGGCCCGAGCGGGCTCCTGCCCTCGGCGTGGCTTCTCTCC
AGCCGGGAGTCCCAGGGCCAGCTAGCCTCCTCCCCTAAAGGGGACGGCCTGTCAGCGCAGTGCCAGAGTCCAGCA
CCGGGAGGAAAGTTTCGGAGTGCGGAGGGAGTTGGGGCCGCCGGAGGAGAAGAGTCTCCACTCCTAGTTTGTTCT
GCCGTCGCCGCGTCCCAGGGACCCCTTGTCCCGAAGCGCACGGCAGCGGGGGGGACTTCAGCCCTCCAGGCGGGG
TGGGTTCCAGGTCCGGGTCCGAGGCGGGCGCTGGAGGCTCGGCCCCAGGCCGGAGAGGAACTCCTTTCGCGAGCT
GTCGCCGTGGGCCCGCATTGTCTGCAGGAACTCTCCGGAATCGGGAGGGGGAGGACTGGATCGCGCTTCCACTGG
GATTCGTCAAGAGTTCCGGCGGCAGCTGCGGCGGTGGCGGAGACTCCCTTTGTCCTCTCAGGACCTCCCTCTCTC
CCTCCCTGTCAGCTGGTGGGTCCCGCTGCCGCAGGCGCCGGCGTCTCAGCTGCTCGCCGCCCCCCACCCCAGAGT
GCGTGCCGGGTGACTCCCGCCACCTTTGCGACCCTCCTGAGCTTAGGGGACTGCGAGCGGGAGGGAGTCTCAGGC

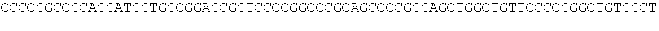

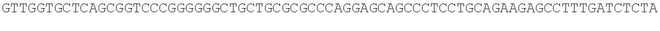

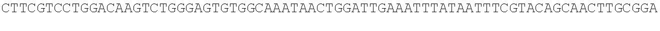

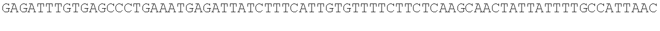

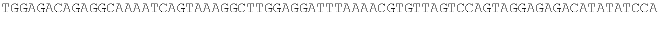

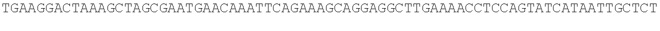

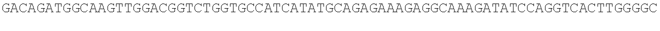

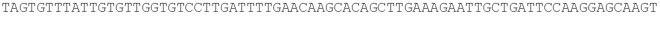

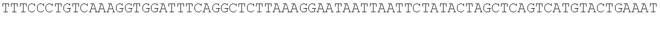

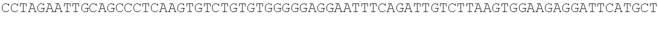

GGGCAGTCGGAATGGCAGTGTTCTCTGCACTTACACTGTAAATGAAACATATACAACGAGTGTAAAACCAGTAAG
TGTACAGCTTAATTCTATGCTTTGTCCTGCACCTATCCTGAATAAAGCTGGAGAAACTCTTGATGTTTCAGTGAG
CTTTAATGGAGGAAAATCTGTCATTTCAGGATCATTAATTGTCACAGCCACAGAATGTTCTAACGGGATCGCAGC
CATCATTGTTATTTTGGTGTTACTGCTACTCCTGGGGATCGGTTTGATGTGGTGGTTTTGGCCCCTTTGCTGCAA
AGTGGTTATTAAGGATCCTCCACCACCACCCGCCCCTGCACCAAAAGAGGAGGAAGAAGAACCTTTGCCTACTAA
AAAGTGGCCAACTGTGGATGCTTCCTATTATGGTGGTCGAGGGGTTGGAGGAATTAAAAGAATGGAGGTTCGTTG
GGGTGATAAAGGATCTACTGAGGAAGGTGCAAGGCTAGAGAAAGCCAAAAATGCTGTGGTGAAGATTCCTGAAGA
AACAGAGGAACCCATCAGGCCTAGACCACCTCGACCCAAACCCACACACCAGCCTCCTCAGACAAAATGGTACAC
CCCAATTAAGGGTCGTCTTGATGCTCTCTGGGCTTTGTTGAGGCGGCAGTATGACCGGGTTTCTTTGATGCGACC
TCAGGAAGGAGATGAGGTTTGTATATGGGAATGTATTGAGAAAGAGCTAACTGCTTGAGTCAGTATAATGGAGGC
AGGGAAATAGTAATAAAAAATGATTTTAAAGCCCTATTGCACTTGAGGAAGGAAAAAAAAAAAA

An anthrax toxin receptor molecule can also encompass ortholog genes, which are genes conserved among different biological species such as humans, dogs, cats, mice, and rats, that encode proteins (for example, homologs (including splice variants), mutants, and derivatives) having biologically equivalent functions as the human-derived protein. Orthologs of an anthrax toxin receptor protein include any mammalian ortholog inclusive of the ortholog in humans and other primates, experimental mammals (such as mice, rats, hamsters and guinea pigs), mammals of commercial significance (such as horses, cows, camels, pigs and sheep), and also companion mammals (such as domestic animals, e.g., rabbits, ferrets, dogs, and cats). An anthrax toxin receptor molecule can comprise a protein encoded by a nucleic acid sequence homologous to the human nucleic acid, wherein the nucleic acid is found in a different species and wherein that homolog encodes a protein similar to an anthrax toxin receptor protein.

The invention utilizes conventional molecular biology, microbiology, and recombinant DNA techniques available to one of ordinary skill in the art. Such techniques are well known to the skilled worker and are explained fully in the literature. See, e.g., Maniatis, Fritsch & Sambrook, “DNA Cloning: A Practical Approach,” Volumes I and II (D. N. Glover, ed., 1985); “Oligonucleotide Synthesis” (M. J. Gait, ed., 1984); “Nucleic Acid Hybridization” (B. D. Hames & S. J. Higgins, eds., 1985); “Transcription and Translation” (B. D. Hames & S. J. Higgins, eds., 1984); “Animal Cell Culture” (R. I. Freshney, ed., 1986); “Immobilized Cells and Enzymes” (IRL Press, 1986): B. Perbal, “A Practical Guide to Molecular Cloning” (1984), and Sambrook, et al., “Molecular Cloning: a Laboratory Manual” (2001).

One skilled in the art can obtain an anthrax toxin receptor molecule, (e.g., ANTXR1, or ANTXR2, or a fusion thereof) in several ways, which include, but are not limited to, isolating the protein via biochemical means or expressing a nucleotide sequence encoding the protein of interest by genetic engineering methods.

The invention provides for an anthrax toxin receptor molecule that is encoded by nucleotide sequences. The anthrax toxin receptor molecule can be a polypeptide encoded by a nucleic acid (including genomic DNA, complementary DNA (cDNA), synthetic DNA, as well as any form of corresponding RNA). For example, an anthrax toxin receptor molecule can be encoded by a recombinant nucleic acid encoding a human anthrax toxin receptor protein, or fragment thereof. The anthrax toxin receptor molecules of the invention can be obtained from various sources and can be produced according to various techniques known in the art. For example, a nucleic acid that encodes an anthrax toxin receptor molecule can be obtained by screening DNA libraries, or by amplification from a natural source. The anthrax toxin receptor molecule of the invention can be produced via recombinant DNA technology and such recombinant nucleic acids can be prepared by conventional techniques, including chemical synthesis, genetic engineering, enzymatic techniques, or a combination thereof. An anthrax toxin receptor molecule of this invention also encompasses variants of the human anthrax toxin receptor proteins. The variants can comprise naturally-occurring variants due to allelic variations between individuals (e.g., polymorphisms), mutated alleles, or alternative splicing forms.

In one embodiment, a fragment of a nucleic acid sequence that comprises an anthrax toxin receptor molecule (such as, e.g., ANTXR1, or ANTXR2, or a fusion thereof) can encompass any portion of about 8 consecutive nucleotides of SEQ ID NOS: 19, 21, 23, 25, 27, 29 (or a fusion thereof that further comprises SEQ ID NO: 2, 4, and/or 17). In one embodiment, the fragment can comprise about 10 nucleotides, about 15 nucleotides, about 20 nucleotides, or about 30 nucleotides of SEQ ID NOS: 19, 21, 23, 25, 27, 29 (or a fusion thereof that further comprises SEQ ID NO: 2, 4, and/or 17). Fragments include all possible nucleotide lengths between about 8 and about 100 nucleotides, for example, lengths between about 15 and about 100 nucleotides, or between about 20 and about 100 nucleotides.

An anthrax toxin receptor molecule, can be a fragment of an anthrax toxin receptor protein, such as, e.g., ANTXR1, or ANTXR2. For example, the anthrax toxin receptor protein fragment can encompass any portion of about 8 consecutive amino acids of SEQ ID NOS: 18, 20, 22, 24, 26, or 28. The fragment can comprise about 10 consecutive amino acids, about 20 consecutive amino acids, about 30 consecutive amino acids, about 40 consecutive amino acids, a least about 50 consecutive amino acids, about 60 consecutive amino acids, about 70 consecutive amino acids, about 80 consecutive amino acids, about 90 consecutive amino acids, about 100 consecutive amino acids, about 110 consecutive amino acids, or about 120 consecutive amino acids of SEQ ID NOS: 18, 20, 22, 24, 26, or 28. Fragments include all possible amino acid lengths between about 8 and 80 about amino acids, for example, lengths between about 10 and about 80 amino acids, between about 15 and about 80 amino acids, between about 20 and about 80 amino acids, between about 35 and about 80 amino acids, between about 40 and about 80 amino acids, between about 50 and about 80 amino acids, or between about 70 and about 80 amino acids.

Recombinant Proteins

One skilled in the art understands that polypeptides (for example ANTXR1, ANTXR2, and the like) can be obtained in several ways, which include but are not limited to, expressing a nucleotide sequence encoding the protein of interest, or fragment thereof, by genetic engineering methods.

In one embodiment, the nucleic acid is expressed in an expression cassette, for example, to achieve overexpression in a cell. The nucleic acids of the invention can be an RNA, cDNA, cDNA-like, or a DNA of interest in an expressible format, such as an expression cassette, which can be expressed from the natural promoter or an entirely heterologous promoter. The nucleic acid of interest can encode a protein, and may or may not include introns. Any recombinant expression system can be used, including, but not limited to, bacterial, mammalian, yeast, insect, or plant cell expression systems.

Host cells transformed with a nucleic acid sequence encoding a ANTXR molecule (such as, e.g., ANTXR1, ANTXR2, or a fusion thereof), can be cultured under conditions suitable for the expression and recovery of the protein from cell culture. The polypeptide produced by a transformed cell can be secreted or contained intracellularly depending on the sequence and/or the vector used. Expression vectors containing a nucleic acid sequence encoding a ANTXR molecule can be designed to contain signal sequences which direct secretion of soluble polypeptide molecules encoded by a ANTXR molecule (such as, e.g., ANTXR1, ANTXR2, or a fusion thereof), through a prokaryotic or eukaryotic cell membrane.

Nucleic acid sequences comprising a ANTXR molecule (such as, e.g., ANTXR1, ANTXR2, or a fusion thereof) that encode a polypeptide can be synthesized, in whole or in part, using chemical methods known in the art. Alternatively, a ANTXR molecule can be produced using chemical methods to synthesize its amino acid sequence, such as by direct peptide synthesis using solid-phase techniques. Protein synthesis can either be performed using manual techniques or by automation. Automated synthesis can be achieved, for example, using Applied Biosystems 431A Peptide Synthesizer (Perkin Elmer). Optionally, fragments of a ANTXR molecule can be separately synthesized and combined using chemical methods to produce a full-length molecule.

A synthetic peptide can be substantially purified via high performance liquid chromatography (HPLC). The composition of a synthetic a ANTXR molecule can be confirmed by amino acid analysis or sequencing. Additionally, any portion of an amino acid sequence comprising a protein encoded by a ANTXR molecule (e.g., ANTXR1, ANTXR2, or a fusion thereof) can be altered during direct synthesis and/or combined using chemical methods with sequences from other proteins to produce a variant polypeptide or a fusion protein.

The invention further encompasses methods for using a protein or polypeptide encoded by a nucleic acid sequence of a ANTXR molecule, such as the sequences shown in SEQ ID NOS: 18, 20, 22, 24, 26, 28 (or a fusion thereof that further comprises SEQ ID NO: 1, 3, and/or 16). In another embodiment, the polypeptide can be modified, such as by glycosylations and/or acetylations and/or chemical reaction or coupling, and can contain one or several non-natural or synthetic amino acids. An example of a ANTXR molecule has the amino acid sequence shown in either SEQ ID NO: 18, 20, 22, 24, 26, 28 (or a fusion thereof that further comprises SEQ ID NO: 1, 3, and/or 16). In certain embodiments, the invention encompasses variants of a human protein encoded by a ANTXR molecule (such as, e.g., ANTXR1, ANTXR2, or a fusion thereof).

Expression Systems

Bacterial Expression Systems.

One skilled in the art understands that expression of desired protein products in prokaryotes is most often carried out in E. coli with vectors that contain constitutive or inducible promoters. Some non-limiting examples of bacterial cells for transformation include the bacterial cell line E. coli strains DH5a or MC1061/p3 (Invitrogen Corp., San Diego, Calif.), which can be transformed using standard procedures practiced in the art, and colonies can then be screened for the appropriate plasmid expression. In bacterial systems, a number of expression vectors can be selected. Non-limiting examples of such vectors include multifunctional E. coli cloning and expression vectors such as BLUESCRIPT (Stratagene). Some E. coli expression vectors (also known in the art as fusion-vectors) are designed to add a number of amino acid residues, usually to the N-terminus of the expressed recombinant protein. Such fusion vectors can serve three functions: 1) to increase the solubility of the desired recombinant protein; 2) to increase expression of the recombinant protein of interest; and 3) to aid in recombinant protein purification by acting as a ligand in affinity purification. In some instances, vectors, which direct the expression of high levels of fusion protein products that are readily purified, may also be used. Some non-limiting examples of fusion expression vectors include pGEX, which fuse glutathione S-tranferase (GST) to desired protein; pcDNA 3.1/V5-His A B & C (Invitrogen Corp, Carlsbad, Calif.) which fuse 6×-His to the recombinant proteins of interest; pMAL (New England Biolabs, MA) which fuse maltose E binding protein to the target recombinant protein; the E. coli expression vector pUR278 (Ruther et al., (1983) EMBO 12:1791), wherein the coding sequence may be ligated individually into the vector in frame with the lac Z coding region in order to generate a fusion protein; and pIN vectors (Inouye et al., (1985) Nucleic Acids Res. 13:3101-3109; Van Heeke et al., (1989) J. Biol. Chem. 24:5503-5509. Fusion proteins generated by the likes of the above-mentioned vectors are generally soluble and can be purified easily from lysed cells via adsorption and binding of the fusion protein to an affinity matrix. For example, fusion proteins can be purified from lysed cells via adsorption and binding to a matrix of glutathione agarose beads subsequently followed by elution in the presence of free glutathione. For example, the pGEX vectors are designed to include thrombin or factor Xa protease cleavage sites so that the cloned target can be released from the GST moiety.

Plant, Insect, and Yeast Expression Systems.

Other suitable cell lines, in addition to microorganisms such as bacteria (e.g., E. coli and B. subtilis) transformed with recombinant bacteriophage DNA, plasmid DNA or cosmid DNA expression vectors containing coding sequences for a ANTXR molecule may alternatively be used to produce the molecule of interest. A non-limiting example includes plant cell systems infected with recombinant virus expression vectors (for example, tobacco mosaic virus, TMV; cauliflower mosaic virus, CaMV) or transformed with recombinant plasmid expression vectors (e.g., Ti plasmid) containing coding sequences for a ANTXR molecule. If plant expression vectors are used, the expression of sequences encoding a ANTXR molecule can be driven by any of a number of promoters. For example, viral promoters such as the 35S and 19S promoters of CaMV can be used alone or in combination with the omega leader sequence from tobacco mosaic virus TMV. Alternatively, plant promoters such as the small subunit of RUBISCO or heat shock promoters, can be used. These constructs can be introduced into plant cells by direct DNA transformation or by pathogen-mediated transfection.

In another embodiment, an insect system also can be used to express a ANTXR molecule. For example, in one such system Autographa californica nuclear polyhedrosis virus (AcNPV) is used as a vector to express foreign genes in Spodoptera frugiperda cells or in Trichoplusia larvae. Sequences encoding a ANTXR molecule can be cloned into a non-essential region of the virus, such as the polyhedrin gene, and placed under control of the polyhedrin promoter. Successful insertion of the nucleic acid sequences of a ANTXR molecule can render the polyhedrin gene inactive and produce recombinant virus lacking coat protein. The recombinant viruses can then be used to infect S. frugiperda cells or Trichoplusia larvae in which a ANTXR molecule can be expressed.

In another embodiment, a yeast (for example, Saccharomyces sp., Pichia sp.) system also can be used to express a ANTXR molecule. Yeast can be transformed with recombinant yeast expression vectors containing coding sequences for a ANTXR molecule.

Mammalian Expression Systems.

Mammalian cells (such as BHK cells, VERO cells, CHO cells and the like) can also contain an expression vector (for example, one that harbors a nucleotide sequence encoding a ANTXR molecule) for expression of a desired product. Expression vectors containing such a nucleic acid sequence linked to at least one regulatory sequence in a manner that allows expression of the nucleotide sequence in a host cell can be introduced via methods known in the art. A number of viral-based expression systems can be used to express a ANTXR molecule in mammalian host cells. The vector can be a recombinant DNA or RNA vector, and includes DNA plasmids or viral vectors. For example, if an adenovirus is used as an expression vector, sequences encoding a ANTXR molecule can be ligated into an adenovirus transcription/translation complex comprising the late promoter and tripartite leader sequence. Insertion into a non-essential E1 or E3 region of the viral genome can be used to obtain a viable virus which is capable of expressing a ANTXR molecule in infected host cells. Transcription enhancers, such as the Rous sarcoma virus (RSV) enhancer, can also be used to increase expression in mammalian host cells. In addition, viral vectors can be constructed based on, but not limited to, adeno-associated virus, retrovirus, adenovirus, lentivirus or alphavirus.

Regulatory sequences are well known in the art, and can be selected to direct the expression of a protein or polypeptide of interest (such as a ANTXR molecule) in an appropriate host cell as described in Goeddel, Gene Expression Technology: Methods in Enzymology 185, Academic Press, San Diego, Calif. (1990). Non-limiting examples of regulatory sequences include: polyadenylation signals, promoters (such as CMV, ASV, SV40, or other viral promoters such as those derived from bovine papilloma, polyoma, and Adenovirus 2 viruses (Fiers, et al., 1973, Nature 273:113; Hager G L, et al., Curr Opin Genet Dev, 2002, 12(2):137-41) enhancers, and other expression control elements. Practitioners in the art understand that designing an expression vector can depend on factors, such as the choice of host cell to be transfected and/or the type and/or amount of desired protein to be expressed.

Enhancer regions, which are those sequences found upstream or downstream of the promoter region in non-coding DNA regions, are also known in the art to be important in optimizing expression. If needed, origins of replication from viral sources can be employed, such as if a prokaryotic host is utilized for introduction of plasmid DNA. However, in eukaryotic organisms, chromosome integration is a common mechanism for DNA replication.

For stable transfection of mammalian cells, a small fraction of cells can integrate introduced DNA into their genomes. The expression vector and transfection method utilized can be factors that contribute to a successful integration event. For stable amplification and expression of a desired protein, a vector containing DNA encoding a protein of interest (for example, a ANTXR molecule) is stably integrated into the genome of eukaryotic cells (for example mammalian cells, such as HEK293 cells), resulting in the stable expression of transfected genes. An exogenous nucleic acid sequence can be introduced into a cell (such as a mammalian cell, either a primary or secondary cell) by homologous recombination as disclosed in U.S. Pat. No. 5,641,670, the contents of which are herein incorporated by reference.

A gene that encodes a selectable marker (for example, resistance to antibiotics or drugs, such as ampicillin, neomycin, G418, and hygromycin) can be introduced into host cells along with the gene of interest in order to identify and select clones that stably express a gene encoding a protein of interest. The gene encoding a selectable marker can be introduced into a host cell on the same plasmid as the gene of interest or can be introduced on a separate plasmid. Cells containing the gene of interest can be identified by drug selection wherein cells that have incorporated the selectable marker gene can survive in the presence of the drug. Cells that have not incorporated the gene for the selectable marker die. Surviving cells can then be screened for the production of the desired protein molecule (for example, a ANTXR molecule).

A host cell strain can be chosen for its ability to modulate the expression of the inserted sequences or to process the expressed ANTXR molecule (such as, e.g., ANTXR1 or ANTXR2) in the desired fashion. Such modifications of the polypeptide include, but are not limited to, acetylation, carboxylation, glycosylation, phosphorylation, lipidation, and acylation. Post-translational processing which cleaves a “prepro” form of the polypeptide also can be used to facilitate correct insertion, folding and/or function. Different host cells which have specific cellular machinery and characteristic mechanisms for post-translational activities (e.g., CHO, HeLa, MDCK, HEK293, and WI38), are available from the American Type Culture Collection (ATCC; 10801 University Boulevard, Manassas, Va. 20110-2209) and can be chosen to ensure the correct modification and processing of the foreign protein.

An exogenous nucleic acid can be introduced into a cell via a variety of techniques known in the art, such as lipofection, microinjection, calcium phosphate or calcium chloride precipitation, DEAE-dextrin-mediated transfection, or electroporation. Electroporation is carried out at approximate voltage and capacitance to result in entry of the DNA construct(s) into cells of interest. Other methods used to transfect cells can also include modified calcium phosphate precipitation, polybrene precipitation, liposome fusion, and receptor-mediated gene delivery.

Animal or mammalian host cells capable of harboring, expressing, and secreting large quantities of a ANTXR molecule of interest into the culture medium for subsequent isolation and/or purification include, but are not limited to, Human Embryonic Kidney 293 cells (HEK-293) (ATCC CRL-1573); Chinese hamster ovary cells (CHO), such as CHO-K1 (ATCC CCL-61), DG44 (Chasin et al., (1986) Som. Cell Molec. Genet, 12:555-556; Kolkekar et al., (1997) Biochemistry, 36:10901-10909; and WO 01/92337 A2), dihydrofolate reductase negative CHO cells (CHOI dhfr−, Urlaub et al., (1980) Proc. Natl. Acad. Sci. U.S.A., 77:4216), and dp12.CHO cells (U.S. Pat. No. 5,721,121); monkey kidney CV1 cells transformed by SV40 (COS cells, COS-7, ATCC CRL-1651); human embryonic kidney cells (e.g., 293 cells, or 293 cells subcloned for growth in suspension culture, Graham et al., (1977) J. Gen. Virol., 36:59); baby hamster kidney cells (BHK, ATCC CCL-10); monkey kidney cells (CV1, ATCC CCL-70); African green monkey kidney cells (VERO-76, ATCC CRL-1587; VERO, ATCC CCL-81); mouse sertoli cells (TM4; Mather (1980) Biol. Reprod., 23:243-251); human cervical carcinoma cells (HELA, ATCC CCL-2); canine kidney cells (MDCK, ATCC CCL-34); human lung cells (WI38, ATCC CCL-75); human hepatoma cells (HEP-G2, HB 8065); mouse mammary tumor cells (MMT 060562, ATCC CCL-51); buffalo rat liver cells (BRL 3A, ATCC CRL-1442); TRI cells (Mather (1982) Annals NY Acad. Sci., 383:44-68); MCR 5 cells; FS4 cells. A cell line transformed to produce a ANTXR molecule can also be an immortalized mammalian cell line of lymphoid origin, which include but are not limited to, a myeloma, hybridoma, trioma or quadroma cell line. The cell line can also comprise a normal lymphoid cell, such as a B cell, which has been immortalized by transformation with a virus, such as the Epstein Barr virus (such as a myeloma cell line or a derivative thereof).

A host cell strain, which modulates the expression of the inserted sequences, or modifies and processes the nucleic acid in a specific fashion desired also may be chosen. Such modifications (for example, glycosylation and other post-translational modifications) and processing (for example, cleavage) of protein products may be important for the function of the protein. Different host cell strains have characteristic and specific mechanisms for the post-translational processing and modification of proteins and gene products. As such, appropriate host systems or cell lines can be chosen to ensure the correct modification and processing of the foreign protein expressed, such as a ANTXR molecule. Thus, eukaryotic host cells possessing the cellular machinery for proper processing of the primary transcript, glycosylation, and phosphorylation of the gene product may be used. Non-limiting examples of mammalian host cells include HEK-293, 3T3, WI38, BT483, Hs578T, CHO, VERY, BHK, Hela, COS, BT2O, T47D, NS0 (a murine myeloma cell line that does not endogenously produce any immunoglobulin chains), CRL7O3O, MDCK, 293, HTB2, and HsS78Bst cells.

Various culturing parameters can be used with respect to the host cell being cultured. Appropriate culture conditions for mammalian cells are well known in the art (Cleveland W L, et al., J Immunol Methods, 1983, 56(2): 221-234) or can be determined by the skilled artisan (see, for example, Animal Cell Culture: A Practical Approach 2nd Ed., Rickwood, D. and Hames, B. D., eds. (Oxford University Press: New York, 1992)). Cell culturing conditions can vary according to the type of host cell selected. Commercially available medium can be utilized.

Cells suitable for culturing can contain introduced expression vectors, such as plasmids or viruses. The expression vector constructs can be introduced via transformation, microinjection, transfection, lipofection, electroporation, or infection. The expression vectors can contain coding sequences, or portions thereof, encoding the proteins for expression and production. Expression vectors containing sequences encoding the produced proteins and polypeptides, as well as the appropriate transcriptional and translational control elements, can be generated using methods well known to and practiced by those skilled in the art. These methods include synthetic techniques, in vitro recombinant DNA techniques, and in vivo genetic recombination which are described in J. Sambrook et al., 201, Molecular Cloning, A Laboratory Manual, Cold Spring Harbor Press, Cold Spring Harbor, N.Y. and in F. M. Ausubel et al., 1989, Current Protocols in Molecular Biology, John Wiley & Sons, New York, N.Y.

Purification of Recombinant Proteins

A ANTXR molecule (such as, e.g., ANTXR1 or ANTXR2) can be purified from any human or non-human cell which expresses the polypeptide, including those which have been transfected with expression constructs that express a ANTXR molecule. A purified ANTXR molecule (such as, e.g., ANTXR1, ANTXR2, or a fusion thereof) can be separated from other compounds which normally associate with the ANTXR molecules, in the cell, such as certain proteins, carbohydrates, or lipids, using methods practiced in the art. For protein recovery, isolation and/or purification, the cell culture medium or cell lysate is centrifuged to remove particulate cells and cell debris. The desired polypeptide molecule (for example, a ANTXR molecule) is isolated or purified away from contaminating soluble proteins and polypeptides by suitable purification techniques. Non-limiting purification methods for proteins include: size exclusion chromatography; affinity chromatography; ion exchange chromatography; ethanol precipitation; reverse phase HPLC; chromatography on a resin, such as silica, or cation exchange resin, e.g., DEAE; chromatofocusing; SDS-PAGE; ammonium sulfate precipitation; gel filtration using, e.g., Sephadex G-75, Sepharose; protein A sepharose chromatography for removal of immunoglobulin contaminants; and the like. Other additives, such as protease inhibitors (e.g., PMSF or proteinase K) can be used to inhibit proteolytic degradation during purification. Purification procedures that can select for carbohydrates can also be used, e.g., ion-exchange soft gel chromatography, or HPLC using cation- or anion-exchange resins, in which the more acidic fraction(s) is/are collected.

Methods of Administration

Nucleic Acid Delivery Methods.

The invention provides methods for treating fibrosis, a fibrotic disease, or an epithelial cancer, or to cause a decrease in fibrosis, or a decrease in tumor cell invasion, or a decrease in metastasis, or a decrease in angiogenesis, or a decrease in tumor growth. In one embodiment, the method can comprise administering to the subject a ANTXR molecule (e.g, a ANTXR polypeptide or a ANTXR polynucleotide).

Various approaches can be carried out to restore the activity or function of a ANTXR molecule (such as, e.g., ANTXR1, ANTXR2, or a fusion thereof) in a subject, such as those carrying an altered ANTXR gene locus. For example, supplying wild-type ANTXR gene function (such as, e.g., ANTXR1, ANTXR2) to such subjects can treat or reduce the symptoms associated with fibrosis, a fibrotic disease, or an epithelial cancer, or cause a decrease in fibrosis, or a decrease in tumor cell invasion, or a decrease in metastasis, or a decrease in angiogenesis, or a decrease in tumor growth. Increasing a ANTXR gene expression level or activity (such as, e.g., ANTXR1 or ANTXR2) can be accomplished through gene or protein therapy.

A nucleic acid encoding a ANTXR molecule can be introduced into the cells of a subject. For example, the wild-type gene (or fragment thereof) can also be introduced into the cells of the subject in need thereof using a vector as described herein. The vector can be a viral vector or a plasmid. The gene can also be introduced as naked DNA. The gene can be provided so as to integrate into the genome of the recipient host cells, or to remain extra-chromosomal. Integration can occur randomly or at precisely defined sites, such as through homologous recombination. For example, a functional copy of an ANTXR molecule can be inserted in replacement of an altered version in a cell, through homologous recombination. Further techniques include gene gun, liposome-mediated transfection, or cationic lipid-mediated transfection. Gene therapy can be accomplished by direct gene injection, or by administering ex vivo prepared genetically modified cells expressing a functional polypeptide.

Delivery of nucleic acids into viable cells can be effected ex vivo, in situ, or in vivo by use of vectors, and more particularly viral vectors (e.g., lentivirus, adenovirus, adeno-associated virus, or a retrovirus), or ex vivo by use of physical DNA transfer methods (e.g., liposomes or chemical treatments). Non-limiting techniques suitable for the transfer of nucleic acid into mammalian cells in vitro include the use of liposomes, electroporation, microinjection, cell fusion, DEAE-dextran, and the calcium phosphate precipitation method (see, for example, Anderson, Nature, supplement to vol. 392, no. 6679, pp. 25-20 (1998)). Introduction of a nucleic acid or a gene encoding a polypeptide of the invention can also be accomplished with extrachromosomal substrates (transient expression) or artificial chromosomes (stable expression). Cells may also be cultured ex vivo in the presence of therapeutic compositions of the present invention in order to proliferate or to produce a desired effect on or activity in such cells. Treated cells can then be introduced in vivo for therapeutic purposes.

Nucleic acids can be inserted into vectors and used as gene therapy vectors. A number of viruses have been used as gene transfer vectors, including papovaviruses, e.g., SV40 (Madzak et al., (1992) J Gen Virol. 73(Pt 6):1533-6), adenovirus (Berkner (1992) Curr Top Microbiol Immunol. 158:39-66; Berkner (1988) Biotechniques, 6(7):616-29; Gorziglia and Kapikian (1992) J Virol. 66(7):4407-12; Quantin et al., (1992) Proc Natl Acad Sci USA. 89(7):2581-4; Rosenfeld et al., (1992) Cell. 68(1):143-55; Wilkinson et al., (1992) Nucleic Acids Res. 20(9):2233-9; Stratford-Perricaudet et al., (1990) Hum Gene Ther. 1(3):241-56), vaccinia virus (Moss (1992) Curr Opin Biotechnol. 3(5):518-22), adeno-associated virus (Muzyczka, (1992) Curr Top Microbiol Immunol. 158:97-129; Ohi et al., (1990) Gene. 89(2):279-82), herpesviruses including HSV and EBV (Margolskee (1992) Curr Top Microbiol Immunol. 158:67-95; Johnson et al., (1992) Brain Res Mol Brain Res. 12(1-3):95-102; Fink et al., (1992) Hum Gene Ther. 3(1):11-9; Breakefield and Geller (1987) Mol Neurobiol. 1(4):339-71; Freese et al., (1990) Biochem Pharmacol. 40(10):2189-99), and retroviruses of avian (Bandyopadhyay and Temin (1984) Mol Cell Biol. 4(4):749-54; Petropoulos et al., (1992) J Virol. 66(6):3391-7), murine (Miller et al. (1992) Mol Cell Biol. 12(7):3262-72; Miller et al., (1985) J Virol. 55(3):521-6; Sorge et al., (1984) Mol Cell Biol. 4(9):1730-7; Mann and Baltimore (1985) J Virol. 54(2):401-7; Miller et al., (1988) J Virol. 62(11):4337-45), and human origin (Shimada et al., (1991) J Clin Invest. 88(3):1043-7; Helseth et al., (1990) J Virol. 64(12):6314-8; Page et al., (1990) J Virol. 64(11):5270-6; Buchschacher and Panganiban (1992) J Virol. 66(5):2731-9).

Non-limiting examples of in vivo gene transfer techniques include transfection with viral (typically retroviral) vectors (see U.S. Pat. No. 5,252,479, which is incorporated by reference in its entirety) and viral coat protein-liposome mediated transfection (Dzau et al., Trends in Biotechnology 11:205-210 (1993), incorporated entirely by reference). For example, naked DNA vaccines are generally known in the art; see Brower, Nature Biotechnology, 16:1304-1305 (1998), which is incorporated by reference in its entirety. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

For reviews of gene therapy protocols and methods see Anderson et al., Science 256:808-813 (1992); U.S. Pat. Nos. 5,252,479, 5,747,469, 6,017,524, 6,143,290, 6,410,010 6,511,847; 8,398,968; and 8,404,653 which are all hereby incorporated by reference in their entireties. For an example of gene therapy treatment in humans see Porter et al., NEJM 2011 365:725-733 and Kalos et al. Sci. Transl. Med. 2011. 201 3(95):95. For additional reviews of gene therapy technology, see Friedmann, Science, 244:1275-1281 (1989); Verma, Scientific American: 68-84 (1990); Miller, Nature, 357: 455-460 (1992); Kikuchi et al., J Dermatol Sci. 2008 May; 50(2):87-98; Isaka et al., Expert Opin Drug Deliv. 2007 September; 4(5):561-71; Jager et al., Curr Gene Ther. 2007 August; 7(4):272-83; Waehler et al., Nat Rev Genet. 2007 August; 8(8):573-87; Jensen et al., Ann Med. 2007; 39(2):108-15; Herweijer et al., Gene Ther. 2007 January; 14(2):99-107; Eliyahu et al., Molecules, 2005 Jan. 31; 10(1):34-64; and Altaras et al., Adv Biochem Eng Biotechnol. 2005; 99:193-260, all of which are hereby incorporated by reference in their entireties.

These methods described herein are by no means all-inclusive, and further methods to suit the specific application is understood by the ordinary skilled artisan. Moreover, the effective amount of the compositions can be further approximated through analogy to compounds known to exert the desired effect.

Protein Delivery Methods.

Protein replacement therapy can increase the amount of protein by exogenously introducing wild-type or biologically functional protein by way of infusion. A replacement polypeptide can be synthesized according to known chemical techniques or may be produced and purified via known molecular biological techniques. Protein replacement therapy has been developed for various disorders. For example, a wild-type protein can be purified from a recombinant cellular expression system (e.g., mammalian cells or insect cells-see U.S. Pat. No. 5,580,757 to Desnick et al.; U.S. Pat. Nos. 6,395,884 and 6,458,574 to Selden et al.; U.S. Pat. No. 6,461,609 to Calhoun et al.; U.S. Pat. No. 6,210,666 to Miyamura et al.; U.S. Pat. No. 6,083,725 to Selden et al.; U.S. Pat. No. 6,451,600 to Rasmussen et al.; U.S. Pat. No. 5,236,838 to Rasmussen et al. and U.S. Pat. No. 5,879,680 to Ginns et al.), human placenta, or animal milk (see U.S. Pat. No. 6,188,045 to Reuser et al.), or other sources known in the art. After the infusion, the exogenous protein can be taken up by tissues through non-specific or receptor-mediated mechanism.

An ANTXR molecule can also be delivered in a controlled release system. For example, the ANTXR molecule can be administered using intravenous infusion, an implantable osmotic pump, a transdermal patch, liposomes, or other modes of administration. In one embodiment, a pump can be used (see Sefton (1987) Biomed. Eng. 14:201; Buchwald et al. (1980) Surgery 88:507; Saudek et al. (1989) N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, (1983) J. Macromol. Sci. Rev. Macromol. Chem. 23:61; see also Levy et al. (1985) Science 228:190; During et al. (1989) Ann. Neurol. 25:351; Howard et al. (1989) J. Neurosurg. 71:105). In yet another embodiment, a controlled release system can be placed in proximity of the therapeutic target thus requiring only a fraction of the systemic dose (see, e.g., Goodson, in Medical Applications of Controlled Release, supra, vol. 2, pp. 115-138 (1984)). Other controlled release systems are discussed in the review by Langer (Science (1990) 249:1527-1533).

Pharmaceutical Compositions and Methods of Administration

In some embodiments, a ANTXR molecule can be supplied in the form of a pharmaceutical composition, comprising an isotonic excipient prepared under sufficiently sterile conditions for human administration. Choice of the excipient and any accompanying elements of the composition comprising a ANTXR molecule can be adapted in accordance with the route and device used for administration. In some embodiments, a composition comprising a ANTXR molecule can also comprise, or be accompanied with, one or more other ingredients that facilitate the delivery or functional mobilization of the ANTXR molecule.

These methods described herein are by no means all-inclusive, and further methods to suit the specific application is understood by the ordinary skilled artisan. Moreover, the effective amount of the compositions can be further approximated through analogy to compounds known to exert the desired effect.

According to the invention, a pharmaceutically acceptable carrier can comprise any and all solvents, dispersion media, coatings, antibacterial and antifungal agents, isotonic and absorption delaying agents, and the like, compatible with pharmaceutical administration. The use of such media and agents for pharmaceutically active substances is well known in the art. Any conventional media or agent that is compatible with the active compound can be used. Supplementary active compounds can also be incorporated into the compositions.

An ANTXR molecule (such as, e.g., ANTXR1, ANTXR2, or a fusion thereof) can be administered to the subject one time (e.g., as a single injection or deposition). Alternatively, a ANTXR molecule can be administered once or twice daily to a subject in need thereof for a period of from about 2 to about 28 days, or from about 7 to about 10 days, or from about 7 to about 15 days. It can also be administered once or twice daily to a subject for a period of 1, 2, 3, 4, 5, 6, 7, 8, 9, 10, 11, 12 times per year, or a combination thereof. Furthermore, a ANTXR molecule can be co-administrated with another therapeutic.

In one embodiment, an ANTXR molecule can be co-administrated with a chemotherapy drug. In one embodiment, the administering is conducted simultaneously. In another embodiment, the administering is conducted sequentially in any order. Some non-limiting examples of conventional chemotherapy drugs include: aminoglutethimide, amsacrine, asparaginase, bcg, anastrozole, bleomycin, buserelin, bicalutamide, busulfan, capecitabine, carboplatin, camptothecin, chlorambucil, cisplatin, carmustine, cladribine, colchicine, cyclophosphamide, cytarabine, dacarbazine, cyproterone, clodronate, daunorubicin, diethylstilbestrol, docetaxel, dactinomycin, doxorubicin, dienestrol, etoposide, exemestane, filgrastim, fluorouracil, fludarabine, fludrocortisone, epirubicin, estradiol, gemcitabine, genistein, estramustine, fluoxymesterone, flutamide, goserelin, leuprolide, hydroxyurea, idarubicin, levamisole, imatinib, lomustine, ifosfamide, megestrol, melphalan, interferon, irinotecan, letrozole, leucovorin, ironotecan, mitoxantrone, nilutamide, medroxyprogesterone, mechlorethamine, mercaptopurine, mitotane, nocodazole, octreotide, methotrexate, mitomycin, paclitaxel, oxaliplatin, temozolomide, pentostatin, plicamycin, suramin, tamoxifen, porfimer, mesna, pamidronate, streptozocin, teniposide, procarbazine, titanocene dichloride, raltitrexed, rituximab, testosterone, thioguanine, vincristine, vindesine, thiotepa, topotecan, tretinoin, vinblastine, trastuzumab, and vinorelbine.

In one embodiment, the chemotherapy drug is an alkylating agent, a nitrosourea, an anti-metabolite, a topoisomerase inhibitor, a mitotic inhibitor, an anthracycline, a corticosteroid hormone, a sex hormone, or a targeted anti-tumor compound.

A targeted anti-tumor compound is a drug designed to attack cancer cells more specifically than standard chemotherapy drugs can. Most of these compounds attack cells that harbor mutations of certain genes, or cells that overexpress copies of these genes. In one embodiment, the anti-tumor compound can be imatinib (Gleevec), gefitinib (Iressa), erlotinib (Tarceva), rituximab (Rituxan), or bevacizumab (Avastin).

An alkylating agent works directly on DNA to prevent the cancer cell from propagating. These agents are not specific to any particular phase of the cell cycle. In one embodiment, alkylating agents can be selected from busulfan, cisplatin, carboplatin, chlorambucil, cyclophosphamide, ifosfamide, dacarbazine (DTIC), mechlorethamine (nitrogen mustard), melphalan, and temozolomide.

An antimetabolite makes up the class of drugs that interfere with DNA and RNA synthesis. These agents work during the S phase of the cell cycle and are commonly used to treat leukemia, tumors of the breast, ovary, and the gastrointestinal tract, as well as other cancers. In one embodiment, an antimetabolite can be 5-fluorouracil, capecitabine, 6-mercaptopurine, methotrexate, gemcitabine, cytarabine (ara-C), fludarabine, or pemetrexed.

Topoisomerase inhibitors are drugs that interfere with the topoisomerase enzymes that are important in DNA replication. Some examples of topoisomerase I inhibitors include topotecan and irinotecan while some representative examples of topoisomerase II inhibitors include etoposide (VP-16) and teniposide.

Anthracyclines are chemotherapy drugs that also interfere with enzymes involved in DNA replication. These agents work in all phases of the cell cycle and thus, are widely used as a treatment for a variety of cancers. In one embodiment, an anthracycline used with respect to the invention can be daunorubicin, doxorubicin (Adriamycin), epirubicin, idarubicin, or mitoxantrone.

In one embodiment, an ANTXR molecule can be co-administrated with an anti-inflammatory drug. In one embodiment, the administering is conducted simultaneously. In another embodiment, the administering is conducted sequentially in any order. Some non-limiting examples of anti-inflammatory drugs include: anti-inflammatory steroids (corticosteroids) (e.g. prednisone), aminosalicylates (e.g., mesalazine), non-steroidal anti-inflammatory drugs (NSAIDs) (e.g. aspirin, ibuprofen, naproxen) and immune selective anti-inflammatory derivatives (ImSAIDs). An anti-inflammatory drug also includes antibodies or molecules that target cytokines and chemokines including, but not limited to, anti-TNFα antibodies (e.g. infliximab (Remicade), adalimumab (Humira), certolizumab pegol (Cimzia), golimumab (Simponi), etanercept (Enbrel)), anti-IL12 antibodies, anti-IL2 antibodies (basiliximab (Simulect), daclizumab (Zenapax), azathioprine (Imuran®, Azasan®), 6-mercaptopurine (6-MP, Purinethol®), cyclosporine A (Sandimmune®, Neoral®), tacrolimus (Prograf®), anti-CSF antibodies, and anti-GM-CSF antibodies.

In one embodiment, an ANTXR molecule can be co-administrated with radiation therapy. In one embodiment, the administering is conducted simultaneously. In another embodiment, the administering is conducted sequentially in any order. Some non-limiting examples of conventional radiation therapy include: external beam radiation therapy, sealed source radiation therapy, unsealed source radiation therapy, particle therapy, and radioisotope therapy.

In one embodiment, an ANTXR molecule can be co-administrated with a cancer immunotherapy. In one embodiment, the administering is conducted simultaneously. In another embodiment, the administering is conducted sequentially in any order. Cancer immunotherapy comprises using the immune system of the subject to treat a cancer. For example, the immune system of a subject can be stimulated to recognize and eliminate cancer cells. Some non-limiting examples of cancer immunotherapy include: cancer vaccines, therapeutic antibodies, such as monoclonal antibody therapy (e.g., Bevacizumab, Cetuximab, and Panitumumab), cell based immunotherapy, and adoptive cell based immunotherapy.

An ANTXR molecule may also be used in combination with surgical or other interventional treatment regimens used for the treatment of a fibrotic disease or an epithelial cancer.

An ANTXR molecule can be administered to a subject by any means suitable for delivering the protein, nucleic acid or compound to cells of the subject. For example, it can be administered by methods suitable to transfect cells. Transfection methods for eukaryotic cells are well known in the art, and include direct injection of the nucleic acid into the nucleus or pronucleus of a cell; electroporation; liposome transfer or transfer mediated by lipophilic materials; receptor mediated nucleic acid delivery, bioballistic or particle acceleration; calcium phosphate precipitation, and transfection mediated by viral vectors.

The compositions of this invention can be formulated and administered to reduce the symptoms associated with a fibrotic disease or an epithelial cancer by any means that produce contact of the active ingredient with the agent's site of action in the body of a human or non-human subject. For example, the compositions of this invention can be formulated and administered to reduce the symptoms associated with fibrosis, a fibrotic disease, or an epithelial cancer, or to cause a decrease in fibrosis, or a decrease in tumor cell invasion, or a decrease in metastasis, or a decrease in angiogenesis, or a decrease in tumor growth. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

Pharmaceutical compositions for use in accordance with the invention can be formulated in conventional manner using one or more physiologically acceptable carriers or excipients. The therapeutic compositions of the invention can be formulated for a variety of routes of administration, including systemic and topical or localized administration. Techniques and formulations generally can be found in Remmington's Pharmaceutical Sciences, Meade Publishing Co., Easton, Pa (20^{th} ed., 2000), the entire disclosure of which is herein incorporated by reference. For systemic administration, an injection is useful, including intramuscular, intravenous, intraperitoneal, and subcutaneous. For injection, the therapeutic compositions of the invention can be formulated in liquid solutions, for example in physiologically compatible buffers, such as PBS, Hank's solution, or Ringer's solution. In addition, the therapeutic compositions can be formulated in solid form and redissolved or suspended immediately prior to use. Lyophilized forms are also included. Pharmaceutical compositions of the present invention are characterized as being at least sterile and pyrogen-free. These pharmaceutical formulations include formulations for human and veterinary use.

Any of the therapeutic applications described herein can be applied to any subject in need of such therapy, including, for example, a mammal such as a dog, a cat, a cow, a horse, a rabbit, a monkey, a pig, a sheep, a goat, or a human.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). The composition must be sterile and fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyethylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, and thimerosal. In many cases, it can be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the ANTXR molecule in the required amount in an appropriate solvent with one or a combination of ingredients enumerated herein, as required, followed by filtered sterilization. Dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional desired ingredient from a previously sterile-filtered solution thereof.

Oral compositions include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as known in the art

A composition of the invention can be administered to a subject in need thereof. Subjects in need thereof can include but are not limited to, for example, a mammal such as a dog, a cat, a cow, a horse, a rabbit, a monkey, a pig, a sheep, a goat, or a human.

A composition of the invention can also be formulated as a sustained and/or timed release formulation. Such sustained and/or timed release formulations can be made by sustained release means or delivery devices that are well known to those of ordinary skill in the art, such as those described in U.S. Pat. Nos. 3,845,770; 3,916,899; 3,536,809; 3,598,123; 4,008,719; 4,710,384; 5,674,533; 5,059,595; 5,591,767; 5,120,548; 5,073,543; 5,639,476; 5,354,556; and 5,733,566, the disclosures of which are each incorporated herein by reference. The pharmaceutical compositions of the invention (e.g., that have a therapeutic effect) can be used to provide slow or sustained release of one or more of the active ingredients using, for example, hydropropylmethyl cellulose, other polymer matrices, gels, permeable membranes, osmotic systems, multilayer coatings, microparticles, liposomes, microspheres, or the like, or a combination thereof to provide the desired release profile in varying proportions. Suitable sustained release formulations known to those of ordinary skill in the art, including those described herein, can be readily selected for use with the pharmaceutical compositions of the invention. Single unit dosage forms suitable for oral administration, such as, but not limited to, tablets, capsules, gel-caps, caplets, or powders, that are adapted for sustained release are encompassed by the invention.

In the methods described herein, a ANTXR molecule, can be administered to the subject either as RNA, in conjunction with a delivery reagent, or as a nucleic acid (e.g., a recombinant plasmid or viral vector) comprising sequences which express the gene product. Suitable delivery reagents for administration of the a ANTXR molecule, include the Mirus Transit TKO lipophilic reagent; lipofectin; lipofectamine; cellfectin; or polycations (e.g., polylysine), or liposomes.

The dosage administered can be a therapeutically effective amount of the composition sufficient to result in treatment of fibrosis, a fibrotic disease, or an epithelial cancer, or to cause a decrease in fibrosis, or a decrease in tumor cell invasion, or a decrease in metastasis, or a decrease in angiogenesis, or a decrease in tumor growth, and can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect desired; and rate of excretion.

In some embodiments, the effective amount of the administered ANTXR molecule is at least about 0.01 μg/kg body weight, at least about 0.025 μg/kg body weight, at least about 0.05 μg/kg body weight, at least about 0.075 μg/kg body weight, at least about 0.1 μg/kg body weight, at least about 0.25 μg/kg body weight, at least about 0.5 μg/kg body weight, at least about 0.75 μg/kg body weight, at least about 1 μg/kg body weight, at least about 5 μg/kg body weight, at least about 10 μg/kg body weight, at least about 25 μg/kg body weight, at least about 50 μg/kg body weight, at least about 75 μg/kg body weight, at least about 100 μg/kg body weight, at least about 150 μg/kg body weight, at least about 200 μg/kg body weight, at least about 250 μg/kg body weight, at least about 300 μg/kg body weight, at least about 350 μg/kg body weight, at least about 400 μg/kg body weight, at least about 450 μg/kg body weight, at least about 500 μg/kg body weight, at least about 550 μg/kg body weight, at least about 600 μg/kg body weight, at least about 650 μg/kg body weight, at least about 700 μg/kg body weight, at least about 750 μg/kg body weight, at least about 800 μg/kg body weight, at least about 850 μg/kg body weight, at least about 900 μg/kg body weight, at least about 950 μg/kg body weight, at least about 1000 μg/kg body weight, at least about 1500 μg/kg body weight, at least about 2000 μg/kg body weight, at least about 2500 μg/kg body weight, at least about 3000 μg/kg body weight, at least about 3500 μg/kg body weight, at least about 4000 μg/kg body weight, at least about 4500 μg/kg body weight, at least about 5000 μg/kg body weight, at least about 5500 μg/kg body weight, at least about 6000 μg/kg body weight, at least about 6500 μg/kg body weight, at least about 7000 μg/kg body weight, at least about 7500 μg/kg body weight, at least about 8000 μg/kg body weight, at least about 8500 μg/kg body weight, at least about 9000 μg/kg body weight, at least about 9500 μg/kg body weight, or at least about 10000 μg/kg body weight.

In one embodiment, a ANTXR molecule is administered at least once daily. In another embodiment, a ANTXR molecule is administered at least twice daily. In some embodiments, a ANTXR molecule is administered for at least 1 week, for at least 2 weeks, for at least 3 weeks, for at least 4 weeks, for at least 5 weeks, for at least 6 weeks, for at least 8 weeks, for at least 10 weeks, for at least 12 weeks, for at least 18 weeks, for at least 24 weeks, for at least 36 weeks, for at least 48 weeks, or for at least 60 weeks. In further embodiments, a ANTXR molecule is administered in combination with a second therapeutic agent.

Toxicity and therapeutic efficacy of therapeutic compositions of the present invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Therapeutic agents that exhibit large therapeutic indices are useful. Therapeutic compositions that exhibit some toxic side effects can be used.

Experimental animals can be used as models for human disease. For example, mice can be used as a mammalian model system. The physiological systems that mammals possess can be found in mice, and in humans, for example. Certain diseases can be induced in mice by manipulating their environment, genome, or a combination of both. For example, the AOM/DSS mouse model is a model for human colon cancer. Other mouse models of carcinogenesis include the two-stage DMBA/TPA model of skin cancer, the DEN/CCL4 model of liver cancer, and the H. felis/MNU model of gastric cancer. In addition, there are numerous genetically engineered models of cancer, such as the KPC model of pancreatic cancer. Non-limiting example of mouse models of mammary cancer include, but are not limited to, MMTV-HER2/Neu or MMTV-Wnt-1 or MMTV-PyV-mT. Additional models are described in Hennighausen (2000) Breast Cancer Res. 2(1): 2-7; and Fantozzi et al., (2006) Breast Cancer Res. 2006; 8(4): 212, each of which are hereby incorporated by reference in their entireties.

Non-limiting example of mouse models of prostate cancer include, but are not limited to, Androgen Receptor Knockout mouse, PB-Cre4×PTEN(loxP/loxP) mouse, TRAMP (for transgenic adenocarcinoma mouse prostate), FG-Tag mouse, PB-Neu, and LADY. Additional models are described in Jeet et al (2010) Cancer Metastasis Rev. 29(1):123-42; Zhou et al., (2010) J Androl. 31(3):235-43; Ahmad et al., (2008) Expert Rev Mol Med. 10:e16; Havens et al., (2008) Neoplasia. 10(4): 371-379; Valkenburg and Williams (2011) Prostate Cancer, Volume 2011, Article ID 895238, doi:10.1155/2011/895238, each of which are hereby incorporated by reference in their entireties.

Non-limiting example of mouse models of lung cancer include, but are not limited to, CC10-Tag/CC10-hASH1, K5-E6/E7, CCRP-H-Ras, and MMTV-TGF-β1 DN. Additional models are described in Meuwissen and Berns (2005) GENES & DEVELOPMENT 19:643-664; Kwon and Berns (2013) Molecular Oncology 7(2):165-177; de Serrano and Meuwissen (2010) Eur Respir J. 35: 426-443, each of which are hereby incorporated by reference in their entireties.

Administration of a ANTXR molecule is not restricted to a single route, but may encompass administration by multiple routes. Multiple administrations may be sequential or concurrent. Other modes of application by multiple routes will be apparent to one of skill in the art.

Monoclonal antibodies have emerged as increasingly important therapeutic entities for the treatment of human diseases such as rheumatoid arthritis, multiple sclerosis and different types of cancers. Monoclonal antibodies specifically recognize foreign antigens or target cells, stimulating the patient's immune system to attack abnormal cells and fight the disease. Therapeutic monoclonal antibodies also act as receptor antagonists by blocking the interaction of a ligand with its receptor, thus directly blocking protein function. These antibodies can be specific to any extracellular or cell surface target. There are a number of ways that monoclonal antibodies can be used for therapy. This type of therapy can destroy malignant tumor cells and prevent tumor growth by blocking specific cell receptors, or it can abrogate malicious signaling pathways leading to the development of serious conditions by blocking antigen(ligand)/receptor binding and subsequently protein function.

The invention relates generally to the field of molecular biology. In particular, this invention relates to isolated polypeptides and methods of use thereof. This invention encompasses the addition of a peptide domain, for example a carboxy-terminal peptide (CTP) domain, that promotes glycosylation and stabilization of the antibodies. It also enhances the antibodies' durability, increases their effectiveness, and improves the bioavailability of the therapeutic.

A need exists for polypeptides, including antibodies, that have increased stability and longer half-lives, making them suitable for therapeutic treatment. The embodiments disclosed herein address this need and provide other benefits.

As discussed herein, in vitro experimental results demonstrate that the fusion of the carboxy-terminal peptide (CTP) domain of human chorionic gonadotropin (hCG) and Fc or Fab fragments can confer stability to antibodies. The CTP domain is important in providing stability and a longer biological half-life to the proteins in the circulation. In one embodiment, Fc or Fab fragments tagged with CTP are “durable Fc” or “durable Fab” moieties.

### General Considerations

The invention is based, at least in part, on the discovery of isolated polypeptides comprising a CTP domain fused to an antibody fragment, and methods of making and using the same. This invention is also based, at least in part, on the discovery that fusing a CTP domain to the ectodomain of a receptor results in increased glycosylation and/or protein stability. The addition of the CTP domain to the ectodomain of a receptor differs from previous studies in that the CTP domain is used to stabilize the receptor rather than enhance the effect of a hormone, such as growth factors. It has been unexpectedly discovered that the addition of a CTP domain to antibody fragments result in isolated polypeptides that are glycosylated. Although not bound by any theory, this glycosylation confers stability and a longer biological half-life to the polypeptides in circulation. The polypeptides described herein improves biosensors by aiding them in identifying pathogens in public places and in locations where pathogen detection can be difficult. The polypeptides described herein can be used to make improved detection and diagnostic kits, as stable proteins can survive in more environments. The polypeptides can be used in both laboratory and non-laboratory applications, such as environmental remediation, e.g., pollutant removal. Finally, the polypeptides are useful for treating diseases that are currently being treated with antibody-based therapeutics, including but not limited to cancer, immune-related disorders, cardiovascular disease, obesity, diabetes, metabolic disorders, and blindness.

In laboratory applications, the polypeptides can be used in assays to detect the presence or level of the proteins in body fluids (e.g., blood, lymph, the mucosal layer of the intestine), or in assays to measure the presence or level of expression of the proteins in particular cells. Techniques employing the polypeptides of the invention include Western blotting to identify cells expressing proteins, immunocytochemistry or immunofluorescence techniques to establish the cellular or extracellular location of proteins, and enzyme-linked immunosorbent assay (ELISA) techniques to detect the presence or quantify the polypeptides in a sample.

The isolated polypeptides described herein enhances the antibodies' durability, increases their effectiveness, and improves the bioavailability of the therapeutic agent. Other advantages of the isolated polypeptides include: lower dose of polypeptides needed for therapeutic effect; less potential for impurities to affect use of the polypeptides; and greater ease of purification.

In 1990, it was reported that the C-terminal domain of the beta subunit of the human chorionic gonadotrophin (hCG) was required for in vivo biological responses [1]. This region, referred to as the CTP, was later added to the C-terminus of follicle-stimulating hormone (FSH), a labile hormone, causing a significant increase in the biological half-life of FSH in circulation [2, 3]. Thus, the CTP domain was shown to confer stability in circulation to proteins, allowing for greater bioactivity. The mechanism of the stability increase is thought to be through the attachment of O-linked glycosylation to the CTP domain.

In 2002, a report on the first use of FSH-CTP in humans was published, establishing that recombinant proteins with CTP additions were tolerated and bioactive in humans [4]. Subsequently, other FSH-CTP fusion proteins were developed and reported to have bioactivity and increased in vivo biological half-life [5, 6]. A variety of CTP fusion proteins have been made and reported in the literature, including human erythropoietin [7], human vascular endothelial growth factor (VEGF) [8], and human growth hormone [9]. In these cases, preclinical studies in mice or rats establish the paradigm that CTP fusion proteins may be “long-acting” based upon increased in vivo circulating levels due to increased biological half-life in circulation.

The isolated polypeptides disclosed herein comprises a CTP domain fused to an antibody fragment, and methods of making and using the same.

### Antibodies

Antibody-based therapeutics are currently used in the treatment of numerous serious diseases (Waldmann, Thomas A. (2003). “Immunotherapy: past, present and future” Nature Medicine 9 (3): 269-277). However, the majority of these antibodies are unstable and have short half-lives.

An antibody is any immunoglobulin (Ig) molecule that includes at least one immunoglobulin variable region, e.g., an amino acid sequence that provides an immunoglobulin variable domain or immunoglobulin variable domain sequence. For example, an antibody can include a heavy (H) chain variable region (V_{H}), and a light (L) chain variable region (V_{L}). In another example, an antibody includes four polypeptide chains, two heavy (H) chains and two light (L) chains, interconnected by disulfide bonds or any functional fragment, mutant, variant, or derivation thereof, which retains the essential epitope binding features of an Ig molecule. The light chains of the immunoglobulin can be of types kappa or lambda.

An antibody can be monoclonal, polyclonal, humanized, chimeric, or fully human, or a binding fragment thereof, directed against a known antigen. An antibody can be naturally produced or recombinantly produced. Antibody fragments can include, but are not limited to, single chain Fv (scFv), diabodies, Fv, and (Fab′)2, triabodies, Fc, Fab, CDR1, CDR2, CDR3, combinations of CDR's, variable regions, tetrabodies, bifunctional hybrid antibodies, framework regions, constant regions, and the like (see Maynard et al., (2000) Ann. Rev. Biomed. Eng. 2:339-76; Hudson (1998) Curr. Opin. Biotechnol. 9:395-402; see also, Janeway et al., (2001) Immunobiology, 5th ed., Garland Publishing).

The Fab (fragment, antigen binding) region can bind to antigens and specific foreign objects. The Fc (Fragment, crystallizable) region ensures that each antibody generates an immune response for an antigen, by binding to Fc receptors or other immune molecules. Useful antibodies include all Ig classes, such as IgM, IgG, IgD, IgE, IgA, and their subtypes. Subtypes of IgG are IgG1, IgG2, IgG3, and IgG4. Thus, the polypeptides disclosed herein include antibody fragments (e.g., single chain antibodies, Fab, F(ab′)₂, Fd, Fv, dAb, and Fc fragments) and complete antibodies, e.g., intact immunoglobulins of types IgA, IgG, IgE, IgD, IgM, and their subtypes. In one embodiment, the antibody is glycosylated, including N-linked or O-linked glycosylated.

Antibodies can be obtained commercially, custom-generated, or synthesized against an antigen of interest according to methods established in the art. See, e.g., Pluckthun (1990), Nature 347:497-498; Huse et al. (1989), Science 246:1275-1289; Chaudhary et al. (1990), Proc. Natl. Acad. Sci. USA 87:1066-1070; Mullinax et al. (1990), Proc. Natl. Acad. Sci. USA 87:8095-8099; Berg et al. (1991), Proc. Natl. Acad. Sci. USA 88:4723-4727; Wood et al. (1990), J. Immunol. 145:3011-3016; and references cited therein. For an overview of antibody techniques, see, e.g., Antibody Engineering, 2nd Ed., Borrebaeck, ed., Oxford University Press, Oxford (1995); Rubinstein et al. (2003), Anal Biochem 314:294-300; Traggia et al. (2004), Nat. Med. 10(8):871-875; and Lanzavecchia et al. (2006), Immunol. Rev., 2 11:303-309.

The Fc region is the C-terminal region of an immunoglobulin heavy chain, which may be generated by papain digestion of an intact antibody. The Fc region may be a native sequence Fc region or a variant Fc region. The Fc region of an immunoglobulin generally comprises two constant domains, a C_{H}2 domain and a C_{H}3 domain, and optionally comprises a C_{H}4 domain. Replacements of amino acid residues in the Fc portion to alter antibody effector function are known in the art (U.S. Pat. Nos. 5,648,260 and 5,624,821).

The Fc portion of an antibody mediates several important effector functions, e.g., cytokine induction, antibody dependent cell mediated cytotoxicity (ADCC), phagocytosis, complement-dependent cytotoxicity (CDC) and biological half-life/clearance rate of antibody and antigen-antibody complexes. Certain human IgG isotypes, such as IgG1 and IgG3, mediate ADCC and CDC via binding to FcyRs and complement Clq, respectively. The dimerization of two identical heavy chains of an immunoglobulin is mediated by the dimerization of C_{H}3 domains and is stabilized by the disulfide bonds within the hinge region (Huber et al. (1976) Nature 264:415-20; Thies et al. (1999) J. Mol. Biol. 293:67-79). Mutation of cysteine residues within the hinge regions to prevent heavy chain-heavy chain disulfide bonds destabilizes dimerization of C_{H}3 domains. Residues responsible for C_{H}3 dimerization have been identified (Dall'Acqua (1998) Biochem. 37:9266-73). Therefore, it is possible to generate a monovalent half-Ig molecule.

Ig half-molecules, which have a dimeric configuration that includes only one light chain and only one heavy chain, have been described as the result of rare deletions in human and murine plasmacytomas. Monovalent half Ig molecules have been found in nature for both IgG and IgA subclasses (Seligman (1978) Ann. Immunol. 129:855-70; Biewenga et al. (1983) Clin. Exp. Immunol. 51:395-400). Some studies show that these half-molecules include IgG1 molecules in which the heavy chain C_{H}1, hinge, and C_{H}2 regions appear normal, whereas deletions are found in the C_{H}3 region. A half Ig molecule can have certain advantages in tissue penetration due to its smaller size compared to that of a regular antibody. In one embodiment, at least one amino acid residue is replaced in the constant region of the binding protein, for example the Fc region, such that the dimerization of the heavy chains is disrupted, resulting in half Ig molecules. The light chain may be either a kappa or lambda type.

The antigen-binding portion of an antibody includes fragments of an antibody that retain the ability to specifically bind to an antigen. Such antibody embodiments may also be bispecific, dual specific, or multi-specific, e.g., it specifically binds to two or more different antigens. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, CL and C_{H}1 domains; (ii) a F(ab′)2 fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment (Ward et al, (1989) Nature 341:544-546), which consists of a V_{H} domain; and (vi) an isolated complementarity determining region (CDR).

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Nati. Acad. Sci. USA 85:5879-5883). Such single chain antibodies are also intended to be encompassed within the antigen-binding portion of an antibody. Other forms of single chain antibodies, such as diabodies are also encompassed. Diabodies are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such antibody binding portions are known in the art (Kontermann and Dubel eds. (2001) Antibody Engineering, Springer-Verlag, New York. pp. 790). In addition, single chain antibodies also include “linear antibodies” comprising a pair of tandem Fv segments (VH—CH1-VH—CH1) which, together with complementary light chain polypeptides, form a pair of antigen binding regions (Zapata et al. (1995) Protein Eng. 8(10): 1057-1062; U.S. Pat. No. 5,641,870).

An antibody or antigen-binding portion thereof may be part of a larger immunoadhesion molecules, formed by covalent or non-covalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mo/. Immunol. 31:1047-1058). Antibody portions, such as Fab andF(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques. Antigen binding portions can be complete domains or pairs of complete domains.

A monoclonal antibody (mAb) is an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each mAb is directed against a single determinant on the antigen. A monoclonal antibody can be produced by hybridoma technology.

### CTP-Antibody Fragments

In one embodiment, the invention discloses an isolated polypeptide comprising a CTP domain having at least about 90% identity to SEQ ID NO: 37, and wherein the CTP domain is fused to an antibody fragment. In other embodiments, the CTP domain has at least about 99%, 98%, 97%, 96%, 95%, 94%, 93%, 92%, or 91% identity to SEQ ID NO: 37. The fusion of the CTP domain to the antibody fragments can promote glycosylation, confer stability, and increase the biological half-life of the polypeptides in circulation.

The nucleic acid sequence of the CTP peptide domain is presented as SEQ ID NO: 36, and the amino acid sequence is presented as SEQ ID NO: 37 in the Sequence Listings, listed below. The underlined, bolded regions of the sequences in SEQ ID NO: 30-35 represent the CTP domain.

### CTP-Fusions

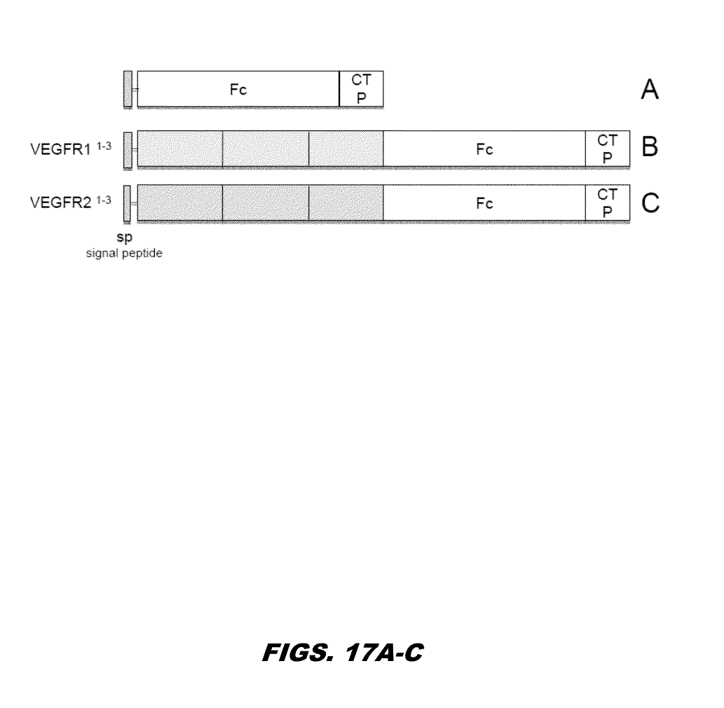

In one embodiment, the invention comprises an isolated polypeptide comprising a carboxy-terminal peptide (CTP) domain comprising SEQ ID NO: 37 fused to an ectodomain of a receptor. For example, fusing a CTP domain encompassing SEQ ID NO: 37 to the ectodomain of a receptor (e.g., a cell surface receptor) results in increased glycosylation and/or protein stability. In one embodiment, the receptor comprises extracellular domains 1-3 of human VEGFR1, as depicted in the shaded regions of FIG. 17B. In another embodiment, the receptor comprises extracellular domains 1-3 of human VEGFR2, as depicted in the shaded regions of FIG. 17B. In further embodiments, the ectodomain of a receptor can further comprise a signal peptide sequence, as depicted in FIGS. 17B-C.

### Sequence Listings

### SEQ ID NO: 30

The nucleic acid sequence of Human Fc (IgG₁)-CTP: hFcCTP

| | |
|---|---|
| 1 | atgtggggct ggaagtgcct cctcttctgg gctgtgctgg tcacagccac tctctgcact |
| 61 | gccaggccag ccccaacctt gcccgaacaa gctcagcagt cgacgcgcgc agatctgggcc |
| 121 | cgggcgagcc caaatcttgt gacaaaactc acacatgccc accgtgccca gcacctgaac |
| 181 | tcctgggggg accgtcagtc ttcctcttcc ccccaaaacc caaggacacc ctcatgatct |
| 241 | cccggacccc tgaggtcaca tgcgtggtgg tggacgtgag ccacgaagac cctgaggtca |
| 301 | agttcaactg gtacgtggac ggcgtggagg tgcataatgc caagacaaag ccgcgggagg |
| 361 | agcagtacaa cagcacgtac cgtgtggtca gcgtcctcac cgtcctgcac caggactggc |
| 421 | tgaatggcaa ggagtacaag tgcaaggtct ccaacaaagc cctcccagcc cccatcgaga |
| 481 | aaaccatctc caaagccaaa gggcagcccc gagaaccaca ggtgtacacc ctgcccccat |
| 541 | cccgggatga gctgaccaag aaccaggtca gcctgacctg cctggtcaaa ggcttctatc |
| 601 | ccagcgacat cgccgtggag tgggagagca atgggcagcc ggagaacaac tacaagacca |
| 661 | cgcctcccgt gctggactcc gacggctcct tcttcctcta cagcaagctc accgtggaca |
| 721 | agagcaggtg gcagcagggg aacgtcttct catgctccgt gatgcatgag gctctgcaca |
| 781 | accactacac gcagaagagc ctctccctgt ctccgggtaa aggatcacca cgcttccagg |
| 841 | actcctcttc ctcaaaggcc cctcctccta gccttccaag cccatcgaga ctcccggggc |
| 901 | cctcggacac tccgatcctc ccacaataa |

### SEQ ID NO:31

The amino acid sequence of Human Fc (IgG₁)-CTP: hFcCTP

| | |
|---|---|
| 1 | MWGWKCLLFW AVLVTATLCT ARPAPTLPEQ AQQSTRADLG PGEPKSCDKT HTCPPCPAPE |
| 61 | LLGGPSVFLF PPKPKDTLMI SRTPEVTCVV VDVSHEDPEV KFNWYVDGVE VHNAKTKPRE |
| 121 | EQYNSTYRVV SVLTVLHQDW LNGKEYKCKV SNKALPAPIE KTISKAKGQP REPQVYTLPP |
| 181 | SRDELTKNQV SLTCLVKGFY PSDIAVEWES NGQPENNYKT TPPVLDSDGS FFLYSKLTVD |
| 241 | KSRWQQGNVF SCSVMHEALH NHYTQKSLSL SPGKGSPRFQ DSSSSKAPPP SLPSPSRLPG |
| 301 | PSDTPILPQ |

### SEQ ID NO:32

The nucleic acid sequence of hVEGFR1¹⁻³-FcCTP

| | |
|---|---|
| 1 | atggtcagct actgggacac cggggtcctg ctgtgcgcgc tgctcagctg tctgcttctc |
| 61 | acaggatcta gttcaggttc aaaattaaaa gatcctgaac tgagtttaaa aggcacccag |
| 121 | cacatcatgc aagcaggcca gacactgcat ctccaatgca ggggggaagc agcccataaa |
| 181 | tggtctttgc ctgaaatggt gagtaaggaa agcgaaaggc tgagcataac taaatctgcc |
| 241 | tgtggaagaa atggcaaaca attctgcagt actttaacct tgaacacagc tcaagcaaac |
| 301 | cacactggct tctacagctg caaatatcta gctgtaccta cttcaaagaa gaaggaaaca |
| 361 | gaatctgcaa tctatatatt tattagtgat acaggtagac ctttcgtaga gatgtacagt |
| 421 | gaaatccccg aaattataca catgactgaa ggaagggagc tcgtcattcc ctgccgggtt |
| 481 | acgtcaccta acatcactgt tactttaaaa aagtttccac ttgacacttt gatccctgat |
| 541 | ggaaaacgca taatctggga cagtagaaag ggcttcatca tatcaaatgc aacgtacaaa |
| 601 | gaaatagggc ttctgacctg tgaagcaaca gtcaatgggc atttgtataa gacaaactat |
| 661 | ctcacacatc gacaaaccaa tacaatcata gatgtccaaa taagcacacc acgcccagtc |
| 721 | aaattactta gaggccatac tcttgtcctc aattgtactg ctaccactcc cttgaacacg |
| 781 | agagttcaaa tgacctggag ttaccctgat gaaaaaaata agagagcttc cgtaaggcga |
| 841 | cgaattgacc aaagcaattc ccatgccaac atattctaca gtgttcttac tattgacaaa |
| 901 | atgcagaaca aagacaaagg actttatact tgtcgtgtaa ggagtggacc atcattcaaa |
| 961 | tctgttaaca cctcagtgca tatatatgat aaagcattca tcactgtgaa acaagatctg |
| 1021 | ggcccgggcg agcccaaatc ttgtgacaaa actcacacat gcccaccgtg cccagcacct |
| 1081 | gaactcctgg ggggaccgtc agtcttcctc ttccccccaa aacccaagga caccctcatg |
| 1141 | atctcccgga cccctgaggt cacatgcgtg gtggtggacg tgagccacga agaccctgag |
| 1201 | gtcaagttca actggtacgt ggacggcgtg gaggtgcata atgccaagac aaagccgcgg |
| 1261 | gaggagcagt acaacagcac gtaccgtgtg gtcagcgtcc tcaccgtcct gcaccaggac |
| 1321 | tggctgaatg gcaaggagta caagtgcaag gtctccaaca aagccctccc agcccccatc |
| 1381 | gagaaaacca tctccaaagc caaagggcag ccccgagaac cacaggtgta caccctgccc |
| 1441 | ccatcccggg atgagctgac caagaaccag gtcagcctga cctgcctggt caaaggcttc |
| 1501 | tatcccagcg acatcgccgt ggagtgggag agcaatgggc agccggagaa caactacaag |
| 1561 | accacgcctc ccgtgctgga ctccgacggc tccttcttcc tctacagcaa gctcaccgtg |
| 1621 | gacaagagca ggtggcagca ggggaacgtc ttctcatgct ccgtgatgca tgaggctctg |
| 1681 | cacaaccact acacgcagaa gagcctctcc ctgtctccgg gtaaaggatc accacgcttc |
| 1741 | caggactcct cttcttcaaa ggcccctcct cctagcttcc caagcccatc ccgactcccg |
| 1801 | gggccctcgg acactcgat cctcccacaa taa |

### SEQ ID NO: 33

The amino acid sequence of hVEGFR1¹⁻³-FcCTP:

| | |
|---|---|
| 1 | MVSYWDTGVL LCALLSCLLL TGSSSGSKLK DPELSLKGTQ HIMQAGQTLH LQCRGEAAHK |
| 61 | WSLPEMVSKE SERLSITKSA CGRNGKQFCS TLTLNTAQAN HTGFYSCKYL AVPTSKKKET |
| 121 | ESAIYIFISD TGRPFVEMYS EIPEIIHMTE GRELVIPCRV TSPNITVTLK KFPLDTLIPD |
| 181 | GKRIIWDSRK GFIISNATYK EIGLLTCEAT VNGHLYKTNY LTHRQTNTII DVQISTPRPV |
| 241 | KLLRGHTLVL NCTATTPLNT RVQMTWSYPD EKNKRASVRR RIDQSNSHAN IFYSVLTIDK |
| 301 | MQNKDKGLYT CRVRSGPSFK SVNTSVHIYD KAFITVKQDL GPGEPKSCDK THTCPPCPAP |
| 361 | ELLGGPSVFL FPPKPKDTLM ISRTPEVTCV VVDVSHEDPE VKFNWYVDGV EVHNAKTKPR |
| 421 | EEQYNSTYRV VSVLTVLHQD WLNGKEYKCK VSNKALPAPI EKTISKAKGQ PREPQVYTLP |
| 481 | PSRDELTKNQ VSLTCLVKGF YPSDIAVEWE SNGQPENNYK TTPPVLDSDG SFFLYSKLTV |
| 541 | DKSRWQQGNV FSCSVMHEAL HNHYTQKSLS LSPGKGSPRF QDSSSSKAPP PSLPSPSRLP |
| 601 | GPSDTPILPQ |

### SEQ ID NO: 34

The nucleic acid sequence of hVEGFR2¹⁻³-FcCTP:

| | |
|---|---|
| 1 | atgcagagca aggtgctgct ggccgtcgcc ctgtggctct gcgtggagac ccgggccgcc |
| 61 | tctgtgggtt tgcctagtgt ttctcttgat ctgcccaggc tcagcataca aaaagacata |
| 121 | cttacaatta aggctaatac aactcttcaa attacttgca ggggacagag ggacttggac |
| 181 | tggctttggc ccaataatca gagtggcagt gagcaaaggg tggaggtgac tgagtgcagc |
| 241 | gatggcctct tctgtaagac actcacaatt ccaaaagtga tcggaaatga cactggagcc |
| 301 | tacaagtgct tctaccggga aactgacttg gcctcggtca tttatgtcta tgttcaagat |
| 361 | tacagatctc catttattgc ttctgttagt gaccaacatg gagtcgtgta cattactgag |
| 421 | aacaaaaaca aaactgtggt gattccatgt ctcgggtcca tttcaaatct caacgtgtca |
| 481 | ctttgtgcaa gatacccaga aaagagattt gttcctgatg gtaacagaat ttcctgggac |
| 541 | agcaagaagg gctttactat tcccagctac atgatcagct atgctggcat ggtcttctgt |
| 601 | gaagcaaaaa ttaatgatga aagttaccag tctattatgt acatagttgt cgttgtaggg |
| 661 | tataggattt atgatgtggt tctgagtccg tctcatggaa ttgaactatc tgttggagaa |
| 721 | aagcttgtct taaattgtac agcaagaact gaactaaatg tggggattga cttcaactgg |
| 781 | gaataccctt cttcgaagca tcagcataag aaacttgtaa accgagacct aaaaacccag |
| 841 | tctgggagtg agatgaagaa atttttgagc accttaacta tagatggtgt aacccggagt |
| 901 | gaccaaggat tgtacacctg tgcagcatcc agtgggctga tgaccaagaa gaacagcaca |
| 961 | tttgtcaggg tccatgaaaa accttttgtt gcttttggaa gtggcatgga atctctggca |
| 1021 | agatctgggc ccgggcgagc ccaaatcttg tgacaaaact cacacatgcc caccgtgccc |
| 1081 | agcacctgaa ctcctggggg gaccgtcagt cttcctcttc cccccaaaac ccaaggacac |
| 1141 | cctcatgatc tcccggaccc ctgaggtcac atgcgtggtg gtggacgtga gccacgaaga |
| 1201 | ccctgaggtc aagttcaact ggtacgtgga cggcgtggag gtgcataatg ccaagacaaa |
| 1261 | gccgcgggag gagcagtaca acagcacgta ccgtgtggtc agcgtcctca ccgtcctgca |
| 1321 | ccaggactgg ctgaatggca aggagtacaa gtgcaaggtc tccaacaaag ccctcccagc |
| 1381 | ccccatcgag aaaaccatct ccaaagccaa agggcagccc cgagaaccac aggtgtacac |
| 1441 | cctgccccca tcccgggatg agctgaccaa gaaccaggtc agcctgacct gcctggtcaa |
| 1501 | aggcttctat cccagcgaca tcgccgtgga gtgggagagc aatgggcagc cggagaacaa |
| 1561 | ctacaagacc acgcctcccg tgctggactc cgacggctcc ttcttcctct acagcaagct |
| 1621 | caccgtggac aagagcaggt ggcagcaggg gaacgtcttc tcatgctccg tgatgcatga |
| 1681 | ggctctgcac aaccactaca cgcagaagag cctctccctg tctccgggta aaggat cacc |
| 1741 | acgcttccag gactcctctt cctcaaaggc ccctcctcct agccttccaa gcccatcccg |
| 1801 | actcccgggg ccctcggaca ctccgatcct cccacaataa |

### SEQ ID NO: 35

The amino acid sequence of hVEGFR2¹⁻³-FcCTP:

| | |
|---|---|
| 1 | MQSKVLLAVA LWLCVETRAA SVGLPSVSLD LPRLSIQKDI LTIKANTTLQ ITCRGQRDLD |
| 61 | WLWPNNQSGS EQRVEVTECS DGLFCKTLTI PKVIGNDTGA YKCFYRETDL ASVIYVYVQD |
| 121 | YRSPFIASVS DQHGVVYITE NKNKTVVIPC LGSISNLNVS LCARYPEKRF VPDGNRISWD |
| 181 | SKKGFTIPSY MISYAGMVFC EAKINDESYQ SIMYIVVVVG YRIYDVVLSP SHGIELSVGE |
| 241 | KLVLNCTART ELNVGIDFNW EYPSSKHQHK KLVNRDLKTQ SGSEMKKFLS TLTIDGVTRS |
| 301 | DQGLYTCAAS SGLMTKKNST FVRVHEKPFV AFGSGMESLL GPGEPKSCDK THTCPPCPAP |
| 361 | ELLGGPSVFL FPPKPKDTLM ISRTPEVTCV VVDVSHEDPE VKFNWYVDGV EVHNAKTKPR |
| 421 | EEQYNSTYRV VSVLTVLHQD WLNGKEYKCK VSNKALPAPI EKTISKAKGQ PREPQVYTLP |
| 481 | PSRDELTKNQ VSLTCLVKGF YPSDIAVEWE SNGQPENNYK TTPPVLDSDG SFFLYSKLTV |
| 541 | DKSRWQQGNV FSCSVMHEAL HNHYTQKSLS LSPGKGSPRF QDSSSSKAPP PSLPSPSRLP |
| 601 | GPSDTPILPQ |

### SEQ ID NO: 36

Nucleic acid sequence of CTP Domain:

ggatcaccacgcttccaggactcctcttcctcaaaggcccctcctcctag
ccttccaagcccatcccgactcccggggccctcggacact ccgatcctc
ccacaataa

### SEQ ID NO: 37

Amino acid sequence of CTP Domain:

GSPRFQDSSSSKAPPPSLPSPSRLPGPSDTPILPQ

Protein glycosylation is an enzymatic process that adds a carbohydrate moiety to a polypeptide. Glycosylation is a post-translational modification for polypeptides involved in cell membrane formation. During this process, the linking of monosaccharide units to the amino acid chains sets up the stage for a series of enzymatic reactions that lead to the formation of glycoproteins. A typical glycoprotein has at least 41 bonds which involve 8 amino acids and 13 different monosaccharide units and includes the glycophosphatidylinositol (GPI) and phosphoglycosyl linkages. Protein glycosylation helps in proper folding of proteins, stability and in cell-to-cell adhesion commonly needed by cells of the immune system. The major sites of protein glycosylation in the body are endoplasmic reticulum (ER), Golgi body, nucleus, and the cell fluid. In certain embodiments, glycosylation can be N-linked or O-linked.

N-linked glycosylation begins with the addition of a 14-sugar precursor to an asparagine in the polypeptide chain of the target protein. The structure of this precursor contains glucose, mannose, and 2 N-acetylglucosamine molecules. A complex set of reactions attaches this branched chain to a carrier molecule called dolichol, and this entity is transferred to the appropriate point on the polypeptide chain as it is translocated into the ER lumen. The motif for an N-linked glycosylation site is Asn-X-Thr/Ser, where X can be any amino acid except proline. Marshall, Glycoproteins. Annu. Rev. Biochem. 41:673-702 (1972). N-linked glycosylation can be important to protein folding.

O-linked glycosylation begins with an enzyme-mediated addition of N-acetyl-galactosamine followed by other carbohydrates (such as galactose and sialic acid) to serine or threonine residues. O-linked glycosylation occurs at later stages of protein processing.

In one embodiment, the CTP peptide domain can differ by at least 1, 2, 3, 4, 5, 6, 7, 8, 9, or 10 conservative amino acid substitutions as compared to SEQ ID NO: 37. In another embodiment, the CTP peptide domain can differ from the native human chorionic gonadotropin CTP by 1, 2, 3, 4, or 5 conservative amino acid substitutions as described in U.S. Pat. No. 5,712,122. In one embodiment, conservative amino acid substitutions can be substitution combinations and their reciprocals as described in Dayhoff et al., ((1978) Atlas of Protein Sequence and Structure, ed. Dayhoff, M. (Natl. Biomed. Res. Found., Silver Spring, Md.), Vol. 5, Suppl. 3, pp. 345-352), which include, but are not limited to: Cys/Ser, Cys/Tyr, Ser/Thr, Ser/Pro, Ser/Ala, Ser/Gly, Ser/Asn, Ser/Asp, Ser/Glu, Ser/Arg, Ser/Lys, Thr/Pro, Thr/Ala, Thr/Gly, Thr/Asn, Thr/Asp, Thr/Glu, Thr/Lys, Thr/Ile, Thr/Val, Pro/Ala, Pro/Gln, Pro/His, Pro/Arg, Ala/Gly, Ala/Asn, Ala/Asp, Ala/Glu, Ala/Gln, Ala/Val, Gly/Asn, Gly/Asp, Gly/Glu, Asn/Asp, Asn/Glu, Asn/Gln, Asn/His, Asn/Arg, Asn/Lys, Asp/Glu, Asp/Gln, Asp/His, Asp/Lys, Glu/Gln, Glu/His, Glu/Lys, Gln/His, Gln/Arg, Gln/Lys, His/Arg, His/Lys, His/Tyr, Arg/Lys, Arg/Met, Arg/Trp, Lys/Met, Met/Ile, Met/Leu, Met/Val, Met/Phe, Ile/Leu, Ile/Val, Ile/Phe, Leu/Val, Leu/Phe, Phe/Tyr, Phe/Trp, and Tyr/Trp.

In one embodiment, the CTP peptide domain comprises at least 1 glycosylation site. In one embodiment, the CTP peptide domain comprises at least 2 glycosylation sites. In one embodiment, the CTP peptide domain comprises at least 3 glycosylation sites. In one embodiment, the CTP peptide domain comprises at least 4 glycosylation sites. In one embodiment, the CTP peptide domain comprises at least 5 glycosylation sites. In some embodiments, SEQ ID NO: 31 comprises at least 1 glycosylation site, at least 2 glycosylation sites, at least 3 glycosylation sites, at least 4 glycosylation sites, at least 5 glycosylation sites, at least 6 glycosylation sites, at least 7 glycosylation sites, at least 8 glycosylation sites, at least 9 glycosylation site, or at least 10 glycosylation sites. In further embodiments, the glycosylation site can be an N-linked glycosylation site, an O-linked glycosylation site, or a combination thereof.

### Isolated Nucleic Acid Molecules

In one aspect, the present invention provides preparations of isolated nucleic acid molecules encoding the isolated polypeptides described herein. These nucleic acids are encoded by SEQ ID NO: 30. In other embodiments, the isolated nucleic acid molecules have at least about 99%, 98%, 97%, 96%, 95%, 94%, 93%, 92%, or 91% identity to SEQ ID NO: 30.

The nucleic acid molecules can be DNA or RNA molecules, hybrid DNA-RNA molecules, or nucleic acid analogs. The nucleic acid analogs can include modified bases (e.g., 2′-halo-2′-deoxynucleosides) and/or modified internucleoside linkages (e.g., peptide nucleic acids, phosphorothioate linkages). The nucleic acids can be sense molecules corresponding to all or a portion of the gene sequence encoding a polypeptide of the invention, or can be antisense molecules which are complementary to all or a portion of a gene sequence encoding a polypeptide of the invention. The nucleic acids can be derived from, or correspond to, genomic DNA or cDNA, or can be synthetic molecules based upon a protein sequence and the genetic code (e.g., synthetic nucleic acids which reflect the codon usage preferences in the host cells used in an expression system).

In some embodiments, the nucleic acid molecules are prepared using PCR techniques and other methods well-known to one skilled in the art. In some embodiments, the procedure involves the ligation of two different DNA sequences (See, e.g., “Current Protocols in Molecular Biology”, eds. Ausubel et al., John Wiley & Sons, 1992). Recombinant DNA techniques are well-known in the art. See, e.g., Perbal, “A Practical Guide to Molecular Cloning,” John Wiley & Sons, New York (1988); Watson et al., “Recombinant DNA,” Scientific American Books, New York; Birren et al. (eds) “Genome Analysis: A Laboratory Manual Series,” Vols. 1-4, Cold Spring Harbor Laboratory Press, New York (1998).

In one embodiment, the nucleic acid molecules are inserted into expression vectors (i.e., a nucleic acid construct) to enable expression of the recombinant polypeptide. In one embodiment, the expression vector includes additional sequences which render this vector suitable for replication and integration in prokaryotes. In one embodiment, the expression vector includes additional sequences which render this vector suitable for replication and integration in eukaryotes. In one embodiment, the expression vector of the present invention includes a shuttle vector which renders this vector suitable for replication and integration in both prokaryotes and eukaryotes. In some embodiments, cloning vectors comprise transcription and translation initiation sequences (e.g., promoters, enhances) and transcription and translation terminators (e.g., polyadenylation signals).

In one embodiment, a variety of prokaryotic or eukaryotic cells can be used as host-expression systems to express the polypeptides of the present invention. In some embodiments, these include, but are not limited to, microorganisms, such as bacteria transformed with a recombinant bacteriophage DNA, plasmid DNA or cosmid DNA expression vector containing the polypeptide coding sequence; yeast transformed with recombinant yeast expression vectors containing the polypeptide coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the polypeptide coding sequence.

In other embodiments, subsets of the nucleic acid sequences are provided for use as primers for nucleic acid amplification reactions, as probes in hybridization assays or arrays to detect sequences in samples, or as probes to distinguish normal or wild-type sequences from abnormal or mutant sequences.

### Isolated Fusion Polypeptide Molecules

In some embodiments, the present invention discloses isolated fusion polypeptide molecules comprising the polypeptides described herein (e.g., a polypeptide comprising SEQ ID NO: 31) attached to a carboxy terminus of a second polypeptide. The second polypeptide molecules include, but are not limited to, a hormone, a receptor, a binding protein, or a soluble factor. For example, the second polypeptide includes, but is not limited to, insulin, CTLA-4, Albutein/albumin, FSH, Activase altiplase/tPA, adenosine deaminase, immune globulin, glucocerebrosidase, OB protein, Peptide X, Growth Hormone, Leukine-sargramostim/GM-CSF, G-CSF, T₃, Venoglobulin-S/IgG, Proleukin aldesleukin, DNase, factor VIII, Helixate, L-asparaginase, WinRho SDF Rh I, Retavase retaplase/tPA, Factor IX, globulin, fibrin, interleukin-11, becaplermin/PDGF, lepirudin/herudin, TNF, Thymoglobulin, factor VIIa, interferon alpha-2a, interferon alpha n−1, interferon alpha-N3, interferon beta-1b, interferon gamma-1b, Interleukin-2, and monoclonal antibodies.

In other embodiments, the present invention discloses isolated fusion polypeptide molecules comprising polypeptides comprising SEQ ID NO: 37 attached to an ectodomain of a receptor. For example, the receptor includes, but is not limited to, insulin, CTLA-4, Albutein/albumin, FSH, Activase altiplase/tPA, adenosine deaminase, immune globulin, glucocerebrosidase, OB protein, Peptide X, Growth Hormone, Leukine-sargramostim/GM-CSF, G-CSF, T₃, Venoglobulin-S/IgG, Proleukin aldesleukin, DNase, factor VIII, Helixate, L-asparaginase, WinRho SDF Rh I, Retavase retaplase/tPA, Factor IX, globulin, fibrin, interleukin-11, becaplermin/PDGF, lepirudin/herudin, TNF, Thymoglobulin, factor VIIa, interferon alpha-2a, interferon alpha n−1, interferon alpha-N3, interferon beta-1b, interferon gamma-1b, Interleukin-2, and monoclonal antibodies. In some embodiments, SEQ ID NO: 37 is fused to a binding protein or a soluble factor.

In one embodiment, polypeptides of the present invention are purified using a variety of standard protein purification techniques, including but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization. In some embodiments, synthetic polypeptides are purified by preparative high performance liquid chromatography (Creighton T. (1983) Proteins, structures and molecular principles. WH Freeman and Co. N.Y.), and the composition of the polypeptides can be confirmed via amino acid sequencing by methods known to one skilled in the art.

In one aspect, the second polypeptide is a vascular endothelial growth factor (VEGFR) receptor, and the isolated polypeptide comprising SEQ ID NO: 31 is added to extracellular domains 1-3 of the VEGFR1 (Protein Accession No. P17948) or VEGFR2 receptor (Protein Accession No. ACF47599.1). In some embodiments, the isolated polypeptide comprising SEQ ID NO: 37 is fused to extracellular domains 1-3 of the VEGFR1. In other embodiments, the isolated polypeptide comprising SEQ ID NO: 37 is fused to extracellular domains 1-3 of the VEGFR2.

In one embodiment, an isolated fusion polypeptide molecule includes VEGFR1 receptor, and is encoded by the nucleic acid sequence of SEQ ID NO: 32. In one embodiment, an isolated fusion polypeptide molecule includes VEGFR1 receptor, encompassing the amino acid sequence of SEQ ID NO: 33. In other embodiments, the isolated fusion polypeptide has at least about 99%, 98%, 97%, 96%, 95%, 94%, 93%, 92%, or 91% identity to SEQ ID NO: 32 or SEQ ID NO: 33.

In another embodiment, an isolated fusion polypeptide molecule includes VEGFR2 receptor, and is encoded by the nucleic acid sequence of SEQ ID NO: 34. In another embodiment, an isolated fusion polypeptide molecule includes VEGFR2 receptor, encompassing the amino acid sequence of SEQ ID NO: 35. In other embodiments, the isolated fusion polypeptide has at least about 99%, 98%, 97%, 96%, 95%, 94%, 93%, 92%, or 91% identity to SEQ ID NO: 34 or SEQ ID NO: 35.

There are three main subtypes of VEGFR receptors: VEGFR1, VEGFR2, and VEGFR3. VEGFR receptors may be membrane-bound (mbVEGFR) or soluble (sVEGFR), depending on alternative splicing. Vascular endothelial growth factors (VEGF) is a signaling protein involved in vasculogenesis (the formation of the circulatory system) and angiogeneis (the growth of blood vessels from pre-existing vasculature. VEGF activity is restricted mainly to cells of the vascular endothelium, although it does have effects on some other cell types, e.g., stimulation of monocyte/macrophage migration. VEGF also enhances microvascular permeability.

In another aspect, the second polypeptide is a Notch receptor. Notch receptors include Notch1 (GenBank Accession No. CAG33502), Notch2 (GenBank Accession No. AAB19224), Notch3 (GenBank Accession No. AAB91371), and Notch4 (GenBank Accession No. AAC63097). The Notch receptor is a single-pass transmembrane receptor protein. It is a hetero-oligomer composed of a large extracellular portion, which associates in a calcium-dependent, non-covalent interaction with a smaller piece of the Notch protein composed of a short extracellular region, a single transmembrane-pass, and a small intracellular region. Stuttfeld et al. (September 2009). IUBMB Life 61 (9): 915-22. In one embodiment, the isolated polypeptide comprising SEQ ID NO: 31 is added to the ectodomain of a Notch receptor (e.g. Notch-1, -2, -3, or -4). In one embodiment, the ectodomain of a Notch receptor ((e.g. Notch-1, -2, -3, or -4) can be fused to a polypeptide comprising SEQ ID NO: 37.

Ligands that bind to the extracellular domain of the Notch receptor induce proteolytic cleavage and release of the intracellular domain. Because most ligands are also transmembrane proteins, the Notch receptor is normally triggered only from direct cell-to-cell contact.

The Notch cascade includes Notch receptor and Notch ligands, and intracellular proteins transmitting the Notch signal to the cell's nucleus. The Notch signaling pathway is important for cell-cell communication, which involves gene regulation mechanisms that control multiple cell differentiation processes during embryonic and adult life.

### Methods Involving the Isolated Polypeptides

In another embodiment, a method of increasing a biological half-life of a polypeptide is disclosed. The method comprises attaching a CTP domain comprising at least about 90% identity to SEQ ID NO: 37, fused to an antibody fragment to the carboxy terminus of the polypeptide, thereby increasing the biological half-life of the polypeptide.

In another embodiment, a method of stabilizing a polypeptide is disclosed. The method comprises attaching a CTP domain comprising at least about 90% identity to SEQ ID NO: 37 fused to an antibody fragment to the carboxy terminus of the polypeptide, thereby stabilizing the polypeptide.

In one embodiment, the invention provides for methods of increasing a biological half-life of a polypeptide where the methods comprise attaching a carboxy-terminal peptide (CTP) domain comprising at least about 90% identity to SEQ ID NO: 37, fused to an ectodomain of a receptor, thereby increasing the biological half-life of the polypeptide.

In yet another embodiment, the invention provides for methods of stabilizing a polypeptide where the methods comprise attaching a carboxy-terminal peptide (CTP) domain comprising at least about 90% identity to SEQ ID NO: 37, fused to an ectodomain of a receptor, thereby increasing the biological half-life of the polypeptide.

Other aspects of the methods comprises further modifying the CTP domain to change the quantity or type of glycosylation.

The polypeptides used in these methods can include an antibody, a fusion protein, a hormone, a receptor, a binding protein, or a soluble factor. The antibody fragment can be an Fab′ fragment, an F(ab′)₂ fragment, an Fv fragment, an Fc fragment, a diabody, and a single-chain variable fragment (scFv).

The antibody fragment can be from any Ig isotype, including IgG, IgA, IgE, IgM, and IgD, and any Ig sub-types. IgG subtypes are IgG1, IgG2, IgG3, and IgG4. In other embodiments, the CTP domain is glycosylated, such as N-link glycosylated, O-linked glycosylated, or a combination thereof.

In other embodiments, the methods described herein comprise attaching a CTP domain comprising at least about 99%, 98%, 97%, 96%, 95%, 94%, 93%, 92%, or 91% identity to SEQ ID NO: 37.

### Pharmaceutical Compositions

In other aspects, the invention discloses pharmaceutical compositions comprising the isolated polypeptides or the isolated fusion polypeptide molecules disclosed herein and a pharmaceutically acceptable carrier. The pharmaceutically compositions or isolated fusion polypeptide molecules can comprise a therapeutically effective amount of one or more isolated polypeptides, formulated with one or more pharmaceutically acceptable carriers, buffers and/or diluents. The pharmaceutical compositions can be specially formulated for administration in solid or liquid form, including those adapted for the following: (1) enteral administration, for example, drenches (aqueous or non-aqueous solutions or suspensions), tablets, boluses, powders, granules, pastes for application to the tongue, suppositories for rectal administration; or (2) parenteral administration, for example, by subcutaneous, intramuscular or intravenous injection as, for example, a sterile solution or suspension. In certain embodiments, the polypeptides can also be simply dissolved or suspended in sterile water.

Wetting agents, emulsifiers and lubricants, as well as coloring agents, release agents, coating agents, sweetening, flavoring and perfuming agents, preservatives and antioxidants can also be present in the compositions.

The enteric and parenteral formulations can conveniently be presented in unit dosage form and can be prepared by any methods well-known in the art. In general, the compositions are prepared by uniformly and intimately bringing into association one or more agents of the present invention with liquid carriers, or finely divided solid carriers, or both, and then, if necessary, shaping the product. The amount of polypeptide which can be combined with a carrier material to produce a single dosage form will vary depending upon the host being treated and the particular mode of administration. The amount of polypeptide that can be combined with a carrier material to produce a single dosage form will generally be that amount of the protein which produces a therapeutic effect. Generally, this amount can range anywhere from 1%-99% of the polypeptide.

Formulations of the invention suitable for oral administration can be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. The polypeptides of the invention can also be administered as a bolus, electuary or paste. Liquid dosage forms for administration of the proteins of the invention include pharmaceutically acceptable emulsions, microemulsions, solutions, and suspensions.

Formulations suitable for parenteral administration may be provided in liquid form or solid form. The liquid form may be a ready-to-use formulation or a concentrated formulation of the protein that is diluted before use. The solid form may be a lyophilized preparation of the protein, such that the lyophilized protein is solubilized prior to injection. In some embodiments, the protein preparation may be an intravenous bolus injection suitable for administration intravenously.

### Transformed Cell Lines

In another aspect, the present invention provides cell lines transformed with the nucleic acid molecules of the invention. Such cell lines can simply propagate these nucleic acids (e.g., when transformed with cloning vectors) or can express the polypeptides encoded by these nucleic acids (e.g., when transformed with expression vectors). Such transformed cell lines can be used to produce the polypeptides of the invention.

The transformed cells can be produced by introducing into a cell an exogenous nucleic acid or nucleic acid analog which replicates within that cell, that encodes a polypeptide sequence which is expressed in that cell, and/or that is integrated into the genome of that cell so as to affect the expression of a genetic locus. The transformation can be achieved by any of the standard methods referred to in the art as transformation, transfection, transduction, electroporation, ballistic injection, and the like. The method of transformation is chosen to be suitable to the type of cells being transformed and the nature of the genetic construct being introduced into the cells.

Useful cell lines for transformation include bacterial cells (e.g., Escherichia coli), yeast cells (e.g., Saccharomyces cerevisiae), insect cells (e.g., Drosophila melanogaster Schneider cells), nematode cells (e.g., Caenorhabditis elegans), amphibian cells (e.g., Xenopus oocytes), rodent cells (e.g., Mus musculus (e.g., murine 3T3 fibroblasts), Rattus rattus, Chinese Hamster Ovary cells (e.g., CHO-K1)), and human cells (e.g., human skin fibroblasts, human embryonic kidney cells (e.g., HEK-293 cells), COS cells).

The cells can be transformed according to any method known in the art appropriate to the cell type being transformed. Appropriate methods include those described generally in, e.g., Sambrook et al. (1989), Molecular Cloning: A Laboratory Manual, Cold Spring Harbor Laboratories, New York; and Davis et al. (1986), Basic Methods in Molecular Biology, Elsevier. Particular methods include calcium phosphate co-precipitation (Graham et al. (1973), Virol. 52:456-467), direct micro-injection into cultured cells (Capecchi (1980), Cell 22:479-488), electroporation (Shigekawa et al. (1988), BioTechniques 6:742-751), liposome-mediated gene transfer (Mannino et al. (1988), BioTechniques 6:682-690), lipid-mediated transduction (Felgner et al. (1987), Proc. Natl. Acad. Sci. USA 84:7413-7417), and nucleic acid delivery using high-velocity microprojectiles (Klein et al. (1987), Nature 327:70-73).

### Diagnostic/Therapeutic Methods

In another aspect, the present invention provides methods of treating or preventing a disorder comprising administering an effective dose of the pharmaceutical composition disclosed herein to a subject in need of treatment or prevention of a disorder selected from the group consisting of a cancer, immune-related disorder, cardiovascular disease, obesity, diabetes, metabolic disorders, and blindness. In some embodiments, the subject is a human.

### EQUIVALENTS

While this invention has been particularly shown and described with references to preferred embodiments thereof, it will be understood by those skilled in the art that various changes in form and details may be made therein without departing from the spirit and scope of the invention as defined by the appended claims. Those skilled in the art will recognize, or be able to ascertain using no more than routine experimentation, many equivalents to the specific embodiments of the invention described specifically herein. Such equivalents are intended to be encompassed in the scope of the claims.

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention.

All publications and other references mentioned herein are incorporated by reference in their entirety, as if each individual publication or reference were specifically and individually indicated to be incorporated by reference. Publications and references cited herein are not admitted to be prior art.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Anthrax Toxin Receptor 2 Functions in ECM Homeostasis of the Murine Reproductive Tract and Promotes MMP Activity

Anthrax Toxin Receptor proteins function as receptors for anthrax toxin, however physiological activity remains unclear. To evaluate the biological role of Antxr2, Antxr2−/− mice were generated. Antxr2−/− mice were viable, however Antxr2 is required for parturition in young females and for preserving fertility in older female mice. Histological analysis of the uterus and cervix revealed aberrant deposition of extracellular matrix proteins such as type I collagen, type VI collagen and fibronectin. A marked disruption of both the circular and longitudinal myometrial cell layers was evident in Antxr2−/− mice. These changes progressed as the mice aged, resulting in a thickened, collagen dense, acellular stroma and the disappearance of normal uterine architecture. To investigate the molecular mechanism underlying the uterine fibrosis, immunoblotting was performed for MMP2 using uterine lysates and zymography using conditioned medium from Antxr2−/− mouse embryonic fibroblasts and found reduced levels of activated MMP2 in both. This prompted investigation of MT1-MMP status, as MMP2 processing is regulated by MT1-MMP. MT1-MMP activity, as measured by MMP2 processing and activation, was enhanced by expression of either ANTXR1 or ANTXR2. An ANTXR2/MT1-MMP complex was identified, which was then demonstrated that MT1-MMP activity is dependent on ANTXR2 expression levels in cells. Thus, ANTXR1 and ANTXR2 function as positive regulators of MT1-MMP activity.

The Anthrax Toxin Receptor (ANTXR) proteins, ANTXR1 and ANTXR2, are cellular receptors that contain a von Willebrand factor type A (vWF) domain, a transmembrane domain and a cytosolic tail with putative signaling motifs. vWF domains are known to facilitate protein-protein interactions when found on extracellular matrix (ECM) constituents or cell adhesion proteins like α-integrin subunits [1] and constitute ligand binding sites on ANTXRs [2]. Both ANTXR1 and ANTXR2 have been demonstrated to interact with ECM proteins in vitro [3,4,5].

To investigate the physiological role of Antxr2, the gene was disrupted and it was discovered that Antxr2 is not essential for normal development, but is required for murine parturition in young pregnant mice and for preserving fertility in aged female mice. Histological analysis of the uterus and cervix revealed aberrant deposition of ECM proteins causing severe disorganization of the cellular composition of these tissues. The molecular mechanism behind these defects was investigated and it was discovered that ANTXR2 is a positive regulator of MT1-MMP activity, a key protein that activates MMP2 and functions in ECM turnover.

Results

Antxr2−/− Mice Exhibit a Failure in Parturition

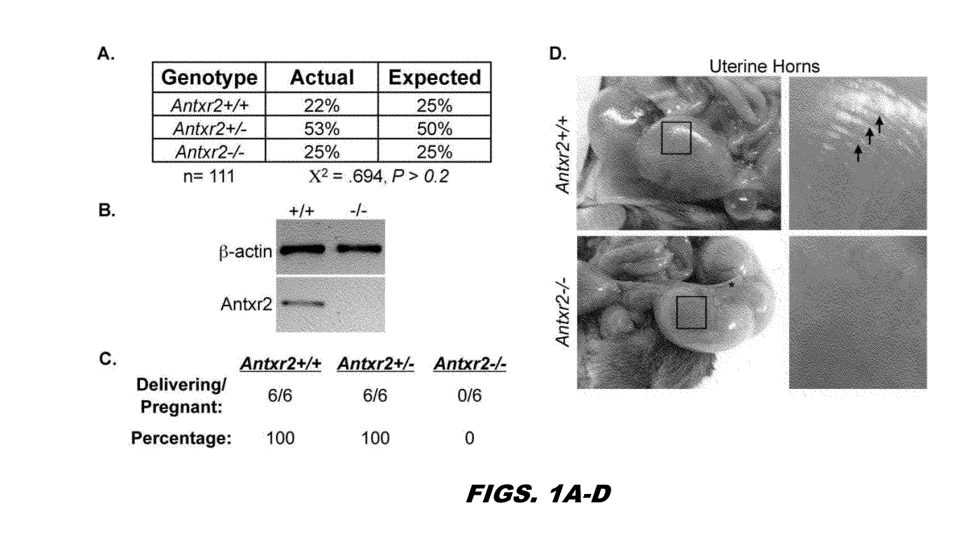

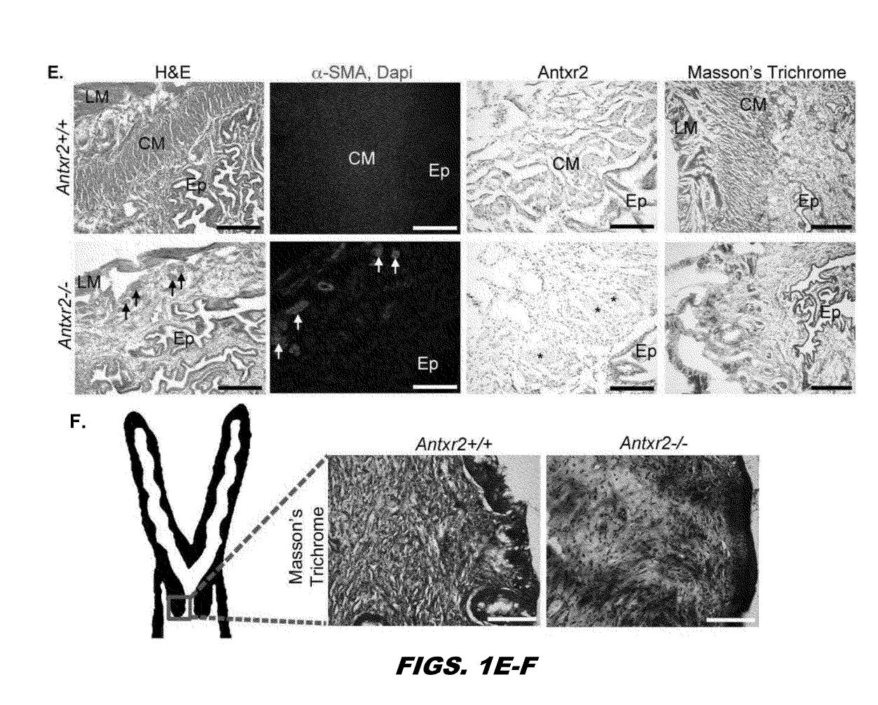

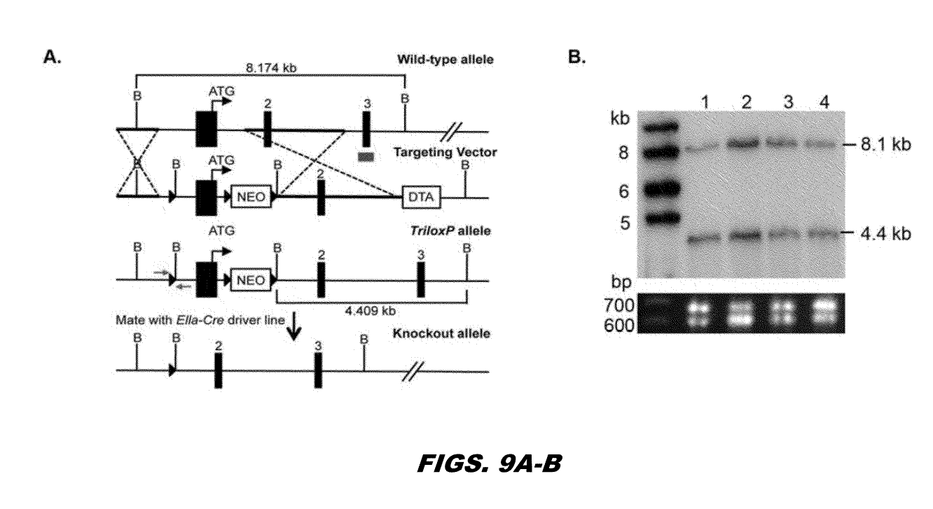

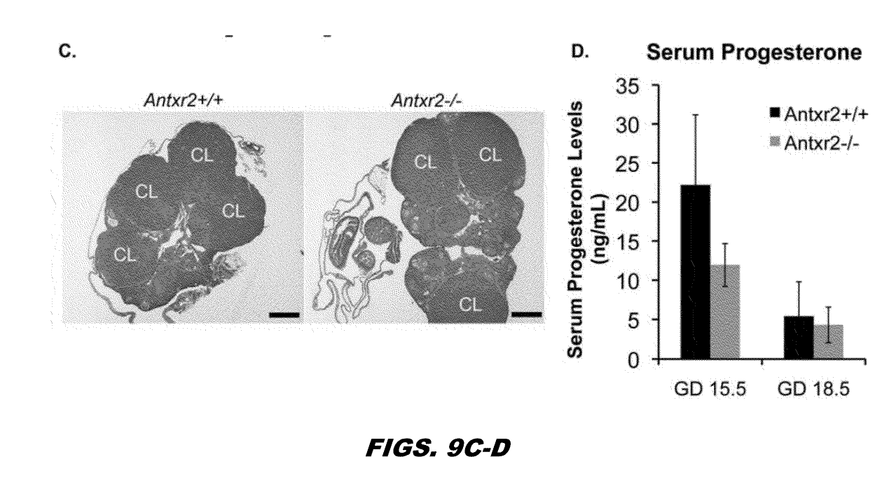

To ascertain the function of Antxr2, a conditional Antxr2 knockout mouse was generated. Exon 1 of Antxr2 encodes the first 50 amino acids of the Antxr2 protein including a 26 amino acid signal peptide and initiating methionine. Thus, exon 1 was targeted for deletion using a triloxP targeting approach (FIG. 9A). Deletion of exon 1 was accomplished by mating triloxP targeted male mice with female Ella-Cre transgenic mice. The maternally derived Cre is more efficient at producing total germline excision of the loxP-flanked exon 1 and NEO cassette (see FIG. 9A) due to the presence of Cre in the oocyte. The Antxr2−/− mice described herein were on a mixed 129XC57BL/6 background. Intercrosses of Antxr2+/− mice produced progeny in the expected Mendelian ratios: 22%+/+, 53%+/−, 25%−/− of 111 offspring analyzed (FIG. 1A), demonstrating that loss of Antxr2 did not result in embryonic lethality. Antxr2−/− mice were viable at birth and developed normally, showing no striking phenotypic difference when compared with their wild type and heterozygous littermates at the macroscopic level. Histological analysis of skin, heart, lung, spleen, kidney, liver, intestine and bone did not reveal differences in organ development or organization at 1 month of age. RT-PCR analysis on total RNA isolated from mouse embryonic fibroblasts (MEFs) confirmed that deletion of exon 1 led to a corresponding loss of Antxr2 mRNA (FIG. 1B).

To evaluate fertility of Antxr2−/− mice, timed matings were established. Young Antxr2−/− males were normal in their reproductive ability in that copulation plugs were detected and they impregnated female mice. 6-week-old Antxr2−/− females were also fertile. Once pregnant, Antxr2−/− females increased in body weight, but all of the mutant mice failed to deliver pups on the expected due date (gestational day 19) and died approximately one week later (FIG. 1C). Necropsies revealed that the pups had died in utero and were beginning to degenerate. To determine if the parturition failure resulted from embryos dying during gestation, embryo viability was analyzed late in gestation. Antxr2−/− intercrosses (n=7) and Antxr2+/+ (n=3) intercrosses were performed and embryos were isolated on gestational day 18.5 (GD 18.5), twelve hours before they were to be born. Regardless of genotype, all embryos were found to be alive on GD18.5, as determined by embryonic movement prior to dissection. No abnormalities were observed in either the size or gross morphology of the Antxr2−/− embryos or the associated placentas when compared to Antxr2+/+ and Antxr2+/− embryos. Furthermore, expression of embryonic Antxr2 did not result in the timely induction of parturition in Antxr2−/− dams. Timed matings were established between Antxr2−/− females (n=3) and Antxr2+/+ males or Antxr2+/+ females (n=3) and Antxr2−/− males to generate Antxr2+/− embryos. Analysis of gestational length demonstrated that Antxr2+/+ females carrying Antxr2+/− embryos gave birth on GD 19 whereas Antxr2−/− females carrying Antxr2+/− embryos consistently failed to give birth. These analyses indicate that abnormal progression of labor in the mother was the mechanism of death for the pregnant Antxr2−/− mice.

Parturition, the process of giving birth, requires the coordinated regulation of multiple signaling pathways in the ovary, uterus and cervix. In mice, pregnancy is maintained by continued synthesis of progesterone in the corpus luteum from fetal or maternal steroid percursors. At term, progesterone synthesis decreases and catabolism increases, producing a fall in serum progesterone, a process termed luteolysis [13]. Histological analysis of ovaries collected from Antxr2+/+ and Antxr2−/− mice on GD 18.5 revealed the normal formation of corpus lutei with no overt structural abnormalities (FIG. 9C). ELISA of serum from Antxr+/+ and Antxr2−/− mice on GD 15.5 and 18.5 revealed that progesterone levels declined in both Antxr2+/+ and Antxr2−/− mice as the pregnancies progressed to term (FIG. 9D).

Parturition requires the onset of rhythmic contractions in the uterus and ripening/dilation of the cervix to allow for delivery of the embryo through the birth canal. The failure of either cervical ripening or adequate uterine contractions causes unsuccessful parturition [14]. To determine if either of these essential processes is disrupted in the Antxr2−/− mice, reproductive tracts were isolated on GD 18.5 and histological analysis was conducted of both the uterus and cervix. Gross inspection of reproductive tracts revealed that Antxr2−/− uterine tissue exhibited poor uterine tone (asterisk in FIG. 1D) and lacked muscle striations. In contrast, the Antxr2+/+ uterus was tightly wrapped around each embryo and exhibited visible muscle striations (arrows in FIG. 1D). H&E staining demonstrated Antxr2−/− uteri lacked both circular (CM) and longitudinal (LM) myometrial cell layers, which was confirmed by alpha-smooth muscle actin (∝-SMA) immunostaining (FIG. 1E). Immunostaining also demonstrated that Antxr2 is highly expressed in the uterine myometrium and confirmed lack of expression in the Antxr2−/− tissue (FIG. 1E). To assess collagen content in the pregnant uterine tissue, Masson's Trichrome staining was performed and increased fibrillar collagen deposition was found in the Antxr2−/− tissue in the area of the uterus where the longitudinal and circular myometrial cell layers normally reside (FIG. 1E).

Normal cervical ripening is characterized by reorganization of the ECM [15]. To assess cervical ripening, GD18.5 cervical collagen content and organization was evaluated by staining with Masson's Trichrome. In Antxr2+/+ cervices, a loose array of collagen fibers was observed, an indicator of compliant tissue. Antxr2−/− cervices exhibited dense, compact, heavily stained collagen fibrils, which are characteristic of inelastic tissue (FIG. 1F). Taken together, the disorganized nature of the myometrium in the uterus and the dense collagen network in the cervix indicates that the parturition defect in the Antxr2−/− mice is due to inadequate uterine contractions and a failure in cervical ripening.

Nulliparous Aged Antxr2−/− Mice Develop Severe Fibrosis in the Uterus and Cervix

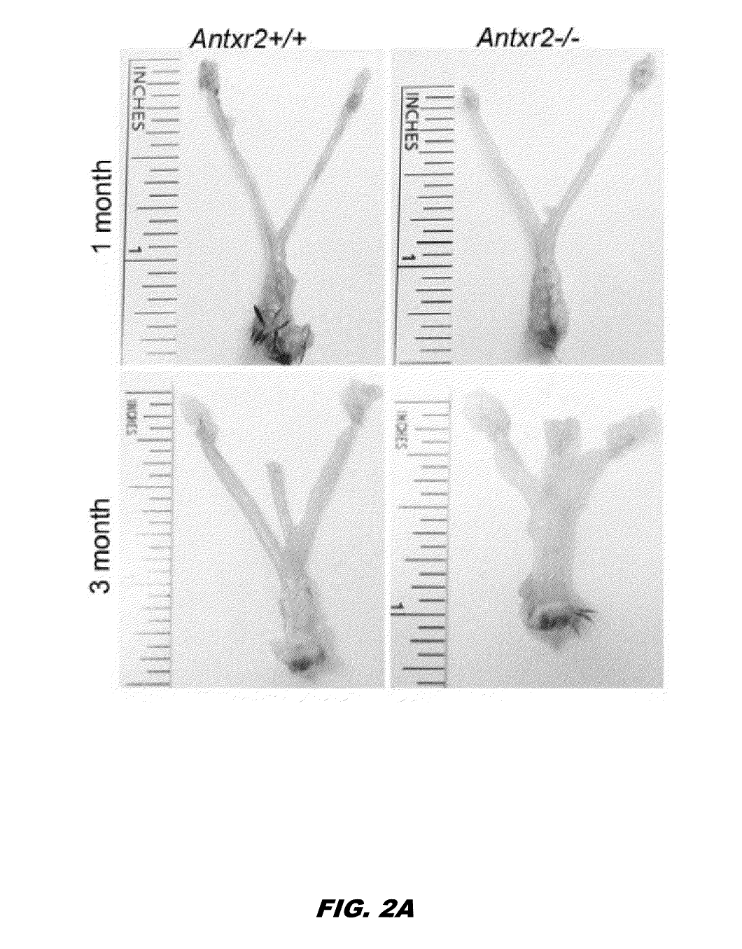

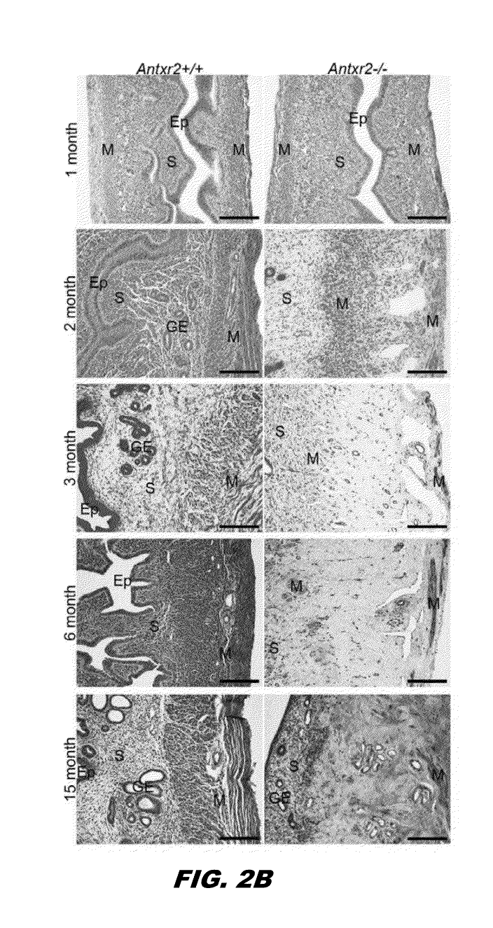

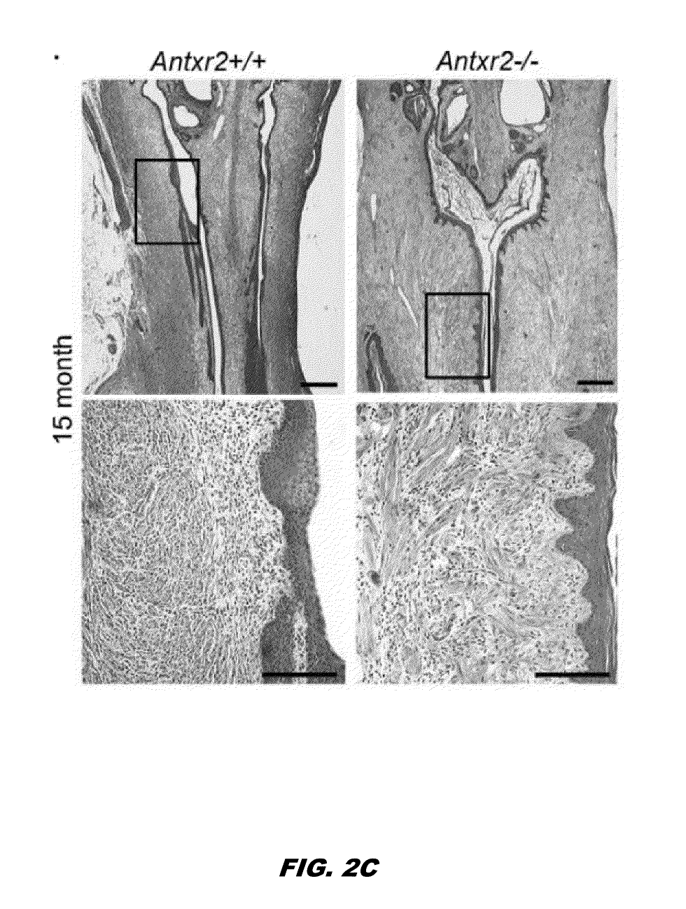

In addition to the parturition defect, older Antxr2−/− females, from 2 months of age and beyond, had problems with fertility. Mating young (6-week-old), sexually mature Antxr2−/− females produced pregnancies that were carried to term but resulted in defective parturition. In contrast, older Antxr2−/− females, aged 2 to 6-months, had difficulty carrying a pregnancy to term. Fertility analysis revealed that these Antxr2−/− females were able to get pregnant as evidenced by plug formation and subsequent weight gain, however, approximately half of the pregnant animals miscarried their litters. Fertility analysis of female mice aged 7-months-old and beyond revealed that they were unable to get pregnant. Consequently, reproductive tracts were isolated from both young and aged nulliparous Antxr2−/− mice for analysis. Reproductive tracts isolated from one-month-old prepubescent mice looked similar in overall appearance (FIG. 2A top panel), but reproductive tracts isolated from sexually mature 3-month-old mice displayed striking differences in morphology (FIG. 2A bottom panel). The Antxr2−/− reproductive tracts had a shortened, thickened shape in comparison to the thin, elongated reproductive tracts from Antxr2+/+ animals. This phenotype was observed for every nulliparous Antxr2−/− female mouse evaluated (n=18, aged 3-15 months). Masson's trichrome staining did not reveal overt structural abnormalities or changes in collagen deposition in prepubescent Antxr2−/− uteri (FIG. 2B, one month panel). However, sexually mature Antxr2−/− uteri were characterized by collagen fibrosis (FIG. 2B, panels 2, 3, 6, 15 month). The fibrosis progressed as the mice aged, resulting in a thickened, collagen dense, acellular stroma and the disappearance of normal uterine architecture (FIG. 2B). Similarly, cervical tissue isolated from 15-month-old Antxr2−/− mice exhibited increased collagen content (FIG. 2C) as compared to Antxr2+/+ tissue. Collagen deposition was also examined in the ovaries of aged mice. Unlike what had been reported for Antxr1−/− mice (9), increased collagen content was not observed in ovaries isolated from either 3-month-old or 6-month-old Antxr2−/− mice. Antxr2−/− ovaries appeared normal with the presence of follicles in various stages of maturation (FIG. 10). The extensive fibrosis throughout the reproductive tract in aged Antxr2−/− mice impairs fertility.

The Myometrium is Disrupted in Nulliparous Aged Antxr2−/− Mice

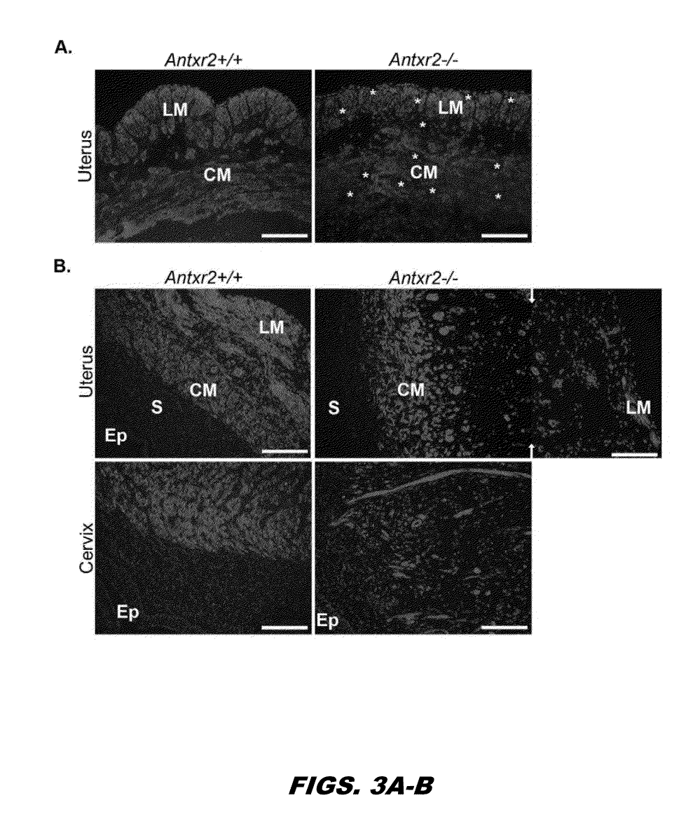

The normal architecture of the uterine wall consists of an inner circular layer of myometrial (CM) cells, an intervening vascular space and an outer longitudinal layer of myometrial (LM) cells as seen in Antxr2+/+ mice (FIG. 3) Immunofluorescent staining with ∝-SMA revealed well-defined, tightly packed CM and LM layers. Uteri isolated from nulliparous Antxr2−/− mice presented disorganized CM and LM layers, similar to that seen in uteri from pregnant Antxr2−/− mice. As early as 6.5 weeks of age, the CM and LM layers were beginning to loosen resulting in increased intercellular space between bundles of muscle cells (see asterisks in FIG. 3A). This loosening progressed as the mice aged. The CM in uteri isolated from 3-month-old Antxr2−/− mice consisted of a poorly defined layer of scattered smooth muscle cells. The space between the CM and LM layers had become greatly distended. This is shown in FIG. 3B where two pictures of Antxr2−/− uterine morphology are placed together in order to capture the same area represented in one picture of Antxr2+/+ tissue. The LM was almost completely ablated in the Antxr2−/− tissue with only a few muscle cell bundles at the periphery of the uterus (FIG. 3B, merged top panel). A similar smooth muscle cell phenotype was observed in the cervix (FIG. 3B, bottom panel). The disappearance of the myometrium was also progressive as the mice aged. TUNEL staining did not reveal an increase in myometrial cell death in the Antxr2−/− tissues analyzed indicating that loss of cells due to apoptosis is gradual over months or not a mechanism of muscle cell loss. However, increased cell death was detected in luminal and glandular epithelial cells in uterine tissue aged 6 months and beyond. These results demonstrate that in both pregnant (FIG. 1E) and non-pregnant (FIG. 3) states, Antxr2 has a critical role in the maturation or maintenance of the myometrium. It is also interesting to note that in the uteri of both pregnant and aged nulliparous Antxr2−/− mice, the loss of myometrial cells is associated with ECM protein accumulation. The myometrium has been demonstrated to produce MMP2 during postpartum involution of the rat uterus [16]. Taking this into account, the data indicates that the myometrium is also important for matrix remodeling in the cycling uterus and during pregnancy.

Vascular Changes and Inflammation Accompany Fibrosis in the Nulliparous Antxr2−/− Reproductive Tract

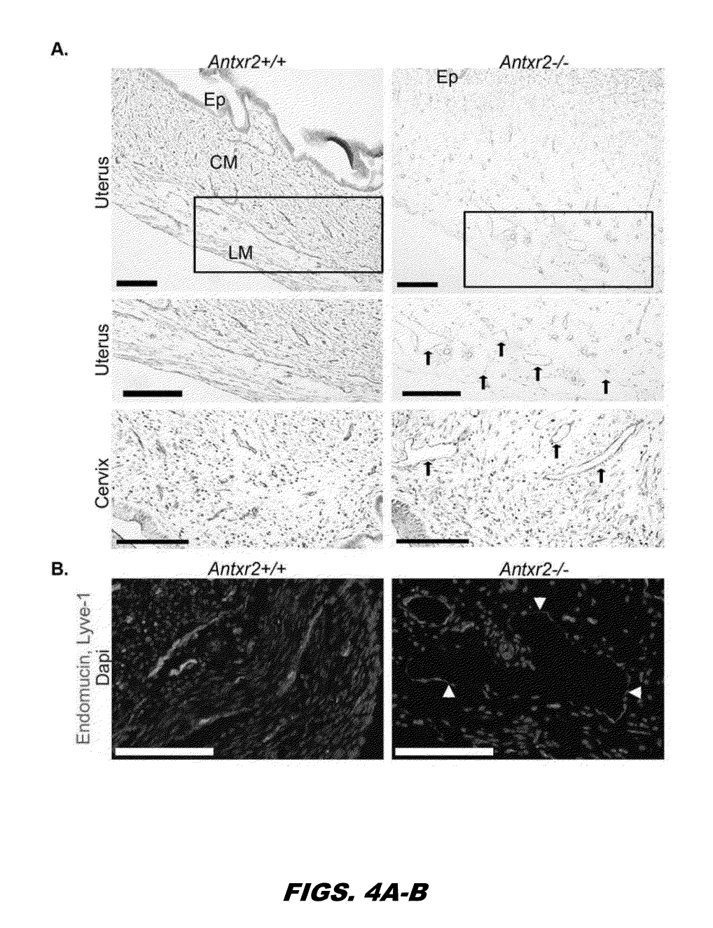

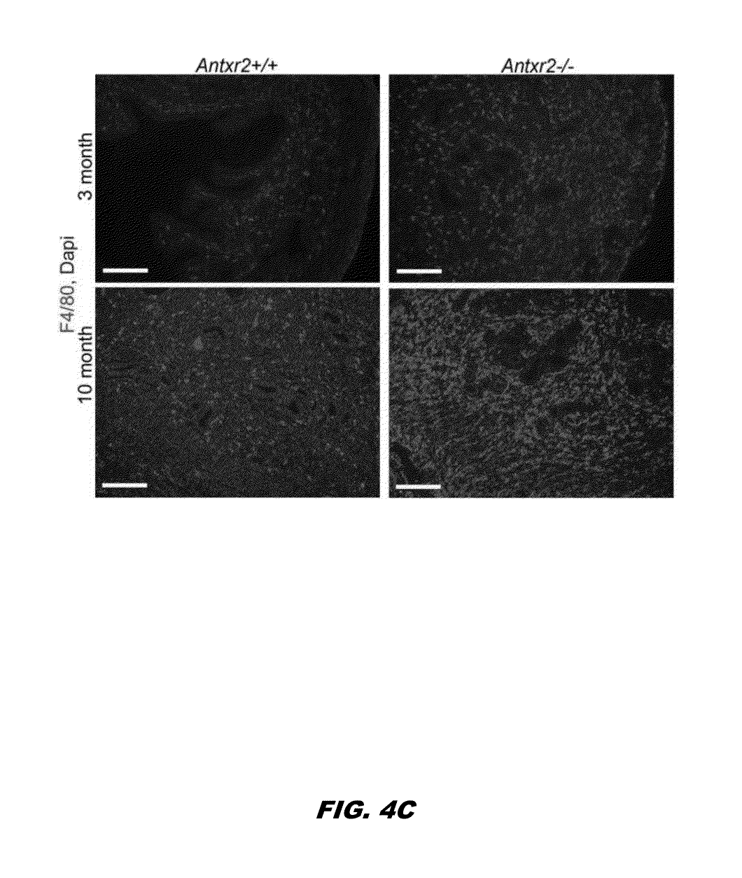

Staining for the endothelial marker, CD31, revealed atypical vessels in the uterus and the cervix of Antxr2−/− mice when compared to that of Antxr2+/+ vessels (FIG. 4A, arrows). When uterine tissue was sectioned in the same orientation, vessels in the Antxr2+/+ tissue had collapsed lumens while vessels in the Antxr2−/− tissue had open lumens. CD31 staining in the Antxr2−/− tissue was also more faint. A reduction in CD31 was detected at the cell surface when performing flow cytometry on human umbilical venous endothelial cells (HUVEC) with ANTXR2 knocked down via RNA interference (RNAi) and there may be reduced CD31 expression on the endothelium in Antxr2−/− tissue.

As CD31 does not differentiate between blood vasculature and lymphatic vasculature, coimmunofluorescence was also performed using the blood endothelial cell marker, endomucin, and the lymphatic endothelial cell marker, lyve-1. In Antxr2+/+ tissue, lymphatic vessels were collapsed and resided within the CM and LM layers (FIG. 4B). In the Antxr2−/− tissue, co-staining demonstrated that the lymphatic vessels were grossly dilated (FIG. 4B, white arrowheads).

In addition to changes in the blood and lymphatic vasculature, there was a far greater infiltration of inflammatory cells, detected as F4/80 positive macrophages (FIG. 4C). There is a resident population of macrophages in the uterus [17], however, if ECM accumulation in the Antxr2−/− reproductive tract is likened to a wound, dilation of blood and lymphatic vessels allows for influx of macrophages into the tissue in order to facilitate tissue repair. These histopathological changes are hallmarks of fibrotic tissue that are secondary to the uterine fibrosis rather than the result of losing Antxr2 expression in blood endothelium, lymphatic endothelium or macrophages. In support of this, mice were generated with deletion of Antxr2 in the blood endothelium using a VE-cadherin Cre driver line. Reproductive tracts from female VE-Cadherin Cre; Antxr2^{fl/fl} mice do not have ECM accumulation nor do they have atypical/open blood vessels.

Uterine Fibrosis in Nulliparous Aged Antxr2−/− Mice is Characterized by Increased Collagen and Fibronectin Content

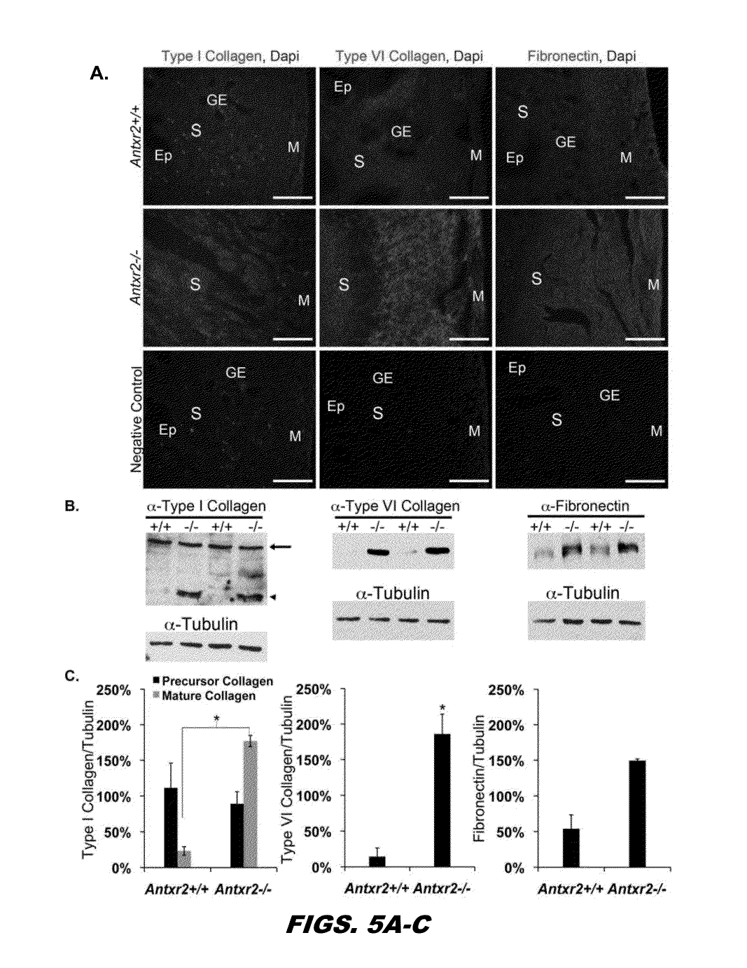

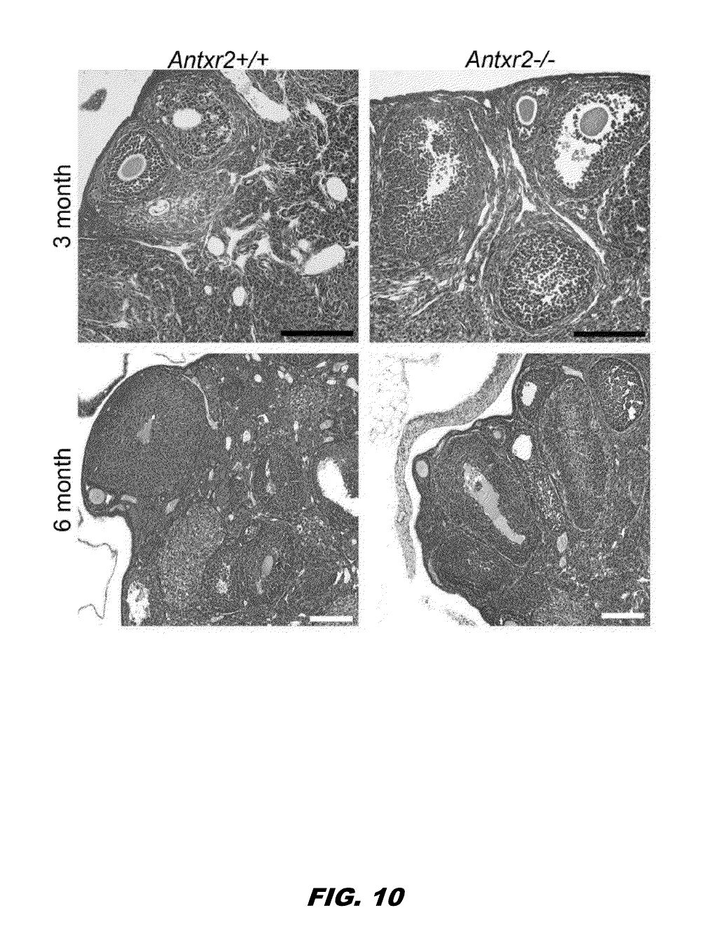

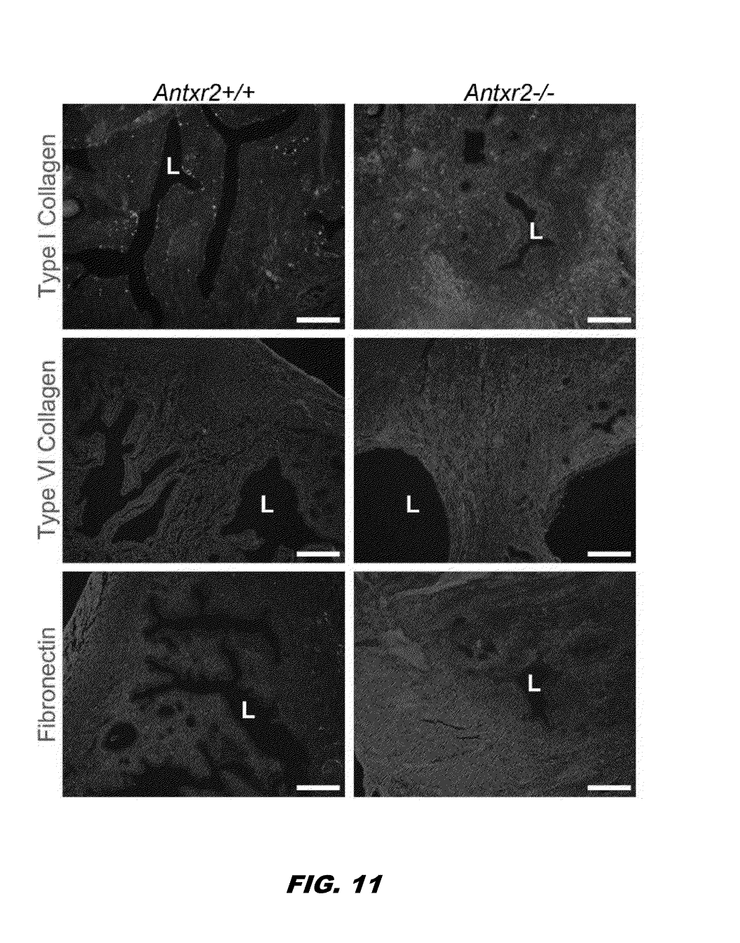

The types and amounts of fibrillar collagens or other ECM proteins present in uterine tissue were assessed, focusing the analysis on predicted ECM ligands for ANTXRs. Immunostaining revealed that type I collagen, type VI collagen and fibronectin content is increased in uteri isolated from 6-month-old Antxr2−/− mice compared to that of Antxr2+/+ (FIG. 5A). In order to quantify the changes in ECM content, uterine lysates from 6-month-old mice were immunoblotted for type I collagen, type VI collagen, fibronectin, and tubulin as a loading control (FIG. 5B) and densitometery was used to quantify protein bands. There was no significant change in the amount of precursor type I collagen present in Antxr2−/− uterine tissue (arrow in FIG. 5B), however, there was a significant 7 fold increase in the amount of mature type I collagen in Antxr2−/− uteri as compared to Antxr2+/+ (P<0.005) (FIG. 5C). Similarly, the amount of type VI collagen present in Antxr2−/− uteri was 13 times that of Antxr2+/+ uteri (P<0.05) (FIG. 5C). There was also a trend towards increased fibronectin content in the Antxr2−/− uteri, however it did not reach significance (P=0.08) when compared to Antxr2+/+ levels (FIG. 5C) Immunostaining revealed that accumulation of these same ECM proteins was more pronounced in 10-month-old Antxr2−/− tissue (FIG. 11). The uterus is a dynamic organ that undergoes extensive ECM remodeling with each round of the estrus cycle [18]. The accumulation of uterine ECM proteins (FIGS. 2, 5 and 11) as Antxr2−/− mice age indicates a defect in the remodeling process.

Matrix Metalloproteinase 2 Activity is Impaired in Cells and Tissue Deficient for Antxr2

The uterine endometrium and associated stroma undergoes extensive remodeling during post-pubertal life in response to the estrus cycle [19]. Part of this remodeling process involves the synthesis and degradation of ECM components, especially interstitial collagens and basement membranes [19]. Matrix metalloproteinases (MMPs) are the prime mediators of ECM protein degradation and their expression is differentially regulated throughout the estrus cycle in the uterus [19]. The gradual accumulation of multiple ECM components in Antxr2−/− uteri indicates that there was a defect in a factor(s) known to degrade multiple and diverse ECM proteins.

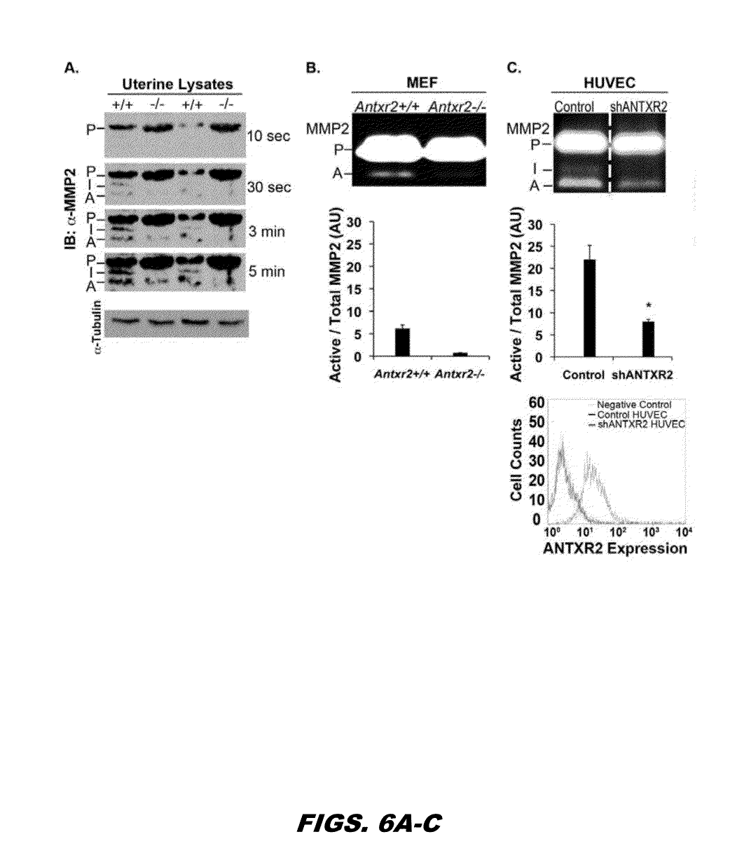

To evaluate MMP status in vivo, uterine lysates were used from 6-month-old Antxr2+/+ (n=2) and Antxr2−/− (n=2) mice and performed western blotting. A short exposure of the film (10 seconds) revealed increased level of proMMP2 in the Antxr2−/− tissue. In the Antxr2+/+ uterine lysates, intermediate and active MMP2 protein was clearly detected in longer film exposures (30 seconds, three minutes and five minutes, FIG. 6A). Despite equal loading of protein lysate, as evidenced by the tubulin loading control, active MMP2 was not readily detectable in the Antxr2−/− tissue until the three and five minute exposure times (FIG. 6A). The intermediate form of MMP2 was not detected in the Antxr2−/− tissue. Thus, MMP2 processing is defective in the uteri of Antxr2−/− female mice.

MMP2 activity was assessed in Antxr2+/+ and Antxr2−/− mouse embryonic fibroblasts (MEFs). Gelatin zymography revealed that there were reduced levels of active MMP2 in conditioned medium from Antxr2−/− MEFs (FIG. 6B). When quantified using densitometry, the ratio of active MMP2 to total MMP2 was eight fold higher in Antxr2+/+MEFs when compared to Antxr2−/− MEFs (FIG. 6B). This difference was almost statistically significant (P=0.06). Without artificial activation by organomercurials, it is very difficult to detect endogenous activation of MMP2 in MEFs. Therefore, the lack of significance is due to the low level of active MMP2 detected from the Antxr2+/+ cells.

RNAi was used to knockdown ANTXR2 in HUVEC, a cell type that requires ANTXR2 for endothelial proliferation and network formation, processes which could be affected by impaired MMP activity [6]. Flow cytometry was performed to detect knockdown of ANTXR2 by shRNA [6] at the cell surface (see histogram FIG. 6C). Similar to the MMP defects seen in MEFs, gelatin zymography showed that MMP2 levels were reduced in knockdown lines compared to control HUVEC (FIG. 6C). Quantification of the zymography bands demonstrated that the ratio of active to total MMP2 was 2.75 times higher in the control cells when compared to the knockdown cells (P=0.003) (FIG. 6C). Thus, two different cell types deficient for ANTXR2 expression had reduced MMP2 activation.

Anthrax Toxin Receptor 2 Regulates Membrane Type I Matrix Metalloproteinase Activity

The classic model for activation of MMP2 is through the formation of a trimolecular complex comprised of MTI-MMP, TIMP-2 and pro MMP2 [20]. MT1-MMP interacts via its N-terminal domain with the N terminus of TIMP-2 and this complex forms a receptor for pro MMP2. Pro MMP2 bound to this receptor is initially cleaved to its intermediate form by an adjacent active MT1-MMP. The second stage of MMP2 processing results in a fully active form and involves an autocatalytic event that requires an active MMP2 protein acting in trans [21,22,23]. Taking this mechanism of MMP2 activation into account, the increase in pro MMP2, the reduction in active MMP2 and the fact that the intermediate form of MMP2 was not detected in Antxr2−/− uterine tissue indicated that Antxr2 might be affecting MT1-MMP function.

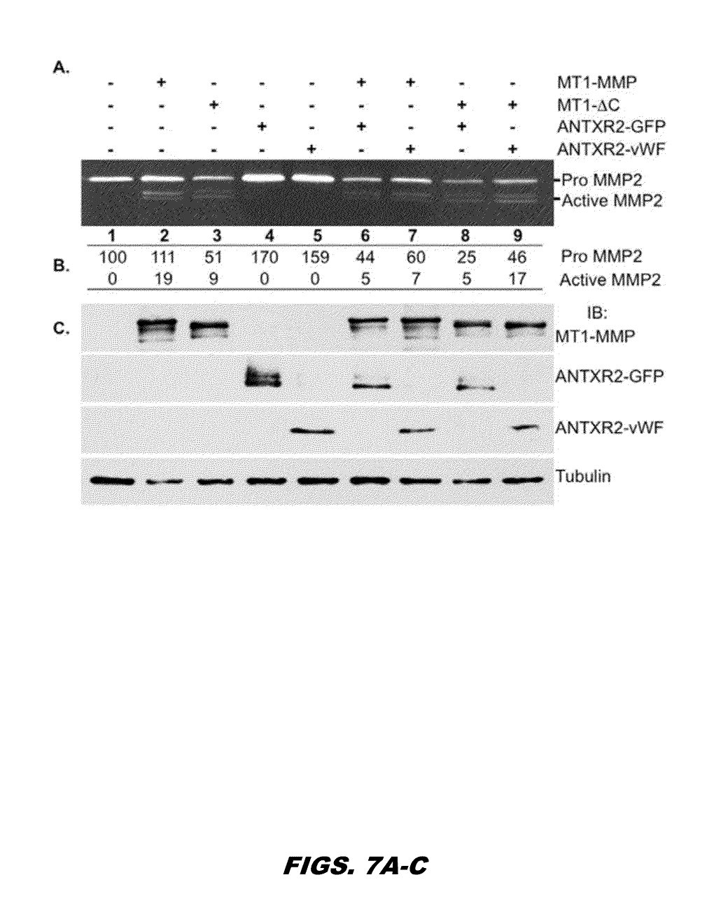

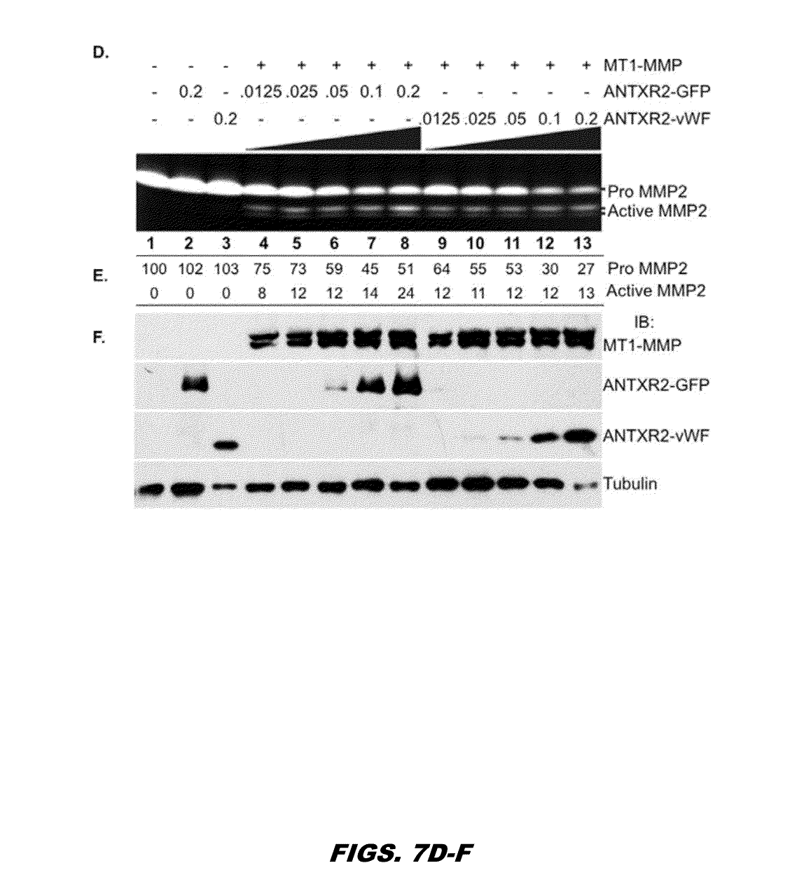

To address this, 293T cells were transfected with either wild type MT1-MMP or a catalytically active variant of MT1-MMP (MT1-ΔC), along with either full length ANTXR2 with a GFP tag at the carboxy terminus (ANTXR2-GFP) or a truncated variant of ANTXR2 consisting of the vWF domain (ANTXR2-vWF). Cell surface MT1-MMP activity was measured as the ability of cells to activate pro MMP2, a known substrate of MT1-MMP, and was evaluated using gelatin zymography. In this system, enhanced MT1-MMP activation was defined as a reduction in the amount of pro MMP2 detected. A corresponding increase in the amount of active MMP2 is more difficult to detect, as the half-life of the activated MMP2 enzyme is very short due to autocatalysis. Tables (FIGS. 7B and 7E) under the zymogram gels indicate densitometric quantification of the pro and active MMP2 bands and numbers are expressed as the percentiles of relative intensity in relation to the pro MMP2 band in the empty vector control (lane 1).

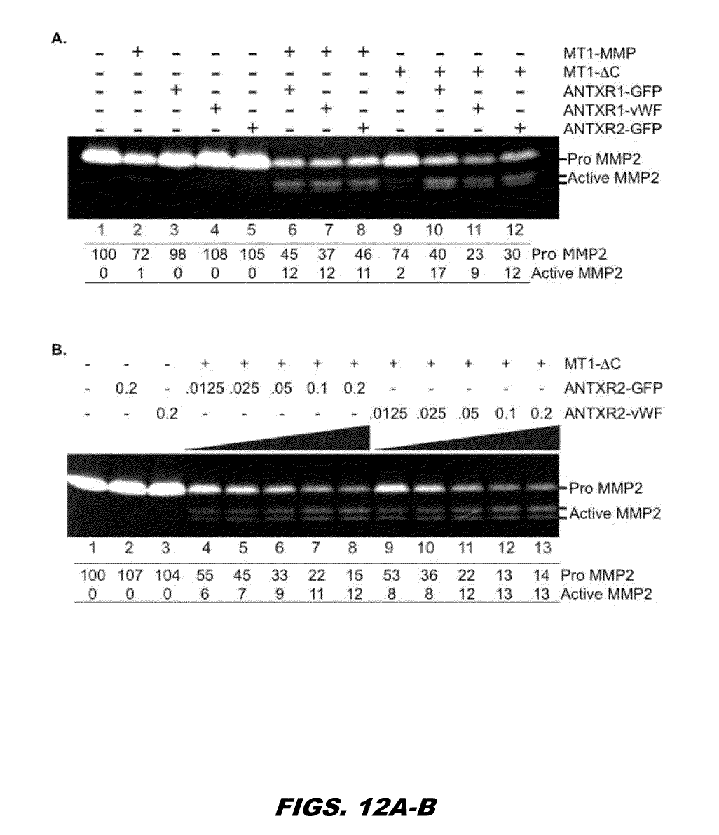

Expression of MT1-MMP in 293T cells showed trace levels of activated MMP2 (FIG. 7A, lane 2) and catalytically active MT1-ΔC showed enhanced pro MMP2 activation over wild type MT1-MMP in the conditioned medium (FIG. 7A, lane 3). Expression of either ANTXR2-GFP or ANTXR2-vWF alone had no affect on pro MMP2 processing (FIG. 7A, lanes 4 & 5). Co-expression of MT1-MMP and either ANTXR2-GFP or ANTXR2-vWF consistently showed greater MMP2 activation than cells expressing MT1-MMP alone (FIG. 7A, compare lane 2 to lanes 6 & 7). The processing of pro MMP2 was further enhanced in cells co-expressing MT1-ΔC and either ANTXR2-GFP or ANTXR2-vWF (FIG. 7A, lanes 8 & 9) Immunoblotting confirmed that the 293T cells were expressing MT1-MMP, MT1-ΔC, ANTXR2-GFP and ANTXR2-vWF and the appropriate combinations thereof (FIG. 7C). Similar results were obtained when 293T cells co-expressed MT1-MMP and the ANTXR2 homolog, ANTXR1 (FIG. 12). Co-expression of MT1-MMP and either ANTXR1-GFP or ANTXR1-vWF consistently showed pro MMP2 activation levels comparable to that achieved by co-expression of MT1-MMP and ANTXR2 (FIG. 12). This data demonstrates that ANTXR1 and ANTXR2 positively regulate MT1-MMP activity. Furthermore, the vWF domain, present on the extracellular side of the ANTXR proteins, is sufficient for promoting this activity.

To provide additional evidence in support of a role for ANTXR2 as a regulator of MT1-MMP activity, MT1-MMP activity was analyzed in response to various doses of ANTXR2-GFP or ANTXR2-vWF. Increased expression of ANTXR2-GFP resulted in a dose dependent decrease in pro MMP2 levels (FIG. 7D, lanes 4-8). A corresponding increase in active MMP2 levels was also detected. Densitometric quantification of the pro and active MMP2 bands confirmed the dose response (FIG. 7E). Co-expression of MT1-MMP with increasing amounts of ANTXR2-vWF also resulted in a dose dependent decrease in pro MMP2 levels (FIG. 7D, lanes 9-13), however, a corresponding increase in active MMP2 levels could not be captured. As mentioned earlier, this may be due to the short half-life of the active enzyme. Alternatively, it can indicate that the ANTXR2-vWF variant has partial function Immunoblotting confirmed that the 293T cells were expressing MT1-MMP and increasing amounts of ANTXR2-GFP and ANTXR2-vWF (FIG. 7C). The dose dependent response was also evident upon evaluation of MT1-ΔC activity (FIG. 12B, ANTXR2-GFP lanes 4-8, ANTXR2-vWF lanes 9-13). Thus, MT1-MMP processing of pro MMP2 is dependent on the ANTXR2 expression levels in cells.

Anthrax Toxin Receptor 2 and Membrane Type I Matrix Metalloproteinase Interact

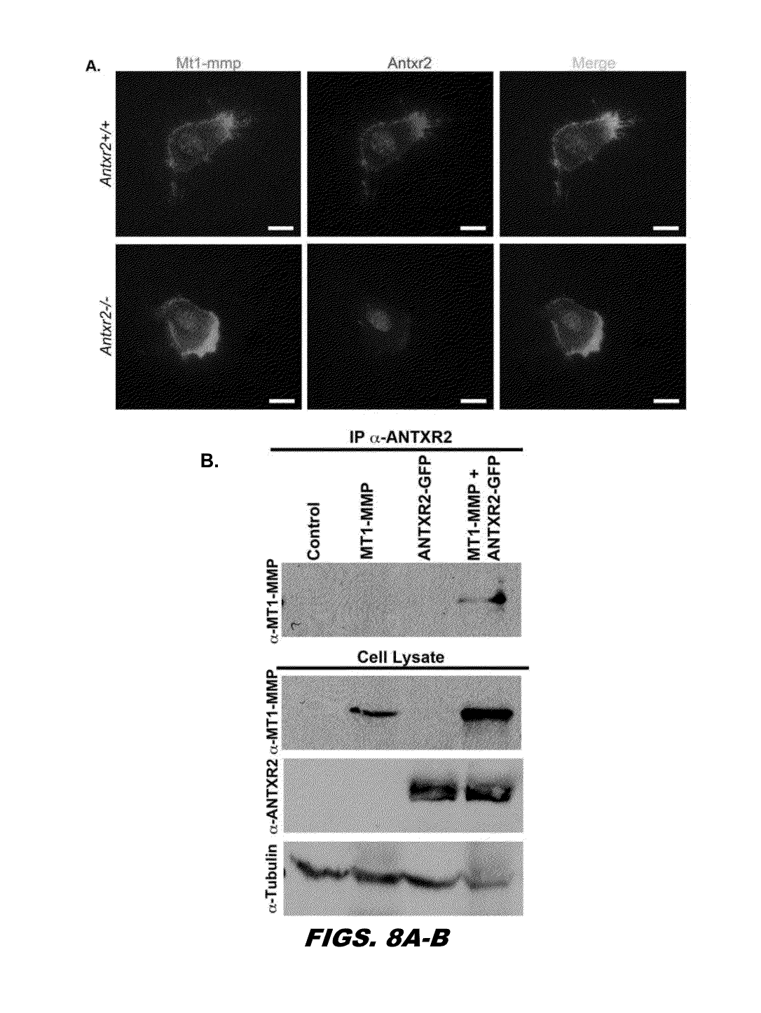

Whether ANTXR2 and MT1-MMP interact in cells was next examined To address this question, expression and interaction of MT1-MMP and ANTXR2 was studied in MEFs and in transfected 293T cells Immunofluorescent double labeling of unpermeabilized MEFs demonstrated that Mt1-mmp protein was present in a punctate membranous staining pattern on the cell surface of both Antxr2+/+ and Antxr2−/− MEFs (FIG. 8A). In Antxr2+/+ MEFs, Antxr2 localized to the cell surface and was found to colocalize with Mt1-mmp (FIG. 8A). Antxr2 was not expressed in Antxr2−/− MEFs as expected (FIG. 8A).

Coimmunoprecipitation experiments were carried out to confirm the association between the two proteins. 293T cells were transfected to express MT1-MMP, ANTXR2-GFP or MT1-MMP and ANTXR2-GFP and cell lysates were subjected to immunoprecipitation with an ANTXR2 antibody. The immunoprecipitated lysate was analyzed by western blotting with anti-MT1-MMP antibody. The experiment revealed that a 60 kDa protein representing MT1-MMP coimmunoprecipitated with ANTXR2 (FIG. 8B), indicating that ANTXR2 can localize to a complex with MT1-MMP.

Discussion

While there is a detailed understanding of ANTXR interaction with the tripartite anthrax toxin, physiological ANTXR activity has remained poorly defined. In order to evaluate endogenous function, an Antxr2 knockout mouse was generated by deleting exon 1 of the Antxr2 gene. Antxr2−/− mice were viable, however, Antxr2 was deemed to be required for parturition in young female mice and for preserving fertility in older female mice. Analysis of Antxr2−/− reproductive defects revealed that Antxr2 is required for myometrial cell viability and ECM homeostasis in the murine uterus and cervix and led to the discovery of a new mechanism of action for ANTXR2 as a positive regulator of MT1-MMP activity. This finding has implications for how ECM levels are regulated in developing, regenerating and pathological tissues.

The reproductive defects in female Antxr2−/− mice varied depending on the age of the mice at time of analysis. Young female Antxr2−/− mice mated immediately after reaching sexual maturity at 6 weeks of age were fertile. They were easily impregnated, carried their litters to term, but exhibited a block in parturition, the process of giving birth. Coordinated uterine contractions and cervical ripening are two processes that are essential to the progression of labor. Both of these processes were defective in the Antxr2−/− mice. Histological evaluation of the pregnant Antxr2−/− uterus at the end of the gestational period revealed loss of the circular and longitudinal myometrial cell layers (FIGS. 1D & E). This loss most likely resulted in nonproductive uterine contractions. Additionally, the Antxr2−/− cervix was found to be collagen dense indicating defective ECM remodeling and by extension defective cervical ripening (FIG. 1F).

Older sexually mature Antxr2−/− female mice, aged 2 to 6 months, demonstrated impaired fertility. Approximately half of the animals that were successfully impregnated would miscarry their litters. The other half carried their litters to term, but could not give birth, exhibiting parturition defects as described above. The underlying cause of impaired fertility in the older Antxr2−/− mice was likely due to defects in uterine receptivity as indicated by the atypical Antxr2−/− uterine morphology observed at the 2 to 6 month time points, which included mild fibrosis and disorganized myometrial muscle layers (FIG. 2B). Future studies focusing on ECM remodeling at various stages of pregnancy such as decidualization and placentation in Antxr2−/− mice can shed light on the fertility defect, however, changes in hormone expression profiles and downstream signaling cascades should not be ruled out.

Antxr2−/− female mice aged 7 months and beyond were infertile. Mating these mice did not result in pregnancies. Analysis of ovaries from older Antxr2−/− mice did not reveal overt changes in ECM content that might interfere with follicular maturation or oocyte production and release. Therefore, without being bound by theory, the uterus is unable to support implantation due to the fact that the pronounced fibrosis in aged Antxr2−/− uterine tissue completely destroys normal uterine architecture (FIG. 2B).

While various reproductive issues in Antxr2−/− female mice have been documented, Liu et. al. reported that female Antxr2−/− mice become pregnant but fail to support normal embryonic development, without further elaboration on the subject [24]. The fertility defects observed in the Antxr2−/− mice discussed in this example depend on the age of the mice at the time of analysis. Thus, the discrepancies between these results and the other study could be due to the age of the mice at the time of analysis, which was not specified in the Liu et. al. paper. In addition, the Liu et. al. group targeted the transmembrane domain of Antxr2 for deletion. This targeting strategy may allow for the production of a secreted variant of Antxr2, which could have functional significance. For instance, this study demonstrates that the extracellular domain alone can influence MMP activity (FIG. 7). This strategy of targeting exon 1 for deletion results in the complete loss of Antxr2 protein expression (FIG. 1).

The gradual accumulation of ECM proteins in the Antxr2−/− uterus indicated defective ECM remodeling, a process that should normally occur during each round of the estrus cycle. This prompted evaluation of MMP activity in the Antxr2−/− mice. ANTXR2 can be found in a complex with MT1-MMP (FIG. 8) and that co-expression of ANTXR2 and MT1-MMP in 293T cells promotes activation of the MT1-MMP/MMP2 proteolytic cascade (FIG. 7). Enhanced MMP2 processing from cells co-expressing ANTXRs and MT1-MMP (FIG. 7) could be attributed to increased levels of MT1-MMP in those cells. At times increased MT1-MMP protein expression in 293T cells that were also expressing ANTXR2 was observed (FIG. 7F and FIG. 8B), but this was not always the case (FIG. 7C). It remains to be determined whether the ANTXRs increase steady state levels of MT1-MMP in cells and this will be the subject of future studies.

The data indicates that ANTXR interaction with ECM components may facilitate multimerization and activation of a pericellular ANTXR/MT1-MMP complex. The fibrosis present in both the pregnant and nonpregnant uterus and cervix of Antxr2−/− mice may be the result of reduced Mt1-mmp activity in these tissues. In addition to its role in processing pro MMP2, MT1-MMP itself can degrade a number of ECM proteins including gelatin, fibronectin, vitronectin, fibrillar collagens and aggrecan [25]. It can also cleave a variety of other substrates, including cell surface receptors, growth factors, and cytokines [26]. Without being bound by theory, in the absence of Antxr2, Mt1-mmp and Mmp2 proteolytic activities are diminished in the uterus and cervix. In support of this, western blots on uterine lysates from Antxr2−/− mice demonstrated increased levels of pro MMP2 and a corresponding decrease in the levels of active Mmp2 in the tissue. It should be noted that while MT1-MMP is regarded as the main activator of MMP2, there are other pathways that regulate MMP2 activity. This is illustrated by the fact that zymographic analysis detected active MMP2 in tissues from Mt1-mmp−/− mice, albeit at greatly reduced levels [27]. Thus, in Antxr2−/− mice, it is likely that defective/reduced Mt1-mmp and Mmp2 activity resulted is an accumulation of type I collagen, type VI collagen, fibronectin and possibly other ECM proteins with each round of the estrus cycle. Mt1-mmp−/− mice have not been evaluated for reproductive defects since approximately 30% of the animals die before weaning with the remaining mutant mice dying between two to three months of age, however, it was noted that the Mt1-mmp−/− mice display no signs of sexual maturation[28].

The reproductive defects in female Antxr2−/− mice highlight the importance of the ANTXR/MT1-MMP complex for proper myometrial cell function. The myometrium has been demonstrated to express MT1-MMP [29] and MMP2 has been localized to the myometrium in the cycling uterus and during postpartum involution [16]. These reports point to myometrial cells as important mediators of ECM turnover in the remodeling uterus. The fact that accumulation of ECM proteins in the Antxr2−/− uterus coincides with the loss of myometrial cells indicates that a functional ANTXR/MT1-MMP complex is necessary for myometrial cells to effectively remodel the surrounding matrix.

Loss of the myometrium in the pregnant and non-pregnant Antxr2−/− uterus indicates that the ANTXR/MT1-MMP complex may also be essential for myometrial cell proliferation and viability. It is well established that the myometrium undergoes gradual changes during pregnancy, including a proliferative burst [30]. In the non-pregnant, sexually mature animal, myometrial cell proliferation is an integral part of the estrus cycle with the proliferative index peaking during proestrus [31]. It has recently been reported that MT1-MMP is a necessary cofactor for proper signaling through the PDGF-B/PDGFRβ axis in vascular smooth muscle cells [32]. Uterine myometrial cells have been demonstrated to express PDGFR, and treatment with PDGF induces a proliferative response in the cells [33]. Therefore, the PDGF signaling pathway may be an important growth factor that stimulates myometrial cell proliferation and survival during pregnancy and in the cycling uterus. It remains to be determined whether myometrial cell proliferation is impaired in the Antxr2−/− mice, but myometrial cell viability is clearly affected in the animals and future studies will determine if Antxr2 regulation of MT1-MMP activity intersects with the PDGFR signaling pathways in the myometrium.

Patients with JHF and ISH, the human diseases caused by mutations in the ANTXR2 gene, develop symptoms after birth and clinical features of the diseases include skin fibromas, gingival hypertrophy, joint contractures, osteoporosis and in the case of ISH, a failure to thrive [8,11]. The skin fibromas are thought to form as a result of excessive ECM accumulation. Remarkably, the phenotype of the Mt1-mmp−/− mouse bears a strong resemblance to the symptoms exhibited by patients with JHF and ISH. Mt1-mmp has been demonstrated to have little or no role in embryonic development, however loss of expression in the mouse results in progressive impairment of postnatal growth and development affecting both the skeleton and soft connective tissue [27,28,34]. Similar to humans with JHF and ISH, aging in the Mt1-mmp−/− mice is associated with generalized fibrosis, progressive craniofacial dysmorphism, joint contractures, severe reduction of bone growth (ostopenia), reduced mobility, and a failure to thrive [27,28]. Thus, the discovery that ANTXR2 positively regulates MT1-MMP activity could explain the phenoytpes associated with JHF and ISH. Antxr2−/− mice did not phenocopy JHF and ISH, nor did they phenocopy Mt1-mmp−/− mice. Activation of MT1-MMP is also regulated by ANTXR1 (FIG. 12), therefore, in some tissues Antxr1 could be compensating for loss of Antxr2 in the mutant mice. This highlights the importance of evaluating the phenotypes associated with Antxr1−/−; Antxr2−/− mice.

It is also interesting to note that a recent paper reported that MT1-MMP cleaves the anthrax toxin binding moiety, protective antigen (PA), leading to shedding of PA proteolytic fragments from cell surfaces [35]. Since PA is a ligand of ANTXRs, that finding not only supports the discovery that ANTXR2 and MT1-MMP interact, but indicates that this interaction might negatively regulate the process of anthrax intoxication. Further investigation can help understand this interaction.

While the mechanistic processes underlying ANTXR2/MT1-MMP interactions require further study, the research establishes a role for ANTXR2 as a regulator of MT1-MMP activity. ANTXR1 functions in a similar manner, which may explain the ECM accumulation observed in various organs of the Antxr1−/− mouse [9]. This novel mechanism of action for ANTXRs sheds light on the phenotypes associated with JHF and ISH and can inform future studies whether they are aimed at targeting anthrax intoxication or tumor growth and metastasis.

Materials and Methods

Generation of Antxr2 Knockout Mice.

Bacterial Artificial Chromosome RP23-162D22 (CHORI), containing the entire mouse Antxr2 gene, was used as a template during BAC recombineering to construct a conditional Anxtr2 targeting vector in which a single loxP site was inserted within the promoter region of the ANTXR2 gene, a floxed neomycin cassette (NEO) was inserted within intron 1 for positive selection and a diptheria toxin A (DTA) cassette was inserted in place of exon 3 for negative selection. The BAC targeting construct was linearized with PI-SCE I, purified by phenol/choloroform extraction and electroporated into 129/SvJ embryonic stem (ES) cells by Columbia University's Herbert Irving Cancer Center Transgenic Mouse Facility. After G418 selection, four hundred ES cell clones were screened by Southern analysis to determine which clones had undergone homologous recombination. Briefly, gDNA isolated from ES cells was digested with BamHI and Southern blots were hybridized with a ³²P-labeled probe to exon 3. This probe was designed to hybridize to a section of the gene outside the targeting vector homology arms in order to distinguish properly targeted recombination events from random integration. Four of the 400 ES cell clones screened had undergone proper targeting yielding a 4.4 kb band for the recombined allele and a 8 kb band for the wild-type allele (FIG. 9B). PCR was also used to detect the presence of the single loxP site upstream of exon 1 (FIG. 9B). Of these four ES cell clones, two were microinjected into host KV1 (129/Svj-057B6 hybrid) blastocysts to generate chimeric animals. Mating the male chimeras with female C57BL/6 mice resulted in germline transmission of the Antxr2 triloxP allele to the F1 generation. Mice heterozygous for the Antxr2 triloxP allele were intercrossed to produce homozygous Antxr2 triloxP mice. Antxr2+/− mice were derived in two mating steps. First male mice heterozygous for the Antxr2 triloxP allele were mated with female Ella-Cre transgenic mice. The maternally derived Cre is more efficient at producing total germline excision of the loxP1 and loxP3 flanked DNA (i.e. deletion of exon 1 and NEO cassette) due to the presence of Cre in the oocyte. As this mating has the potential to produce mosaic offspring, genotyping was performed to detect the various recombination products and the Cre allele in order to identify mice that were heterozygous for both the Antxr2 allele and the Cre allele. To segregate the Cre allele, Antxr2+/−; Cre mice were next mated with wild type C57BL/6. Once Antxr2+/− mice were obtained, intercrosses were set-up to produce Antxr2−/− mice.

Genotyping.

Mice were genotyped by PCR amplification of genomic DNA from tails. Primers for genotyping the conditional Antxr2 allele (Antxr2 floxed) were Forward 5′-CAGAACTCTAGGTCAGGGGC-3′ (SEQ ID NO: 5) and Reverse 5′-CTTATGCCTCATCCCTCCGC-3′ (SEQ ID NO: 6). This primer set yielded a 672 bp band to indicate the presence of the loxP site and a 600 bp band corresponding to the wild-type allele. Triplex PCR with three primers was used to detect knockout and wild-type Antxr2 alleles simultaneously; a common Forward primer 5′-CGGTCACCCTGGAGCTATGC-3′ (SEQ ID NO: 7) and allele-specific Reverse primers wild-type 5′-CTTATGCCTCATCCCTCCGC-3′ (SEQ ID NO: 8) and knockout 5′-GAGGAAACGAGCTGCAGGTG-3′ (SEQ ID NO: 9) were used. This primer set yielded a 316 bp band to indicate the presence of the Antxr2 knockout allele and a 488 bp band corresponding to the wild-type allele.

Animal Use.

Mice were housed under a 12 hr light cycle at 22° C. All Antxr2−/− mice and littermates were on a mixed C57BL/6-129SvJ background. Timed matings were performed by housing one male and two females in a cage. Each morning, females were evaluated for the presence of a plug and noon on the day a mating plug was detected was considered gestational day 0.5.

Isolation of Mouse Embryonic Fibroblasts.

Embryos were collected from the uteri of pregnant mice on gestational day 13.5. The heads and livers were removed and the carcasses were minced and trypsinized Fibroblasts from the embryos were cultured in DMEM supplemented with 10% FBS and 50 mg/ml penicillin and streptomycin (GIBCO) in 5% CO₂ at 37° C. gDNA isolated from embryo yolk sacs was used for genotyping PCR.

Reverse Transcription PCR.

Total RNA was isolated from MEFs using the RNeasy kit (Qiagen, Valencia, Calif.). First strand cDNA synthesis was performed using random hexamers and Superscript II reverse transcriptase (Invitrogen, Carlsbad, Calif.). PCR for mouse β-actin and mouse Antxr2 was performed using PCR primers as follows: mouse Antxr2 exon1 Forward 5′-CTCTTGCAAAAAAGCCTTCG-3′ (SEQ ID NO: 10) and Reverse 5′-TTCTTTGCCTCGTTCTCTGC-3′ (SEQ ID NO: 11); mouse Antxr2 exon2 Forward 5′-GTCTGGCAGTGTAGC-3′ (SEQ ID NO: 12) and Reverse 5′-TTCTTTGCCTCGTTCTCTGC-3′ (SEQ ID NO: 13); mouse β-actin Forward 5′-CGAGGCCCAGAGCAAGAGAG-3′ (SEQ ID NO: 14) and Reverse 5′-CTCGTAGATGGGCACAGTGTG-3′(SEQ ID NO: 15).

Histologic Evaluation of Mouse Tissue.

Analysis of the parturition defect was conducted using three Antxr2+/+ and seven Antxr2−/− female mice. Reproductive tracts were isolated on GD18.5, fixed in 4% paraformaldehyde (PFA) and routinely processed for embedding in either OCT or paraffin. 5-μm serial sections were stained with H&E and Masson's Trichrome. See below for immunostaining. Reproductive tracts were isolated from nulliparous Antxr2+/+ and Antxr2−/− mice at age 1 month to 15 months. At the time of collection, a small portion of each uterine horn was snap frozen in liquid nitrogen for immunoblotting analysis (see below). Tissue was analyzed from three animals per genotype for each age group. The tissues were treated as specified above.

Colorimetric IHC.

For immunohistochemical studies evaluating Antxr2 expression in pregnant uterine tissue, fixed frozen 5-μm serial sections were post-fixed in acetone, blocked in phosphate-buffered saline (PBS) containing 3% bovine serum albumin and 2% rabbit serum (Sigma). Primary antibody goat anti-mouse Antxr2 (R&D) was incubated overnight at 4° C. Negative controls were left with blocking solution. Incubation with biotinylated secondary antibody rabbit anti-goat (Vector Laboratories) was performed for one hour at room temperature and followed by incubation with avidin and horseradish-peroxidase conjugated biotin in PBS (Vectastain Standard ABC Elite kit, Vector Laboratories). The color reaction was performed using DAB (diaminobenzidine tetrahydrochloride), the peroxidase substrate (Vector Laboratories). Tissues were counterstained with hematoxylin (Fisher).

Immunofluorescent IHC.

Immunostaining was performed as described above until application of primary and secondary antibodies. Primary antibodies used were: mouse anti-∝SMACy3 (Sigma), biotinylated rabbit anti-type VI collagen (Rockland), rabbit anti-type I collagen (Millipore), rabbit anti-fibronectin (Abcam), rat anti-mouse CD31 (BD Pharminogen), rat anti-endomucin (Santa Cruz), goat anti-lyve-1 (R&D), rat anti-mouse F4/80 (Abcam). Sections were incubated with Alexa Fluor tagged secondary antibodies (Molecular Probes), which were specific to each primary antibody. DAPI (4,6-diamidino-2-phenylindole) (Sigma) was used to visualize nuclei. Negative controls were treated with secondary antibody alone. Images were obtained on Nikon ECLIPSE E 800 microscope (Nikon Inc.).

Serum Progesterone Measurements.

Progesterone levels were measured in the sera of mice on gestational days 15.5 and 18.5. Sera were collected from three Antxr2+/+ mice and five Antxr2−/− mice. Blood was drawn via cardiac puncture, allowed to clot at room temperature for 30 minutes and centrifuged to remove red blood cells. The sera were stored at −80° C. until time of analysis. Serum progesterone levels were measured using a mouse progesterone ELISA kit (Cusabio Biotech Co.) following manufacturer instructions.

ANTXR2 Gene Silencing and Cell Surface Receptor Expression Analysis

ANTXR2 gene silencing in HUVEC cell lines has been described [6]. Flow cytometry analysis of ANTXR2 expression on the cell surface has been described [36].

DNA Constructs.

ANTXR2-GFP and ANTXR2-vWF constructs have been described [37].

ANTXR1-GFP and ANTXR1-vWF constructs have been described [37,38]. All of these constructs were engineered into retroviral vector pHyTCX for the experiments described herein. Wild-type MT1-MMP and C-terminally truncated MT1-MMP (MT1-ΔC) constructs have been described [39].

Transfections and Gelatin Zymography.

Gelatin Zymography analysis was performed as previously described [40,41]. 5×10⁴ 293 Ts were seeded in 400 ul of DMEM with 10% fetal bovine serum in a 24 well plate. Cells were transfected with Effectene (Qiagen) according to the manufacturer's protocol. After transfection, cells were washed with PBS, and cultured in DMEM with 5% fetal bovine medium (the source of pro MMP2). After 16-24 hours, the condition medium was harvested and cleared by centrifugation at 12,000 rpm for 10 minutes and subjected to analysis by SDS-substrate gel electrophoresis (zymography) under non-denaturing conditions in 8.0% SDS-polyacrylamide gels impregnated with 1 mg/ml gelatin as previously described [40,41]. The gels were incubated at 37° C. overnight in 50 mM Tris (pH 7.5), 5 mM CaCl₂, 1 mM ZnCl₂ and stained with Coomassie Brilliant Blue R25. Destained gel images were captured by Kodak EL Logic 100 Imaging System. For MEF and 293T zymography, experimental samples were tested in duplicate. For HUVEC zymography, all of the experimental samples were tested in quadruplicate. All of the experiments were repeated twice. ImageJ 1.45s (NIH) was used to quantify zymography band intensities.

Tissue Lysate Preparation and Immunoblotting.

Uterine tissues were homogenized on ice in 500 mL RIPA buffer (50 mM Tris-HCl, pH 7.5, 10 mM EDTA, 150 mM NaCl, 1% Nonidet P-40, and protease inhibitor cocktail). Homogenized lysate was clarified by centrifugation at 12,000 rpm at 4° C. for 10 minutes. Protein concentration was determined using Bradford reagent (BioRad). Lysates containing 10 μg of protein were electrophoresed in the appropriate percentage SDS-polyacrylamide gel (6% for type I collagen, type VI collagen, fibronectin; 10% for MMP2, MT1-MMP, MT1-ΔC, ANTXR2-GFP, and ANTXR2-vWF). Protein was transferred to nitrocellulose by electroblotting and then blocked for 1 hour at 22° C. in PBST (1×PBS, 0.2% Tween) containing 3% bovine serum albumin. Blots were incubated with appropriate primary antibodies in blocking solution overnight at 4° C. Antibodies used were biotinylated rabbit anti-type VI collagen (Rockland), rabbit anti-type I collagen (Millipore), rabbit anti-fibronectin (Abcam), rabbit anti-MMP2 (Abcam), rabbit anti-MT1-MMP (Epitomics), goat anti-ANTXR2 (R&D). The blots were washed three times for 10 minutes each in PBST and incubated in the appropriate HRP secondary antibodies for 1 hour at 22° C. The blots were washed as above and then incubated for 5 minutes in enhanced chemiluminescence reagents (Fisher) and exposed to film (Kodak).

Immunocytochemistry.

To visualize Mt1-mmp and Antxr2 on cell surfaces, MEFs were seeded on gelatin-coated coverslips in 24 well plates. The next day cells were washed twice with ice cold PBS and stained with rabbit anti-MT1-MMP (Epitomics) and goat anti-Antxr2 (R&D) for one hour at 4° C. The cells were washed three times in ice cold PBS and fixed in 4% PFA for 10 minutes at room temperature. After fixation, the cells were incubated in PBS containing 3% bovine serum albumin and 2% donkey serum for 30 minutes at room temperature and then stained with donkey anti-rabbit alexa fluor 488 and donkey anti-goat alex fluor 594 for 30 minutes at room temperature. Following three washes with PBS, coverslips were mounted in Vectashield containing DAPI. Images were obtained on Nikon ECLIPSE E 800 microscope. To reveal colocalization of the two proteins, the images were processed and merged in Adobe PhotoShop software.

Immunoprecipitation.

Transfected cells were lysed in RIPA buffer (50 mM Tris-HCl, pH 7.5, 10 mM EDTA, 150 mM NaCl, 1% Nonidet P-40, and protease inhibitor cocktail) for 30 minutes at 4° C. Cell extracts were cleared by centrifugation at 12,000 rpm for 10 minutes and the supernatant was incubated at 4° C. with goat anti-ANTXR2 (R&D) for 2 hours Immune complexes were immobilized on protein-A/G beads for 3 hours, washed three times with lysis buffer, and subjected to Western-blotting analysis with rabbit anti MT1-MMP antibody (Epitomics).

Statistical Analysis

Statistical significance was evaluated using the unpaired Student's t test with P value less than 0.05 considered statistically significant.

### REFERENCES

1. Whittaker C A, Hynes R O (2002) Distribution and evolution of vonWillebrand/integrin A domains: widely dispersed domains with roles in cell adhesion and elsewhere. Mol Biol Cell 13: 3369-3387.
2. Young J A, Collier R J (2007) Anthrax toxin: receptor binding, internalization, pore formation, and translocation. Annu Rev Biochem 76: 243-265.
3. Nanda A, Carson-Walter E B, Seaman S, Barber T D, Stampfl J, et al. (2004) TEM8 interacts with the cleaved C5 domain of collagen alpha 3(VI). Cancer Res 64: 817-820.
4. Hotchkiss K A, Basile C M, Spring S C, Bonuccelli G, Lisanti M P, et al. (2005) TEM8 expression stimulates endothelial cell adhesion and migration by regulating cell-matrix interactions on collagen. Exp Cell Res 305: 133-144.
5. Bell S E, Mavila A, Salazar R, Bayless K J, Kanagala S, et al. (2001) Differential gene expression during capillary morphogenesis in 3D collagen matrices: regulated expression of genes involved in basement membrane matrix assembly, cell cycle progression, cellular differentiation and G-protein signaling. J Cell Sci 114: 2755 2773.
6. Reeves C V, Dufraine J, Young J A, Kitajewski J (2009) Anthrax toxin receptor 2 is expressed in murine and tumor vasculature and functions in endothelial proliferation and morphogenesis. Oncogene 6: 789-801.
7. Rmali K A, Puntis M C, Jiang W G (2005) TEM-8 and tubule formation in endothelial cells, its potential role of its vW/TM domains. Biochem Biophys Res Commun 334: 231-238.
8. Dowling O, Difeo A, Ramirez M C, Tukel T, Narla G, et al. (2003) Mutations in capillary morphogenesis gene-2 result in the allelic disorders juvenile hyaline fibromatosis and infantile systemic hyalinosis. Am J Hum Genet 73: 957-966.
9. Cullen M, Seaman S, Chaudhary A, Yang M Y, Hilton M B, et al. (2009) Host-derived tumor endothelial marker 8 promotes the growth of melanoma. Cancer Res 69: 6021 6026.
10. Stucki U, Spycher M A, Eich G, Rossi A, Sacher P, et al. (2001) Infantile systemic hyalinosis in siblings: clinical report, biochemical and ultrastructural findings, and review of the literature. Am J Med Genet 100: 122-129.
11. Hanks S, Adams S, Douglas J, Arbour L, Atherton D J, et al. (2003) Mutations in the gene encoding capillary morphogenesis protein 2 cause juvenile hyaline fibromatosis and infantile systemic hyalinosis. Am J Hum Genet 73: 791-800.
12. Tanaka K, Ebihara T, Kusubata M, Adachi E, Arai M, et al. (2009) Abnormal collagen deposition in fibromas from patient with juvenile hyaline fibromatosis. J Dermatol Sci 55: 197-200.
13. Nathanielsz P W (1978) Endocrine mechanisms of parturition. Annu Rev Physiol 40: 411-445.
14. O'Brien W F (1995) Cervical ripening and labor induction: progress and challenges. Clin Obstet Gynecol 38: 221-223.
15. Leppert P C (1995) Anatomy and physiology of cervical ripening. Clin Obstet Gynecol 38: 267-279.
16. Rudolph-Owen L A, Hulboy D L, Wilson C L, Mudgett J, Matrisian L M (1997)
Coordinate expression of matrix metalloproteinase family members in the uterus of normal, matrilysin-deficient, and stromelysin-1-deficient mice. Endocrinology 138: 4902-4911.
17. Hunt J S, Manning L S, Mitchell D, Selanders J R, Wood G W (1985) Localization and characterization of macrophages in murine uterus. J Leukoc Biol 38: 255-265.
18. Wood G A, Fata J E, Watson K L, Khokha R (2007) Circulating hormones and estrous stage predict cellular and stromal remodeling in murine uterus. Reproduction 133: 1035-1044.
19. Fata J E, Ho A T, Leco K J, Moorehead R A, Khokha R (2000) Cellular turnover and extracellular matrix remodeling in female reproductive tissues: functions of metalloproteinases and their inhibitors. Cell Mol Life Sci 57: 77-95.
20. Strongin A Y, Collier I, Bannikov G, Marmer B L, Grant G A, et al. (1995) Mechanism of cell surface activation of 72-kDa type IV collagenase. Isolation of the activated form of the membrane metalloprotease. J Biol Chem 270: 5331-5338.
21. Sato H, Takino T, Okada Y, Cao J, Shinagawa A, et al. (1994) A matrix metalloproteinase expressed on the surface of invasive tumour cells. Nature 370: 61 65.
22. Hernandez-Barrantes S, Toth M, Bernardo M M, Yurkova M, Gervasi D C, et al. (2000) Binding of active (57 kDa) membrane type 1-matrix metalloproteinase (MT1 MMP) to tissue inhibitor of metalloproteinase (TIMP)-2 regulates MT1-MMP processing and pro-MMP-2 activation. J Biol Chem 275: 12080-12089.
23. Atkinson S J, Crabbe T, Cowell S, Ward R V, Butler M J, et al. (1995) Intermolecular autolytic cleavage can contribute to the activation of progelatinase A by cell membranes. J Biol Chem 270: 30479-30485.
24. Liu S, Crown D, Miller-Randolph S, Moayeri M, Wang H, et al. (2009) Capillary morphogenesis protein-2 is the major receptor mediating lethality of anthrax toxin in vivo. Proc Natl Acad Sci USA 106: 12424-12429.
25. Ohuchi E, Imai K, Fujii Y, Sato H, Seiki M, et al. (1997) Membrane type 1 matrix metalloproteinase digests interstitial collagens and other extracellular matrix macromolecules. J Biol Chem 272: 2446-2451.
26. Overall C M, McQuibban G A, Clark-Lewis I (2002) Discovery of chemokine substrates for matrix metalloproteinases by exosite scanning: a new tool for degradomics. Biol Chem 383: 1059-1066.
27. Zhou Z, Apte S S, Soininen R, Cao R, Baaklini G Y, et al. (2000) Impaired endochondral ossification and angiogenesis in mice deficient in membrane-type matrix metalloproteinase I. Proc Natl Acad Sci USA 97: 4052-4057.
28. Holmbeck K, Bianco P, Caterina J, Yamada S, Kromer M, et al. (1999) MT1-MMP deficient mice develop dwarfism, osteopenia, arthritis, and connective tissue disease due to inadequate collagen turnover. Cell 99: 81-92.
29. Morikawa A, Ohara N, Xu Q, Nakabayashi K, DeManno D A, et al. (2008) Selective progesterone receptor modulator asoprisnil down-regulates collagen synthesis in cultured human uterine leiomyoma cells through up-regulating extracellular matrix metalloproteinase inducer. Hum Reprod 23: 944-951.
30. Shynlova 0, Oldenhof A, Dorogin A, Xu Q, Mu J, et al. (2006) Myometrial apoptosis: activation of the caspase cascade in the pregnant rat myometrium at midgestation. Biol Reprod 74: 839-849.
31. Burroughs K D, Fuchs-Young R, Davis B, Walker C L (2000) Altered hormonal responsiveness of proliferation and apoptosis during myometrial maturation and the development of uterine leiomyomas in the rat. Biol Reprod 63: 1322-1330.
32. Lehti K, Allen E, Birkedal-Hansen H, Holmbeck K, Miyake Y, et al. (2005) An MT1 MMP-PDGF receptor-beta axis regulates mural cell investment of the microvasculature. Genes Dev 19: 979-991.
33. Rossi M J, Chegini N, Masterson B J (1992) Presence of epidermal growth factor, platelet-derived growth factor, and their receptors in human myometrial tissue and smooth muscle cells: their action in smooth muscle cells in vitro. Endocrinology 130: 1716-1727.
34. Holmbeck K, Bianco P, Yamada S, Birkedal-Hansen H (2004) MT1-MMP: a tethered collagenase. J Cell Physiol 200: 11-19.
35. Rozanov D V, Golubkov V S, Strongin A Y (2005) Membrane type-1 matrix metalloproteinase (MT1-MMP) protects malignant cells from tumoricidal activity of re-engineered anthrax lethal toxin. Int J Biochem Cell Biol 37: 142-154.
36. Scobie H M, Wigelsworth D J, Marlett J M, Thomas D, Rainey G J, et al. (2006) Anthrax toxin receptor 2-dependent lethal toxin killing in vivo. PLoS Pathog 2: ell 1.
37. Scobie H M, Rainey G J, Bradley K A, Young J A (2003) Human capillary morphogenesis protein 2 functions as an anthrax toxin receptor. Proc Natl Acad Sci USA 100: 5170-5174.
38. Scobie H M, Thomas D, Marlett J M, Destito G, Wigelsworth D J, et al. (2005) A soluble receptor decoy protects rats against anthrax lethal toxin challenge. J Infect Dis 192: 1047-1051.
39. Pei D, Weiss S J (1996) Transmembrane-deletion mutants of the membrane-type matrix metalloproteinase-1 process progelatinase A and express intrinsic matrix degrading activity. J Biol Chem 271: 9135-9140.
40. Wang X, Yi J, Lei J, Pei D (1999) Expression, purification and characterization of recombinant mouse MT5-MMP protein products. FEBS Lett 462: 261-266.
41. Wang X, Wilson M J, Slaton J W, Sinha A A, Ewing S L, et al. (2009) Increased aggressiveness of human prostate PC-3 tumor cells expressing cell surface localized membrane type-1 matrix metalloproteinase (MT1-MMP). J Androl 30: 259-274.

### Example 2

Mammographically dense breast tissue, which is characterized by increases in the extracellular matrix protein, collagen, is a risk factor for developing breast cancer. On the other hand, myoepithelial cells that surround mammary ducts and aveoli are thought to have a role in tumor and metastasis suppression due to the fact that they form a natural barrier between the luminal epithelial cells (the cells from which tumor form) and the surrounding environment. Myoepithelial cells also secrete proteins that limit cancer growth, invasiveness and blood vessel formation. Nevertheless, the role of both the extracellular matrix and myoepithelial cells during tumor progression remains poorly defined and warrants further investigation.

Utilizing mouse embryonic fibroblasts, a protein called Anthrax Toxin Receptor 2 (ANTXR2) has a role in remodeling extracellular matrix proteins, such as collagen, via interaction with a group of proteins called matrix metalloproteinases. Addressing whether regulation of matrix metalloproteinases by ANTXR2 is important in the human breast led to the discover of dysregulation of ANTXR2 in human breast cancer. In normal human breast tissue, ANTXR2 is expressed on blood vessels and on myoepithelial cells surrounding mammary ducts. In human breast cancer, the Oncomine cancer gene expression database reveals that ANTXR2 expression is reduced more than 3 fold in invasive ductal breast carcinoma when compared to normal breast tissue. Thus, ANTXR2 expression is found in myoepithelial cells of normal breast tissue but may be lost during transition to invasive ductal breast carcinoma. Without being bound by theory, ANTXR2 contributes to the tumor suppressive function of myoepithelial cells by regulating the activity of matrix metalloproteinases, which remodel the peri ductal extracellular matrix.

Expression levels of ANTXR2 can be evaluated in clinical specimens of human non-invasive and invasive ductal carcinoma as compared to normal breast tissue. First, it can be determined if there is a relationship between ANTXR2 expression, tumor grade, and size. Second, it can be determined if there is a relationship between reduced ANTXR2 levels, myoepithelial cell content and extracellular matrix changes in the samples. In order to determine whether ANTXR2 contributes to the tumor suppressive function of myoepithelial cells, ANTXR2 activity can be characterized in myoepithelial cells in culture and then whether a loss of ANTXR2 expression in myoepithelial cells contributes to tumor development using a mouse model of human breast cancer can be determined.

This study can investigate the incredibly complex connection between the tumor microenvironment and the progression of cancer. In addition, there will be a better understanding of ANTXR2 function in myoepithelial cells and its significance in breast cancer which can ultimately lead to new mechanisms of intervention or therapeutic approaches that target epithelial-stromal interactions.

### BACKGROUND

This example builds upon a new discovery made during the investigation of the mechanism of action for the Anthrax Toxin Receptor 2 protein and relates to interests in cell types that contribute to breast cancer progression and the contribution of the microenvironment to breast cancer.

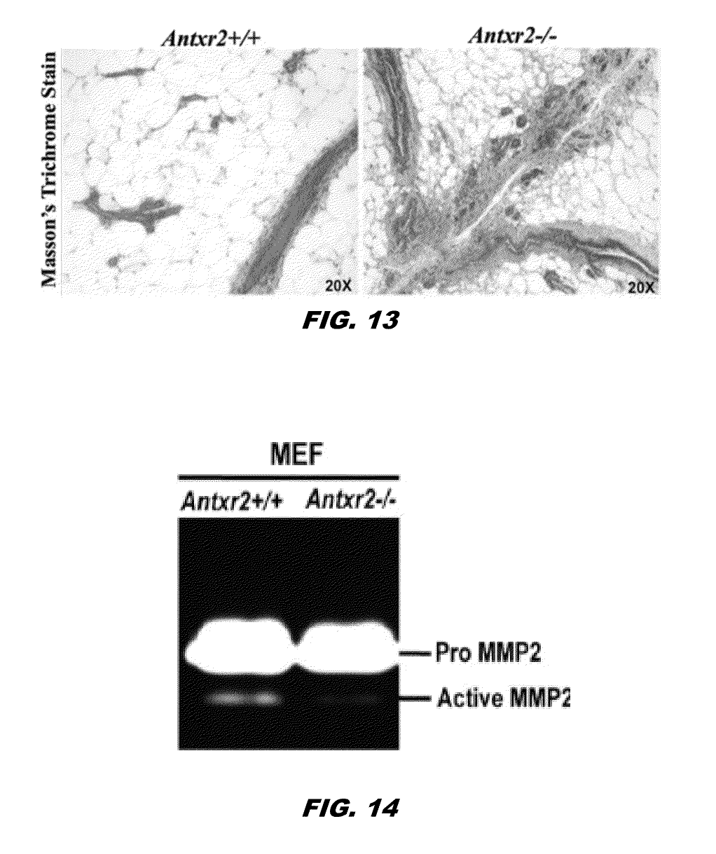

ANTXR2 functions to regulate matrix metalloproteinases: a new function for this receptor. This discovery arose when the function of ANTXR2 was ascertained in vivo by generating Antxr2−/− mice. Assessment of aged (3-15 month) Antxr2−/− mice has revealed smooth muscle cell defects and increased deposition of extracellular matrix (ECM) proteins in several organs including the mammary tissue (FIG. 13).

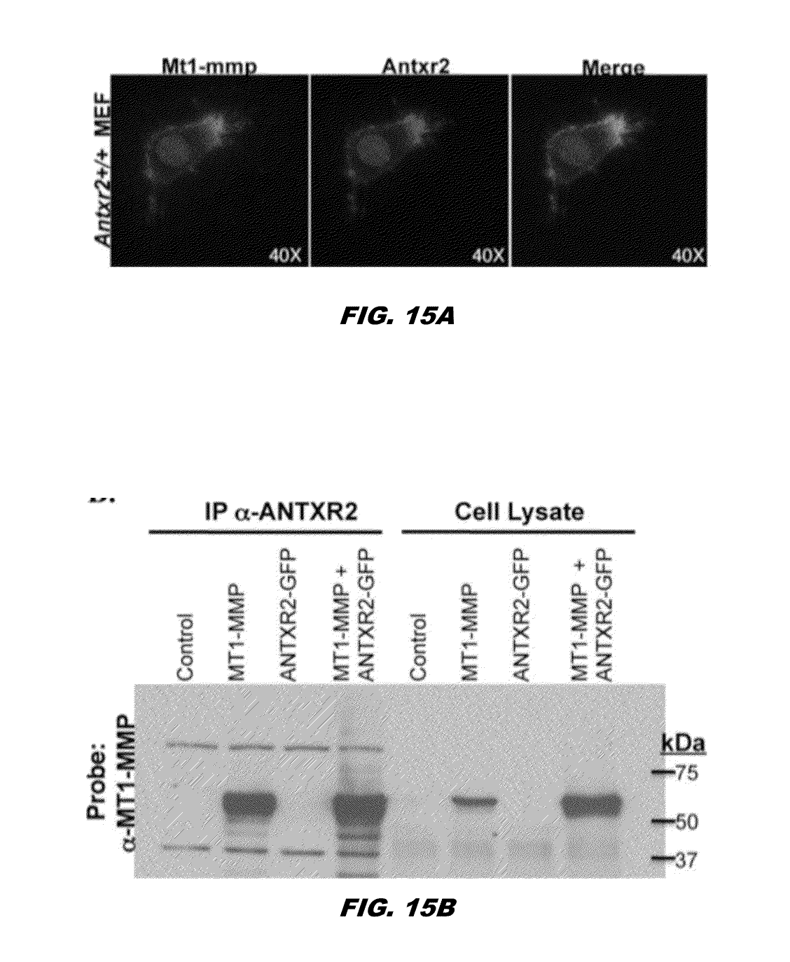

ECM proteins are degraded by Matrix Metalloproteases (MMPs). Therefore, MMP activity was assessed in the Antxr2−/− mice. Initial data demonstrated a decrease in the amount of active MMP-2 produced by Antxr2−/− mouse embryonic fibroblasts (MEFs), as compared to Antxr2+/+ MEFs (FIG. 14). Biochemical analyses also demonstrated that ANTXR2 and Membrane Type 1 Matrix Metalloprotease (MT1-MMP) colocalize in MEFs (FIG. 15A) and physically interact when overexpressed in 293T cells (FIG. 15B).

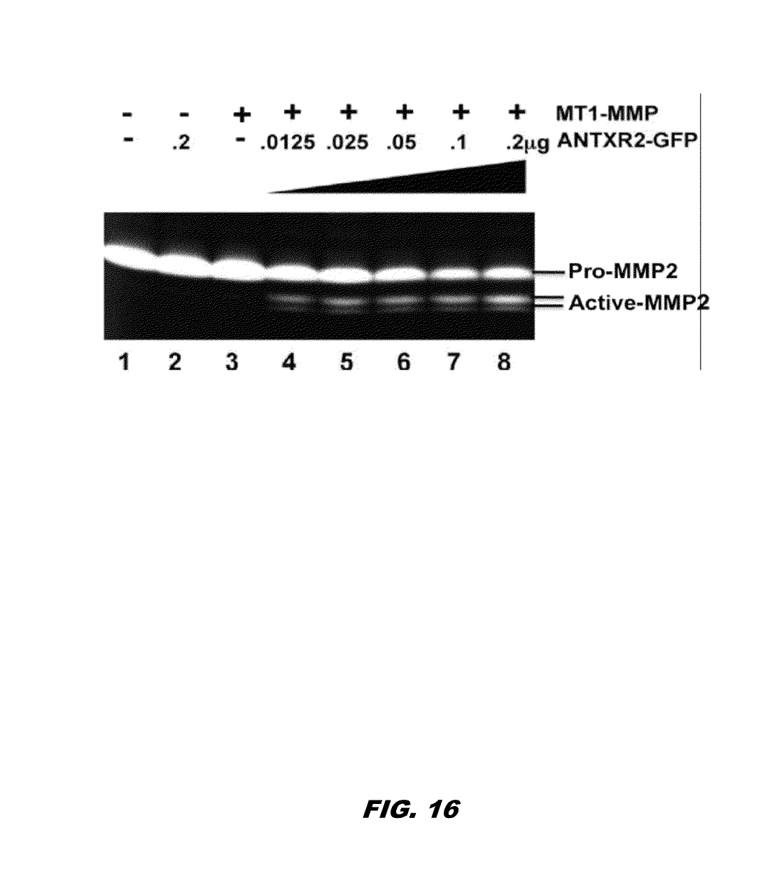

Furthermore, MTI-MMP and ANTXR2 co-expression in cells increased the level of activated MMP-2 in an ANTXR2 dose dependent manner (FIG. 16).

Taking into account that MT1-MMP is known to regulate the activation of MMP-2 (S8), ANTXR2 is localized in a complex with MT1-MMP at the cell surface and facilitates activation of MMP-2 thereby regulating ECM homeostasis.

Abnormal ECM homeostasis and tissue organization increases the chance of tumor initiation. Mammographically dense breast tissue, which is characterized by increased stromal collagen, is a risk factor for developing breast cancer (S9). Additionally, over-expression of collagen in mice has been shown to promote breast tumor progression (S10). Based on these observations, it was determined whether the regulation of MMPs by ANTXR2 is important in the human breast. Addressing this question led to the discovery of dysregulation of ANTXR2 in human breast cancer. In normal human breast tissue, myoepithelial cells surrounding mammary ducts and blood vessels were found to express ANTXR2 (S11). In human breast ductal carcinoma in situ (DCIS) specimens, ANTXR2 expression was absent from the tumor cells but was localized to three sites of expression: i) the myoepithelial cells surrounding tumors; ii) cells within the tumor stroma; and iii) blood vessels throughout both the tumor and stroma (S11). It was investigated how ANTXR2 expression is altered during invasive carcinoma utilizing the Oncomine cancer gene expression microarray database (http://www.oncomine.org). Four different microarray studies revealed that levels of ANTXR2 mRNA are reduced more than 3 fold in invasive ductal breast carcinoma when compared with normal breast tissue (S12-S15). These datasets analyzed a combined total of 18 normal breast samples and 233 tumor samples. Thus, ANTXR2 expression is found in myoepithelial cells of normal breast and in DCIS but may be lost during transition to invasive ductal breast carcinoma.

Myoepithelial cells (MECs) are thought to have an endogenous program of tumor and metastasis suppression due to the fact that MECs form a natural barrier between the luminal epithelial cells and the surrounding stroma. MECs also secrete proteins that limit cancer growth, invasiveness and neovascularization (S16). Still, the role of MECs during tumor progression remains underappreciated and poorly defined. For example, it has been generally accepted that there is a loss of MECs in invasive carcinoma, however, current studies report on the presence of morphologically identifiable MECs in breast cancers that express a subset of the markers used to define a MEC (S17, S18). This indicates a dysregulation of the MEC differentiation program during breast cancer progression. Clearly the role of the myoepithelial cell during breast tumorigenesis warrants further investigation.

Taken together, the discovery that ANTXR2 regulates MMP activity, the marked expression of ANTXR2 on the myoepithlial cells in normal human mammary tissue and the reduced expression of ANTXR2 in invasive ductal carcinoma have led to the following: ANTXR2 contributes to the tumor suppressive function of myoepithelial cells by interacting with MT1-MMP to regulate the activation of secreted MMPs in periductal stroma. In recent years, MMPs have been demonstrated to be very diverse in their function including roles in both tumor promotion and tumor inhibition. Therefore, it is necessary to explore MMP function is various cell types. For example, ANTXR2 contributes to the tumor suppressive function of myoepithelial cells by interacting with MT1-MMP to regulate the activation of secreted MMPs in periductal stroma.

To Evaluate the Expression Levels of ANTXR2 in Human Noninvasive and Invasive Ductal Carcinoma as Compared to Normal Breast Tissue.

Oncomine microarray studies identify ANTXR2 as a gene with downregulated expression in invasive ductal carcinoma as compared to normal human breast tissue. However, follow-up immunohistochemical studies of ANTXR2 protein expression have not been performed. To validate the microarray data, ANTXR2 expression can be evaluated in human breast cancer tissues. Fresh frozen sections can be acquired from the Herbert Irving Comprehensive Cancer Center Tumor Bank (Columbia University). The samples can represent histologically normal breast tissue from reduction mammoplasties (n=20), ductal carcinoma in situ (DCIS) (n=50) and invasive ductal carcinoma (IDC) (n=50). Clinical information, such as tumor size, grade and stage can be obtained from pathology reports. ANTXR2 expression can be assessed semiquantitatively by giving the tissue scores for ANTXR2 staining intensity and distribution in each of 10 high-powered fields. Staining intensity (strength of signal) can be evaluated as negative (0), weak (1), moderate (2), strong (3) Staining distribution can be defined as the percentage of positive cells in each field and categorized as follows: 0=0% to 5%, 1=6% to 25%, 2=26% to 50%, 3=51% to 75%, and 4=76% to 100%. A staining index can be determined by multiplying staining intensity and distribution and a score (0-12) can be obtained for each sample. ANTXR2 expression can be categorized as negative (0-3), moderate (4-8), or strong (9-12) using this calculated score. Evaluation of the samples can be performed under the guidance of a pathologist who is blinded to the clinicopathologic parameters. To determine whether ANTXR2 staining indices differ by tumor type (DCIS versus IDC), a Student's t-test can be performed. It can also be determined if there is a correlation between ANTXR2 expression and clinicopathological features such as tumor size, grade or stage by calculating the Spearman rank correlation coefficient (rs). Differences can be considered statistically significant at P<0.05.

After assigning an ANTXR2 staining index score to each sample, 10 samples can be selected from each ANTXR2 staining category (negative, moderate and strong) for further analysis. It can be determined if there is a relationship between ANTXR2 expression levels and myoepithelial cell content or stromal protein changes in these samples. To identify myoepithelial cells in the tissues, co-immunofluorescence can be performed using antibodies against the myoepithelial cell markers, smooth muscle actin (SMA) and p63. SMA can stain stromal fibroblasts and vascular smooth muscle cells, in addition to myoepithelial cells. Thus, cells that stain positive for both p63 and SMA can be defined as myoepthelial cells. Since activated stroma is often associated with increased collagen deposition (S19), samples for changes in fibrillar collagen can also be analyzed by staining with Masson's Trichrome. Analysis of the myoepithelial staining and Masson's Trichrome staining can consist of generating staining indices as described above for ANTXR2. It can then be determined if there is a correlation between ANTXR2 expression and either myoepithelial cell content or collagen deposition by calculating the Spearman rank correlation coefficient (O. Without being bound by theory, toss of ANTXR2 expression can be correlated with a loss of myoepithelial cell content but an increase in fibrillar collagen deposition.

Alternative Approaches:

There are a variety of MEC markers, such as CK5, CK14, CK17, CD10, S100, smooth muscle myosin heavy chain and calponin. Given the heterogeneity in MEC marker expression in IDC, staining can be performed with some of these additional markers in order to identify MECs in the samples.

To Determine the Mechanism of ANTXR2 Action in Myoepithelial Cells In Vitro.

Investigating Antxr2 function in myoepithelial cells (MECs) can be fundamental to understanding the endogenous activity of Antxr2. Utilizing mouse embryonic fibroblasts (MEFs), ANTXR2 is localized in a complex with MT1-MMP at the cell surface and facilitates activation of MMP-2. The presence and activity of this ANTXR2/MT1-MMP complex can be evaluated in MECs using a series of in vitro assays. First, mammary derived myoepithelial cells (MECs) can be isolated from Antxr2+/+ (WT) and Antxr2−/− (KO) mice using a recently published protocol entitled “Isolation, Culture and Analysis of Mouse Mammary Epithelial Cells” (S20). Myoepithelial cells can be sorted from luminal epithelial cells via flow cytometry and collected for culture in vitro (S20). Once isolated cells are confirmed as being MECs, by immunostaining with myoepithelial cell markers, SMA and cytokeratin 14, the cells can be used with the following assays:

Zymography:

In order to evaluate MMP activity, equivalent numbers of WT and KO MECs can be seeded in 24 well plates and cultured in serum free medium (SFM) containing 1% fetal bovine serum (FBS). Conditioned medium (CM) can be collected at 24 and 48 hours after seeding. Equal volumes of medium can be loaded onto gelatin-containing gels and zymography can be performed to assess levels of Mmp-2 activation as previously described (S21).

Rescue of MMP Defects:

If KO MECs exhibit reduced Mmp-2 activity, a rescue experiment can be performed in which the KO MECs are transfected with an expression vector that encodes human ANTXR2 with a GFP tag at the C-terminus (ANTXR2-GFP). Zymography with CM collected from the transfected cells can be used to determine if re-establishing ANTXR2 expression in KO MECs can restore MMP-2 activation to wild-type levels. To ensure that the transfected cells are expressing the protein of interest, cell extracts can be isolated and immunoblotting performed with an anti-ANTXR2 or anti-GFP antibody.

Coimmunoprecipitation:

In order to determine if Antxr2 and Mt1-Mmp interact in the MECs, protein can be isolated from confluent plates of WT and KO MECs and co-immunoprecipitation experiments can be performed in which protein lysate can be incubated with antibody against Antxr2. Protein A beads can be used to pull down the immuno-complex and the resulting eluate can be run on a 10% SDS-PAGE gel and probed with antibody against Mt1-mmp. This same experiment can be performed to evaluate Antxr2 interaction with Timp2 and Mmp-2.

Extracellular Matrix Accumulation:

WT and KO MECs can be seeded at equal densities on polylysinecoated coverslips in 24-well plates. After 2, 5, or 9 days of culture, the cells and extracellular matrix (ECM) can be fixed in 4% PFA. A survey of ECM protein deposition can be performed using immunofluorescence staining with anti-type I collagen, anti-type III collagen, anti-type IV collagen, anti-type VI collagen, antilaminin or anti-fibronectin antibodies. Reduced MMP activity in KO MECs may lead to an increase in ECM protein accumulation as compared to that WT MECs.

Proliferation and Viability:

ANTXR2 function may not be limited to regulating MMP activation in cells. MEC proliferation can be evaluated by seeding equivalent numbers of WT and KO MECs in a 24 well plate. The cells can be cultured in SFM containing 1% FBS. Cell numbers can be assessed on day 0 and day 5 with WST-8 (Dojindo). WST-8 is a formazon dye that produces a yellow color when cleaved by mitochondrial dehydrogenase in viable cells. The color change is detected via a spectrophotometer and OD readings are plotted against a calibration curve from known numbers of cells. The affect of Antxr2 deletion on proliferation can be calculated based on normalizing the relative cell number of the Antxr2+/+ line to 100%. MEC viability can be analyzed by TUNEL assays. Defects observed in proliferation or viability may be secondary to changes in MMP activity or through other means of molecular regulation.

Luminal Epithelial Cell Polarity:

Normal MECs have been demonstrated to re-establish polarity of luminal epithelial cells in 3D collagen-I gels in vitro such that co-culturing the two cell types results in the formation of double-layered acini that are very similar to those found in the normal breast (S22). To determine whether Antxr2−/− MECs differ in their ability to interact with luminal epithelial cells, WT luminal epithelial cells can be cultured in the presence of either WT or KO MECs in 3D collagen-I gels as previously described (S22). To characterize the polarity of the resulting acini, the gels can be frozen, sectioned and subjected to coimmunofluorescence using anti-sialomucin as an apical membrane marker and anti-beta4 integrin, as a basolateral membrane marker.

All assays listed above can be performed with two different WT and KO cell lines. Statistical significance can be evaluated using the unpaired Student's t-test with P-value<0.05 considered statistically significant.

Alternative Approaches:

This relies solely on the ability to isolate MECs from murine mammary glands using a recently published protocol (S20). If the protocol does not work, human MECs can be isolated according to the protocol from the Bissell lab (S22) and shRNA can be utilized to generate cell lines with knockdown of ANTXR2. These cell lines can then be used to perform the assays listed above.

To Determine the Role of ANTXR2 in Myoepithelial Cells During Breast Tumorigenesis.

Here, the contribution of myoepithelial ANTXR2 expression to normal breast morphogenesis can be evaluated as well breast cancer progression using the mouse as a model system. Similar experiments described herein can be conducted to evaluate the progression of other cancers using the corresponding mouse model for the cancer of interest. Various mouse models of human cancer have been previously discussed (see paragraphs 00208, 00209, 00210).

In order to explore ANTXR2 function in MECs, conditional Antxr2 flox/flox mice can be crossed with transgenic mice expressing the Cre recombinase in mammary epithelium (MMTV-Cre). This cross can generate mice with deletion of Antxr2 in MECs. In order to determine whether mammary gland development precedes normally upon deletion of Antxr2 MECs, hematoxylin-stained mammary gland wholemounts can be prepared that are derived from virgin female MMTV-Cre; Antxr2 flox/flox mice (n=5) and littermate controls (n=5) at 4 and 12 weeks of age; ductal outgrowth and branching morphogenesis can then be evaluated. Isolated mammary glands can also be paraffin-embedded, sectioned and MEC content can be evaluated by performing immunofluorescence staining with anti-SMA and anti-p63. Antxr2 deletion in MECs can be confirmed by immunostaining embedded mammary tissue with anti-Antxr2.

The MMTV-Cre; Antxr2 flox/flox mice can then be mated to MMTV-PyMT transgenic mice. In the MMTV-PyMT mouse model of human breast cancer, expression of the polyoma middle T antigen results in rapid and widespread malignant transformation in the mammary epithelium (S23). Female mice develop hyperplasia with 100% penetrance and display identifiable mammary tumor stages from benign in situ proliferative lesions to invasive carcinomas with a high frequency of distant metastases (S23). The tumor stages mimic biomarker expression that is characteristic of human mammary tumors with poor prognosis (S24). MMTV-PyMT; MMTV-Cre; Antxr2 flox/flox mice can be derived in two mating steps: i) heterozygous male MMTV-PyMT mice in the C57B1/6 background can be crossed with homozygous MMTV-Cre; Antxr2 flox/flox female mice (also in the C57B1/6 background) and ii) male MMTV-PyMT; MMTV-Cre; Antxr2 flox/+ progeny (heterozygous for all three alleles) can be crossed with heterozygous MMTV-Cre; Antxr2 flox/+ females to yield female MMTVPyMT; MMTV-Cre; Antxr2+/+ and MMTV-PyMT; MMTV-Cre; Antxr2 flox/flox mice. Genotypes can be determined by PCR. To assess whether myoepithelial Antxr2 deletion affects overall tumor onset and growth, tumors in MMTV-PyMT; MMTV-Cre;Antxr2+/+ (n=20) and MMTV-PyMT; MMTV-Cre; Antxr2 flox/flox mice (n=20) can be followed by weekly palpations of all 10 mammary glands starting at 8 weeks of age. It has been reported that mammary tumors can be detected in wild-type MMTV-PyMT mice with a median onset of 96.5 days (14 weeks) in the C57B1/6 background (S25) Immediately after tumors are first detected, the tumor volume (assuming that tumors take the shape of an ellipsoid) can be calculated using the formula: V=(π/6)×W²×L, where L=length and W=width. Tumor growth curves and Kaplan-Meier survival curves can be generated. Mice can be sacrificed when tumors reach the maximal size allowed by institutional guidelines, or when the mice become moribund. The inguinal and thoracic mammary fat pads can be removed, fixed, paraffin embedded, and serial sectioned. The lungs can also be removed for determination of the metastasis burden. Serial sections of mammary gland tumors can be stained with haematoxylin and eosin (H&E) in order to compare tumor histopathology between the genotypes. In addition, co-immunofluorescence with anti-SMA and anti-p63 can identify myoepithelial cells in the tissue. A myoepithelial staining index can be generated in order to determine if there are differences in myoepithelial cell content between the genotypes. Masson's trichrome stain can be utilized to assess fibrillar collagen content and the area covered by collagen can be calculated. Differences in myoepithelial cell or collagen content between the two genotypes can be evaluated using the unpaired Student's t-test with P-value<0.05 considered statistically significant.

Alternative Approaches:

If the two crosses required to generate transgenic MMTV-PyMT mice with deletion of Antxr2 specifically in myoepithelial cells becomes overly complex, the MMTV-PyMT mice can be mated with general Antxr2−/− mice. This can simplify the mating strategy and allow to address the same questions, while taking into account that other cells in the microenvironment have deletion of Antxr2.

### REFERENCES

S1. Bradley, K. A., Mogridge, J., Mourez, M., Collier, R. J. & Young, J. A. Identification of the cellular receptor for anthrax toxin. Nature 414, 225-229 (2001).
S2. Scobie, H. M., Rainey, G. J., Bradley, K. A. & Young, J. A. Human capillary morphogenesis protein 2 functions as an anthrax toxin receptor. Proc Natl Acad Sci USA 100, 5170-5174 (2003).
S3. Whittaker, C. A. & Hynes, R. O. Distribution and evolution of von Willebrand/integrin A domains: widely dispersed domains with roles in cell adhesion and elsewhere. Mol Biol Cell 13, 3369-3387 (2002).
S4. Young, J. A. & Collier, R. J. Anthrax toxin: receptor binding, internalization, pore formation, and translocation. Annu Rev Biochem 76, 243-265 (2007).
S5. Nanda, A. et al. TEM8 interacts with the cleaved C5 domain of collagen alpha 3(VI). Cancer Res 64, 817-820 (2004).
S6. Hotchkiss, K. A. et al. TEM8 expression stimulates endothelial cell adhesion and migration by regulating cell-matrix interactions on collagen. Exp Cell Res 305, 133-144 (2005).
S7. Bell, S. E. et al. Differential gene expression during capillary morphogenesis in 3D collagen matrices: regulated expression of genes involved in basement membrane matrix assembly, cell cycle progression, cellular differentiation and Gprotein signaling. J Cell Sci 114, 2755-2773 (2001).
S8. Strongin, A. Y. et al. Mechanism of cell surface activation of 72-kDa type IV collagenase. Isolation of the activated form of the membrane metalloprotease. J Biol Chem 270, 5331-5338 (1995).
S9. Guo, Y. P. et al. Growth factors and stromal matrix proteins associated with mammographic densities. Cancer Epidemiol Biomarkers Prey 10, 243-248 (2001).
S10. Provenzano, P. P. et al. Collagen density promotes mammary tumor initiation and progression. BMC Med 6, 11 (2008).
S11. Reeves, C. V., Dufraine, J., Young, J. A. & Kitajewski, J. Anthrax toxin receptor 2 is expressed in murine and tumor vasculature and functions in endothelial proliferation and morphogenesis. Oncogene 29, 789-801 (2010).
S12. Sorlie, T. et al. Gene expression patterns of breast carcinomas distinguish tumor subclasses with clinical implications. Proc Natl Acad Sci USA 98, 10869-10874 (2001).
S13. Sorlie, T. et al. Repeated observation of breast tumor subtypes in independent gene expression data sets. Proc Natl Acad Sci USA 100, 8418-8423 (2003).
S14. Perou, C. M. et al. Molecular portraits of human breast tumours. Nature 406, 747-752 (2000).
S15. Richardson, A. L. et al. X chromosomal abnormalities in basal-like human breast cancer. Cancer Cell 9, 121-132 (2006).
S16. Sopel, M. The myoepithelial cell: its role in normal mammary glands and breast cancer. Folia Morphol (Warsz) 69, 1-14 (2010).
S17. Zhang, R. R. et al. A subset of morphologically distinct mammary myoepithelial cells lacks corresponding immunophenotypic markers. Breast Cancer Res 5, R151-156 (2003).
S18. Gudjonsson, T., Adriance, M. C., Sternlicht, M. D., Petersen, O. W. & Bissell, M. J. Myoepithelial cells: their origin and function in breast morphogenesis and neoplasia. J Mammary Gland Biol Neoplasia 10, 261-272 (2005).
S19. Ronnov-Jessen, L., Petersen, O. W. & Bissell, M. J. Cellular changes involved inconversion of normal to malignant breast: importance of the stromal reaction. Physiol Rev 76, 69-125 (1996).
S20. Smalley, M. J. Isolation, culture and analysis of mouse mammary epithelial cells. Methods Mol Biol 633, 139-170 (2010).
S21. Troeberg, L. & Nagase, H. Zymography of metalloproteinases. Curr Protoc Protein Sci Chapter 21, Unit 21 15 (2004).
S22. Gudjonsson, T. et al. Normal and tumor-derived myoepithelial cells differ in their ability to interact with luminal breast epithelial cells for polarity and basement membrane deposition. J Cell Sci 115, 39-50 (2002).
S23. Lin, E. Y. et al. Progression to malignancy in the polyoma middle T oncoprotein mouse breast cancer model provides a reliable model for human diseases. Am J Pathol 163, 2113-2126 (2003).
S24. Maglione, J. E. et al. Transgenic Polyoma middle-T mice model premalignant mammary disease. Cancer Res 61, 8298-8305 (2001).
S25. Hebbard, L. W. et al. T-cadherin supports angiogenesis and adiponectin association with the vasculature in a mouse mammary tumor model. Cancer Res 68, 1407-1416 (2008).

### Example 3

### Human Fc (IgG₁)-CTP (hFcCTP) Polypeptide

This example describes one embodiment of an isolated polypeptide described herein.

The CTP domain of the beta-subunit human chorionic gonadotropin (hCG) was fused in frame to the terminus of human Fc fragments, creating an hFcCTP polypeptide. An Fc fragment from IgG1 subtype was used.

Cloning.

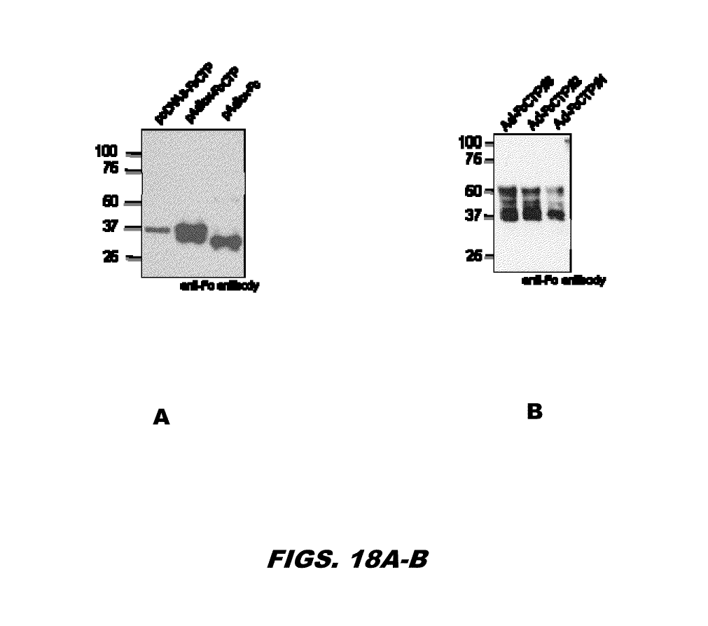

The CTP domain was cloned using pBluescriptII-Fc plasmid (human IgG Fc) and the upstream sense primer (5′TAGAGATCCCTCGAGGGCGC3′) [SEQ ID NO: 38] and the downstream anti-sense primer (5′TCTCAGCGCCGGCGACCTTATTGTGGGAGGATCGGAGTGTCCGAGGGCCCCGG GAGTCGGGATGGGCTTGGAAGGCTAGGAGGAGGGGGCCTTTGAGGAAGAGGAG TCCTGGAAGCGTGGTGATCCTTTACCCG3″ [SEQ ID NO: 39]). The PCR product was ligated into pDrive cloning vector (Qiagen). The resultant clones were verified by diagnostic digests followed by nucleotide sequencing. The insert was then sub-cloned into pBluescriptII using Not I and Xho I restriction enzymes, and the resultant clones were verified by restriction enzyme digestion and nucleotide sequencing. The insert was subsequently subcloned into pAdlox and pcDNA3. Protein expression was verified by transfection of pAdlox-FcCTP and pcDNA3-FcCTP plasmids into 293 cells and western blotting. FIG. 18A is a Western blot showing the detection of Fc and FcCTP expression in 293 T-cells transfected with pcDNA3-FcCTP, pAdlox-FcCTP, and pAdlox-Fc. Anti-human Fc antibody was used for detection.

Adenovirus Production.

Adenovirus production is described in Hardy et al., Construction of adenovirus vectors through Cre-lox recombination, Journal of Virology, 1997, 71: 1842-1849. 293 T-cells (stably expressing Cre recombinase) were transfected with pAdlox-FcCTP plasmid (linearized with SfiI) and Psi helper virus DNA (a donor virus that supplies the viral backbone). The virus was passed through 293 T-cells twice. Next, the virus was used for a plaque assay. Virus infected 293 cells were overlayed with Seaplaque agarose. Plaques which appeared as small clear zones were picked and used to infect 293 cells. Ad-FcCTP expression was then assessed by western blotting, and one plaque was selected for large-scale purification. FIG. 18B is a Western blot showing the detection of FcCTP expression in 293 cells transduced with three Ad-FcCTP positive plaques. Anti-human Fc HRP was used for detection.

The nucleic acid sequence of the hFcCTP polypeptide is presented as SEQ ID NO:30, and the amino acid sequence is presented as SEQ ID NO: 31 in the Sequence Listings. The nucleic acid sequence of the CTP peptide domain is presented as SEQ ID NO: 36, and the amino acid sequence is presented as SEQ ID NO: 37 in the Sequence Listings.

A schematic representation of this hFcCTP polypeptide is shown in FIG. 17A.

### Example 4

### hVEGFR1¹⁻³-FcCTP Polypeptide

This example describes one embodiment of an isolated fusion polypeptide molecule described herein.

The CTP domain of the beta-subunit hCG was fused in frame to the terminus of human Fc fragments, creating an hFcCTP polypeptide, as described in Example 3. The hFcCTP is added to extracellular domains 1-3 of VEGFR1 receptor, resulting in an hVEGFR¹⁻³-FcCTP polypeptide.

Cloning.

The CTP domain is cloned using pBluescriptII-Fc plasmid (human IgG Fc) and the upstream sense primer (5′TAGAGATCCCTCGAGGGCGC3′) [SEQ ID NO: 38] and the downstream anti-sense primer (5′TCTCAGCGCCGGCGACCTTATTGTGGGAGGATCGGAGTGTCCGAGGGCCCCGG GAGTCGGGATGGGCTTGGAAGGCTAGGAGGAGGGGGCCTTTGAGGAAGAGGAG TCCTGGAAGCGTGGTGATCCTTTACCCG3″) [SEQ ID NO: 39]. The PCR product is ligated into pDrive cloning vector (Qiagen). The resultant clones are verified by diagnostic digests followed by nucleotide sequencing. The insert is then subcloned into pBluescriptII using Not I and Xho I restriction enzymes, and the resultant clones are verified by restriction enzyme digestion and nucleotide sequencing. The insert is subsequently subcloned into pAdlox and pcDNA3. Protein expression is verified by transfection of pAdlox-FcCTP and pcDNA3FcCTP plasmids into 293 cells and western blotting.

Adenovirus Production.

Adenovirus production is described in Hardy et al., Construction of adenovirus vectors through Cre-lox recombination, Journal of Virology, 1997, 71: 1842-1849. 293 T-cells (stably expressing Cre recombinase) are transfected with pAdlox-FcCTP plasmid (linearized with SfiI) and Psi helper virus DNA (a donor virus that supplies the viral backbone). The virus is passed through 293 T-cells twice. Next, the virus is used for a plaque assay. Virus-infected 293 cells are overlayed with Seaplaque agarose. Plaques which appeared as small clear zones are picked and used to infect 293 cells. Ad-FcCTP expression is then assessed by western blotting, and one plaque is selected for large-scale purification.

The nucleic acid sequence of the hVEGFR¹⁻³-FcCTP polypeptide is presented as SEQ ID NO: 32, and the amino acid sequence is presented as SEQ ID NO: 33 in the Sequence Listings.

A schematic representation of this hFcCTP polypeptide is shown in FIG. 17B.

### Example 5

### hVEGFR2¹⁻³-FcCTP Polypeptide

This example describes one embodiment of an isolated fusion polypeptide molecule described herein.

The CTP domain of the beta-subunit hCG was fused in frame to the terminus of human Fc fragments, creating an hFcCTP polypeptide, as described in Example 3. The hFcCTP is added to extracellular domains 1-3 of VEGFR2 receptor, resulting in an hVEGFR2¹⁻³-FcCTP polypeptide.

Cloning.

The CTP domain is cloned using pBluescriptII-Fc plasmid (human IgG Fc) and the upstream sense primer (5′TAGAGATCCCTCGAGGGCGC3′) [SEQ ID NO: 38] and the downstream anti-sense primer (5′TCTCAGCGCCGGCGACCTTATTGTGGGAGGATCGGAGTGTCCGAGGGCCCCGG GAGTCGGGATGGGCTTGGAAGGCTAGGAGGAGGGGGCCTTTGAGGAAGAGGAG TCCTGGAAGCGTGGTGATCCTTTACCCG3″) [SEQ ID NO: 39]. The PCR product is ligated into pDrive cloning vector (Qiagen). The resultant clones are verified by diagnostic digests followed by nucleotide sequencing. The insert is then subcloned into pBluescriptII using Not I and Xho I restriction enzymes, and the resultant clones are verified by restriction enzyme digestion and nucleotide sequencing. The insert is subsequently subcloned into pAdlox and pcDNA3. Protein expression is verified by transfection of pAdlox-FcCTP and pcDNA3FcCTP plasmids into 293 cells and western blotting.

Adenovirus Production.

Adenovirus production is described in Hardy et al., Construction of adenovirus vectors through Cre-lox recombination, Journal of Virology, 1997, 71: 1842-1849. 293 T-cells (stably expressing Cre recombinase) are transfected with pAdlox-FcCTP plasmid (linearized with SfiI) and Psi helper virus DNA (a donor virus that supplies the viral backbone). The virus is passed through 293 T-cells twice. Next, the virus is used for a plaque assay. Virus-infected 293 cells are overlayed with Seaplaque agarose. Plaques which appeared as small clear zones are picked and used to infect 293 cells. Ad-FcCTP expression is then assessed by western blotting, and one plaque is selected for large-scale purification.

The nucleic acid sequence of the hVEGFR2¹⁻³-FcCTP polypeptide is presented as SEQ ID NO: 34, and the amino acid sequence is presented as SEQ ID NO: 35 in the Sequence Listings.

A schematic representation of this hFcCTP polypeptide is shown in FIG. 17C.

Other aspects, modifications, and embodiments are within the scope of the following claims.

### REFERENCES

1. Matzuk M M, Hsueh A J, Lapolt P, Tsafriri A, Keene J L, Boime I: The biological role of the carboxyl-terminal extension of human chorionic gonadotropin [corrected] beta-subunit. Endocrinology 1990, 126:376-383.
2. LaPolt P S, Nishimori K, Fares F A, Perlas E, Boime I, Hsueh A J: Enhanced stimulation of follicle maturation and ovulatory potential by long acting follicle-stimulating hormone agonists with extended carboxyl-terminal peptides. Endocrinology 1992, 131:2514-2520.
3. Fares F A, Suganuma N, Nishimori K, LaPolt P S, Hsueh A J, Boime I: Design of a long-acting follitropin agonist by fusing the C-terminal sequence of the chorionic gonadotropin beta subunit to the follitropin beta subunit. Proc Natl Acad Sci USA 1992, 89:4304-4308.
4. Bouloux P M, Handelsman D J, Jockenhovel F, Nieschlag E, Rabinovici J, Frasa W L, de Bie J J, Voortman G, Itskovitz-Eldor J: First human exposure to FSH-CTP in hypogonadotrophic hypogonadal males. Hum Reprod 2001, 16:1592-1597.
5. Klein J, Lobel L, Pollak S, Ferin M, Xiao E, Sauer M, Lustbader J W: Pharmacokinetics and pharmacodynamics of single-chain recombinant human follicle-stimulating hormone containing the human chorionic gonadotropin carboxyterminal peptide in the rhesus monkey. Fertil Steril 2002, 77:1248-1255.
6. Klein J, Lobel L, Pollak S, Lustbader B, Ogden R T, Sauer M V, Lustbader J W: Development and characterization of a long-acting recombinant hFSH agonist. Hum Reprod 2003, 18:50-56.
7. Fares F, Ganem S, Hajouj T, Agai E: Development of a long-acting erythropoietin by fusing the carboxyl-terminal peptide of human chorionic gonadotropin beta-subunit to the coding sequence of human erythropoietin. Endocrinology 2007, 148:5081-5087.
8. Trousdale R K, Pollak S V, Klein J, Lobel L, Funahashi Y, Feirt N, Lustbader J W: Single-chain bifunctional vascular endothelial growth factor (VEGF)-follicle-stimulating hormone (FSH)-C-terminal peptide (CTP) is superior to the combination therapy of recombinant VEGF plus FSH-CTP in stimulating angiogenesis during ovarian folliculogenesis. Endocrinology 2007, 148:1296-1305.

9. Fares F, Guy R, Bar-Ilan A, Felikman Y, Fima E: Designing a long-acting human growth hormone (hGH) by fusing the carboxyl-terminal peptide of human chorionic gonadotropin beta-subunit to the coding sequence of hGH. Endocrinology 2010, 151:4410-4417.

1-86. (canceled)

87. An isolated polypeptide comprising an Anthrax Toxin Receptor (ANTXR) or a vWF domain thereof fused to an Fc domain, a carboxy-terminal peptide (CTP) domain, an Fc-CTP domain, or a combination thereof.

88. The isolated polypeptide of claim 87, wherein the ANTXR is ANTXR1 or ANTXR2 or extracellular domain of ANTXR1 or ANTXR2.

89. The isolated polypeptide of claim 87, wherein the Fc domain is about 95% identical to SEQ ID NO: 1, or the CTP domain is about 95% identical to SEQ ID NO: 3, or the Fc-CTP domain is about 95% identical to SEQ ID NO: 16.

90. The isolated polypeptide of claim 87, wherein the CTP domain, Fc domain, Fc-CTP domain, or combination thereof, is fused to the C-terminus of the ANTXR, or the N-terminus of the ANTXR, or both.

91. A method of treating or preventing a disease or disorder in a subject, the method comprising administering to a subject the polypeptide of claim 87 or a nucleic acid encoding the polypeptide of claim 87, thereby treating or preventing the disease or disorder, wherein the treatment or prevention is selected from the group consisting of decreasing fibrosis in a tissue of the subject, treating or preventing a fibrotic disease, treating or preventing an epithelial cancer in a subject, decreasing or preventing tumor cell invasion into a tissue free from tumor cells, decreasing or preventing cancer metastasis, and decreasing or preventing angiogenesis in a tumor.

92. An isolated polypeptide comprising a carboxy-terminal peptide (CTP) domain having at least about 90% identity to SEQ ID NO: 37, and wherein the CTP domain is fused to an antibody fragment or the ectodomain of a receptor.

93. The isolated polypeptide of claim 92, wherein the antibody fragment is selected from the group consisting of an Fab′ fragment, an F(ab′)2 fragment, an Fv fragment, an Fc fragment, a diabody, a single-chain variable fragment (scFv), an immunoglobulin isotype consisting of IgG, IgA, IgE, IgM, and IgD antibody, and any combination thereof.

94. The isolated polypeptide of claim 92, wherein the CTP domain or the antibody fragment or both is glycosylated.

95. The isolated polypeptide of claim 92, wherein the CTP domain is the CTP domain of human chorionic gonadotropin or comprises a polypeptide at least about 95% identity or at least about 98% identity or at least about 99% identity or 100% identity to SEQ ID NO: 37.

96. An isolated fusion polypeptide molecule, comprising the polypeptide of claim 92 attached to a carboxy terminus of a second polypeptide.

97. A method of stabilizing a polypeptide or increasing the biological half-life of a polypeptide, the method comprising:
a. attaching a CTP domain comprising at least about 90% identity to SEQ ID NO: 37, fused to an antibody fragment to the carboxy terminus of the polypeptide, thereby stabilizing the polypeptide or increasing the biological half-life of the polypeptide.

98. The method of claim 97, the method further comprising:
b. further modifying the CTP domain to change the quantity or type of glycosylation.

99. The method of claim 97, wherein the polypeptide is an antibody, a fusion protein, a hormone, a receptor, a binding protein, or a soluble factor.

100. The method of claim 97, wherein the CTP domain comprises at least about 95% identity or 98% identity or 99% identity to SEQ ID NO: 37.

101. A pharmaceutical composition comprising the isolated polypeptide of claim 96 and a pharmaceutically acceptable carrier.

102. A method of treating or preventing a disorder comprising administering an effective dose of the pharmaceutical composition of claim 101 to a subject in need of treatment or prevention of a disorder selected from the group consisting of a cancer, immune-related disorder, cardiovascular disease, obesity, diabetes, metabolic disorders and blindness.
